SEQUENCE LISTING (108 sequences)

SEQ ID NO: 1 - protein, 1288 aa, synthetic construct
MIMENIKNNYQLSKTLRFGLTQKQNGNSSNTDNVYHSHSALKELVDISENRIKKNVSTEGATEMQLSIESIRKCMIMIEQFIKDWKRVYYRSDQISLDKDFYKKLSKKIGFEAFWFERNKKTQQRIKKPQSCIIALSELSKRDNFGKERQEYIVEYWENNLLKSTERYEEVSEKLEQFELALKINRTDNRPNEVELRKMFLSLVNMVREVVEPLCLGQISFPKLEKLADNSKNKQLRKFATDYQSKSDLLTQISELKKYFEENGGNVPYCRATLNPLTAVKNPKSTDSSILDEIKKLKLDVILRDYQSVALFDNSIRDLTASQKMQLLNQNNEGLIKRGLLFKYKPIPAIVQYEIAKVLSAELNKDEQELRNFLRDIGQVKSPAKDYAELQDKKDFDINHYPLKVAFDFAWESLAKSVYHPDIDFPEEQCKTLLREVFQVDENNENFKFYAQLLELRSLLATLEHGKPTEVITIENEVKKILENIDWSKFGDRGKNYKSAIENWIHNRNKKDFKGDYFKKAKQQIGLTRGRQKNLIKKYDEITKSYKDIAMKMGKTFAEMRDKITGAAELNKVSHYAMIVEDTNQDKYVLLQEFVENNKDRIYAKSTPQNEDFKAYSVNSVTSSAIAKMIRKIRIDKLQANERNNNRQQAPELSETQKEARNIKEWKDFIAEKRWNYEFDLKLDNKNFEQIKKEIDSKCFKLETKYMSEEVLVDLVKNQNCLLLPIINQDLAKKIKSESNQFTKDWNAIFAQNTPWRLTPEFRVSYRKPTPNYPKSERGDKRYSRFQMIGHFLCDFIPKTADYISNREMIANFKDDEKQKQTIINFHERLNPKSENEKMNMLLAKFGNKNSNQSKETKKEEKFYVFGIDRGQKELATLCVIDQDKKIIGDFNIYTRSFNTQNKQWEHQLLDKRHILDLSNLRVETTIVIDGKPDVRKVLVDLSEVKVKDKNGNYTKTDKMQVKMQQLAYIRKLQFQMQTNPDTVLEWYNKNQTKEAILNNFVDKPNGEKGLVSFYGSAVEELKDTLPIERIEKMLQQFKSLKNEEKEGKDVKAEIDKLIQLEPVDNLKAGVVANMVGVIAHLLKEFNYQVYISLEDLSNPFGSHVIDGTTGTHSKTNKGEGKRADVEKYAGLGLYNFFEMQLLKKLFRIQQDSQNILHLVPAFRAVKNYENIIAGKDKIKNQFGIVFFVDANSTSKMCPVCNSTNETNREYPNAKKGTSKDDKEVWVERDKSNGDDIIRCFVCGFDTTKKYEENPLKFIKSGDDNAAYIISAFGIKAYELAKSVIDNK

SEQ ID NO: 2 - protein, 1294 aa, synthetic construct
MEAIKNNYQLSKTLRFGLTQKSKTRKDGFTGEIYQSHNELKDLVKSSEDRIKKSVSTDEKSEMSLSVDKIRCCLVMISDFLSSWQQVYSRADQIALDKDYYKILCKKIGFDGFWVDERYDRKNDKTVRTKKPQSRTINLSELDKKDDKGIERRQYLLTYWRDNLINAADKFEVVTEKLKQFEDALNINRTYNKPNEVELRKLFLSLTNIVQETLQPLCLGQICFPKLEKIDDSRTENKHLIDFATDYQSKSDLLSEISELKKYFEENGGNVPYCRATLNQKTAVKNPNSTDNSIDSEIKKLGLDKILKENKDALYFANKIYSLSAKEKLSKLDDKTTGLIERSLLFKYKPVPAIVQYEIAKTLSETINKSEEDLLEFLRSIGQTKSPTKDYADLQDKNDFDLDAYPLKVAFDFAWENLARSIYHSDADIPVAVCEGFLKKNFGIDKSNADFKLYAQLQELKAVLATLEYGNPTNRQTFINEATKLLSPISWDKIGRNGNQNKYSIEKWLKTLTKDDKDYKDAKQQIALFRGRLKNNIKTFDDITKYFKSVAMEMGRTFAQMRDKITGAAELNKVTHYAIIIEDQNFDKYVLLQEFVDKKENRIYAKTDRHHSDFTTYSVNSVTSGSIAKMLRKKRMDELNRNNRNSFEQKPELSEEQKEQRNIREWKEFIEDKRWDLEFQLNLSNKTFEQIKKEVDAKCYELDINYISQETLSDLVNKKGCLLLPIVSQDIAKENKTEGNQFTKDWNAIFTQETPWRLTPEFRVSYRKPTPNYPVSDKGDKRYSRFQMIAHFLCDYLPKSDSYISNWEQIANYKDDKLQEKAVKEFNADLRGRTEEEKQSESVNALLAHFGNQNKKQKPVERPKEKFYVFGIDRGQKELATLCVIDQDKKIVGDFDIYTRSFNSEAKQWEHKLLEKRVILDLSNLRVETTIVIDGKPEKKKVLVDLSEVKVKDKDGKYSKPNKMQVKMQQLAYIRKLQFQMQTNPDAVLEWYANNKTKEQIMSNFVDNENGDKGLVSFYGTAVEELNETLPIDKIEEILKKFQELKDKEKQGETVKLEIDKLVQLEPVDNLKNGVVANMVGVIAYLLQNLDYQVYISLEDLSKPFSGQIIGGIAGVPTKTNKEEGRRADVEKYAGLGLYNFFEMQLLKKLFRIQQDSQNILHLVPPFRAMKNYDHVAVGKGKVKNQFGIVFFVDADATSKTCPCCGSSNNKPNLKMYPNAKKGLSKEGKEVWVERDKSEGNDIIRCFVCGFDTTKDYSENPLRYIKSGDDNAAYLISAEGIKAYELATTLVNNK

SEQ ID NO: 3 - protein, 1237 aa, synthetic construct
METIEYQFSKTLRFGLTLKDHERKNKTHQTFNDLIGVSAQRIKEDASRDHDKTEQQLVTSVAACVKLMHQYLDAWEKIYRRTDQLALTKDFYKQMAKKACFDAYWLDNKERKQPQSQIIAISSLRKKHDEKERKDYILDYWADNISITKQRIHEFEKVIDQFKKALENKSMAHNKPHLVDFRKMFLSLTRLCNETLIPICNDSICFPALDKLQDNARHEAIKTFASEDEREERKGLSLSIKDIKEYFEENGGYVPLGKVTLNRYTAEQKPNNFKEDIKKKINDLRLTDLIQKLINLSDEEIKEYFEFNGKQKKQLIDDTRLSVVERVQLFKYKPIPAAVRFMLAEYLHKNNLLDKERVMTLFEIIGKPRSIGEEYTKLKDTSDFDLFQYPLKPAFDYAWENVARNLRNDKANAYPKEQCIRFLENIFEVSTSTTAFILYADLLFIKDNLSTLEHEKNSPKDKDQFIENIKRTFKNINYGIEQKEYIKHQQTILDWINKKEDAQKELKNANDNSYKNYENAKQQFGLLRGRQKNSIRQYKDLTETFKTLAVSFGKNFAELREKLREENEINKITHFGIIVEDSKLERYVLLSKLDEDKTLSIDHLLIDEPKGELKSYQVKSLMPKSLEKIIKNKGGYKDFHTSSKYINFIEMKRDWANYKNKPELVDYVKDCLQHSTMSKDQHWAAFGCDFTTCNSYEAIERELEIKAYRLKASHISITTVQKLVNEENCILLPFVNQDITSAKRELKNQFSKDWDMLFENNNDYRLHPEFRIVYRQPTPDYPLNKRYSRFQLIAHLMCEYIPQSVEYISRKQQIQIFNDKNEQKKQVDAFNERVKPSGEYYVLGIDRGLKQLATLCVLNNEGQIQGGFEVFTRTFDSVKQEWKHNLLEKRAILDLSNLRVETTVNGDKVLVDLASILVKDEQHNYTKDNQQKIKLKQLAYIRKLQFQMQHEPQKVLNFIKDYTTPQAVGEKIGELITPYKEGTHYDDLPIEKIYDMLQQFHQFTDEGNETAKKELTELDSADDLKTGVVANMVGVIAFMLEKYHYNAFVSLENLCRAFGFAKDGLNGELLVSTAVDNTVDFKDQENLVLAGLGTYHYFEMQLLKKLFRIQTGNDIIHLVPAFRSVDNYETIRKLSTKTKDALYTCKPFGVVHFIDPMYTSKKCPACDSINVSRFSKQGDIISCNKCGFQTRWDTQTTLKNNALLQSYKKQNLNLHYILNGDDNGAYRIALKTFANLT

SEQ ID NO: 4 - protein, 1393 aa, synthetic construct
MTKYQLTKTLRFGLTKVRKKTKLVAGKEVDAKYLSHEELDDLVMRSEYNLIKRNVLEWSKKKESDKSNYIFDELDKRFEEIIEIEDRDQRNAEYVEFLNEFFKEVNHQTINQLDESTFINKIGDCSKAIKEYLLSWGKVCRRIDKITVRKDYFKILARKTFFKYESKVGKKRTPLPSEVKLSGQKGNNYFDEPINEGISQFWQNRVAKALNLHSQLESMLFDYKKAIETEKHNQENPKGDNGSFDKLHLVDFRKMFLSVCSLVMDSLRPIVNELIIVSDNVSKDEDKYILDFVNDKKTQWDLFQQIENLQTICKDNGENIFFGKATFNKYTSEQAPNHRNNDIAKVLRELKIEKFVSDYIDLDQEAINRKIYQSTQSRLENLNNPQISPIIRAQYFKYKPIPTLVRFGLAKELAKQQGKKYSDRLKGIQELFRIFGSSKSPALDYKNNRTDFSLDNYPIKVAFDYAWEMCARSEYAQKPVDFPKSICEKFLEKFFECKSNEKYQQSFVTYARLLKINEDLATLEHFENEPPKDIESIYQDAQRYLDEVGNLCSNEDRAAIAKWYEEYNKLWTKGDHKKLKEWIVSKSSIITNFTQAKMHLGQKRGSQKTFVLKSYFHSSYGKIRDNNRFVNSNVTEVFKTIASTFGKSFATIREYFNEESEVNKIEYGAVIIKDKNGDKYLLLQKKNEGGIDMPIFNKSDENGDCDLYQVKSLTSKTVRKIIASPNKYNDFFVNNDGKKIIYPDKTDFKYKINPYDKEEVKKRKRELYNNDLVRPIIYSLTQSKFANKQNFEKYFDWTKALKQCSNIEQLYKTIDQKGYSLNPSKISKEQIADLVNNMNCYLLPIVNQNITAKTKNDTNQFTKDWNKIFNEVDKDYRIHPEFTMFYRYPTPDYPKFGEKRYSRFQMNVNFLMEVIPADGEYCSRKEQIEIYNAPKDNENCQKNVVERFNNKIKALKPSYFIGIDRGINELATLCVIDKEGKIVGDFEIYKREFDSNLKRQKYTSIETRDILDLSYLRVEKDENGESRLVDLSESEVWIDSLDHDKGKRANKQIVHLKHLYYLRCIAHLLQSSDYKSIVLEKLKDCNNLKDEEIKKVFEKDKFVDSYKGGEAYTDLPYDEIRKLISDYQEIEQSNQTESEKSKALNTLCQLDASEYLKKGVVANMIGVVVYVLKKYNYDAYISLENLCYAYGYSKDTLSGYSITSTKEDPYLDFKDQENAKLAGLGTYSFFEVQLLKKLFKLQIEENTELIPAFRSVDNYEKIFLLKNIDNKIYQFGIVYFVDPKYTSLCCPICGEHGKKNVDRKKHTKKYDEDELVCKQCGFHTNLSHIETRVMEDKTIKNSYDECNLKAIVSGDANAAYNIAIRLGKNIYSTIADKVKDLHHEGKKYIIVKG

SEQ ID NO: 5 - protein, 1370 aa, synthetic construct
MAGKEVDAKYLSHEELDDLVMRSEYNLIKRNVLEWSKKKESDKSNYIFDELDKRFEEIIEIEDRDQRNAEYVEFLNEFFKEVNHQTINQLDESTFINKIGDCSKAIKEYLLSWGKVCRRIDKITVRKDYFKILARKTFFKYESKVGKKRTPLPSEVKLSGQKGNNYFDEPINEGISQFWQNRVAKALNLHSQLESMLFDYKKAIETEKHNQENPKGDNGSFDKLHLVDFRKMFLSVCSLVMDSLRPIVNELIIVSDNVSKDEDKYILDFVNDKKTQWDLFQQIENLQTICKDNGENIFFGKATFNKYTSEQAPNHRNNDIAKVLRELKIEKFVSDYIDLDQEAINRKIYQSTQSRLENLNNPQISPIIRAQYFKYKPIPTLVRFGLAKELAKQQGKKYSDRLKGIQELFRIFGSSKSPALDYKNNRTDFSLDNYPIKVAFDYAWEMCARSEYAQKPVDFPKSICEKFLEKFFECKSNEKYQQSFVTYARLLKINEDLATLEHFENEPPKDIESIYQDAQRYLDEVGNLCSNEDRAAIAKWYEEYNKLWTKGDHKKLKEWIVSKSSIITNFTQAKMHLGQKRGSQKTFVLKSYFHSSYGKIRDNNRFVNSNVTEVFKTIASTFGKSFATIREYFNEESEVNKIEYGAVIIKDKNGDKYLLLQKKNEGGIDMPIFNKSDENGDCDLYQVKSLTSKTVRKIIASPNKYNDFFVNNDGKKIIYPDKTDFKYKINPYDKEEVKKRKRELYNNDLVRPIIYSLTQSKFANKQNFEKYFDWTKALKQCSNIEQLYKTIDQKGYSLNPSKISKEQIADLVNNMNCYLLPIVNQNITAKTKNDTNQFTKDWNKIFNEVDKDYRIHPEFTMFYRYPTPDYPKFGEKRYSRFQMNVNFLMEVIPADGEYCSRKEQIEIYNAPKDNENCQKNVVERFNNKIKALKPSYFIGIDRGINELATLCVIDKEGKIVGDFEIYKREFDSNLKRQKYTSIETRDILDLSYLRVEKDENGESRLVDLSESEVWIDSLDHDKGKRANKQIVHLKHLYYLRCIAHLLQSSDYKSIVLEKLKDCNNLKDEEIKKVFEKDKFVDSYKGGEAYTDLPYDEIRKLISDYQEIEQSNQTESEKSKALNTLCQLDASEYLKKGVVANMIGVVVYVLKKYNYDAYISLENLCYAYGYSKDTLSGYSITSTKEDPYLDFKDQENAKLAGLGTYSFFEVQLLKKLFKLQIEENTELIPAFRSVDNYEKIFLLKNIDNKIYQFGIVYFVDPKYTSLCCPICGEHGKKNVDRKKHTKKYDEDELVCKQCGFHTNLSHIETRVMEDKTIKNSYDECNLKAIVSGDANAAYNIAIRLGKNIYSTIADKVKDLHHEGKKYIIVKG

SEQ ID NO: 6 - protein, 1335 aa, synthetic construct
MEQYQLTKTIRFGLTKVRKEKKHLSHEELDELVMVSEERIKKEHPQAENQLDEQSFVKKIGDCSKAIKEYLLSWGKVCRRIDKITVRKEFFKILARKTFFKYESKVGKKRTPLPSEVKLSGQKGNNYYDEPINEGISQFWQNRVSKALKLHSQLESMLFDYKKAIETEKHNQENPKENNDQFDKLHLVDFRKMFLSVCSLVMDSLRPIVNELIIVSDNVSKDEDKYILDFVNDKSKQWDLFKQIEDLQNLCKDNGGNIPFGKATFNKYTSEQAPNHRDNDIHKVIRELKIEEFVSDFIGLEQEDIYRKIYQSTQHSLVNLNKPSISPIIRAQFFKYKPIPVLVRFGLANYLNKQQGKKYSNRLKDIQELFRIFGTSKSPELDYSDKNNRTEFSLDKYPIKVAFDYAWERCARSKYAQKPVDFPKEICVTFLETYFEYNSKEKNREAFEIYAHLLKVNECLATLEHFENEPPKDIKSLWQDVQNHLDKVGKLCSNEDRKAITQWYEEYKNLWAKGNYKKLKKWIESKSYTVTNFTQAKMHLGQKRGSQKTMVLKSYFHPTYGKIKDGNRFINSNVTEVFKNIASTFGKSFATIRDYFNEESEVNKIEYGAVIIKDKKGDKYLLLQKKNEGGIDMPVFNESDGNGDYDVYQVQSLTYKTVNKIYNSTKYPEFFAINGEKAIYAPNRPQRFKDDQEKNTFNEKKLQSLKKCLTESDFMTNTTENYLQKFNWTEEINNCTDFEPLAKIVDQKGYYLKSYKISKEQIAELVNNQNCYLLPIVNQNITAKTKNDTNQFTKDWNKIFNDEYKDYRLHPEFTMFYRYPTPDYPCPGEKRYSRFQMNVNFLMEVIPSEGEYVSRKEQIESFNTPKQDKEDNDNENSQAKKVAQFNDNINTKKPSYIIGIDRGINELATLCVINSEGKIVAVDENGLIKDEFDIYVKHFDKDNKCWIHNIKPKTATDNKPRTILDLSNLRVETTIDGKQVLVDLSSDENGIQVNSKQIVHLKRLYYLRCLSYLLQSSDYKSIILEKLKDVNNMTDDAIYEVFKNDKFIDSYKGGVQYTDLPYDEIRHLISTYQEIDQSNKTDSEKQSELNTLCQLDATESLKKGVVANMIGVVVYILKKLNYDAYISLENLCRALYFSKDSLSGYTIENTSVNPDLDFKDQENAKLAGLGTYSYFEIQLLKKLFKLQIDEKQFLVPGFRSVENYEKIVKLGKVKHSIYQFGVVHFVEPANTSLKCPICGANGKRIKYNPNYDEDELVCKKCGFRSNISKIQNSKIMEDSVIKTYYDNHNYKAIISGDTNAGFNIALRLLMNLNTQIENAINHLHKTGKNYHSVNK

SEQ ID NO: 7 - protein, 1248 aa, synthetic construct
MKNLTQFVNLYQLSKTLKFGLTLRNKIRKNGFEGEIYESHTELQELIKISEQKIIKETTDKNKEIMTFTELPLDEIRKCLDDMHKYLDDWEQFYNRYDQIAVLKDYYRKLERKARFDGFWREKNIKNKIQNNESETIKKPQSQVIKLSSLNNEYENKKRRDYITDYWNENIQKAKRKFYEVNSVLKQFEVANEQNRDDKKLNEVVLRKLFLSFTNLINDTLEPLCNGSICFPDIEKLTNSKTDEQLQRFVFDDGFKKILSEQIENLKIYFAINGGYVQYGRVTLNKYTALQQPNKVDEDIKNIIKELGLLEFVKKYENTEQIINYIKNIKDKKQELNANNLSLIEKVQLFKYKTIPAGVQPSLIAYLARTEKKDKKTLRELFYAIGQPQSPSKDYKELQNKTDFNLYKYPLKVAFDYAWESLAKSKYNPHIDFPDVKCKEFLKDIFGTDISVNDNFKLYSALLFVRENLATLDHGNPNDKNIHVNKVENTFKEIKDRLAKKEYKKEYKALEIICKWHKNSATIEQSEYEAAKQTIYEAAKQTIGQLRGRQKNQISKFKELTDSFKKLAPKFGKAFANLRDKFNEEYEINKISHCGVIVEDRNNDQYLLLSQLNDNRENASDIFELEADPNGELKIYQVKSLTSKTLLKFLKNKKGSNTGFHINENWTFPKGKWDVINKDKIFLNYVIQRITNSSMAKEQKWSNFKWDFRRCDSYEAIAKEVDAKGYILESVNISKLTLNKLITEKKCLLLPIVNQDLTRQDKKTKNQFTKDWIKIFESNNCYRLHPEFKISYRYPTPNYPKPEEKRYSRFQMISHLLCEYIPQNDNYKSRKEQVKIFNDKVAQKESVEQFNQQFEITDDYYIFGIDRGIKQLATLCILNKNGQIQGDFEIYTREFDKVNKQWKHTILEKRNILDLSDLRVETTVEGKKVLVDLSKVKLHSGNENKQTIKLKRLAYIRMLQYQMQHEQDKVLRFINQYKTIDEIEKNIRDLISPFKEGKQYADLPTEKIKDMLIQFGELSKNDSDKSKKELCTLCELDAVDDFKTGAVANMIGVIAYLLEKYKYNVYISLEDLTRAFRLQRDRLTNNILQSTNKDNTVDFKDQENLVLAGLGTYHYFEIQLLRKLFRIQRNSEGDILHLVPTFRSVDNYEKIVRRDKKTDNDKYVNYPFGIVRFVDPKYTSKKCPICDKTNTTRKDNVLICNSCNAVSGEYETDNENRHYITNGDDNGAYHIALKALSLRKSKNLEKKK

SEQ ID NO: 8 - protein, 1242 aa, synthetic construct
MEKYQITKTVRFGLTATNSNLYSDELKDLIETSEIKIKESLKNKSHNSLQIEQLRSCLNGVKEYLKTWNNVYSQIDFLGISKDYYKVISRKARFDFDNKGLGSEVKLASLQSKYNSKKRIQYILDFWEDNFQKTEILYRKSDELLKVFEEAEKQKRDDKKLNEVELRKTFLSLFNLVNESLKPLVEGNLFTINDDKIDSRNQNHEVIADFISNTKVRTELYESITELQNFFRDNGGYVPFGRATFNQWTALQKADKNGEREIDKIIKQLNLETVSMANIDYKYNTFTKNFEQGGQVWKIKQNAKSVIELCQFFKYKKVSITTRLNLAKRLNKTNNFLSEFGISKSPALDYKKDKENFNLANYPLKVAFDYAWENCAKAKHESITFPELQCKDYLHNVFGVDANKDKNGKIKNEELNKYADLLQFKILLGRLKAEFHKAAEETNKNNIRKLKNIFENLDYSGVQDFNKNKIKEIVEVWFANKEKNIGKKKEEMIPLTEKKKDDFSKAMQIIGQERGGLKSRIKKYKTLTEMFKVCASRFGKLFADLRDYFNEAHEVDKIKYRSWILEDGKQNRFVLLVDKAKDLELENEENGELKLYEVKSLTSKSLIKFIKNKGAYPDFHSLNSFNSDEIKKNWTNHKANINFLKNLKSALENSLMAINQNWKEFNFDFSRCDTYEQIEKEIDRKGYILKQQNISLNTIKKSINEEKSEKINNSKKLPSLLFPIVNQDINREAKQEKNQFTKDWFEIFAEENNLHKKRLHPEFHLFYRFPTKNYPNTKFKNGKEKSKRYSRFQMLAHFGLEVFPQGDYISKKEQIEIFNDDKKQKEAVEKYNNSIVSEVEYIIGIDRGIKQLATLCVLNKNGVIQSGFQIYTPSFNHDTKQWEHSFLGKRNILDLSNLRVETTIKNEKVLVDLASIQTKKGENQQKIKLKQLAYIRELQYSMQTRQVELLEYAKTLNSAEDITEEKIKIFISPFKEGSHYEHLPKQEIYNLLNEWQNADETRKRKIQELDPTDSLKSGIVANIVGVIAFFCEKYNYKVRISLEDLTRAFSIQKDALTGTPIHRNDEDFKEQENRRLAGVGTMQFLEMQLLKKLFKLQSEKNKHLIPAFRSVANYEKIVRRDKENGGDEFVNYPFGIVTFVDPRNTSQKCPYCNNIARKEDDAFYRNAGENKNSLLCKKCGLSTIKGKENKSNQDDSKNQFNIHFITDGDQNGAYHIALKTLENLHRLNTPKVTKHTKTKWKK

SEQ ID NO: 9 - protein, 1201 aa, synthetic construct
METNKTTKAINEYQTQKTIRFGLTVTNNNLYSENIVKLLKCSEEKIKEQLKKTQTDDLQNQRLRCCLIEIKEYLKTWNNVFSQIDFLAITKDYYKVLSRKAKFDYDKGNGSEIKLSSLQSKQSKYNDKKRYQYILDFWHENFIKVENLYRKSDDLLKVFEEAANQNQDDKKLNKVDLRKTFLSLFNLVNETLKPLIEGNLFIVNDDKIDEHNSKHNFVSDFIVKTEERKQLHDCITDLQDLFKANGGYVPFGRATINKWTALQKSNHKDDEIKRIIRELKIENISMQNIDYKYKYDSFAENFKQIYNKEGEKVWVLQFDANSVIKVCQYFKYKKVPINARLNIAERLIKEKSWQREKKNDFLSEFGISKSPALDYKNDKENFNLANYPLKVAFDYAWENCAKAIYETTTFPKEHCEKYLKEVFDLDIANNACFTKYALLLRFKILICRIKSEETTQIQNIEAVRGILDEINKNISGRQDFSKAKIITEINNWLSFKEKQTDKKEKYSNQDNFSLAMQIIGQERGGLKSRIEKYKTLTDMFKVCASKFGKQFADLREYFQEAYEVDKIKYRAWIIEDEKQNRFVLFANKEREIDLTSEEGNLYFYEVKSLTSKSLVKFIKNRGAYADFHKLKNNFNYEKIKRDWQYYKNDKYFIQNLKDALRNSKMAIDQNWAEFKFDFTKFNTYEDIEKEIDRKGYKLVCKTVSLNTLKDFVENKGCLLLPIINQDINKDDKQAKNQFTKDWNSIYDNKKRLHPEFNLFYRFPTQDYPNTKFSNGTEKTKRYSRFQMLAHFGCESVPKGDYLSKKEQIAIFNDDAKQKDAVEKFNNSIASDFEYIIGIDRGIKQLATLCVLNKNGQIQGDFEIYTRTFENKQWKHTLSEKRNILDLSNLRVETTIDGNKVLVDLASITTKNGENQQKIKLKQLAYIRELQYSMQTRRDDLLDFAKGLQSADDILKDIRNFIVPFKEGGQYADLPNERIYNLLKEWRDADDEAKRKIAELDPAQDLKSGIVANMIGVVAFLCEKYGYKVRISLEDLTRAFGIQKDALSGIAIAPNDEDFKEQENRRLAGVGTYQFFEMQLLKKLFKTQVDKNLHLVPAFRSVDNYEKIVRRDKKTNGDEYVNYPFGIVRFIDPKYTSKRCPKCGKTDVNRNQKTNIVKCNNCEYETKAGNSSEANNIHFITDGDQNGAYHIAQKALKIQKEQ

SEQ ID NO: 10 - protein, 1304 aa, synthetic construct
MKQIKNQYQLSKTLRFGLTQKNKTKKENYAGEIYKSHSELSDLVEISEQRIKDSVSTNKNSESSLPVDAIGKCLNQISEFLKGWQQVYQRTDQIALDKDYYKILCKKIGFDGFWFDKKNGRKTKKPQARIISLLELEKKDDKETERKQYILDYWQENFINAVEKYNVVSEKLKQFEVALKINRTDNKPNEVEFRKLFLSLVNIICDTLKPLCFQQICFPRLEKIDNSKIDNKNLIDFAIDYQSKNELLSLISKLKSYFEENGGNVPYCRATLNPKTAVKNPESTDNSIESEIKKLGLDKIIKNNKDAFSFSYNLYNNTAEDKKSKLKDDENGGLIERSLLFKYKSIPATVRFEIAKTLSKPDGKTEEEILEFLRDIGQLESPAKDYADLKEKDNFNIEKYPLKVAFNFAWEGLARAKYHPEAVFPTEICKQYLKNHFKITEDNKDFVMYAKLLELNAVLSTLEKAKPTDEKKFSVAAKKLLEEIEWEKVGKNGSKNKEAITKWLQTKSKTDKNFKSAKQEIGLFRGRIKNNIRIKNNIKSEYSEITNVFKNIAEEMGKTFAEMRDKISGAAESNKISHYAMIIEDNNKDKYVLLQEFVENKNERIYAKSDSQKSDFKAYSVNSITSGAIVKMLKKIRTDKLKESNNFANTQPELTSKEKEKRNIKEWKKFINEKGWNLEFGLKLENKTLEEIKKEVDAKCYKFDIKYFDKETLSDLVKNKNCLLLPIVNQDLAKKEKNESNQFTKDWNAVFPQDTPWRLTPEFRISYRKPTPNYPKSDKGDKRYSRFQMIGHFLCDYIPKTDSFISNRQQIENYKDDERQELAVKKFNAALRGRTKNEEYKEQLNELAAKYSKNGQQKINVKTNEKFYVFGIDRGQKELATLCIIDQDKKIIGPHKIYTRSFNSEKKQWEHKFLEERHILDLSNLRVETTVFIDGKPEKTKVLVDLSEVKVKDKVTGEYTKPDKMQIKMQQLAYIRKLQFKMQNEPEAVLAWYEKNSTEDLILKNFVDNEDGTNNGLVSFYGAAIEELKETLPIERIVDMLKEFKTIKKEEGKLTKEDEEGREKNKRKMDKLVQLEPVDNLKNGVVANMVGVIAFLLQKFDYQVYISLEDLSKPFSSKIISGIDGVPIRVEKEEGRRADVEKYAGLGLYNFFEMQLLKKLFRIQQDSENILHLVPAFRAMKNYDHIAVGKGKVKNQFGIVFFVDAEATSKTCPRCGSTNQKPNKKDYPNAQQARLSNDKEGWIDRDKSNGNDIIRCFVCGFDTTKEYTENPLKYINSGDDNAAYLISAEGVKAYELATTLADNI

SEQ ID NO: 11 - protein, 1292 aa, synthetic construct
MKNITNKYQITKTLRFGLSQKGKTKKEGFDGEIYQSHQEFNKLVSVSEARIKKSVTTEQKTELALSIDNVARCLNNISDFLINWQRVYYRTDQIALDKDYYKIMCKKIGFEGFWFETNRRTQQKIKKPQSRIISLSALDKKDGLGKERKQYILDYWKENLLSAAEKYEVVSEKLKQFQDALNINRTDNKPNEIELRKLFLSLTHIVYDILQPLCYGQICFPKIEKLDNTKEDNKKLIEFASDYQSKSDLLSEIAELKQYFEENGGNVPFCRATLNPKTLVKNPKSTDNSINEEIKDLGLKEILKTYKDVLNYNNYLESLSAKQKLQLLNDRNTSIITRSLLFKYKPISANVQFDIAKTLSPEVGKGEEDLRAFLRGIGQPKSPAKDYADLQNKSDFNIEAYPLKVAFDFAWESLARAIYHADSDLPMDACKNFLQDNFKVKNDDTNLKLYAQLQELKAVLSTLENGNPNNAAAFRLKATNLLNEIPWKTVGNYGQQNKDEISKWLNNGKNKDDYKKAKQQIGLFRGRLKNNIQGFDNITQTNKNIAMKMGRTFATMRDKITGAAELNKVSHYAMIIEDRNTDRYVLLQPFTENEQDRIYSQTDYNNGDYTTYEVNSITSGAIAKMLRKARIDELSKNDNNRNLTSQPELTEEKKEKRNIKEWKNFIENKRWDLEFQLKLNEKNFEQIKKEVDTKCYNLRTKKINKTTLEDLVNKSDCLLLPIVNQDLAKEEKTNGNQFTKDWNSIFAQNTPWRLTPEFRVSYRKPTPDYPISDKGDKRYSRFQMIGHFLCDYIPKSDKYISNREQILNYKNDELQKKAVKDFHEDLKGKTEEENQNESMNALMAKFGNVNKKQKATTVEKPKEKFYVFGIDRGQKELATLCVIDQDKKIVGDFDIYTRSFNSERKEWEHTFFEKRHILDLSNLRVETTASIDGKAEKKKVLVDLSEIKVKDKNGNYSKPDKMQIKMQQLAYIRKLQFQMQTNPEGVLAWFKENSTKDLIINNLVDKKNGEKGLISFYGSAIEKMEDTLPVDRIEEMLQKFAALKKQEKEGEDVKLSIDQLVQLEPVDNLKNGVVANMVGVIAYLLQKFNYQVYISLEDLSNPFGSQITGGIAGVPLKQGKDEGRRMDVEKYAGLGLYNFFEMQLLKKLFRIQQDSCNILHLVPAFRAQKNYDHVAVGKEKVKGQFGIVFFVDANATSKTCPVCGTTNNKPNNQKYPNAKKGLSADGKEVWLERDKSNGNDIIRCFVCNFDTTKEYTENPLKYIKSGDDNAAYLISAAGIKAYELATTLINNQ

SEQ ID NO: 12 - protein, 1261 aa, synthetic construct
METLNQFTGLYSLSKTMRFGLTLKEKKPKNDSIAVESLYQSHQDLKELVELSDKRIIEEKKPEPPVENLGNPPIEKLRDCLNSMQKYLNDWRKVYTRYDQLAVLKDFYRKLERKARFDGFWKDKKGQNQPQSQEIKLSSLKHKSGEKEIKDCIVTYWGENIRKANEKWHQVDSVLKQFEEAKRKNRDDKKLNQVELRKLFLSLANLVNDTLVPLCQRSITFPNADKLSDNARDKSVLDFIGDNEIREHLLDKITKLKEYFQDNGGYVPFGRVTLNQYTAMQKPNKTDKEIEDAIKNLGLSIIKSQNFDAFEHIEEATDKVERLNTVSLPLVERAQYFKDKTIPVGVRDSLAKYLAKDDTAKEKELIDLFEKIGMPKRPAKDYSDPTLKEKFDLRKYPLKVAFDYAWETVASKELHDDILKNKCKKYLKDIFDVDTDKSIFFNIYSDLNYMKIILSRIEYPTQNQLSKDNFLEWNRKVITILDGDDFSHFNKNADGSTDKKMNTAKTYVKTWLDKLEANIEQFDGQDFKKFYEDFKKKNKNSCKDFDDAKRDIGLKRGGLKQIIEETETFTDKKTGKQKPKYKDSKYKELTEAFKSIAVDFGKHFATLRDKFNEENEINKIEYYGVIVEDENADRYLLLSKLSESREEIKNIFPDKAEGLKTYKVKSLTSKTLTKLVKNKGAYKDFHISDMRVDFKKIKEEWSAYKNDQAFLKYLKKCLTDSSMAQAQNWSEFGLDFDKCNTYEEVEKELDGKAYLLQETRLSKATITNLVKNKGCYLLPIINQDLAREDRTAKNQFTKDWKQIFENKKHYRLHPEFNMAYRQPTPNYPNSEIGDKRYSRFQMIANFMCEIVPQSTSYATRKEQIQTFNDNNKQQKAVKDFDSKFKLSDSYFIFGIDRGIKQLATLCVLDQGGVIRGGFEIYTRHFDGNKKQWVHTSLERRNILDLTNLRAETTIDGKKVLVDLSKVEIKNQTDNKQNIKLKQLAYIRKLQYQMQTNPEKVKNMSDEDIENDLKDIITPYKEGTHYADLPIENIKAMLDRFKVLYGKTDQQSKQELKELCELDAADNLKGGIVANMVGVIAHLMEQYNYRVKISLENLTTSFVNQSDGLNEYFISRGMDFKEQENAALAGLGTYQFFEMQLLKKIFRIQQDDGNVLHLVPAFRSKEDYEKIIRRDKNDGDEYVNYPFGLVTFVDPRYTSRKCPICGKTDVKRNDNIITCKKCGAVSGKYSFDDKNRQFITNGDENGAYHIALKTRKEVHNEN

SEQ ID NO: 13 - protein, 1223 aa, synthetic construct
MDKENSFKGFTNLYEVRKTVRFGLTQPNKKGELKTHLEFDDLINKSFENIKKDVKSRDKPNFKEKELIEKINQFINGLEKQLGNWKQIYERYDVISVNKDYYKILARKAKFDAFKKDKKPQASQIKLSSLQKDNRKDNIIRYWGNIITRSDYLINIFKPKLEQYLNAVNNPNNSSHTKPDLIDFRKVFLQFLKVNEEYLQPLFDKSIQFETGKKENSEEIKKINTFSGDENNKEINYLIDLGKEIREYFEANGSQVPYGKVSLNYYTALQKPNNFGEDIRKGVENLGIIKFLNKSEEDIKNYLKQNSKEKINLLNNAKNHYFIELIHLFKPKTIPFSVKYNLAKYLEKNFNLKYEDILNKFDLLGKSVDIGKDYLECKEKEKFSLEKYPIKSAFDYSWENLARNLKRDVDFPKSVCEKFLKDNFDIIINNSSFNLYANLLFIAENLATIEYGNPNNENEIIESIKNTFDDIKFESNKQEYDGYKKEILNILNQEKSKRNYKNILTAKQRLGLLRGQQKNKISKYYNLTQSFKKIASFIGKTLATIREGLKEENELNKITDYGIIIEDKNQDKYILTLKLDGKDIREKIKSKLWDGEYKVFEINSFTSRALNKFIKNPLGEDSKKFHGDYKYKHKEVSIYKDVKWIGYKEEFLIHLKDSLVNSQIAKEQNWKAFGWNFDNFNTYEKIEKEIDKKGYKLIKNSISKENLEYLINEEKCLLFPLINQDISSKKEQNKNEFTKDFNKAFLGIGYRIHPEFSIFYRQPDEENKKINKSGIINRFGRLQLLANIGIEYIPQNNDYKTRKEQNKISLDQTNQNELVQNFNKEKVNKYFDSLDDYYIFGIDRGIKQLATLCITNKNGIIQSYEIYTKYFNNNSKKWEYKKNRIEGILDLTNLKIESDKDGNKFLVDLSLFEAKDENGNSTGTNKQNIKLKQLAYIRKLQYQMSSNEKGVLNFLKKYQTKEERQNNIKELITPYKEGHHFEDLPVNIFEEMFENYEKLKNDKTLSEIEKQNLMKLTIELDSSEDLKKGVIANMIGVIVYLMKKYDYKVKIAVENLNQSFMGQNDGLNNSYISIKTNFKDQENGALAGMGTYHFFENQLLRKLYKVSVEEGILHLVPFFNSLDNVNKLNFEKEKILWVQTENYRKFGIVSFVRPHNTSKRCPICKSINVKRKDNITTCSDCGFITGKDNNIVIKKYKKEGLNLDLIKNGDDNGAYNICCKIGL

SEQ ID NO: 14 - protein, 1239 aa, synthetic construct
MRFGLTQPNKKGELKTHIEFSDLVNKSFENIKKEVNSKDKSKFDTRKELIDKINQFISGLENQLGDWKNMYERYDLISVNKDYYKILARKAKFDAFKKDKKGVKQPQANQIKLSSLRYNKELIINYWGNIISRSDYLINVFKPKLEQYLNAVNNPNNSSHTKPDLIDFRKVFLQLLKISEEYLQPLFNKSIQFETGKKENSGDIKRVNDFSGNENNKEINDLLDLGKEIREYFEANGSQVPYGKVSLNYYTAVQKPNNFDKEIKEGIKDLGIIEFLKKSEEDIKNYLKQDSKEKIYLLNNSKNPYSIELIQLFKPKTIPFSVKYNLSKYLEKNYNLKYEDILNKFDLLGKSVDIGKDYLECKDKEKFSLEKYPIKSAFDYSWENLARSLKRDVDFPKNVCEKYLNDNFNINVGNSSFNLYANLLFIAENLATIEYGKPNNEKEIIDSIKETFLELSDEIEKNNKKNEVENIIKYLNLNTDERKNIKDLQKKYFKNLDTKEQNILNIFDSFTKSKQSLGLLRGQQKNKIDKYRNLTQKLVDKKDSHIGIASFIGRTLASIREGLKEENELNKITDYGIIIEDKNQDKYILTLKLNGKDTREKIKNNLGNGEYKVFEINSFTSKALNKFIKNPLGEDSKKFHGYFQYKHREVSIYDENEKWVGYKEEFLKHLKHSLINSQIAVEQNWKDFGWNFDNCDTYEKIEKEVDKKGYKLIETSISKENLENLIHKEDCLLFPLINQDISSKKEENKNDFTKNFEKVFLGDGYRIHPEFSIFYRQPNEENLKPNKSGIINRFGRLQLLANIGVEYIPQNNDYTTRKEQNKISIDQTKQNESVQKFNKEKVNPYFDSLEDYYIFGIDRGIKQLATLCITNKKGVIQNFDIYTKHFNDNSKNWEYKNNRTEGILDLTNLKVESDKEGNKYLVDLSLFEAKDENGNLTGTNKQNVKLKQLAYIRKLQYQMSSNEEGVLSFLNKYKTKEERQNNIKELITPYKEGHHFEDLPMNIFEEMFENYEKLKNNKTLSEGEKQNLMKLTTELDASEDLKKGVVANIIGVIVHLMKEYDYKVKIAIEDLSNAWYFSKDGLSGDSILNSKIDEEMDLKKQDNLALAGVGTYHFFEMQLFKKLFKISVEKGILHLVPSFGNVRNYTDLLKEKYKYQYQQFGVIYFISPKFTSSKCPICGKGGKKHIKRENNVITCKECGFVSGKDNSINIKNNKKEGLNLDLIKNGDDNGSYNIGGKIK

SEQ ID NO: 15 - protein, 1232 aa, synthetic construct
MLHAFTNQYQLSKTLRFGATLKEDEKKCKSHEELKGFVDISYENMKSSATIAESLNENELVKKCERCYSEIVKFHNAWEKIYYRTDQIAVYKDFYRQLSRKARFDAGKQNSQLITLASLCGMYQGAKLSRYITNYWKDNITRQKSFLKDFSQQLHQYTRALEKSDKAHTKPNLINFNKTFMVLANLVNEIVIPLSNGAISFPNISKLEDGEESHLIEFALNDYSQLSELIGELKDAIATNGGYTPFAKVTLNHYTAEQKPHVFKNDIDAKIRELKLIGLVETLKGKSSEQIEEYFSNLDKFSTYNDRNQSVIVRTQCFKYKPIPFLVKHQLAKYISEPNGWDEDAVAKVLDAVGAIRSPAHDYANNQEGFDLNHYPIKVAFDYAWEQLANSLYTTVTFPQEMCEKYLNSIYGCEVSKEPVFKFYADLLYIRKNLAVLEHKNNLPSNQEEFICKINNTFENIVLPYKISQFETYKKDILAWINDGHDHKKYTDAKQQLGFIRGGLKGRIKAEEVSQKDKYGKIKSYYENPYTKLTNEFKQISSTYGKTFAELRDKFKEKNEITKITHFGIIIEDKNRDRYLLASELKHEQINHVSTILNKLDKSSEFITYQVKSLTSKTLIKLIKNHTTKKGAISPYADFHTSKTGFNKNEIEKNWDNYKREQVLVEYVKDCLTDSTMAKNQNWAEFGWNFEKCNSYEDIEHEIDQKSYLLQSDTISKQSIASLVEGGCLLLPIINQDITSKERKDKNQFSKDWNHIFEGSKEFRLHPEFAVSYRTPIEGYPVQKRYGRLQFVCAFNAHIVPQNGEFINLKKQIENFNDEDVQKRNVTEFNKKVNHALSDKEYVVIGIDRGLKQLATLCVLDKRGKILGDFEIYKKEFVRAEKRSESHWEHTQAETRHILDLSNLRVETTIEGKKVLVDQSLTLVKKNRDTPDEEATEENKQKIKLKQLSYIRKLQHKMQTNEQDVLDLINNEPSDEEFKKRIEGLISSFGEGQKYADLPINTMREMISDLQGVIARGNNQTEKNKIIELDAADNLKQGIVANMIGIVNYIFAKYSYKAYISLEDLSRAYGGAKSGYDGRYLPSTSQDEDVDFKEQQNQMLAGLGTYQFFEMQLLKKLQKIQSDNTVLRFVPAFRSADNYRNILRLEETKYKSKPFGVVHFIDPKFTSKKCPVCSKTNVYRDKDDILVCKECGFRSDSQLKERENNIHYIHNGDDNGAYHIALKSVENLIQMK

SEQ ID NO: 16 - DNA, 3885 bp, synthetic construct
  source 1..3885: mol_type = other DNA
atggaagcaataaaaaacaactatcaattaagcaaaaccctgcgttttggattaacacaaaaaagtaaaacaagaaaagatggttttacaggagaaatctaccaaagtcataacgaattaaaagacttggtaaaaagttctgaagaccgaattaaaaaatccgtatcaacagacgaaaaatccgagatgagtttgtctgttgataaaattagatgttgtcttgtgatgatttcggattttcttagtagctggcaacaagtttattctcgtgccgaccagattgccttagataaagactattacaaaatcctttgcaaaaaaattggctttgacggtttctgggttgatgagcgatatgatagaaaaaacgacaaaacagttagaacaaaaaagccacaatcccgaaccataaatctttccgaattagacaagaaagatgacaaaggcatcgaacgcagacaatatctcctcacttattggcgggataatctgataaatgcagcagataaatttgaagtagttactgaaaagttgaaacagtttgaagatgctttgaatattaaccgaacctataataaaccgaatgaagtagaattgcgaaaactattcttgtcattgacgaatattgttcaagaaacactgcaaccgctttgtctgggacaaatttgttttcctaaattggaaaaaatagatgattcaagaactgaaaataaacacttgattgattttgcaaccgattatcaatccaaaagtgatttgctttctgaaatttcagaattaaaaaaatactttgaagaaaatggtggtaacgtgccttattgccgagcaactcttaatcaaaaaacagctgtaaagaatcccaattctaccgacaatagcatagactctgaaattaaaaagctcggtcttgacaaaatattgaaagagaataaagatgctttgtattttgccaataaaatttacagtttgtctgccaaagaaaaactctcaaaattagatgataaaactaccggattgatagaacgtagcttactgtttaaatataagcctgttcctgctattgtacaatacgaaatagctaaaactttaagcgaaaccatcaataaaagcgaagaagatttattggaatttcttcgcagtattggacaaacgaaaagtcctacaaaggattatgccgatttacaggataaaaacgatttcgatttagatgcttatccgctaaaagtagcatttgatttcgcttgggagaacttggcaagaagtatttatcatagtgatgcggatattccggttgctgtttgtgaagggtttcttaaaaaaaactttggcattgataaaagcaatgcagatttcaagttatatgcacaattacaggaattaaaggctgtgttggcaacattggaatacggaaatccaacaaataggcaaacgtttataaatgaagcaacaaaattgttatccccaatatcttgggataaaatcggtcgaaacggaaatcaaaataaatattctattgaaaaatggctgaaaactctaacaaaagatgataaagattataaagatgccaaacagcaaatagccttgtttagaggacgattgaagaataatattaagacttttgatgacattacgaaatattttaaatccgttgctatggaaatgggcagaacctttgcccaaatgcgtgataaaataaccggtgcggcagaacttaacaaagttacccattatgccataattatcgaagaccaaaattttgataaatatgttttattacaggagtttgtcgataaaaaggagaatagaatatatgcaaaaacagacagacatcacagcgattttacgacttattcggttaattcggttacttcaggctctattgccaaaatgctcaggaaaaagagaatggatgaattgaatagaaataacagaaatagttttgaacagaaacctgaattatctgaagaacaaaaagaacagcgtaacattagagaatggaaagagtttatagaagataaacgctgggacttggaattccaattaaatctcagcaataaaacgtttgagcaaattaaaaaagaagttgatgccaaatgctatgaattggatataaactatatcagccaagaaactttatccgatttggtaaataaaaagggttgtttgctattgccgattgttagtcaggacatagcaaaagaaaataaaacagaaggcaatcaatttaccaaagattggaacgctattttcactcaagaaactccttggagactaactccggaatttagagtttcttatagaaaaccaacgccaaattatccggtttccgacaaaggagacaaacgctactcccgcttccaaatgatagctcatttcttatgcgattatcttccaaaatccgatagctatatttctaattgggaacaaattgcaaattataaagatgataaattgcaggaaaaagcggtaaaagaatttaatgcagatttgagaggacgaacggaagaagaaaaacaaagtgaatcggtgaatgcgttgcttgcccattttggaaaccagaacaaaaaacaaaaacctgttgagcgacctaaagaaaaattttatgtctttggcattgaccgcggacaaaaagaattggctacgctgtgtgtaatagaccaagacaagaaaattgtcggtgattttgatatttatacccgttcatttaactccgaagcaaaacaatgggaacataaattacttgagaaacgtgttattctcgatttatctaatttgcgtgtggaaacaactattgtaatagacggaaagccggaaaagaaaaaggttttggtggatttgagcgaagtaaaggtaaaagacaaagatggtaagtattcaaagccgaataaaatgcaggtaaaaatgcagcagttggcgtatattcgtaaactgcaattccaaatgcagaccaatccggatgctgtattagaatggtatgcaaacaacaagaccaaagaacaaatcatgtctaactttgtagataatgaaaacggtgataaagggttggtttctttctatggaactgccgttgaggaactgaatgaaaccttgccgattgataaaatcgaagaaatactcaaaaaatttcaagagctgaaagataaagaaaaacaaggtgaaaccgttaaattggaaattgataaacttgttcagctagagccggtagataatttaaaaaacggagtggttgccaatatggttggcgttattgcttatttacttcaaaatcttgattatcaggtctatatttcactcgaagatttatcaaaaccattcagtggtcaaattataggaggaatcgccggtgtgccaacaaaaacaaataaagaggaaggtcggcgtgccgatgtggaaaaatatgccggattaggtctttacaatttttttgaaatgcaactgctcaaaaaactattccgcattcagcaagacagccaaaatattttgcatttagtgccgcctttcagagcaatgaaaaattatgaccatgttgccgttggcaaaggcaaagtaaaaaatcagtttggtatagtcttttttgtggatgctgatgccacttctaaaacctgtccatgctgcggttcatcaaataataagccaaatctgaagatgtatccaaacgctaaaaagggactatcaaaagaagggaaggaagtttgggtagagcgtgataaatcggaaggtaatgacattatcagatgcttcgtttgcgggtttgataccacaaaagattattccgaaaatccgcttagatacattaaaagcggagatgataatgccgcctatctgatttctgcagaaggaattaaggcttatgaattagcaacaacattagtgaacaacaaataa

SEQ ID NO: 17 - DNA, 3861 bp, synthetic construct
  source 1..3861: mol_type = other DNA
atggaaaatatcaaaaacaattatcaattatccaaaacacttcgttttggcttaacacaaaaacaaaatggaaatagctcaaatacagataatgtttatcatagccatagtgctttaaaagaattagtggacatttccgaaaacagaattaaaaaaaatgtttctacagagggagcaaccgaaatgcaattatccattgaaagcattagaaaatgtatgattatgattgagcagtttattaaagactggaaaagagtatattatagatcagaccaaatttctttagataaagatttttacaaaaagctaagtaagaaaattgggtttgaagcattttggtttgaaagaaataaaaaaactcaacaaaggataaaaaaacctcaatcttgcataattgctttgtctgaactctcaaaaagagataattttggaaaagaacgtcaagaatatattgttgaatattgggaaaataacttgctaaaatctacagaaagatacgaagaagtaagtgaaaaattagaacagtttgaattagctcttaaaatcaatcgaacagacaatcgcccgaatgaggtagaattgcgaaaaatgtttctatcgcttgtaaatatggttcgagaagtagttgagccactctgtttgggacaaatttcttttcctaaattagagaaattagcagacaattctaaaaataaacaactacgaaaatttgcaacagattatcaatcaaaaagtgatttattgacacaaatttctgaattgaaaaaatattttgaagagaacggtggcaatgtgccgtattgcagagctacgctcaatccgcttactgctgtaaaaaatcctaaatctactgatagtagtattcttgatgaaatcaagaaattaaaattagatgttatcttaagagactaccaaagtgttgctctttttgataattccattcgagatttgacagcatcacagaaaatgcaattgcttaaccaaaataacgaaggccttataaagcgtggtttactattcaagtacaaacccattcctgctattgtgcaatatgaaattgcaaaagtattgagtgctgaactcaacaaagacgaacaagaattacgaaattttttaagagatattggtcaggttaaaagccctgccaaagactacgcagaattgcaagataaaaaagattttgacatcaatcattatcctttaaaagttgcttttgactttgcgtgggaatcgcttgctaaatctgtttatcatcccgatatagatttcccagaggaacaatgcaaaacattactaagggaagttttccaagtagatgaaaacaacgaaaattttaaattttatgcccaacttttagagctacggtcgttacttgccactttagaacacggaaaaccaacagaggtaataacaattgaaaacgaagtaaagaaaattctcgaaaatattgactggagtaaatttggagatagaggcaaaaactacaaatcggctattgaaaattggatacacaatagaaacaaaaaagattttaaaggcgactatttcaaaaaagccaagcaacagataggtttaacacgtggtagacaaaaaaatttaataaaaaagtatgacgaaataacaaagtcttacaaagacattgcaatgaaaatgggcaaaacttttgccgaaatgcgggataaaattaccggtgcagccgaactcaataaagtatcgcattacgcaatgattgtggaagataccaatcaagacaagtatgttttattgcaagaatttgtagaaaataataaggatagaatttatgcaaaaagtactcctcaaaatgaagattttaaagcatattcggtcaattctgttacttcttcggctatcgcaaaaatgattagaaaaataagaattgacaagctacaagcaaatgaacgaaacaataatagacaacaagcaccagaactatcagaaactcaaaaagaggcaagaaatatcaaagaatggaaagattttattgcggaaaaacgatggaattatgagttcgatttaaaattggacaataaaaattttgagcaaataaaaaaagaaatagactcgaaatgctttaaattagaaaccaaatatatgagcgaggaagtacttgttgatttggtcaaaaatcaaaattgcctgcttttaccaattatcaatcaagatttagctaaaaaaataaaatccgagagtaaccagtttaccaaagattggaatgccatttttgcacaaaacacaccttggcgacttacgccagaatttagagtgtcttacagaaaacctactccaaattatcccaaatccgaaagaggtgataaaagatattctcgttttcaaatgataggtcatttcttatgcgattttatccctaaaactgctgattatatttcaaatagagagatgattgctaattttaaagatgatgaaaaacaaaaacaaacgattataaattttcatgaaagattaaaccctaaatctgaaaatgaaaaaatgaacatgttgttagctaaatttggaaataaaaattcaaatcaatctaaagaaacaaaaaaagaagaaaaattttatgtgtttggaattgaccgcgggcaaaaagaacttgcaacactttgcgtaatagaccaagataaaaaaattattggagattttaatatttacacccgctccttcaacacccaaaacaagcaatgggagcatcaacttttagacaaaagacatattttagacttatctaatctgcgagtcgaaacaactattgtaattgatggtaagcctgatgtgcgaaaagtattagttgatttgagcgaagtgaaagtgaaagacaaaaatgggaattacacgaaaacggataaaatgcaagtgaaaatgcaacagttagcatacatacgcaaacttcaatttcagatgcaaacgaatcctgatactgtgttggaatggtacaataaaaatcaaacaaaggaggcgattttaaataactttgttgataaaccaaatggcgaaaaaggcttagtctctttttatgggtctgctgttgaggaattaaaagatactttgccgattgaaagaattgaaaagatgctgcaacaatttaagtctctaaaaaatgaagaaaaagaagggaaagatgtaaaggctgaaattgacaaattgatacaacttgaacctgtagataacttgaaagcaggtgtagtggctaatatggttggtgtgattgctcatttattaaaagaatttaattatcaagtatatatatcattagaagatttatctaatccttttggtagccatgttatagatggaaccaccggaactcattcaaaaacgaataaaggtgaaggtaaaagagccgatgtagaaaaatatgcagggctgggtttgtataacttttttgaaatgcaattactcaaaaaactgttccgaatacagcaagatagccaaaacattttacatttagtacccgcatttagagccgtaaaaaattatgaaaacatcattgcgggaaaagataaaattaaaaaccaatttggaatagtattttttgtagatgccaattctacttcaaaaatgtgtcctgtttgtaattctaccaatgaaactaatagagagtacccaaatgcaaaaaaaggaacttctaaagatgataaagaagtttgggtagaacgagataaatcaaacggagacgacataattcgctgttttgtgtgtgggtttgacacaactaaaaaatatgaagaaaatccactaaaattcattaaaagtggtgatgataatgcagcgtatataatttctgcttttggcataaaggcttatgaattagctaaatcagtaattgataacaagtaa

SEQ ID NO: 18 - DNA, 3747 bp, synthetic construct
  source 1..3747: mol_type = other DNA
atgaaaaatttaacacaatttgtaaatttgtaccaactctcaaaaacattgaaattcggattaacattacgaaataaaataagaaaaaatggttttgaaggagaaatttatgaaagtcatactgaattacaggaacttataaaaatttcagagcaaaaaatcattaaggaaacaactgataaaaataaagaaataatgacatttacagaattgcccttagatgaaattcgtaaatgtcttgacgacatgcataaatatctcgatgattgggaacaattttataacagatatgaccaaatagcagtacttaaagattattatcgaaaattggaacgtaaagcaagatttgacggtttttggagagaaaaaaatattaagaacaaaatacaaaataatgaatctgaaactatcaaaaaaccgcagtctcaagttatcaaattgtcaagtttgaacaatgaatatgaaaataagaaacgtcgagattacataaccgactactggaatgaaaatattcaaaaagcaaaacgaaaattttatgaagttaattctgtattaaaacaatttgaagtagctaatgaacaaaatagagacgataaaaaattgaacgaagtagtgttacgtaaattatttttatcttttacaaatcttattaacgatacacttgaacctctttgtaatggctctatttgttttcctgatattgaaaaattaacaaacagcaaaactgacgaacaattacagagattcgtttttgatgacggattcaaaaaaatattatcagaacaaatagaaaatttgaaaatttactttgcaataaacggcggatatgtccaatatggtagagtaacattaaataaatataccgcgttacaacaacccaataaggtagatgaagatataaagaatataattaaagaactgggtttgttggaatttgtaaaaaagtatgaaaatactgaacaaatcattaactatataaaaaatattaaagataaaaaacaagaactaaacgctaataatttgtcattgatagaaaaagtacaattatttaaatacaaaactattccagctggagtacaaccttcgcttatagcatatctggcacgaacagaaaaaaaagataaaaaaacactcagagaactattttacgcaatcggtcagccacaaagtccgtcaaaagattataaagaattacaaaataaaacggattttaatttgtacaaatatcctttgaaggttgcattcgattatgcgtgggagtcattggcaaaaagtaaatataacccacatatagattttccagatgtcaaatgtaaagaatttttaaaagatatttttggtacggatatatctgttaatgataatttcaagttatattccgcacttttgtttgttcgtgaaaatcttgcaacattagatcatggtaatccaaacgataaaaatattcatgttaacaaagtagaaaatacatttaaagagatcaaagatagattggcaaaaaaagaatataaaaaagaatataaagccttagaaattatttgtaaatggcataaaaattcagcaaccattgaacaatcagaatatgaggcagcgaaacaaaccatatatgaggcagcgaaacaaaccattggacaattaagagggcggcaaaaaaaccaaatatctaaatttaaagaattgacggattcattcaaaaagttggctccaaaatttggtaaagcatttgctaatcttagggataaatttaacgaagaatatgaaataaataaaatttctcattgcggagttattgtagaagatcgcaataacgatcaatatttgttattgtctcaattaaatgataatagagaaaatgcatctgatatttttgagttagaagctgatcccaacggtgagttgaaaatttatcaggtaaagtcattgacctctaaaacgttgttgaaatttctcaaaaacaaaaaaggttccaatactggatttcatatcaatgaaaattggacgtttccaaaagggaaatgggatgttattaataaggataaaatttttcttaattatgtaatacaacgtattacaaattcgagtatggcgaaagagcaaaaatggagtaactttaaatgggattttaggcgatgtgattcatacgaagcgatagccaaagaagtagatgccaaaggatatattttagaatctgtcaatatttctaaattgacactgaacaaattgataacagaaaagaaatgtctgttactacctattgttaatcaagacttaacaagacaagataaaaaaacaaaaaatcaatttacgaaagattggataaagatttttgagagtaataattgttaccgattacatcctgaatttaaaatatcttatcgatatccaactcctaattatcctaaaccggaagaaaagcgttattcccgttttcagatgatttcgcatttactttgcgaatatattccacaaaacgataattataaatcacgtaaagaacaagttaaaatctttaatgataaagttgctcaaaaagaatctgtagaacaatttaaccaacaatttgaaataacagatgattattatatttttggaattgatcgcggcataaaacaattagcaacactttgtatattaaataaaaatggacaaatacaaggagactttgaaatatatactcgtgaatttgataaagtcaataaacaatggaaacacactattcttgaaaaacgaaatattttagatttgtctgatttacgggttgagacaacagttgagggtaaaaaagtattggtcgatctgagtaaagtgaagttacacagtggaaatgaaaataagcaaactataaaacttaaacggttggcatatattcgtatgttacaatatcaaatgcagcatgaacaagataaagtattaagatttataaatcaatacaaaacaattgatgagatagaaaaaaatattagagatttaatttcaccttttaaggaaggaaaacaatatgccgacttacctacagaaaaaataaaagatatgcttatacaatttggtgaattatcaaagaatgatagcgataaatctaaaaaagaattgtgtacactttgtgaattagatgccgtagatgattttaaaaccggtgctgttgctaatatgataggtgtaattgcttatctactagaaaaatataaatataacgtttatatttcgttagaagatttgactcgtgcatttagactacaaagggatagattaacaaataatattttacaaagtaccaataaagacaatactgtagatttcaaagatcaagaaaatttagtattagcaggattgggaacttatcactattttgaaatacaattactaagaaaattgtttcgtatccagcgaaatagtgaaggagacattttacatttagttccgacatttcgtagcgtagataattacgaaaaaattgttcgcagagataaaaaaacagataatgataaatatgtgaactatccctttggaattgtgcggtttgttgatccgaaatatacttctaaaaaatgtcctatttgtgataaaacgaacaccacaaggaaagataatgttctgatttgtaattcttgcaatgcagtatctggagaatatgaaacagataatgaaaatagacattatattaccaatggcgatgataatggtgcataccatatagctttaaaagcattaagtttaaggaagtcaaaaaatcttgaaaagaaaaagtaa

SEQ ID NO: 19 - DNA, 4182 bp, synthetic construct
  source 1..4182: mol_type = other DNA
atgactaagtatcaattaacaaagaccctacgatttggactcaccaaggtgagaaaaaaaactaaacttgtggcagggaaagaggtggatgcaaaatacctcagccatgaggagttggatgacttggtgatgaggtcggagtataatctcatcaaaaggaatgtgcttgaatggtctaaaaaaaaggaaagcgacaaatctaattatatctttgatgaacttgacaagagatttgaagaaataatcgaaatcgaagacagagatcaacgcaatgccgaatacgttgaatttttgaatgagttcttcaaagaggtaaaccatcagactataaaccaattggatgaatcgacttttataaataaaattggtgattgtagtaaagctataaaggagtatttgttaagttggggaaaagtatgcaggcgcattgataaaatcacagttagaaaggattacttcaagatacttgctcgtaagacttttttcaaatacgaatcaaaagttgggaaaaagaggacgcctctgccttccgaagtcaaactatcgggacaaaaaggcaataattattttgatgaaccgataaacgaaggcatttctcaattttggcaaaatagagttgcaaaagcattaaacctacattcgcaacttgagtcaatgctttttgattacaaaaaagcaatagaaacagaaaaacacaatcaggaaaacccgaaaggagataatggttcatttgacaagctgcaccttgtagattttagaaaaatgtttctatcagtatgcagtcttgttatggatagtttgcgcccaattgttaatgaacttatcatagtgtcggataatgtttcgaaagacgaggataagtatattttggactttgtcaatgacaaaaaaacgcaatgggatttgttccaacaaatagagaatttacagactatatgcaaagataacggtgaaaatatcttctttgggaaagcaacgttcaataaatacacttctgaacaagccccaaatcatcgaaataatgatattgccaaggttctccgagaactgaaaattgaaaaatttgtgtctgattatattgacttggatcaagaagcaataaatcgaaaaatttatcaatcaacccaatcccgtttggagaacttgaataatccacagatttctcctattatccgcgcacagtatttcaaatacaaaccaattccaacattagtaagattcggacttgcgaaagaattggccaaacagcaaggaaaaaaatattccgatcgattgaaaggcatccaagaattgtttagaatttttggatcttcaaaaagtccggcattggattacaaaaacaatagaactgatttctcattggacaattatccaatcaaagttgcatttgattatgcatgggaaatgtgtgcccgttctgaatacgctcaaaaaccagtcgattttccaaaatcaatttgtgaaaaattcttggaaaaattttttgaatgcaaaagtaacgaaaaatatcaacaatcatttgtgacttatgcccgtttattgaaaatcaatgaagacttggcaactttggaacattttgaaaatgagccacccaaagatatcgaatcaatttaccaagatgcgcaaagatatctcgatgaagtggggaatttatgctcaaatgaggatagagcagcaatcgcaaaatggtacgaagaatacaacaagttatggacaaaaggcgaccataaaaagttaaaagaatggattgtgtcaaaatcatctatcattactaattttacgcaggcgaagatgcatttgggacaaaaaagaggaagccaaaaaacttttgttcttaaatcatatttccattcttcttatggaaaaattagagataacaatcgatttgtaaactctaatgttaccgaagtgtttaaaacaatagccagcactttcggcaaatcgtttgccacaatcagagagtattttaacgaagaaagtgaagtaaacaaaatagaatacggcgctgtgattatcaaagataaaaacggtgacaaatatcttttgcttcaaaagaaaaatgaaggtggtattgatatgcctatattcaataaatctgatgaaaatggtgattgtgacctttatcaggttaaatctctgacatccaaaacagttaggaaaattattgcatcacccaacaaatataacgatttttttgttaacaatgatggcaaaaaaatcatttatccagacaaaacagatttcaaatacaagattaatccatatgacaaagaagaagtcaaaaaacgcaaaagagaattgtacaataatgatttagtgcgaccaattatatatagtttgactcaatctaaatttgccaacaaacaaaattttgagaaatattttgattggactaaggctttaaaacaatgctcaaacattgagcaattgtacaaaacaatcgaccagaaaggctattccttgaacccttccaaaatcagcaaagagcaaatcgcggatttggttaacaatatgaattgttatctcttgccgattgtaaatcaaaatattacagcaaagacaaagaacgacacaaatcaattcaccaaagattggaataagattttcaatgaggtagataaggattatcgtattcatcctgaatttacgatgttctatcgttatcctacgccggattatcctaaatttggagaaaagaggtattcccgtttccaaatgaatgtaaactttctaatggaagttattccggctgatggcgaatactgttcacgaaaggagcaaatagaaatatacaatgctcccaaagacaacgaaaattgtcaaaaaaatgtagttgagagattcaacaataaaattaaagccctaaagccatcgtatttcataggcattgaccgtggcattaacgaattggcgacattgtgcgtaatagataaagaaggtaaaatcgttggcgattttgagatatacaaaagagaatttgactcaaatctgaaacgtcagaaatatacatcaatagagactcgcgacattttagacctgtcgtacttgcgtgttgaaaaggacgaaaatggtgaatctcgtttagtagatttgtcagaatcggaggtatggatagacagccttgatcacgacaaaggaaaaagagccaacaaacaaatagttcacctcaaacatctgtactatttgcgttgcatcgcacatttgttgcagtcgtcagattacaaatctattgtattagaaaaactcaaagattgcaacaatctgaaagatgaggaaataaaaaaggtattcgagaaagataaatttgttgattcctacaaaggtggcgaagcatatacagatttgccatacgatgaaatcagaaagttaatttctgattatcaggaaattgagcaatcaaaccaaacagaatcggagaagtccaaagcattaaatacactttgccagttggacgcatctgaatatctcaaaaagggtgttgttgccaatatgataggtgttgtcgtatatgttttgaaaaagtataattacgatgcgtatatctctctcgaaaatctttgctatgcatacggatatagcaaagacacattgtcaggatattcaattacaagtactaaggaagatccatatttagatttcaaagatcaagaaaacgcgaaattagcaggattaggaacttatagtttctttgaggttcaacttctgaaaaaacttttcaaacttcaaattgaagaaaatacagagttgattccagctttccgcagcgtggacaactacgagaaaatatttttgttaaaaaatatagataacaaaatttatcagttcggtattgtgtattttgttgatccaaaatatacaagcctatgttgccctatttgtggtgaacatggcaaaaaaaatgttgataggaagaaacatacaaaaaaatatgacgaagatgaattagtatgcaagcaatgcggttttcatacaaatctttcccatatcgaaacaagggttatggaagacaaaacaataaaaaatagttacgatgagtgtaatttgaaagccattgtttctggagatgcaaatgccgcttataacattgcaattaggttgggcaaaaacatatactcaacaattgcagacaaagtaaaggatttgcatcacgaagggaaaaaatacattatcgtaaaaggataa

SEQ ID NO: 20 - DNA, 4113 bp, synthetic construct
  source 1..4113: mol_type = other DNA
gtggcagggaaagaggtggatgcaaaatacctcagccatgaggagttggatgacttggtgatgaggtcggagtataatctcatcaaaaggaatgtgcttgaatggtctaaaaaaaaggaaagcgacaaatctaattatatctttgatgaacttgacaagagatttgaagaaataatcgaaatcgaagacagagatcaacgcaatgccgaatacgttgaatttttgaatgagttcttcaaagaggtaaaccatcagactataaaccaattggatgaatcgacttttataaataaaattggtgattgtagtaaagctataaaggagtatttgttaagttggggaaaagtatgcaggcgcattgataaaatcacagttagaaaggattacttcaagatacttgctcgtaagacttttttcaaatacgaatcaaaagttgggaaaaagaggacgcctctgccttccgaagtcaaactatcgggacaaaaaggcaataattattttgatgaaccgataaacgaaggcatttctcaattttggcaaaatagagttgcaaaagcattaaacctacattcgcaacttgagtcaatgctttttgattacaaaaaagcaatagaaacagaaaaacacaatcaggaaaacccgaaaggagataatggttcatttgacaagctgcaccttgtagattttagaaaaatgtttctatcagtatgcagtcttgttatggatagtttgcgcccaattgttaatgaacttatcatagtgtcggataatgtttcgaaagacgaggataagtatattttggactttgtcaatgacaaaaaaacgcaatgggatttgttccaacaaatagagaatttacagactatatgcaaagataacggtgaaaatatcttctttgggaaagcaacgttcaataaatacacttctgaacaagccccaaatcatcgaaataatgatattgccaaggttctccgagaactgaaaattgaaaaatttgtgtctgattatattgacttggatcaagaagcaataaatcgaaaaatttatcaatcaacccaatcccgtttggagaacttgaataatccacagatttctcctattatccgcgcacagtatttcaaatacaaaccaattccaacattagtaagattcggacttgcgaaagaattggccaaacagcaaggaaaaaaatattccgatcgattgaaaggcatccaagaattgtttagaatttttggatcttcaaaaagtccggcattggattacaaaaacaatagaactgatttctcattggacaattatccaatcaaagttgcatttgattatgcatgggaaatgtgtgcccgttctgaatacgctcaaaaaccagtcgattttccaaaatcaatttgtgaaaaattcttggaaaaattttttgaatgcaaaagtaacgaaaaatatcaacaatcatttgtgacttatgcccgtttattgaaaatcaatgaagacttggcaactttggaacattttgaaaatgagccacccaaagatatcgaatcaatttaccaagatgcgcaaagatatctcgatgaagtggggaatttatgctcaaatgaggatagagcagcaatcgcaaaatggtacgaagaatacaacaagttatggacaaaaggcgaccataaaaagttaaaagaatggattgtgtcaaaatcatctatcattactaattttacgcaggcgaagatgcatttgggacaaaaaagaggaagccaaaaaacttttgttcttaaatcatatttccattcttcttatggaaaaattagagataacaatcgatttgtaaactctaatgttaccgaagtgtttaaaacaatagccagcactttcggcaaatcgtttgccacaatcagagagtattttaacgaagaaagtgaagtaaacaaaatagaatacggcgctgtgattatcaaagataaaaacggtgacaaatatcttttgcttcaaaagaaaaatgaaggtggtattgatatgcctatattcaataaatctgatgaaaatggtgattgtgacctttatcaggttaaatctctgacatccaaaacagttaggaaaattattgcatcacccaacaaatataacgatttttttgttaacaatgatggcaaaaaaatcatttatccagacaaaacagatttcaaatacaagattaatccatatgacaaagaagaagtcaaaaaacgcaaaagagaattgtacaataatgatttagtgcgaccaattatatatagtttgactcaatctaaatttgccaacaaacaaaattttgagaaatattttgattggactaaggctttaaaacaatgctcaaacattgagcaattgtacaaaacaatcgaccagaaaggctattccttgaacccttccaaaatcagcaaagagcaaatcgcggatttggttaacaatatgaattgttatctcttgccgattgtaaatcaaaatattacagcaaagacaaagaacgacacaaatcaattcaccaaagattggaataagattttcaatgaggtagataaggattatcgtattcatcctgaatttacgatgttctatcgttatcctacgccggattatcctaaatttggagaaaagaggtattcccgtttccaaatgaatgtaaactttctaatggaagttattccggctgatggcgaatactgttcacgaaaggagcaaatagaaatatacaatgctcccaaagacaacgaaaattgtcaaaaaaatgtagttgagagattcaacaataaaattaaagccctaaagccatcgtatttcataggcattgaccgtggcattaacgaattggcgacattgtgcgtaatagataaagaaggtaaaatcgttggcgattttgagatatacaaaagagaatttgactcaaatctgaaacgtcagaaatatacatcaatagagactcgcgacattttagacctgtcgtacttgcgtgttgaaaaggacgaaaatggtgaatctcgtttagtagatttgtcagaatcggaggtatggatagacagccttgatcacgacaaaggaaaaagagccaacaaacaaatagttcacctcaaacatctgtactatttgcgttgcatcgcacatttgttgcagtcgtcagattacaaatctattgtattagaaaaactcaaagattgcaacaatctgaaagatgaggaaataaaaaaggtattcgagaaagataaatttgttgattcctacaaaggtggcgaagcatatacagatttgccatacgatgaaatcagaaagttaatttctgattatcaggaaattgagcaatcaaaccaaacagaatcggagaagtccaaagcattaaatacactttgccagttggacgcatctgaatatctcaaaaagggtgttgttgccaatatgataggtgttgtcgtatatgttttgaaaaagtataattacgatgcgtatatctctctcgaaaatctttgctatgcatacggatatagcaaagacacattgtcaggatattcaattacaagtactaaggaagatccatatttagatttcaaagatcaagaaaacgcgaaattagcaggattaggaacttatagtttctttgaggttcaacttctgaaaaaacttttcaaacttcaaattgaagaaaatacagagttgattccagctttccgcagcgtggacaactacgagaaaatatttttgttaaaaaatatagataacaaaatttatcagttcggtattgtgtattttgttgatccaaaatatacaagcctatgttgccctatttgtggtgaacatggcaaaaaaaatgttgataggaagaaacatacaaaaaaatatgacgaagatgaattagtatgcaagcaatgcggttttcatacaaatctttcccatatcgaaacaagggttatggaagacaaaacaataaaaaatagttacgatgagtgtaatttgaaagccattgtttctggagatgcaaatgccgcttataacattgcaattaggttgggcaaaaacatatactcaacaattgcagacaaagtaaaggatttgcatcacgaagggaaaaaatacattatcgtaaaaggataa

SEQ ID NO: 21 - DNA, 4113 bp, synthetic construct; identical to SEQ ID NO: 20
  source 1..4113: mol_type = other DNA

SEQ ID NO: 22 - DNA, 3747 bp, synthetic construct; identical to SEQ ID NO: 18
  source 1..3747: mol_type = other DNA

SEQ ID NO: 23 - DNA, 3729 bp, synthetic construct
  source 1..3729: mol_type = other DNA
atggaaaaataccagattactaagacagtgagatttgggttgacagctacaaattcaaatttgtattccgatgaattaaaagatttgattgaaacttcggaaattaaaatcaaagaatcattaaaaaataaaagtcacaattctctacaaatcgaacagttaaggagttgtttgaacggagtaaaggaatatctgaaaacttggaataatgtttatagccaaattgattttttgggaatatctaaagactattataaagtaatttcaagaaaagcaagatttgattttgataacaaaggacttggttccgaagtcaaacttgcttctctgcaatcaaagtataatagcaaaaaaaggattcaatatattttggatttctgggaagacaatttccagaaaacagaaattctatatcgcaagtcagatgaattattgaaagtttttgaagaagcagaaaagcaaaaacgtgatgataaaaaactgaatgaggtagaattacgtaaaacttttttaagtttatttaatttggtgaatgaaagtttaaaacctttagtagagggaaatttatttactataaacgatgataagatagatagcagaaaccagaatcatgaagtaatcgccgattttatttcaaacactaaagtcagaactgaattatatgagagtataaccgaattacaaaattttttcagagataacggtggttatgttccctttggtagagcgaccttcaatcaatggacagctttgcaaaaagcagataaaaatggagaaagggaaatagataaaattataaaacaactgaatctagaaaccgtttcaatggcgaacattgattataaatataatactttcacaaaaaattttgaacaaggaggacaagtttggaaaatcaaacaaaatgcaaaatctgttattgaactctgccagtttttcaaatataaaaaggtatcgattacaactcgtttaaatcttgccaagcgactgaataagactaataactttttaagtgaatttggaatttcaaaatcacctgctcttgactacaaaaaagataaagaaaacttcaatctagcaaattatccactgaaagtagcatttgattacgcttgggaaaattgtgcaaaagcaaaacacgaaagcattacatttccagagctgcaatgtaaggattatttgcataatgtgtttggtgtggatgcgaataaggataaaaatggaaaaataaaaaatgaagagttaaacaaatatgcagatttattgcaattcaaaatattgcttggaagactaaaagcagaatttcacaaagcagctgaagaaaccaacaaaaataatattcgaaagctaaaaaatatttttgaaaatttagattacagtggtgtgcaagattttaataaaaataaaatcaaagagattgttgaagtctggtttgccaataaagaaaagaatattggaaaaaagaaagaggaaatgattcctttaacagaaaaaaaaaaagatgatttttccaaagccatgcaaattatcggacaagagcgtggggggctgaaaagcagaatcaaaaaatacaaaacattaacggaaatgtttaaggtttgtgcttcaagatttgggaaattatttgccgatttacgagactattttaatgaggcacatgaagttgataaaataaaatatcgttcttggattctagaagatggaaaacaaaatcgatttgttttacttgtcgataaagcaaaagacttggagttagaaaatgaagaaaatggtgaattgaaactttatgaagtaaaaagtttaacttccaaatcactaataaaatttattaaaaacaaaggagcctatcctgattttcacagtttaaatagcttcaattctgatgaaataaagaaaaattggacaaaccataaagccaatataaactttctaaaaaatttgaaatctgcattagaaaattccctaatggctattaaccaaaattggaaagaatttaattttgatttttcaaggtgtgatacttatgaacaaattgaaaaagaaattgatagaaaaggatatattcttaaacaacaaaatatttcattgaatactatcaaaaaatcaatcaatgaagaaaaatcggagaaaataaacaacagcaaaaaattaccaagtttattatttccaattgtaaaccaagatataaacagagaagccaagcaagaaaaaaatcaattcacaaaagattggtttgaaatatttgctgaagaaaataatttacacaaaaagcgtttgcatccagagtttcatttattctatcgctttccaacaaaaaactatccaaatacaaaatttaaaaacggcaaagaaaaatcaaaacgatattctcgctttcaaatgcttgcacattttggtttagaggtatttcctcagggtgattatataagtaaaaaagaacaaatcgaaatttttaatgatgataagaagcaaaaagaagcagttgaaaaatacaataatagtattgtttctgaagttgaatatatcattggtattgatagaggaataaagcaattagccacactttgcgtattaaataaaaatggggtgattcaaagtggttttcaaatttacacacccagttttaatcatgacacaaaacaatgggaacattcttttttaggaaaaagaaatatattagatttatctaatttaagagtggagactactattaaaaacgaaaaagttctagtagatttagcaagtattcaaacaaagaaaggagaaaatcagcaaaaaatcaaacttaaacaactcgcttacattcgtgagttgcaatattctatgcaaaccagacaagtggaattgttagaatatgcaaagactttaaattccgcagaagatattaccgaagaaaaaattaaaatctttatttcaccatttaaagagggaagtcattatgaacatttacccaaacaagaaatatataatttattgaatgaatggcaaaatgcagatgaaacgagaaaacgcaagatacaagaactagaccccactgatagtttgaaatctggaattgtggcaaatatagttggagtgattgctttcttttgtgaaaaatacaactacaaagttcgaatttcattagaagatttaactcgtgcttttagcattcaaaaagatgctttaactggtactcccattcacagaaatgatgaagatttcaaagaacaagaaaatcgaagacttgcaggtgtaggaacaatgcagtttttagaaatgcaactcttgaagaagttgtttaaacttcaatctgaaaaaaataaacatttaattcctgcgtttagaagtgttgctaactatgaaaaaattgttcgtagagataaagagaacggtggtgatgaatttgttaattatccttttggaatagtaacttttgttgatccaagaaatactagtcaaaaatgtccttattgcaataatatagcacgaaaagaagatgatgcattttatagaaatgcaggggaaaataaaaattctctgttatgcaagaaatgtggtttatcaactattaaaggaaaagaaaataagagcaaccaagatgatagtaaaaatcagtttaatattcatttcatcactgacggagaccaaaatggagcttatcatattgctttgaagactttagagaatcttcatcgtttaaatacacctaaagtaacaaagcatactaaaacaaaatggaaaaaatga

SEQ ID NO: 24 - DNA, 3606 bp, synthetic construct
  source 1..3606: mol_type = other DNA
atggagactaacaaaacaacaaaagcaattaatgagtaccaaactcaaaaaacgattagattcggactaacagttacaaataacaatttgtattccgaaaacattgtaaaattattaaaatgctctgaagaaaaaataaaagagcaattaaagaaaacacaaaccgatgatttacaaaaccagaggttaagatgttgtttgattgaaatcaaagaatatctaaaaacgtggaataatgttttttcacaaattgattttttggcaataacaaaagattattacaaagtattatcaagaaaagcaaagtttgattatgataaaggcaatggttcggaaatcaaactttcttccttacaatcaaaacaatcaaagtataatgacaaaaaacgttatcagtatattctggatttttggcatgaaaattttattaaagttgaaaatttgtatcgcaaatcagacgatttattaaaagtatttgaagaagccgcaaatcaaaatcaagatgacaaaaaacttaataaagtggatttgcgaaaaacttttttgagtttattcaatctcgtaaatgaaactctaaaaccgctgattgagggtaatttatttattgtcaatgacgataaaattgacgaacataattcaaagcacaattttgtatcagattttattgtaaaaacagaagaaagaaaacaattgcatgattgtattactgatttacaggatttgttcaaggctaatggcggatatgtgccttttggcagggcgaccattaacaaatggactgctctgcaaaaatctaatcataaagatgatgaaattaaaagaattatcagagagttaaagattgaaaatatctcaatgcaaaatattgattataaatataaatacgatagttttgctgaaaattttaagcaaatatataataaagaaggggagaaagtttgggttttacaatttgatgctaattctgttatcaaagtatgccaatacttcaaatataaaaaagtgccaattaatgctcgtctaaacattgcagaaaggctgataaaagaaaaaagttggcaaagggaaaaaaagaatgattttttaagcgaatttggcatttcaaaatcacctgctttagactacaaaaacgataaagaaaacttcaatctcgcaaactatccgttaaaagttgcttttgattatgcttgggaaaattgtgcaaaagcaatttacgaaacaacaacattcccaaaagaacattgtgaaaaatatttgaaagaggtttttgatttagatatagcaaacaatgcttgttttacaaaatatgctttattgttaagattcaaaatcttaatttgcagaataaaatctgaagaaactactcaaatacaaaatattgaggcagtaagaggtattttagacgaaatcaataaaaatattagtggtaggcaagatttttcaaaagccaaaattattacagaaatcaataattggctttcctttaaagaaaaacaaaccgacaagaaagaaaaatactctaatcaagataacttttctttggcaatgcaaattattgggcaagaacgcggtggtttaaaaagtagaattgaaaaatacaaaactttaacggatatgtttaaagtatgcgcttcaaaatttggaaaacaatttgccgatttgcgcgaatatttccaagaagcgtatgaagttgataaaatcaaatatcgcgcttggattattgaagacgaaaagcaaaaccgttttgttttatttgcgaacaaagaaagggaaattgatttaacgtctgaagaaggtaatttgtatttttatgaagtaaaaagtttaacttctaaatctctcgtgaagtttatcaaaaacagaggtgcttatgcggattttcataaactaaaaaataatttcaattatgaaaaaataaagagagattggcaatactataaaaatgacaagtattttatccaaaatctgaaagatgctttacgcaactccaaaatggctattgaccaaaattgggcagagtttaaatttgatttcacaaagtttaatacttacgaagatatagaaaaagaaattgatagaaaggggtataaacttgtctgcaagacagtttcacttaatacgcttaaagattttgttgaaaacaaaggatgtctcttgttgccaattataaatcaagatattaataaagacgataagcaagccaaaaatcaatttacaaaagattggaatagtatttatgataataaaaagcgtttgcaccctgaatttaacttattctatcgcttcccaacccaagattatccgaatacaaaattcagcaacggaacggaaaagacaaaacgatattcgcgtttccaaatgcttgctcattttggttgtgaatctgttccgaaaggagattatctaagcaaaaaagagcagattgccattttcaacgatgacgcgaaacaaaaagatgcagttgaaaaattcaacaatagcattgcttcagattttgagtatattatcgggattgaccgaggcataaaacaacttgcaacgctttgtgttttgaataaaaacggacagatacaaggagattttgaaatatatacccgaacatttgaaaataaacagtggaaacatactttatcggaaaagcgtaacattttagatttatcgaatttaagagttgaaacaacgattgacggcaacaaagttttagttgatttggcgagtattacgacaaaaaacggtgaaaatcagcaaaaaataaaactcaaacaactcgcttatatcagggaattgcaatattcaatgcaaacaaggagagatgatttgcttgattttgcaaaaggattgcaatctgccgatgatattttgaaagatataagaaatttcattgtgccattcaaggaaggagggcaatatgcagatttacccaacgaaagaatctataatttactaaaagaatggcgagatgccgatgatgaagcaaaacgcaaaatagcagaacttgaccctgcgcaagatttgaaatccggaattgttgccaatatgattggcgtggttgcatttctctgcgaaaaatatggatataaagttcgtatttctttggaggatttaacgagagcatttggcattcaaaaagacgctttaagcggaatagcaattgctccaaatgatgaagattttaaagaacaagaaaatcgtaggcttgccggtgtaggaacttatcagttttttgaaatgcagttgttgaaaaagttattcaaaacgcaggttgataaaaatttacatttagttcctgcttttcgaagtgtggataattatgagaaaattgttcgtagagataaaaagacgaatggcgatgaatatgtaaattaccctttcggtattgtgcgatttattgacccaaaatatacttcaaaaagatgcccgaaatgtggaaaaacagatgttaatcgaaatcaaaagaccaatattgtaaaatgcaataattgtgagtatgaaacaaaagcaggaaattcttccgaagctaataacattcattttattacagatggcgaccaaaatggagcatatcatattgcccaaaaagcattaaaaattcaaaaagaacaataa

SEQ ID NO: 25 - DNA, 3915 bp, synthetic construct
  source 1..3915: mol_type = other DNA
atgaaacagattaaaaatcaatatcaattatcaaaaacgttgcgttttggattgacacaaaaaaacaaaacaaaaaaagaaaattatgctggggaaatctataagagccacagtgaattatctgatttagtagaaatctcagagcaaaggattaaagattctgtatcaacaaataaaaactcagagtcgagtttacctgttgatgctattggtaaatgtcttaatcaaatttctgagttccttaaaggctggcaacaagtatatcagcgtaccgaccaaatagcattagataaggattattataaaatcctttgcaaaaaaattggttttgatggtttttggtttgataagaaaaacggaagaaagacaaaaaagccacaagctcgtattattagccttttagaattggaaaaaaaggacgacaaagaaactgaacgtaaacaatatattcttgattattggcaagaaaattttataaatgctgtggagaaatacaatgttgtcagcgaaaaattaaaacaatttgaagttgcgctaaagataaatagaacggacaataaacccaatgaagtggaatttaggaaactatttctatcgttagtaaatattatttgcgacacacttaaaccgctttgttttcaacagatttgttttccaaggttggaaaaaatagacaattcgaaaatagacaataaaaatttaattgattttgcgatagattaccagtccaaaaacgaattgctttcgctaatatccaaactcaagagttactttgaagaaaacggaggtaatgtgccgtattgccgagctacactcaatcccaaaacagcagttaaaaatccggaatctacagataatagcatagaatcagagataaaaaaacttggacttgataaaattattaaaaataataaagatgcattttccttttcctataatttatacaacaatactgccgaagataaaaaatcaaaattaaaagatgatgaaaatggtggattgatagaacgcagtttactgtttaaatacaagtcgattcctgcaactgttcgattcgaaatagccaaaacattgagcaaaccagacggtaaaaccgaagaagagatattggaatttttacgcgatataggacaactagaaagtcctgcaaaagattatgctgatttaaaggaaaaagacaattttaacatagagaagtatccactgaaagttgcctttaattttgcatgggaaggacttgcccgagcaaaatatcatccagaagctgtttttccgaccgaaatatgtaaacaatatctcaaaaatcattttaagattacagaggataataaagattttgtgatgtatgcaaaactgctggagttgaatgctgttttatctacattagagaaagcaaagcctaccgatgaaaagaaatttagtgttgccgcaaaaaaattattggaagaaatagaatgggaaaaagttgggaaaaatggatccaaaaataaagaagctataacaaaatggttgcagacaaaatctaagacagacaaaaattttaaatcagccaaacaagaaatcggtttgtttagaggtagaataaaaaacaatattagaataaaaaacaatattaagagtgaatactctgaaattacgaatgtatttaagaacatagcagaggaaatgggtaaaacatttgccgaaatgcgcgataaaataagcggtgcggcagaatcaaacaagatttcgcattatgctatgattatagaggataacaataaggacaaatatgttctactacaagaatttgttgaaaataaaaatgaacgaatatatgcaaaatcagatagccaaaagagtgattttaaggcatattcggttaattctatcacttcaggtgcaattgtcaaaatgctcaaaaaaattagaactgacaaattgaaggaaagtaataattttgccaatacacaaccggaattgactagcaaggaaaaagaaaaacgcaatattaaagaatggaaaaagttcattaatgaaaaaggatggaacttggaatttggactaaaattagaaaataaaactttagaagaaattaagaaagaagttgatgccaaatgctataaatttgatattaagtattttgacaaagagactctttccgatttagtgaaaaacaaaaattgtttattactaccaattgtcaatcaagatttggcgaaaaaagaaaaaaacgaaagtaaccagtttacgaaagattggaatgctgtttttcctcaagatacgccttggcgtttaactccggagttcagaatttcttatcgcaagcccacacctaattaccctaaatcggataagggcgataagcgttattcgcgttttcagatgattggacatttcctttgcgattatattccgaaaactgatagctttatttccaaccgccaacaaattgaaaattataaagatgatgaacggcaagaattagcagtaaaaaaatttaatgcagctttgcgagggagaacaaaaaatgaggaatataaagagcaattaaatgaattggcggcaaagtattctaagaatggacagcagaaaataaatgtaaaaactaacgaaaaattttacgttttcggtatcgaccgagggcaaaaagaattggcaacgctttgtattattgaccaagacaaaaaaattatcggtccccataaaatttatacccgttcgttcaactctgaaaaaaaacaatgggaacataaatttttagaagaacggcatattcttgacttgtctaatttgcgtgttgaaactaccgtttttattgatggaaaaccagaaaaaacaaaagtgctggttgatttgagcgaggtgaaagtgaaagacaaagttaccggagaatataccaaacccgacaaaatgcagattaaaatgcagcaacttgcctacattcgtaaacttcaattcaaaatgcagaacgaaccggaagctgtattggcatggtatgaaaaaaattctacagaagatttgattttgaaaaattttgtagataacgaagatggtacgaataacggattggtttctttttatggtgcagccatagaagaactgaaagagactttgcccatagagcgaattgttgatatgcttaaggaattcaaaactataaaaaaagaagagggtaaactcactaaagaggatgaagagggaagggaaaaaaataagcgcaaaatggataaattggtacaattagagcctgttgataatttgaaaaacggtgtcgttgcgaatatggttggtgtgattgcctttttgcttcaaaaatttgattatcaagtttatatctccctcgaagatttgtcaaaaccatttagcagtaaaattatcagtggtattgacggtgttccaattagagttgaaaaagaagaaggacgccgtgctgatgttgaaaaatatgccggactcggactttataatttctttgagatgcaattgctgaaaaaacttttccgcattcaacaggacagtgagaatattctacatttggtaccggctttcagagctatgaaaaattacgaccatatagccgttggaaagggtaaagtaaaaaatcaatttggtattgtgttttttgtagatgcggaggctacttcaaaaacttgtccgcgctgcggttcgactaatcaaaaaccaaacaaaaaagattatcctaatgctcaacaagcaaggttaagcaatgacaaagaagggtggattgaccgtgacaagtcaaatggcaacgatattattcgttgttttgtatgcggtttcgatacaacaaaggaatataccgaaaatccattgaaatacataaatagtggtgatgacaatgcggcgtatttgatttctgccgaaggcgtcaaagcttatgaattggcaacaacgttagctgataatatataa

SEQ ID NO: 26 - DNA, 3879 bp, synthetic construct
  source 1..3879: mol_type = other DNA
atgaaaaacattacaaacaagtatcaaattactaagacattacgttttggcctatcacaaaaagggaaaacaaaaaaagaaggatttgatggagaaatttatcaaagccatcaagaatttaataaattggttagcgtttctgaagcaaggattaaaaaaagtgtaacgacagaacaaaaaacagaattggctttatcaattgataatgttgcacgttgtttaaataacataagtgattttcttataaattggcaacgggtgtattaccgaactgaccaaattgcattagacaaagattattacaaaattatgtgtaagaagattggatttgaaggattctggtttgaaacaaatagacgcactcaacaaaaaataaagaagccacaatcacgtataatcagtctttctgcgcttgataaaaaggatggtttaggcaaggaacgcaaacaatacattttagattattggaaagagaatcttttgtctgccgctgaaaaatatgaagttgttagcgaaaaattgaagcaatttcaagatgcattaaatattaatagaacggacaataaaccaaacgaaattgagcttcgtaaactattcttatcgctcacccatattgtttatgatatacttcagccactttgttatggtcaaatttgttttcccaaaatcgagaaacttgacaacacaaaagaagacaacaaaaagttgattgaatttgcttccgattatcaatcaaaaagcgatttactatccgaaatcgcagaattgaaacaatattttgaggaaaatggtggtaatgtacccttttgccgagccacattaaatccaaaaacacttgtaaaaaatccaaaatcaactgacaatagtattaatgaagaaataaaggatttaggattaaaagagatcttgaaaacatacaaagatgtcttaaactacaacaactatctcgaaagtctatccgcaaaacaaaagctccaattgcttaacgacagaaacacaagtataataacacgcagtttgctgttcaaatataaaccaatttcagccaatgtacaatttgatatagctaaaactctaagcccggaagttggcaagggtgaagaagatttgcgtgctttcttacgtggaattggtcaacctaaaagccctgcaaaagattatgctgatttacaaaacaaatccgatttcaatattgaagcctacccgcttaaagtagctttcgattttgcatgggaaagtttagcaagagcaatatatcatgccgattcagacttgcctatggatgcatgcaaaaattttcttcaagacaattttaaagtaaaaaacgatgatacaaacctcaaattatatgctcaactgcaagaattgaaagctgtgctatcaacattggaaaatggaaatccgaataatgcggctgcttttcgactaaaagctacaaatttgttgaacgagataccttggaagacggttggaaattatggacaacaaaataaagacgaaatttccaaatggttaaataatggtaaaaacaaggatgactataaaaaagcaaaacaacaaataggattattcagaggccgattaaaaaacaacattcaaggttttgataacatcacccaaacgaacaaaaacattgccatgaaaatgggcagaacctttgccacaatgcgcgataaaataaccggcgcagccgaactcaacaaagtgagccactatgctatgattattgaagacagaaacaccgacagatatgttttattgcagccgtttacagaaaacgagcaagacagaatctattcacaaacagattacaacaacggcgattacaccacatacgaagtaaattctataacgtcaggtgcaattgccaaaatgctacgtaaagcaagaatagacgagttgagcaaaaatgacaataacagaaacctcacttcgcaacccgaactgaccgaagaaaaaaaagaaaaacgcaatattaaagagtggaaaaattttattgaaaataaacgttgggatttagaatttcagttaaaattaaatgagaaaaattttgagcaaatcaagaaagaagttgatactaaatgttataatttgagaactaaaaaaattaataaaacaacgcttgaagatttagtaaacaaaagtgattgtttgctgttgcctattgtaaatcaggatttagcgaaagaagaaaaaactaacggcaatcaatttactaaagattggaattcaatttttgcacaaaacactccgtggcgtttgacaccggaattcagggtttcgtatcgtaaaccaactcccgattatccaatatcggataagggtgacaaacgttattctcgtttccaaatgataggtcattttctttgtgattatattccgaaaagtgataaatacatttcaaatagagaacaaattttaaactacaaaaacgacgagttacaaaagaaggcagtcaaagattttcatgaagatttaaaagggaaaaccgaagaagaaaaccaaaatgaatcgatgaatgcgctaatggctaaatttggcaatgtcaataaaaaacagaaagcaacaaccgtagaaaagcccaaagaaaaattttatgtatttggtattgaccgtggacaaaaggaattggctacgctttgcgtgattgaccaagacaaaaagattgtgggcgattttgatatttacacccgcagttttaattcagaacgtaaagaatgggaacatacattctttgaaaaacgccatattttggatttgtccaatttgcgagtggaaaccactgcttcaattgatggaaaagcggaaaagaaaaaagttttggtcgatttgagcgaaattaaagtcaaggataaaaacggcaactattccaaacccgacaaaatgcaaataaaaatgcagcaattagcatacattcgcaagttgcagtttcagatgcagacaaaccctgaaggtgttttagcgtggttcaaagagaattcaacgaaggacttaattattaataatctcgttgataaaaaaaatggtgaaaaaggtttgatttcgttctacggttcggctattgaaaaaatggaagacactttgcccgttgacagaattgaagaaatgcttcaaaaatttgcagctttgaaaaaacaggaaaaagaaggtgaagatgtaaaactatccattgatcaacttgtgcaattagagcctgttgataatttgaaaaacggcgtcgtagcaaatatggttggcgtaatcgcctatctgttgcaaaaatttaattatcaagtatatatatcattagaagacttatcgaatccatttggaagtcaaataacaggtggaattgcaggcgtaccgttgaagcaaggtaaagacgaaggaagacggatggatgtagaaaaatatgccggtcttggtctgtataatttctttgaaatgcaactactcaaaaagctattccgtattcaacaagatagttgtaatattttacatttggttcctgcttttagagcccaaaaaaactatgaccacgttgccgtaggaaaagaaaaggtaaaagggcaatttggaatcgttttctttgttgatgccaatgccacttcaaagacttgccctgtttgcggaacgacaaataataagccaaacaaccaaaagtatcctaatgcgaaaaaaggactttcagcagatggaaaagaagtttggttggaacgcgacaaatcgaatggaaatgatattatccgttgttttgtttgtaactttgatactacaaaagaatatacagaaaaccctcttaaatacattaaaagcggcgacgacaatgcagcttatctaatttcggcggctggaataaaagcatacgaattagcaacaacactcataaacaaccaataa

SEQ ID NO: 27 - DNA, 3786 bp, synthetic construct
  source 1..3786: mol_type = other DNA
atggaaacattaaatcaattcacgggactttattccctgtcaaaaactatgcggtttggtttgacgctcaaagaaaagaaacccaaaaacgattccatagcagtggaatccctctatcaaagccaccaagatttgaaagagttggttgagttatcggacaagagaattatcgaagaaaaaaagccagaaccacctgttgaaaatcttggcaatccgccgattgagaaactacgcgattgtctgaattcgatgcaaaagtatctcaatgattggcgaaaagtctatacaagatatgaccaactcgcagtcttgaaggatttttatagaaaattagaaagaaaagcaagatttgacggattttggaaagacaaaaaaggacaaaatcagccccaatcgcaagagataaaactctcctcactcaagcacaaaagcggagaaaaggaaatcaaagattgcattgtcacatattggggagaaaacatacgaaaggcaaacgagaaatggcatcaggttgattcggttttaaagcaatttgaagaggcaaagcgcaaaaacagagatgacaaaaaactcaatcaagttgaacttcgtaagttgtttctgtcattggcgaaccttgtcaatgatacacttgtaccgttatgtcagagatctatcactttcccaaatgcggataaactctccgacaatgcaagagacaaaagcgtactcgactttattggtgacaacgagatcagagaacatctgcttgataagattaccaagctcaaagagtattttcaagacaatggtggctatgtgccatttggtcgcgtaactctcaaccaatatacggccatgcagaaaccaaacaagaccgacaaagagatagaggacgcaatcaaaaacttaggactttcaatcataaaatcacaaaactttgatgcctttgagcacatagaagaggcgacagacaaagtggaaaggcttaatacggtatctttgccccttgtggagcgggcgcaatacttcaaggacaaaacgattcctgtcggagttcgtgattcattggcaaaatatttggcgaaagacgacactgctaaagaaaaagaacttatcgatttgtttgaaaaaataggcatgcccaaaagacccgcaaaagactacagtgatccaactctcaaagagaagtttgacctgcgcaaatatccgctcaaggttgcatttgattacgcttgggagacagtagcaagcaaagagttacacgatgatattttgaaaaacaaatgcaaaaaatatttgaaagatatttttgacgtcgatactgataaatccatatttttcaacatctattccgatcttaattatatgaaaatcattttatcaagaatcgaatacccaactcaaaatcaactatcaaaagataattttcttgaatggaatagaaaagtaataactatcttagatggcgacgactttagccacttcaataaaaatgcagatggctcaaccgacaagaaaatgaatacagcaaaaacctatgtcaaaacgtggcttgacaaacttgaagccaacatagaacaattcgacggacaagacttcaaaaaattttatgaggattttaaaaagaaaaataagaattcatgtaaagattttgatgacgcaaaaagggatataggattaaagcgcggcggattaaaacaaatcattgaagagacagaaacttttacggacaaaaaaacaggaaaacaaaaaccaaaatacaaagacagcaaatacaaggaattaaccgaggcattcaagagtattgccgtggattttggcaaacattttgccacccttcgcgacaaattcaatgaagaaaacgaaatcaacaagattgaatactacggtgttatcgtcgaagatgaaaatgccgatcgctatcttttgctttcaaaactaagcgaaagtcgcgaggagataaaaaatatctttcctgataaagcagaggggttgaaaacctacaaggtaaaatctctaacctcaaagacactaacaaagctcgtcaaaaacaagggggcatacaaagattttcatatatccgacatgcgcgtagattttaaaaaaatcaaagaagagtggagtgcctacaaaaacgatcaggcttttttgaaatacctcaaaaaatgcctcaccgattccagcatggcacaagctcagaattggtctgaatttggcttggattttgacaaatgcaacacttacgaagaggtagaaaaagagctcgacggcaaagcatatctgctgcaagaaacgcgcctctccaaagcaacaatcactaacttggtcaaaaacaaaggctgctacctcttgcctatcatcaatcaagatttggcgcgagaagaccgcacggcaaaaaatcaatttaccaaagattggaagcagatatttgaaaacaaaaaacattatcgcctgcatccggagttcaatatggcatacagacagccgaccccgaactaccccaactcagagatcggcgacaaaagatattcgcgctttcagatgattgcaaattttatgtgcgagatagttccgcaaagcacaagctacgctacgcgcaaagagcaaatccaaaccttcaacgacaacaataaacaacaaaaagccgttaaagactttgacagcaaatttaaactctccgacagctattttatctttggtatcgacagaggcatcaaacagttggctacgctttgcgtattggatcaaggcggagttatacggggtggatttgaaatctacacgcgacatttcgacggtaataaaaagcagtgggtccatacctctctggagaggcgaaatattttggacttaacaaatctgcgtgcggaaaccacaatagatggtaaaaaagtgctagtagatttgagcaaagtagagatcaaaaatcaaacagacaacaagcaaaatatcaaactcaagcagcttgcttatatcagaaaattgcaatatcaaatgcaaaccaaccccgaaaaagtaaagaatatgtctgatgaagatatcgaaaatgacctaaaagatattattactccatataaagaaggaactcattatgctgatttgccgatagagaatatcaaagcaatgctggatcgcttcaaagttctctacggcaaaaccgaccagcagtccaaacaagaactgaaagagctttgcgagctggatgccgcagataatctcaaaggcggaatagtggcaaacatggtgggtgtcattgcgcatctgatggagcaatacaactacagggtcaagatttcactcgaaaacctaacaacatcatttgtcaaccaatcagatgggcttaacgagtatttcatttcgcgaggtatggatttcaaagaacaagagaatgcggcattggcaggtttgggaacataccaattttttgagatgcaactgctcaaaaaaatattccgcatacaacaagatgatggtaatgttttacatttagttccggcatttagaagcaaagaggattatgaaaaaatcattcggagagacaaaaatgatggcgatgagtatgtaaattatccgtttgggctggtaacttttgttgatccaagatatactagtcgcaaatgccctatatgcggaaaaacagatgtaaaaagaaatgataatataatcacttgcaaaaaatgcggtgccgtatcaggaaaatactcattcgatgataaaaataggcaattcatcaccaacggcgacgaaaacggcgcatatcatatagcattaaaaacaagaaaggaggtgcacaatgaaaactaa

SEQ ID NO: 28 - DNA, 3672 bp, synthetic construct
  source 1..3672: mol_type = other DNA
atggataaagaaaatagttttaaagggtttacgaatttgtatgaagtaaggaaaacagtgaggtttggactaacacaaccaaataaaaaatgagaattaaaaactcatttagaatttgatgatttaataaataaatcttttgaaaacataaaaaaagatgtaaaatcaagagataaaccaaattttaaagaaaaagaactaattgaaaaaataaatcagtttattaattgattagaaaaacaattatgaaattggaaacaaatttatgaaagatatgatgtaatatctgtaaataaagattactataaaatacttgcaagaaaagcaaaatttgatgcttttaaaaaagataaaaaaccacaagcaagtcaaattaaattatcatcattacagaaagataatagaaaagataatataataagatattggtgaaacattattacaagaagtgattatttaataaatatttttaaaccaaaattagaacaatatttaaatgctgttaataatccaaataatagttctcatactaaacctgatttaatagattttagaaaagtatttttacaatttttaaaagtaaatgaagaatatttacaacctctatttgataaatctatacaatttgaaacttgaaaaaaagaaaattctgaagagatcaaaaaaattaatactttttcttgagatgaaaataataaagaaattaattatttgattgatttatgaaaagaaattagagaatattttgaagcaaattgaagtcaagtgccttactgaaaagtgagtttaaattattatacagcattacaaaaaccaaataattttggtgaagatattcgaaaatgagttgaaaatttatgaataataaaatttttgaataaaagtgaagaagatataaaaaattatttaaaacaaaattcaaaagaaaaaataaatttattaaataatgcaaaaaatcattactttattgaattaatacatctttttaagccaaaaacaattcctttttcagtaaagtataatttagcaaaatatttagaaaaaaattttaatttaaaatatgaagatattttaaataaatttgatttactttgaaagtcagttgatatttgaaaagattatcttgaatgtaaagaaaaagaaaagttttcacttgaaaaatatcctattaaatcagcttttgattattcttgggaaaacttagcaagaaatttaaaaagagatgttgattttccaaaaagtgtttgtgaaaagtttttaaaagataattttgatataattattaataatagtagctttaatttatatgcaaatttgctttttattgctgaaaatttggcaacaatagaatattgaaatccaaataacgaaaatgaaattatagagagtataaaaaatacatttgatgatataaaatttgaatctaataaacaagagtatgattgatataaaaaagaaattttaaatattctaaatcaagagaaaagtaaaagaaattataaaaatatattaacagcaaaacaaagattatgattattaagatgacaacaaaaaaataaaatttcaaaatattataatttaacacaatctttcaaaaaaatagcaagttttatttgaaaaactttggctacaataagagaatgattaaaagaagaaaacgaattaaataaaattactgattattgaataataattgaggataaaaatcaagataaatatattttaactttaaaacttgattgaaaagatataagagaaaaaataaaaagtaagttatgggattgagaatataaagtttttgaaattaattcttttacatcaagggcactcaataaatttataaaaaatcccttatgagaagactcaaaaaaatttcattgagattataaatataaacataaggaagtttcaatttacaaagatgtaaaatggatttgatataaagaagaatttttaattcatttaaaagattctttagtaaattctcaaattgcaaaagaacaaaattggaaagctttttgatggaattttgataattttaatacttatgaaaaaattgaaaaagaaattgataaaaaatgatataaattaataaaaaactctatttcaaaagaaaacttagaatacttaataaatgaagaaaaatgtttattatttccattaataaatcaagatatttcaagtaaaaaagaacaaaataaaaatgaatttacaaaagattttaacaaagcatttttatgaatttgatatagaatacatccagaatttagtattttttacagacaacctgatgaagaaaacaaaaaaataaataaatcttgaattataaaccgtttctgaagattgcaattacttgcaaatatttgaattgaatatattccacaaaataatgattataaaacaagaaaagaacaaaataaaatttcattagaccaaacaaatcaaaacgaattagttcaaaattttaataaagaaaaagtaaataaatattttgatagtttagatgattattatattttttgaattgataggtgaataaaacaattagctacattatgtattacaaacaaaaattgaattattcaaagttatgagatttatacaaaatattttaataataattctaaaaaatgggaatataaaaagaatagaattgaatgaattttagatttaacaaatttaaaaattgaatcagataaagattgaaataaatttttagttgatttgtctctatttgaagcaaaagatgaaaattgaaattcaacttgaacaaataaacaaaatataaaattaaagcaattagcttatataagaaagttacaatatcaaatgtcttcaaatgaaaaatgagttttgaattttttaaaaaagtatcaaacaaaagaagaaagacaaaataatataaaagaattaataactccttacaaagaatgacatcattttgaagatttgccagtaaatatttttgaagaaatgtttgaaaactatgaaaagttgaaaaatgataaaactttatcagaaatagaaaaacaaaatttaatgaaacttacaattgagcttgattctagtgaagatttaaaaaaatgagttattgcaaatatgatttgagtgatagtttatttaatgaaaaaatatgattataaagtaaaaattgcagttgaaaatttaaatcaatcttttatgtgacaaaatgattgattaaataacagttatatttcaataaaaacaaattttaaagatcaagaaaattgagctttagcttgaatgtgaacttatcatttttttgaaaatcagttattaagaaagttatataaagtttcggttgaagaatgaatattacatttagttccattttttaattctttagataatgtaaataaattaaattttgaaaaagaaaaaattttatgggttcaaactgaaaactatagaaagttttgaatagttagttttgtaagaccacataacacaagtaaaagatgtcctatttgtaaatcaataaatgtaaagagaaaagataatattacaacttgtagtgactgttgatttataacttgaaaagataataatatagttataaaaaaatataaaaaagaatgattaaatttagatttaattaaaaattgagatgataattgagcttataatatatgttgtaaaatttgactctaa

SEQ ID NO: 29 - DNA, 3720 bp, synthetic construct
  source 1..3720: mol_type = other DNA
gtgaggtttgggcttacccaaccaaataaaaaatgagaattaaaaactcatatagaatttagtgatttagtaaataaatcttttgagaatataaaaaaagaggtaaactcaaaagataaatcaaaatttgatactagaaaagaattgattgataaaataaatcagtttatttcttgattagaaaatcagttatgagactggaagaatatgtatgaaagatatgatttaatatctgtaaataaagattattataaaatacttgcaagaaaagcaaaatttgatgcttttaagaaagataaaaaatgagttaaacaaccacaagctaatcaaattaagttgtcatcattaagatataataaagaattaataataaattattggtgaaatatcatttcaagaagtgattatttaataaatgtttttaaaccaaaattagaacaatatctaaatgctgttaataatccaaataatagttctcatacaaaacctgatttaatagattttagaaaagtatttttacaactgttaaaaataagtgaagaatatttacaacctttatttaataaatctatacaatttgaaacatgaaaaaaagaaaattcttgagatattaaaagagtgaatgatttttcttgaaatgaaaataataaagaaattaatgatttgcttgatttatgaaaagaaattagagaatactttgaagcaaattgaagtcaagttccttattgaaaagttagtttaaactattatacagcagttcaaaaaccaaataattttgataaagaaatcaaagaatgaattaaagatttatgaataatagagtttttaaagaaaagcgaagaggatataaaaaattatttaaaacaagattcaaaagaaaaaatatatttattaaataattcaaaaaatccttactctattgagttaatacaactttttaaaccaaaaacaattcctttttcggtaaaatataatttatctaaatatttagagaagaattataatttaaaatatgaagatattttaaataaatttgatttactttgaaaatctgttgatatttgaaaagattatcttgaatgcaaagataaagaaaaattttcacttgaaaaatatcctattaaatcagcttttgattattcttgggaaaatttagcaagaagtctaaaaagagatgttgattttccaaaaaatgtttgtgaaaaatacttaaatgataattttaatataaatgtttgaaattcaagttttaatttatatgcaaatttactttttattgctgaaaatttagcaacaatagagtattgaaaaccaaataatgaaaaagaaattattgatagtattaaagaaactttcttagaattatcagatgaaatagaaaaaaataataaaaaaaatgaagttgaaaatattataaaatacttaaatttaaacaccgatgaaagaaaaaatattaaagacttacaaaaaaagtattttaaaaatttagatactaaagaacaaaatattctaaatatatttgatagttttacaaaatcaaagcaatctttatgacttctaagatgacaacaaaaaaataaaattgataaatatagaaatttaacacaaaagttagttgataaaaaggattctcatatttgaatagcaagttttatttgaagaactttggcttcaataagggaatgattaaaagaagaaaatgaactaaataaaattactgactattgaataataattgaagataaaaatcaagacaaatatattttaactctaaaacttaacggaaaagatacaagagaaaaaataaaaaataatttatgaaattgagaatataaagtttttgaaataaattcttttacatcaaaagcactcaataaatttataaaaaatcctttatgagaagattcaaagaaatttcattgatattttcaatataaacatagagaagtttcaatatatgatgaaaatgaaaaatgggtttgatataaagaagagtttttgaaacatttgaaacattctttaataaattctcaaattgcagtagaacaaaattggaaagatttttgatggaattttgataattgtgatacttatgaaaagattgaaaaagaagttgataaaaaatgatataaattaatagaaacctctatttcaaaagaaaatttagagaatttaatacataaagaagattgtttattatttccattgataaatcaagatatttctagcaaaaaagaggaaaataaaaatgactttacaaaaaattttgaaaaagtatttttatgagattgatatagaatacatccagagtttagtatattttatagacaaccaaatgaagaaaatttaaaaccaaacaaatcttgaattataaatcgtttttgaagattacaattacttgcaaatatttgagttgagtatattccacaaaacaatgattacacaacaagaaaagaacaaaataaaatttcaatagatcaaacaaaacaaaatgaatcagttcaaaaatttaacaaagaaaaagtaaatccatattttgatagtttagaagattattatattttttgaattgatagatgaattaaacaacttgcaactttgtgtattacaaataaaaaatgagttattcaaaactttgatatttatacaaaacattttaatgataattctaaaaattgggaatataaaaataatagaacagaatgaattttggatttaacaaatttaaaagttgagtcagacaaagaatgaaataaatatttagttgatttatctttatttgaagcaaaagatgaaaattgaaatctaacttgaacgaataagcaaaatgtaaaattaaagcagttagcttatattagaaaacttcaatatcaaatgtcttccaatgaagaatgagttttaagttttttaaataaatataaaacaaaagaagaaagacaaaataatataaaagagttaataacaccatataaagagtgacatcattttgaagacttaccgatgaatatttttgaagaaatgtttgaaaattatgaaaaattgaaaaataataaaactttatcagaatgagaaaaacaaaatttaatgaaactaacaactgaacttgatgcaagtgaagatttgaaaaaatgagttgttgcaaatataatttgagtaatagttcatttaatgaaagaatatgattataaagtaaaaattgcaattgaagatttatcaaatgcttggtatttttcaaaagattgattatcttgagattcaatactaaattccaaaattgatgaagaaatggatttaaaaaaacaagataatttggctttagcttgagtttgaacttaccatttttttgaaatgcagttatttaaaaaattatttaaaatttctgttgaaaaatgaattttacatttagttccaagtttttgaaatgtaagaaattatacagatttattgaaagaaaaatacaaataccaatatcaacaattttgagttatttattttataagcccaaagtttacaagttcaaagtgtcctatctgctgaaaatgatgaaaaaaacatattaagagagaaaacaatgtaataacttgtaaagaatgctgatttgtttcttgaaaagataattcaataaatattaagaacaataaaaaagaatgactaaatttagatttaattaaaaattgagatgataatggatcttataatatttgatgaaaaattaagtaa

SEQ ID NO: 30 - protein, 31 aa, synthetic construct
  VARIANT 2: note = Any amino acid
  VARIANT 3: note = Any amino acid
  VARIANT 4: note = Any amino acid
  VARIANT 5: note = Y, F, or L
  VARIANT 6: note = Any amino acid
  VARIANT 7: note = Any amino acid
  VARIANT 8: note = Any amino acid
  VARIANT 9: note = D, G, or N
  VARIANT 10: note = Q, L, F, or M
  VARIANT 11: note = I, L, V, or M
  VARIANT 12: note = Any amino acid
  VARIANT 13: note = L, I, or V
  VARIANT 14: note = Any amino acid
  VARIANT 16: note = D, E, or S
  VARIANT 17: note = Y or F
  VARIANT 19: note = K, R, L, or S
  VARIANT 20: note = Any amino acid
  VARIANT 21: note = L, I, or M
  VARIANT 22: note = Any amino acid
  VARIANT 23: note = K, R, or S
  VARIANT 24: note = K or E
  VARIANT 25: note = A, I, L, or V
  VARIANT 26: note = Any amino acid
  VARIANT 28: note = D, E, N, or V
  VARIANT 29: note = A, G, F, or V
  VARIANT 30: note = F, M, or I
WXXXXXXXXXXXXXKXXYXXXXXXXXFXXXW

SEQ ID NO: 31 - skipped

SEQ ID NO: 32 - protein, 20 aa, synthetic construct
  VARIANT 2: note = N, S, or D
  VARIANT 3: note = L or I
  VARIANT 4: note = Any amino acid
  VARIANT 5: note = K, N, H, or A
  VARIANT 8: note = I or L
  VARIANT 10: note = V or S
  VARIANT 13: note = D or N
  VARIANT 14: note = Y or F
  VARIANT 15: note = A or S
  VARIANT 18: note = Any amino acid
  VARIANT 19: note = L, C, or V
FXXXXYPXKXAFXXXWEXXA

SEQ ID NO: 33 - skipped

SEQ ID NO: 34 - protein, 13 aa, synthetic construct
  VARIANT 1: note = Y, C, or S
  VARIANT 2: note = Any amino acid
  VARIANT 3: note = I or V
  VARIANT 4: note = Any amino acid
  VARIANT 6: note = F, L, I, or V
  VARIANT 9: note = Any amino acid
  VARIANT 10: note = Any amino acid
  VARIANT 11: note = L or I
  VARIANT 12: note = Any amino acid
XXXXSXTSXXXXK

SEQ ID NO: 35 - skipped

SEQ ID NO: 36 - protein, 9 aa, synthetic construct
  VARIANT 1: note = L, S, or F
  VARIANT 3: note = L, F, or V
  VARIANT 5: note = I, F, or L
  VARIANT 6: note = I or V
  VARIANT 8: note = Q or K
XLXPXXNXD

SEQ ID NO: 37 - protein, 9 aa, synthetic construct
  VARIANT 1: note = L or I
  VARIANT 2: note = H or T
  VARIANT 6: note = Any amino acid
  VARIANT 7: note = I, V, L, or M
  VARIANT 8: note = F, S, or T
XXPEFXXXY

SEQ ID NO: 38 - skipped

SEQ ID NO: 39 - protein, 14 aa, synthetic construct
  VARIANT 4: note = R or S
  VARIANT 5: note = G or W
  VARIANT 6: note = I, L, or Q
  VARIANT 7: note = K or N
  VARIANT 8: note = E or Q
  VARIANT 11: note = T or V
  VARIANT 14: note = I, L, or V
GIDXXXXXLAXLCX

SEQ ID NO: 40 - protein, 25 aa, synthetic construct
  VARIANT 1: note = R or E
  VARIANT 2: note = Any amino acid
  VARIANT 7: note = S or T
  VARIANT 8: note = N, D, or Y
  VARIANT 10: note = R or K
  VARIANT 11: note = V, I, or A
  VARIANT 13: note = T, S, or K
  VARIANT 14: note = T or D
  VARIANT 15: note = Any amino acid
  VARIANT 16: note = E, D, N, or K
  VARIANT 17: note = G, K, or N
  VARIANT 18: note = K, N, E, or T
  VARIANT 19: note = K, S, or Q
  VARIANT 20: note = V, R, F, or Y
  VARIANT 24: note = L or Q
  VARIANT 25: note = S or A
XXILDLXXLXXEXXXXXXXXLVDXX

SEQ ID NO: 41 - skipped

SEQ ID NO: 42 - protein, 22 aa, synthetic construct
  VARIANT 1: note = E or Q
  VARIANT 3: note = D or E
  VARIANT 4: note = Any amino acid
  VARIANT 5: note = Any amino acid
  VARIANT 6: note = D, E, or Q
  VARIANT 7: note = N, D, Y, or S
  VARIANT 8: note = L or F
  VARIANT 10: note = Any amino acid
  VARIANT 12: note = V, I, or A
  VARIANT 13: note = V or I
  VARIANT 16: note = M or I
  VARIANT 17: note = I or V
  VARIANT 19: note = V or I
  VARIANT 20: note = I or V
  VARIANT 21: note = A, V, or N
  VARIANT 22: note = Y, F, or H
XLXXXXXXKXGXXANXXGXXXX

SEQ ID NO: 43 - skipped

SEQ ID NO: 44 - protein, 16 aa, synthetic construct
  VARIANT 2: note = G or W
  VARIANT 3: note = L or V
  VARIANT 4: note = G, W, or E
  VARIANT 5: note = T or L
  VARIANT 6: note = Y or M
  VARIANT 7: note = Any amino acid
  VARIANT 8: note = F or Y
  VARIANT 9: note = F, L, or M
  VARIANT 11: note = Any amino acid
  VARIANT 12: note = Q or L
  VARIANT 14: note = L or V
  VARIANT 15: note = Any amino acid
AXXXXXXXXEXXLXXK

SEQ ID NO: 45 - protein, 21 aa, synthetic construct
  VARIANT 2: note = Any amino acid
  VARIANT 3: note = Any amino acid
  VARIANT 5: note = I or V
  VARIANT 6: note = I, F, or V
  VARIANT 7: note = Any amino acid
  VARIANT 8: note = F or Y
  VARIANT 9: note = V, I, or T
  VARIANT 10: note = Any amino acid
  VARIANT 11: note = P or A
  VARIANT 12: note = Any amino acid
  VARIANT 13: note = Any amino acid
  VARIANT 15: note = S or T
  VARIANT 16: note = Any amino acid
  VARIANT 17: note = Any amino acid
  VARIANT 20: note = Any amino acid
FXXGXXXXXXXXXTXXXCPXC

SEQ ID NO: 46 - protein, 12 aa, synthetic construct
  VARIANT 2: note = Any amino acid
  VARIANT 3: note = Any amino acid
  VARIANT 4: note = G or W
  VARIANT 6: note = D, Q, or E
  VARIANT 7: note = N or S
  VARIANT 8: note = G or A
  VARIANT 10: note = Y or F
  VARIANT 11: note = H, L, I, or N
IXXXDXXXAXXI

SEQ ID NO: 47 - RNA, 14 nt, synthetic construct
  source 1..14: mol_type = other RNA
  misc_difference 6: note = a, c, u, g, unknown or other
  misc_difference 7: note = a, c, u, g, unknown or other
  misc_difference 8: note = a, c, u, g, unknown or other
tctacnnngtagat

SEQ ID NO: 48 - RNA, 15 nt, synthetic construct
  source 1..15: mol_type = other RNA
  misc_difference 6: note = a, c, u, g, unknown or other
  misc_difference 7: note = a, c, u, g, unknown or other
  misc_difference 8: note = a, c, u, g, unknown or other
  misc_difference 9: note = a, c, u, g, unknown or other
tctacnnnngtagat

SEQ ID NO: 49 - RNA, 16 nt, synthetic construct
  source 1..16: mol_type = other RNA
  misc_difference 6: note = a, c, u, g, unknown or other
  misc_difference 7: note = a, c, u, g, unknown or other
  misc_difference 8: note = a, c, u, g, unknown or other
  misc_difference 9: note = a, c, u, g, unknown or other
  misc_difference 10: note = a, c, u, g, unknown or other
tctacnnnnngtagat

SEQ ID NO: 50 - DNA, 14 bp, synthetic construct
  source 1..14: mol_type = other DNA
  misc_difference 6: note = a, c, t, g, unknown or other
  misc_difference 7: note = a, c, t, g, unknown or other
  misc_difference 7: note = a, c, t, g, unknown or other
tctacnnngtagat

SEQ ID NO: 51 - DNA, 15 bp, synthetic construct
  source 1..15: mol_type = other DNA
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
tctacnnnngtagat

SEQ ID NO: 52 - DNA, 16 bp, synthetic construct
  source 1..16: mol_type = other DNA
  misc_difference 6: note = a, c, u, g, unknown or other
  misc_difference 7: note = a, c, u, g, unknown or other
  misc_difference 8: note = a, c, u, g, unknown or other
  misc_difference 9: note = a, c, u, g, unknown or other
  misc_difference 10: note = a, c, u, g, unknown or other
tctacnnnnngtagat

SEQ ID NO: 53 - DNA, 24 bp, Oryza sativa
  source 1..24: mol_type = genomic DNA
tggagcaacacctgaaggaaggct

SEQ ID NO: 54 - DNA, 3744 bp, synthetic construct
  source 1..3744: mol_type = other DNA
atggcccccaagaagaagcggaaagtgatgctgcacgccttcaccaaccagtaccagctgagcaagaccctgagatttggcgccacactgaaggaggacgagaagaagtgtaagtctcacgaggagctgaagggcttcgtggatatcagctatgagaacatgaagagctccgccacaatcgccgagtctctgaacgagaatgagctggtgaagaagtgcgagcggtgttacagcgagatcgtgaagtttcacaatgcctgggagaagatctactataggaccgatcagatcgccgtgtacaaggacttctatcgccagctgtccaggaaggcccgctttgacgccggcaagcagaactcccagctgatcacactggcctctctgtgcggcatgtatcagggcgccaagctgtctcggtacatcaccaactattggaaggataatatcacaagacagaagagcttcctgaaggacttttcccagcagctgcaccagtacaccagagccctggagaagtccgataaggcccacaccaagccaaacctgatcaacttcaacaagaccttcatggtgctggccaacctggtgaatgagatcgtgatccccctgtctaacggcgccatcagcttccctaatatctccaagctggaggatggcgaggagagccacctgatcgagtttgccctgaatgactactcccagctgtctgagctgatcggcgagctgaaggatgccatcgccaccaacggcggctatacaccattcgccaaggtgaccctgaatcactacacagccgagcagaagccccacgtgtttaagaacgacatcgatgccaagatccgggagctgaagctgatcggcctggtggagaccctgaagggcaagtctagcgagcagatcgaggagtacttttctaatctggataagttcagcacctataacgacaggaatcagtccgtgatcgtgcgcacacagtgtttcaagtataagcccatcccttttctggtgaagcaccagctggccaagtacatctccgagccaaacggatgggacgaggatgcagtggcaaaggtgctggacgcagtgggagccatccggtctcctgcccacgattatgccaacaatcaggagggcttcgacctgaaccactacccaatcaaggtggcctttgattatgcctgggagcagctggccaatagcctgtacaccacagtgaccttcccccaggagatgtgcgagaagtacctgaatagcatctatggctgtgaggtgtccaaggagcccgtgttcaagttctacgccgacctgctgtatatcagaaagaacctggccgtgctggagcacaagaacaatctgccaagcaatcaggaggagttcatctgcaagatcaacaatacctttgagaacatcgtgctgccctacaagatctcccagttcgagacatataagaaggacatcctggcctggatcaatgatggccacgaccacaagaagtacaccgatgccaagcagcagctgggctttatcaggggaggcctgaagggacgcatcaaggcagaggaggtgagccagaaggacaagtatggcaagatcaagtcctactatgagaacccctacaccaagctgacaaatgagttcaagcagatctcctctacctatggcaagacattcgccgagctgagggataagtttaaggagaagaacgagatcaccaagatcacacactttggcatcatcatcgaggataagaatcgggacagatacctgctggcctccgagctgaagcacgagcagatcaaccacgtgtctaccatcctgaataagctggacaagagctccgagttcatcacatatcaggtgaagagcctgacctccaagacactgatcaagctgatcaagaaccacaccacaaagaagggcgccatctccccttacgccgacttccacacctctaagacaggctttaacaagaatgagatcgagaagaactgggataattacaagcgcgagcaggtgctggtggagtatgtgaaggattgcctgaccgactctacaatggccaagaaccagaattgggccgagttcggctggaactttgagaagtgtaatagctatgaggatatcgagcacgagatcgaccagaagtcttacctgctgcagagcgacaccatctctaagcagagcatcgcctccctggtggagggaggatgcctgctgctgccaatcatcaaccaggatatcacatctaaggagaggaaggataagaaccagttcagcaaggactggaatcacatctttgagggctccaaggagttccggctgcacccagagtttgccgtgtcctacagaaccccaatcgagggctaccccgtgcagaagcggtatggcagactgcagttcgtgtgcgcctttaacgcccacatcgtgccccagaacggcgagttcatcaatctgaagaagcagatcgagaactttaatgacgaggatgtgcagaagcgcaatgtgaccgagttcaacaagaaagtgaatcacgccctgagcgataaggagtatgtggtcatcggcatcgacaggggcctgaagcagctggccacactgtgcgtgctggataagcgcggcaagatcctgggcgacttcgagatctacaagaaggagtttgtgagggccgagaagcggagcgagtctcactgggagcacacccaggccgagacaaggcacatcctggacctgtccaacctgcgcgtggagaccacaatcgagggcaagaaggtgctggtggatcagtctctgaccctggtgaagaagaacagggatacccccgacgaggaggccacagaggagaataagcagaagatcaagctgaagcagctgtcttatatccgcaagctgcagcacaagatgcagacaaacgagcaggatgtgctggacctgatcaacaatgagcccagcgacgaggagttcaagaagcggatcgagggcctgatctctagctttggcgagggccagaagtacgccgatctgcctatcaataccatgagggagatgatcagcgacctgcagggcgtgatcgcaaggggcaacaatcagacagagaagaacaagatcatcgagctggatgccgccgacaatctgaagcagggcatcgtggccaacatgatcggcatcgtgaattacatcttcgccaagtacagctataaggcctatatctccctggaggacctgtctagggcatacggaggagcaaagtccggatacgatggcagatatctgcctagcacctcccaggacgaggatgtggactttaaggagcagcagaaccagatgctggccggcctgggcacctaccagttctttgagatgcagctgctgaagaagctgcagaagatccagagcgacaatacagtgctgcggttcgtgcctgcctttagatccgccgataactatcggaatatcctgagactggaggagaccaagtacaagtccaagccattcggcgtggtgcactttatcgaccctaagttcacctctaagaagtgccccgtgtgcagcaagacaaacgtgtacagagacaaggacgatatcctggtgtgcaaggagtgtggcttccggtctgatagccagctgaaggagagagagaacaatatccactatatccacaacggcgacgataatggcgcctaccacatcgccctgaagagcgtggagaatctgatccagatgaagcaccaccaccaccaccactaa

SEQ ID NO: 55 - protein, 1256 aa, synthetic construct
MNQEVQGKQYQFSKTLRFGLTSTNQNLYSEETMRLLKVSQEKIEKQVKKENNNTDKTNQLRNCLVQIKEYLKTWDNTYPQIDFLAITKDYYKVISRKARFDFDKGNGSEIKLSSLQSMYNNKKRYQYITDFWKENLHKTENLYRKSDDLLRIFEEAEKQNREDKKLNKVELRKTFLSLFNLVNESLKPLIEGNLFIVNDEKIDEQNPKHNYVSDFILKAEARKPLYNCIGNLQNYFKDNGGYVPFGRVTLNKWTALQKSNNRDTKINRIIKELKINSFLIKNINYKYNEFTSNFKEKKDKKGKIVKNKDGDIVWELEPNDKSVIELCQFFKYKKIPINACLNLAKRLIKENKLEKEKENTFLSELGVSKSPALDYKKDQSNFSLTNYPLKVAFDYAWENCAKAKYEDIPFPKKQCEKYLRDVFDLDIETNADFAKYALLLRFKILIGRIKVEETTRIENIATIKEFFNDVKSNLTKEKDKTVAEINNWLTFKENQTDKKAKYSNQDEFSEAMKTIGEERGGLKSKISRYKALTDMFKVCSSKFGKQFADLRDYFNEAYEVDKIKYRAWIIEDDKKNRFVLLADKGKEVGLTSGNGDLYFYEVKSLTSKSLVKFIKNKGAYPDFHNKKSEDGFCQIYLNSENKENKDRFIDDVKIHWSTYKNDQEFLKKLKECLKNSKMAIEQNWNEFNFDFSECDNYEKLEKEIDRKGYKFERKAISLTDITDLVENKECLLLPIVNHDINKEKQTENQSQFTKDWFAIFKNKKHLHPEFNIFYRFQTKDYLKTKFKNGTEKTKRYSRFQMLAHFGCEVIPQGDYLSKKEQIAIFNDDEKQKKEVENFKENISSDFDYVIGIDRGIKQLATLCVLDKKGVIQGDFQIFTRKFNDITKKWEHKELEKRNILDLSNLRVETTIAGEKVLVDLASIKTKKGENQQKIKLKELAYIRELQYAMQTRKDELLDFANKINSADDITEDSIKNFISPYKEGTRYADLPKSEFFNRLTEWKNADDKGKLKVAELDSADNLKSGIVANMIGVIAFLCEKYKYKVRISLEDLTRAYGIQKDALSGTAIYQNDEDFKEQENRRLAGVGTMQFFEMQLLRKLFKIQIDEKLCLIPSFRSVANYEKIVRRDRKSSGDKFVNYPFGIVCFVDPSYTSQKCPYCDNKHKKNDKETGKKAFYRDKGENKNSLLCKQCGVSTIKGQEKPSNKNDSKKQFNIHFITNGDENGAYHIAKKTLNNLIPNNKNNKNQPSDFPIGTCT

SEQ ID NO: 56 - protein, 1256 aa, synthetic construct; identical to SEQ ID NO: 55

SEQ ID NO: 57 - DNA, 24 bp, synthetic construct
  source 1..24: mol_type = other DNA
tggatcaacacctgaaggaaggct

SEQ ID NO: 58 - DNA, 24 bp, synthetic construct
  source 1..24: mol_type = other DNA
tggatcaacatctgaaggaaggct

SEQ ID NO: 59 - DNA, 24 bp, synthetic construct
  source 1..24: mol_type = other DNA
tggatcaacatctgtaggaaggct

SEQ ID NO: 60 - DNA, 24 bp, synthetic construct
  source 1..24: mol_type = other DNA
ttgatcaacatctgtaggaaggct

SEQ ID NO: 61 - DNA, 24 bp, synthetic construct
  source 1..24: mol_type = other DNA
cctacgccagcagctccaactacc

SEQ ID NO: 62 - DNA, 24 bp, synthetic construct
  source 1..24: mol_type = other DNA
cctacgcctgcagctccaactacc

SEQ ID NO: 63 - DNA, 24 bp, synthetic construct
  source 1..24: mol_type = other DNA
cctacgcgtgcagctccaactacc

SEQ ID NO: 64 - DNA, 24 bp, synthetic construct
  source 1..24: mol_type = other DNA
gcccgcccaaaatctgtgatcttg

SEQ ID NO: 65 - DNA, 24 bp, synthetic construct
  source 1..24: mol_type = other DNA
gcgcgcccaaaatctgtgatcttg

SEQ ID NO: 66 - DNA, 24 bp, synthetic construct
  source 1..24: mol_type = other DNA
gggcgcccaaaatctgtgatcttg

SEQ ID NO: 67 - protein, 20 aa, synthetic construct
FSLDNYPIKVAFDYAWEMCA

SEQ ID NO: 68 - protein, 20 aa, synthetic construct
FSLDKYPIKVAFDYAWERCA

SEQ ID NO: 69 - protein, 20 aa, synthetic construct
FDINHYPLKVAFDFAWESLA

SEQ ID NO: 70 - protein, 20 aa, synthetic construct
FNIEKYPLKVAFNFAWEGLA

SEQ ID NO: 71 - protein, 20 aa, synthetic construct
FDLDAYPLKVAFDFAWENLA

SEQ ID NO: 72 - protein, 20 aa, synthetic construct
FNIEAYPLKVAFDFAWESLA

SEQ ID NO: 73 - protein, 20 aa, synthetic construct
FNLANYPLKVAFDYAWENCA

SEQ ID NO: 74 - protein, 20 aa, synthetic construct
FSLTNYPLKVAFDYAWENCA

SEQ ID NO: 75 - protein, 20 aa, synthetic construct
FSLEKYPIKSAFDYSWENLA

SEQ ID NO: 76 - protein, 20 aa, synthetic construct
FDLNHYPIKVAFDYAWEQLA

SEQ ID NO: 77 - protein, 20 aa, synthetic construct
FDLRKYPLKVAFDYAWETVA

SEQ ID NO: 78 - protein, 20 aa, synthetic construct
FDLFQYPLKPAFDYAWENVA

SEQ ID NO: 79 - protein, 20 aa, synthetic construct
FNLYKYPLKVAFDYAWESLA

SEQ ID NO: 80 - protein, 25 aa, synthetic construct
RDILDLSYLRVEKDENGESRLVDLS

SEQ ID NO: 81 - protein, 25 aa, synthetic construct
RTILDLSNLRVETTIDGKQVLVDLS

SEQ ID NO: 82 - protein, 31 aa, synthetic construct
RHILDLSNLRVETTIVIDGKPDVRKVLVDLS

SEQ ID NO: 83 - protein, 31 aa, synthetic construct
RHILDLSNLRVETTVFIDGKPEKTKVLVDLS

SEQ ID NO: 84 - protein, 31 aa, synthetic construct
RVILDLSNLRVETTIVIDGKPEKKKVLVDLS

SEQ ID NO: 85 - protein, 31 aa, synthetic construct
RHILDLSNLRVETTASIDGKAEKKKVLVDLS

SEQ ID NO: 86 - protein, 25 aa, synthetic construct
RNILDLSNLRVETTIKNEKVLVDLA

SEQ ID NO: 87 - protein, 25 aa, synthetic construct
RNILDLSNLRVETTIDGNKVLVDLA

SEQ ID NO: 88 - protein, 25 aa, synthetic construct
RNILDLSNLRVETTIAGEKVLVDLA

SEQ ID NO: 89 - protein, 25 aa, synthetic construct
EGILDLTNLKIESDKDGNKFLVDLS

SEQ ID NO: 90 - protein, 25 aa, synthetic construct
EGILDLTNLKVESDKEGNKYLVDLS

SEQ ID NO: 91 - protein, 25 aa, synthetic construct
RHILDLSNLRVETTIEGKKVLVDQS

SEQ ID NO: 92 - protein, 25 aa, synthetic construct
RNILDLTNLRAETTIDGKKVLVDLS

SEQ ID NO: 93 - protein, 25 aa, synthetic construct
RAILDLSNLRVETTVNGDKVLVDLA

SEQ ID NO: 94 - protein, 25 aa, synthetic construct
RNILDLSDLRVETTVEGKKVLVDLS

SEQ ID NO: 95 - protein, 22 aa, synthetic construct
QLDASEYLKKGVVANMIGVVVY

SEQ ID NO: 96 - protein, 22 aa, synthetic construct
QLDATESLKKGVVANMIGVVVY

SEQ ID NO: 97 - protein, 22 aa, synthetic construct
QLEPVDNLKAGVVANMVGVIAH

SEQ ID NO: 98 - protein, 22 aa, synthetic construct
QLEPVDNLKNGVVANMVGVIAF

SEQ ID NO: 99 - protein, 22 aa, synthetic construct
QLEPVDNLKNGVVANMVGVIAY

SEQ ID NO: 100 - protein, 22 aa, synthetic construct
ELDPTDSLKSGIVANIVGVIAF

SEQ ID NO: 101 - protein, 22 aa, synthetic construct
ELDPAQDLKSGIVANMIGVVAF

SEQ ID NO: 102 - protein, 22 aa, synthetic construct
ELDSADNLKSGIVANMIGVIAF

SEQ ID NO: 103 - protein, 22 aa, synthetic construct
ELDSSEDLKKGVIANMIGVIVY

SEQ ID NO: 104 - protein, 22 aa, synthetic construct
ELDASEDLKKGVVANIIGVIVH

SEQ ID NO: 105 - protein, 22 aa, synthetic construct
ELDAADNLKQGIVANMIGIVNY

SEQ ID NO: 106 - protein, 22 aa, synthetic construct
ELDAADNLKGGIVANMVGVIAH

SEQ ID NO: 107 - protein, 22 aa, synthetic construct
ELDSADDLKTGVVANMVGVIAF

SEQ ID NO: 108 - protein, 22 aa, synthetic construct
ELDAVDDFKTGAVANMIGVIAY

(19) United States
(12) Patent Application Publication
(10) Pub. No.: US 2026/0085301 A1
(43) Pub. Date: Mar. 26, 2026

(54) COMPOSITIONS AND METHODS FOR MODIFYING GENOMES

(71) Applicant: Confluence Genetics, LLC, St. Louis, MO (US)

(72) Inventors: Matthew Brett Begemann, St. Louis, MO (US); Emma Elizabeth January, St. Louis, MO (US); Gina C. Neumann, St. Louis, MO (US); Allison Jane Newton Antonakos, St. Louis, MO (US); Anna Singer, St. Louis, MO (US); Erin Zess, St. Louis, MO (US)

(73) Assignee: Confluence Genetics, LLC, St. Louis, MO (US)

(21) Appl. No.: 19/334,505

(22) Filed: Sep. 19, 2025

Related U.S. Application Data
(60) Provisional application No. 63/697,245, filed on Sep. 20, 2024.

(51) Int. Cl. C12N 9/22; C12N 15/11; C12N 15/82; C12N 15/86; C12N 15/90

(52) U.S. Cl. CPC C12N 9/226; C12N 15/11; C12N 15/8213; C12N 15/86; C12N 15/902; C12N 15/907; C12N 2310/20; C12N 2750/14143; C12N 2800/80

(57) ABSTRACT

Compositions and methods for targeting pre-determined DNA sequences in cells of interest are provided. The methods result in the targeted elimination of cells that comprise the pre-determined DNA sequence(s). Compositions comprise DNA constructs comprising nucleotide sequences that encode a Cas12a2 protein operably linked to a promoter that is operable in the cells of interest. Methods to use these DNA constructs to selectively target and eliminate cells that harbor the targeted DNA sequence(s) are described herein.

### CROSS REFERENCE TO RELATED APPLICATIONS

This application claims priority to U.S. Provisional Application No. 63/697,245 filed on Sep. 20, 2024, the content of which is incorporated herein by reference in its entirety.

### FIELD OF THE INVENTION

The present invention relates to compositions and methods for selectively killing prokaryotic or eukaryotic cells in a sequence-specific manner.

### SEQUENCE LISTING

This application contains a Sequence Listing which is submitted herewith in electronically readable format. The Sequence Listing file was created on Nov. 13, 2025, is named “B88552_1680US_SL.xml” and its size is 227,937 bytes. The entire contents of the Sequence Listing file are incorporated by reference herein.

### BACKGROUND OF THE INVENTION

Modification of genomic DNA is of immense importance for basic and applied research. Genomic modifications have the potential to elucidate and in some cases to cure the causes of disease and to provide desirable traits in the cells and/or individuals comprising said modifications. Genomic modification may include, for example, modification of plant, animal, fungal, and/or prokaryotic genomic modification. The most common methods for modifying genomic DNA tend to modify the DNA at random sites within the genome, but recent discoveries have enabled site-specific genomic modification. Such technologies rely on the creation of a DSB at the desired site. This DSB causes the recruitment of the host cell's native DNA-repair machinery to the DSB. The DNA-repair machinery may be harnessed to insert heterologous DNA at a pre-determined site, to delete native genomic DNA, or to produce point mutations, insertions, or deletions at a desired site. Of particular interest for site-specific genomic modifications are Clustered, Regularly Interspersed Short Palindromic Repeat (CRISPR) nucleases. CRISPR nucleases use a guide molecule, often a guide RNA molecule, that interacts with the nuclease and base pairs with the targeted DNA, allowing the nuclease to produce a double-stranded break (DSB) at the desired site. The production of DSBs requires the presence of a protospacer adjacent motif (PAM) sequence; following recognition of the PAM sequence, the CRISPR nuclease is able to produce the desired DSB. Cas12a2 CRISPR nucleases are a class of CRISPR nucleases that have certain desirable properties relative to other CRISPR nucleases such as Cas9 nucleases.

CRISPR systems have been proposed as a possible technology that may be adapted to selectively eliminate unwanted and/or harmful cells (Gomaa et al (2014) mBio e00928-13), with a focus on Type I CRISPR systems because these CRISPR systems have a processive DNase activity wherein the CRISPR nuclease hybridizes with the target sequence, then processively degrades DNA following this hybridization, sometimes resulting in the near complete elimination of the targeted DNA molecule (e.g., a targeted plasmid, viral DNA molecule, circular bacterial genome, or other DNA molecule).

While these properties of Type I CRISPR systems may be desirable in some applications, Type I CRISPR systems also have some drawbacks. For instance, Type I CRISPR systems are typically large, multi-component systems. Their size can make packaging of Type I CRISPR systems in commonly used plasmids, viral vectors, and other vectors difficult. Furthermore, Type I CRISPR systems may not show optimal activity in some cells that may be desirable to eliminate. While CRISPR systems show promise in their ability to target and eliminate undesirable cells, viruses, or pests, alternatives to Type I CRISPR systems would be valuable. Cas9-based CRISPR systems have been explored for their ability to selectively eliminate bacteria (Citorik et al (2014) Nat Biotechnol 32:1141-1145; Bikard et al (2014) Nat Biotechnol 32:1146-1150; U.S. patent application Ser. No. 14/475,785); however these systems may be hampered by the mechanism of Cas9 nucleases. Because Cas9 nucleases make a single DSB, repair of this DSB may result in survival of the unwanted or harmful cell.

Some Type V CRISPR enzymes have been shown to harbor a primary, sequence-specific, activity against a particular type of substrate; following this sequence-specific primary activity, the Type V enzyme is then able to access a secondary, collateral activity in a non-sequence-specific manner. As an example, Cpf1 (Cas12a) has been shown to harbor primary double-stranded break production activity against double-stranded DNA (dsDNA). After Cpf1 hybridizes with and cleaves its primary target, the protein is then capable of cleaving single-stranded DNA (ssDNA) in a non-sequence-specific manner (Chen et al (2018) Science 360:436-439). Other Type V CRISPR enzymes have been shown, for example, to harbor a primary activity against RNA, with secondary activities directed against RNA and ssDNA (Yan et al (2019) Science 363:88-91). Accordingly, the secondary activities of Cas12a2-like enzymes, a group of Type V CRISPR enzymes, may be used to promote cell death of an unwanted prokaryotic (e.g., bacterial cells) or eukaryotic cell (e.g., undesirable cells in or on plants or mammals).

### SUMMARY OF THE INVENTION

Compositions and methods for modifying genomic DNA sequences using Cas12a2 CRISPR systems are provided herein. The CRISPR enzymes of the invention are orthologues belonging to the Cas12a2 family of nucleases, e.g. a Cas12a2 ortholog. Further provided are compositions and methods for modifying genomic DNA sequences and selectively killing cells using Cas12a2 CRISPR systems. In some embodiments, the methods result in genome modification and/or cell death for cells that harbor particular pre-determined and targeted DNA sequences leaving other cells that do not comprise the targeted DNA sequences unharmed. The compositions include DNA constructs comprising nucleotide sequences that encode a Cas12a2 protein operably linked to a promoter that is operable in the cells of interest. In some embodiments, the compositions further comprise nucleotide sequences that encode at least one guide RNA that can interact with a Cas12a2 protein of the invention and can guide the Cas12a2 protein to bind with a pre-determined DNA sequence. The DNA constructs comprising polynucleotide sequences that encode the Cas12a2 proteins of the invention, or the Cas12a2 proteins of the invention themselves, can be used to direct the Cas12a2 protein to hybridize with genomic DNA in a cells of interest at pre-determined genomic loci, with this hybridization in turn leading to Cas12a2-mediated cell death. Methods to use these DNA constructs to selectively target and eliminate target cells (e.g., bacterial cells or eukaryotic cells associated with disease, such as cancer cells) are described herein.

In one aspect, the present disclosure provides a composition comprising a Cas12a2 polypeptide, wherein the Cas12a2 polypeptide shares at least 85% identity with a sequence selected from the group consisting of any one of SEQ ID NOs: 1-14 and 55.

In one aspect, the present disclosure provides a composition comprising a polynucleotide encoding a Cas12a2 polypeptide, wherein the Cas12a2 polypeptide shares at least 85% identity with a sequence selected from the group consisting of any one of SEQ ID NOs: 1-14 and 55. 24. In one aspect, the present disclosure provides a composition comprising: (i) a Cas12a2 polypeptide, wherein the Cas12a2 polypeptide shares at least 85% identity with a sequence selected from the group consisting of any one of SEQ ID NOs: 1-14 and 55, or a polynucleotide encoding a Cas12a2 polypeptide, wherein the Cas12a2 polypeptide shares at least 85% identity with a sequence selected from the group consisting of any one of SEQ ID NOs: 1-14 and 55, and (ii) a guide polynucleotide, or a polynucleotide encoding a guide polynucleotide, wherein said guide polynucleotide is designed to bind said Cas12a2 polypeptide and hybridize with a target sequence in one or more cells of interest, wherein said target sequence is located adjacent to a PAM sequence that is recognized by said Cas12a2 polypeptide. In some embodiments, the PAM sequence comprises TTTC, TCTC, TTCC, TGAA, CACC, or TGGT. In some embodiments, the guide polynucleotide comprises a spacer comprising a nucleic acid sequence that is fully complementary to the target sequence, or that is partially complementary differing by no more than 4 nucleotides from the nucleic acid sequence fully complementary to the target sequence.

In some embodiments of the compositions disclosed herein, said Cas12a2 polypeptide shares at least 90% identity with a sequence selected from the group consisting of any one of SEQ ID NOs: 1-14 and 55. In some embodiments, said Cas12a2 polypeptide shares at least 95% identity with a sequence selected from the group consisting of any one of SEQ ID NOs: 1-14 and 55. In some embodiments, said Cas12a2 polypeptide comprises a sequence selected from the group consisting of any one of SEQ ID NOs: 1-14 and 55.

In some embodiments, said Cas12a2 polypeptide comprises one or more amino acid motifs having at least 90% sequence identity with a sequence selected from the group consisting of SEQ ID NOs: 30-46. In some embodiments, said Cas12a2 polypeptide comprises one or more amino acid motifs selected from the group consisting of SEQ ID NOs: 30-46.

In some embodiments of the compositions disclosed herein, said one or more cells of interest is the cell of one or more pest of interest. In some embodiments of the compositions disclosed herein, said one or more pest of interest is a pathogenic bacterial species. In some embodiments of the compositions disclosed herein, said one or more cells of interest is one or more bacterial cells.

In some embodiments of the compositions disclosed herein, the target sequence is a target sequence specific to the pest of interest. In some embodiments of the compositions disclosed herein, the target sequence is a target sequence specific to the pathogenic bacterial species. In some embodiments, said one or more pathogenic bacterial species is a pathogenic bacterial species associated with plants. In some embodiments, said one or more pathogenic bacterial species is a pathogenic bacterial species associated with mammals. In some embodiments, said one or more pathogenic bacterial species is a pathogenic bacterial species associated with humans. In some embodiments, said pathogenic bacterial species is selected from the group consisting of Xanthomonas sp., Escherichia sp., Pseudomonas sp., Erwinia sp., Xylella sp., Clavibacter sp., Ralstonia sp., Pectobacterium sp., Streptomyces sp., Burkholderia sp., Phytoplasma sp., Acidovorax sp., Pantoea sp., Agrobacterium sp., Spiroplasma sp., Candidatus Liberibacter sp., Dickeya sp., Serratia sp., Sphingomonas sp., Rhizobacter sp., Rhizomonas sp., Xylophilus sp., Rickettsia sp., Bacillus sp., Clostridium sp., Arthrobacter sp., Curtobacterium sp., Leifsonia sp., Rhodococcus sp., Phytoplasma sp., Enterobacter sp., Citrobacter sp., Klebsiella sp., Hafnia sp., Corynebacterium sp., Mycoplasma sp., Serratia sp., Pasteurella sp., Proteus sp., Campylobacter sp., Salmonella sp., Pseudomonas sp., Brucella sp., Staphylococcus sp., Streptococcus sp., Trueperella sp., Clostridium sp., Listeria sp., Anthrax sp., Bartonella sp., Capnocytophaga sp., Streptobacillus sp., Rickettsia sp., Anaplasma sp., Shigella sp., Borrelia sp., Actinomyces sp., Bacteroides sp., Bordetella sp., Chlamydia sp., Chlamydophila sp., Ehrlichia sp., Enterococcus sp., Francisella sp., Haemophilus sp., Helicobacter sp., Klebsiella sp., Legionella sp., Leptospira sp., Mycobacterium sp., Neisseria sp., Nocardia sp., Treponema sp., Vibrio sp., Yersinia sp., Coxiella sp., Wolbachia sp., Liberibacter sp., Aeromonas sp., Edwardsiella sp., Flavobacterium sp., Tenacibaculum sp., Renibacterium sp., Piscirickettsia sp., Enterobacterium sp., Lactococcus sp., Aerococcus sp., and Hepatobacter sp.

In some embodiments of the compositions disclosed herein, said one or more cells is one or more eukaryotic cells. In some embodiments, said one or more eukaryotic cells belongs to one or more plant pathogen. In some embodiments, said one or more plant pathogens is a plant parasitic nematode, an insect, a fungus, a virus, a mollusk, a spider, a scorpion, a caterpillar, an animal, a mite, a tick, or a combination thereof. In some embodiments, the target sequence is a target sequence specific to said one or more plant pathogen. said one or more eukaryotic cells is one or more mammalian cells. In some embodiments, said one or more mammalian cells is one or more human cells. In some embodiments, said one or more mammalian cells is one or more cancer cells. In some embodiments of the compositions disclosed herein, said target sequence is a cancer cell-specific target sequence.

In some embodiments of the compositions disclosed herein, said guide polynucleotide is a guide RNA. In some embodiments, said polynucleotide encoding a Cas12a2 polypeptide and said polynucleotide encoding a guide polynucleotide are part of a vector. In some embodiments, said vector is selected from the group consisting of phages, phagemids, and conjugative plasmids In some embodiments, said phage or phagemid is derived from a phage selected from the group consisting of M13, lambda, p22, T7, Mu, T4 phage, PBSX, P1Puna-like, P2, 13, Bcep 1, Bcep 43, Bcep 78, T5 phage, phi, C2, L5, HK97, N15, T3 phage, P37, MS2, Q.beta., or Phi X 174, T2 phage, T12 phage, R17 phage, M13 phage, G4 phage, Enterobacteria phage P2, P4 phage, N4 phage, Pseudomonas phage .PHI.6, .PHI.29 phage and 186 phage. In some embodiments, said vector is a viral vector. In some embodiments, the viral vector is an adeno-associated virus (AAV) vector.

In some embodiments of the compositions disclosed herein, said polynucleotide encoding a Cas12a2 polypeptide and said polynucleotide encoding a guide polynucleotide are part of the same polynucleotide.

In one aspect, provided is a method for binding a target sequence in one or more cells of interest comprising delivering to said one or more cells of interest the composition provided herein, thereby binding said target sequence with the Cas12a2 polypeptide of the composition.

In a further aspect, provided is a method for cleaving and/or modifying a target sequence in one or more cells of interest comprising delivering to said one or more cells of interest the composition provided herein, wherein the Cas12a2 polypeptide of the composition cleaves or modifies said target sequence.

In some embodiments of the methods disclosed herein, said one or more cells of interest is the cell of one or more pest of interest. In some embodiments of the methods disclosed herein, said one or more pest of interest is a pathogenic bacterial species. In some embodiments, the one or more cells of interest is one or more bacterial cells. In some embodiments, said one or more bacterial cells is a pathogenic bacterial species. In some embodiments, said one or more pathogenic bacterial species is a pathogenic bacterial species associated with plants. In some embodiments, said one or more pathogenic bacterial species is a pathogenic bacterial species associated with mammals. In some embodiments, said one or more pathogenic bacterial species is a pathogenic bacterial species associated with humans.

In some embodiments of the methods disclosed herein, said pathogenic bacterial species is selected from the group consisting of Xanthomonas sp., Escherichia sp., Pseudomonas sp., Erwinia sp., Xylella sp., Clavibacter sp., Ralstonia sp., Pectobacterium sp., Streptomyces sp., Burkholderia sp., Phytoplasma sp., Acidovorax sp., Pantoea sp., Agrobacterium sp., Spiroplasma sp., Candidatus Liberibacter sp., Dickeya sp., Serratia sp., Sphingomonas sp., Rhizobacter sp., Rhizomonas sp., Xylophilus sp., Rickettsia sp., Bacillus sp., Clostridium sp., Arthrobacter sp., Curtobacterium sp., Leifsonia sp., Rhodococcus sp., Phytoplasma sp., Enterobacter sp., Citrobacter sp., Klebsiella sp., Hafnia sp., Corynebacterium sp., Mycoplasma sp., Serratia sp., Pasteurella sp., Proteus sp., Campylobacter sp., Salmonella sp., Pseudomonas sp., Brucella sp., Staphylococcus sp., Streptococcus sp., Trueperella sp., Clostridium sp., Listeria sp., Anthrax sp., Bartonella sp., Capnocytophaga sp., Streptobacillus sp., Rickettsia sp., Anaplasma sp., Shigella sp., Borrelia sp., Actinomyces sp., Bacteroides sp., Bordetella sp., Chlamydia sp., Chlamydophila sp., Ehrlichia sp., Enterococcus sp., Francisella sp., Haemophilus sp., Helicobacter sp., Klebsiella sp., Legionella sp., Leptospira sp., Mycobacterium sp., Neisseria sp., Nocardia sp., Treponema sp., Vibrio sp., Yersinia sp., Coxiella sp., Wolbachia sp., Liberibacter sp., Aeromonas sp., Edwardsiella sp., Flavobacterium sp., Tenacibaculum sp., Renibacterium sp., Piscirickettsia sp., Enterobacterium sp., Lactococcus sp., Aerococcus sp., and Hepatobacter sp.

In some embodiments, said contacting comprises contacting said one or more cells of interest with a phage or a phagemid engineered to comprise: (i) a polynucleotide encoding said Cas12a2 polypeptide, and (ii) a polynucleotide encoding a guide polynucleotide.

In some embodiments, the one or more cells of interest is one or more eukaryotic cells. In some embodiments, said one or more eukaryotic cells belongs to one or more plant pathogens.

In some embodiments, said one or more plant pathogens is a plant parasitic nematode, an insect, a fungus, a virus, a mollusk, a spider, a scorpion, a caterpillar, an animal, a mite, a tick, or a combination thereof.

In some embodiments, said one or more eukaryotic cells is one or more mammalian cells. In some embodiments, said one or more mammalian cells is one or more cancer cells.

In some embodiments, said contacting comprises contacting said one or more cells of interest with a viral vector engineered to comprise: (i) a polynucleotide encoding said Cas12a2 polypeptide, and (ii) a polynucleotide encoding a guide polynucleotide. In some embodiments, the viral vector is an adeno-associated virus (AAV) vector.

In some embodiments of the methods disclosed herein, said contacting comprises contacting said one or more cells of interest with a phage or a phagemid engineered to comprise: (i) a polynucleotide encoding said Cas12a2 polypeptide, and (ii) a polynucleotide encoding a guide polynucleotide. In some embodiments, said phage or a phagemid is derived from a phage selected from the group consisting of M13, lambda, p22, T7, Mu, T4 phage, PBSX, P1Puna-like, P2, 13, Bcep 1, Bcep 43, Bcep 78, T5 phage, phi, C2, L5, HK97, N15, T3 phage, P37, MS2, Q.beta., or Phi X 174, T2 phage, T12 phage, R17 phage, M13 phage, G4 phage, Enterobacteria phage P2, P4 phage, N4 phage, Pseudomonas phage .PHI.6, .PHI.29 phage and 186 phage.

In one aspect, the present disclosure provides a method of inhibiting one or more eukaryotic cells comprising contacting one or more eukaryotic cells with any of the compositions disclosed herein. In some embodiments, said one or more eukaryotic cells belongs to one or more plant pathogens. In some embodiments, said one or more plant pathogens is a plant parasitic nematode, an insect, a fungus, a virus, a mollusk, a spider, a scorpion, a caterpillar, an animal, a mite, a tick, or a combination thereof. In some embodiments, said one or more eukaryotic cells is one or more mammalian cells. In some embodiments, said one or more mammalian cells is one or more cancer cells. In some embodiments, said contacting comprises contacting said one or more cells of interest with a viral vector engineered to comprise: (i) a polynucleotide encoding said Cas12a2 polypeptide, and (ii) a polynucleotide encoding a guide polynucleotide. In some embodiments, the viral vector is an adeno-associated virus (AAV) vector.

In one aspect, the present disclosure provides a method for increasing resistance or tolerance of a plant to one or more plant pathogens, the method comprising: contacting a plant, plant part, or plant cell with a composition comprising the composition of any one of claims 3 to 30 to produce a modified plant, plant part, or plant cell; wherein the at least one guide polynucleotide is capable of binding the Cas12a2 polypeptide and hybridizing to a target sequence in one or more cells of each corresponding plant pathogen, thereby increasing resistance or tolerance of the plant to the one or more plant pathogens, as compared to resistance or tolerance of a control plant to the one or more plant pathogens.

In one aspect, the present disclosure provides a method for producing a modified plant with increased resistance or tolerance to one or more plant pathogens, the method comprising: contacting a plant, plant part, or plant cell with a composition comprising the composition of any one of claims 3 to 30 to produce a modified plant, plant part, or plant cell; and selecting for a modified plant, plant part, or plant cell that expresses the Cas12a2 polypeptide and the at least one guide polynucleotide; wherein the at least one guide polynucleotide is capable of binding the Cas12a2 polypeptide and hybridizing to a target sequence in one or more cells of each corresponding plant pathogen; thereby producing a modified plant with increased resistance or tolerance to the one or more plant pathogens, as compared to resistance or tolerance of a control plant to the one or more plant pathogens. In some embodiments, the selecting comprises growing the plant, plant part, or plant cell in media comprising a selectable agent. In some embodiments, the selectable agent is an herbicide, an antibiotic, a carbohydrate, an amino acid, or a metabolite.

In some embodiments of the methods disclosed herein, the control plant is a corresponding plant or population of plants that does not comprise the composition. In some embodiments, the one or more plant pathogens comprises a plant parasitic nematode, an insect, a fungus, a virus, a mollusk, a spider, a scorpion, a caterpillar, an animal, a mite, a tick, or a combination thereof. In some embodiments, the modified plant comprises an improved agronomic trait as compared to the control plant. In some embodiments, the improved agronomic trait comprises biomass yield and/or seed yield. In some embodiments, said contacting comprises contacting with a virus or viral nucleic acid molecule comprising the composition, microinjection, electroporation, Agrobacterium-mediated transformation, direct gene transfer, particle mediated delivery, topical application, silicon carbide fiber mediated delivery, delivery via cell-penetrating peptides, or a combination thereof. In some embodiments, said contacting comprises introducing into the plant cell, and culturing the plant cell to regenerate a plant or plant part comprising the composition. In some embodiments, the plant, plant part, or plant cell is corn (Zea mays), Brassica species, Brassica napus, Brassica rapa, Brassica juncea, rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

In a further aspect, provided is a modified cell (e.g., plant cell, eukaryotic cell, or bacterial cell) produced by the method provided herein (i.e., by binding, cleaving, and or modifying a target sequence in a cell with a Cas12a2 composition provided herein).

In one aspect, the present disclosure provides a modified plant produced by any of the methods disclosed herein.

In one aspect, the present disclosure provides a modified bacterial cell produced by any of the methods disclosed herein.

In one aspect, the present disclosure provides a modified eukaryotic cell (e.g., mammalian cell, human cell, and/or cancer cell) produced by any of the methods disclosed herein.

In one aspect, the present disclosure provides a plant, plant part, plant cell, or population of plants comprising any of the compositions or vectors disclosed herein.

In one aspect, the present disclosure provides a mammalian cell comprising any of the compositions or vectors disclosed herein.

In one aspect, the present disclosure provides a bacterial cell comprising any of the compositions or vectors disclosed herein.

In one aspect, the present disclosure provides an amino acid motif, and fragments and variants thereof. In some embodiments, the amino acid motif is a consensus motif. In some embodiments, the consensus motif exhibits nuclease activity. In some aspects, the consensus motif is comprised within a polypeptide. In some embodiments, the consensus motif is selected from any one of SEQ ID NOs: 30-46. In one embodiment, the consensus motif is SEQ ID NO: 30. In one embodiment, the consensus motif is SEQ ID NO: 31. In one embodiment, the consensus motif is SEQ ID NO: 32. In one embodiment, the consensus motif is SEQ ID NO: 33. In one embodiment, the consensus motif is SEQ ID NO: 34. In one embodiment, the consensus motif is SEQ ID NO: 35. In one embodiment, the consensus motif is SEQ ID NO: 36. In one embodiment, the consensus motif is SEQ ID NO: 37. In one embodiment, the consensus motif is SEQ ID NO: 38. In one embodiment, the consensus motif is SEQ ID NO: 39. In one embodiment, the consensus motif is SEQ ID NO: 40. In one embodiment, the consensus motif is SEQ ID NO: 41. In one embodiment, the consensus motif is SEQ ID NO: 42. In one embodiment, the consensus motif is SEQ ID NO: 43. In one embodiment, the consensus motif is SEQ ID NO: 44. In one embodiment, the consensus motif is SEQ ID NO: 45. In one embodiment, the consensus motif is SEQ ID NO: 46.

In some embodiments, the consensus motif has at least 90% identity (e.g., 90%, 91%, 92%, 93%, 94%, 95%, 96%, 97%, 98%, 99% or more identity) with a sequence selected from the group consisting of any one of SEQ ID NOs: 30-46 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 30 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 30 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 31 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 32 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 33 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 34 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 35 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 36 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 37 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 38 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 39 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 40 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 41 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 42 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 43 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 44 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 45 (e.g., wherein the consensus motif, or polypeptide comprising the consensus motif, retains nuclease activity). In one embodiment, the consensus motif has at least 90% identity (e.g., 90%, 91%, 92%, 93%, 94%, 95%, 96%, 97%, 98%, 99% or more identity) with SEQ ID NO: 46 (e.g., wherein the consensus motif, or polypeptide comprising the consensus motif, retains nuclease activity).

In one aspect, the present disclosure provides polypeptides comprising at least one consensus motif disclosed herein. In some instances, a polypeptide can comprise more than one consensus motif. In some instances, the polypeptide encodes a Cas12a2 protein or fragment or variant thereof. The polypeptide comprising at least one consensus motif can encode a Cas12a2 protein or fragment or variant thereof having Cas12a2 activity. For example, the polypeptide can encode any Cas12a2 protein or fragment or variant thereof, wherein said Cas12a2 protein or fragment or variant thereof comprises a consensus motif disclosed herein and has Cas12a2 activity.

In some embodiments, the polypeptide comprises a consensus motif having at least 90% identity (e.g., 90%, 91%, 92%, 93%, 94%, 95%, 96%, 97%, 98%, 99% or more identity) with a sequence selected from the group consisting of any one of SEQ ID NOs: 30-46 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 30 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 31 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 32 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 33 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 34 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 35 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 36 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 37 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 38 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 39 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 40 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 41 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 42 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 43 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 44 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 45 (e.g., wherein the polypeptide retains nuclease activity). In one embodiment, the polypeptide comprises a consensus motif having at least 90% identity (e.g., 90%, 91%, 92%, 93%, 94%, 95%, 96%, 97%, 98%, 99% or more identity) with SEQ ID NO: 46 (e.g., wherein the polypeptide retains nuclease activity).

### BRIEF DESCRIPTION OF THE DRAWINGS

FIGS. 1A-IC show amino acid sequence alignments identifying conserved residues within three domains of the Sulf-type Cas12a2 proteins corresponding to SEQ ID NOs: 1-15 and 55. FIG. 1A shows alignment corresponding to amino acid residues 370 to 389 of SuCas12a2.

FIG. 1B shows alignment corresponding to amino acid residues 896 to 919 of SuCas12a2. FIG. 1C shows alignment corresponding to amino acid residues 1028 to 1049 of SuCas12a2.

FIG. 2 shows a phylogeny tree of Cas12a2 peptides.

FIG. 3 graphically depicts the results of a toxicity assay in E. coli evaluating the toxicity of Unk97 and SuCas12a2 when targeting the Oryza sativa CAO1 gene in the cell using the CAO1-1 guide RNA.

FIG. 4 depicts activity of the indicated Cas12a2 peptides targeting a region adjacent to various PAM sequences indicated in a toxicity assay in E. coli.

FIG. 5 depicts activity of the indicated Cas12a2 peptides at the target sequence in the CAO1 gene using a guide RNA with 0 to 4 mismatches n a toxicity assay in E. coli.

FIG. 6 graphically depicts the effects at the target sequence in the KRAS-1 gene (“cancer target”) and off-target effects at a wild-type sequence (“WT”) of the Cas12a2 system using the nuclease Unk109 or SuCas12a2 and the indicated guide RNA with 0 to 2 mismatches in a toxicity assay in E. coli. “NS” indicates no significant reduction (toxicity) over the non-targeted baseline.

FIG. 7 graphically depicts the effects at the target sequence in the EGFR-3 gene (“cancer target”) and off-target effects at a wild-type sequence (“WT”) of the Cas12a2 system using the nuclease Unk109 or SuCas12a2 and the indicated guide RNA with 0 to 2 mismatches in a toxicity assay in E. coli. “NS” indicates no significant reduction (toxicity) over the non-targeted baseline.

### DETAILED DESCRIPTION OF THE INVENTION

Methods and compositions are provided herein for the genome modification and/or the selective targeting and elimination of target cells that harbor certain pre-determined DNA target sequences through the use of the CRISPR-Cas12a2 system and components thereof. The CRISPR enzymes of the invention are selected from orthologs belonging to the Cas12a2 family of nucleases, e.g. a Cas12a2 ortholog. Cas12a2 is alternatively referred to herein as Cms1, which is an abbreviation for CRISPR from Microgenomates and Smithella, and is so named because some bacterial species in these groups encode Cms1 nucleases; the terms Cas12a2, Csm1, and Cms1 may be used interchangeably. Cms1 nucleases may also be referred to as Cas12f nucleases. The methods and compositions include nucleic acids to bind target DNA sequences. This is advantageous as nucleic acids are much easier and less expensive to produce than, for example, peptides, and the specificity can be varied according to the length of the stretch where homology is sought. Complex 3-D positioning of multiple fingers, for example is not required. In some embodiments, the nucleic acids are guide polynucleotides such as guide RNAs (gRNAs; alternatively CRISPR RNAs or crRNAs) that are capable of interacting with a Cas12a2 enzyme and of hybridizing with a nucleotide sequence through base pairing. As used herein, guide RNAs that are capable of interacting or that are designed to interact with a Cas12a2 polypeptide can bind, associate with, or otherwise form a complex with the Cas12a2 polypeptide. Methods of measuring interaction of gRNAs with Cas12a2 polypeptide are well known in the art. The target sequences bound be a target sequence specific to any pest of interest disclosed herein. In some instances, the target sequence is within one or more cells of interest. The cells of interest can be the cell of one or more pest of interest, which can be a pathogenic bacterial species.

Also provided are nucleic acids encoding the Cas12a2 polypeptides, as well as methods of using Cas12a2 polypeptides to target specific DNA or RNA sequences of target cells, including bacterial cells and eukaryotic cells. The targeted nucleotide sequences may be present in genomic DNA, plasmid DNA, other DNA elements, or RNA such as mRNA harbored within the targeted cells. The Cas12a2 polypeptides interact with specific guide polynucleotides such as guide RNAs (gRNAs), which direct the Cas12a2 endonuclease to a specific target site. Without being limited by theory, the Cas12a2-gRNA complex hybridizes with the targeted nucleotide sequence (the “initial hybridization event”), at which site the Cas12a2 endonuclease introduces a double-stranded break (DSB). This process of hybridization and DSB production leads to a change in the structure of the Cas12a2 protein, resulting in a protein that is capable of degrading double-stranded DNA (dsDNA) and/or RNA in a non-sequence-specific manner, leading to cell death. Since the specificity of the initial hybridization event is provided by the guide RNA, the Cas12a2 polypeptide is universal and can be used with different guide RNAs to target different genomic sequences. Cas12a2-associated CRISPR arrays are processed into mature crRNAs without the requirement of an additional trans-activating crRNA (tracrRNA). Cas12a2 proteins can process crRNA arrays that include multiple spacer sequences; the compositions of the invention include, in some embodiments, crRNA arrays with multiple spacer sequences designed to target multiple different loci within the cells species of interest. Cas12a2-gRNA systems can target DNA sequences adjacent to a variety of protospacer adjacent motif (PAM) sequences, with the PAM sequence located immediately 5′ or 3′ of the DNA sequence targeted by Cas12a2. “Adjacent” or “immediately adjacent” refers to the target DNA sequence being about 1 nucleotide to 50 nucleotides, about 5 nucleotides to 45 nucleotides, or about 7 nucleotides to 40 nucleotides either upstream (5′) or downstream (3′) of the PAM sequence. In some embodiments, the target DNA sequence is adjacent or immediately adjacent to the PAM sequence when it is 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides upstream (5′) or downstream (3′) of the PAM sequence. For example, The PAM can be located 1, 2, 3, 4, 5, 6, 7, 8, 9, or 10 nucleotides 5′ side (upstream) or 3′ side (downstream) of the target sequence.

The initial hybridization event is sequence-specific with limited off target effects, resulting in sequence-specific killing of cells of interest without harming cells that do not harbor the sequence(s) of interest.

Amino acid motifs, and fragments and variants thereof, exhibiting nuclease activity are also envisaged for use in the invention. The amino acid motifs disclosed herein exhibit nuclease activity, and as such, can be used alone or can be comprised within, or operably linked to, another molecule, such as a polypeptide for use in targeting sequences within cells of interest. Amino acid motifs can include, but are not limited to, amino acid consensus motifs selected from the group consisting of SEQ ID NOs: 30-46 (e.g., wherein the amino acid motif exhibits nuclease activity). The amino acid motifs can be comprised within a polypeptide sequence, such that the polypeptide sequence comprises at least one consensus motif having at least 90% identity (e.g., 90%, 91%, 92%, 93%, 94%, 95%, 96%, 97%, 98%, 99% or more identity) with a sequence selected from the group consisting of any one of SEQ ID NOs: 30-46. The present disclosure also provides polypeptides comprising at least one consensus motif disclosed herein. Due to the presence of the at least one consensus motif, the polypeptides will exhibit nuclease activity.

### I. Cas12a2 Endonucleases and Guide Polynucleotides

Provided herein are Cas12a2 endonucleases, and fragments and variants thereof, for use in targeting sequences within cells (e.g., bacterial cells or eukaryotic cells), for example within genomic DNA, plasmids, or other DNA-containing elements found in cells. As used herein, the term Cas12a2 endonucleases or Cas12a2 polypeptides refers to homologs, orthologs, and variants of the Cas12a2 polypeptide sequences set forth in SEQ ID NOs:1-14 and 55. Typically, Cas12a2 endonucleases can act without the use of tracrRNAs, requiring on a single gRNA for sequence specificity. In general, a Cas12a2-gRNA complex can perform an initial hybridization event to target a particular sequence. Without being limited by theory, following this initial hybridization event, the Cas12a2 protein is then able to perform a secondary collateral activity directed against double-stranded DNA (dsDNA) or RNA without any sequence specificity. This collateral activity results in cell death in those cells in which the Cas12a2-gRNA complex undergoes an initial hybridization event. In general, Cas12a2 polypeptides comprise at least one RNA recognition and/or RNA binding domain. RNA recognition and/or RNA binding domains interact with guide RNAs. Typically the guide RNA comprises a region with a stem-loop structure that interacts with the Cas12a2 polypeptide. This stem-loop often comprises the sequence UCUACN₃₋₅GUAGAU (SEQ ID NOs: 47-49, encoded by SEQ ID NOs: 50-52), with “UCUAC” and “GUAGA” base-pairing to form the stem of the stem-loop. N3-5 denotes that any base may be present at this location, and 3, 4, or 5 nucleotides may be included at this location. Some CRISPR nucleases have been shown to function with guide polynucleotides in which some of the ribonucleotide residues have been replaced by deoxyribonucleotide residues (Yin et al (2018) Nat Chem Biol 14:311-316; U.S. Pat. No. 9,650,617); the present invention also encompasses embodiments in which the guide polynucleotide is a guide RNA, embodiments in which the guide polynucleotide is a guide DNA, and embodiments in which the guide polynucleotide comprises both DNA and RNA residues. In specific embodiments, a Cas12a2 polypeptide, or a polynucleotide encoding a Cas12a2 polypeptide, comprises: an RNA-binding portion that interacts with the DNA-targeting RNA, and an activity portion that exhibits site-directed enzymatic activity, such as a RuvC endonuclease domain. Without being limited by theory, the RuvC endonuclease domain may also exhibit secondary, collateral activity directed against dsDNA and/or RNA in a non-sequence-specific manner.

The Cas12a2 endonucleases, and fragments and variants thereof, can be used for targeting sequences within any one or more cells of interest disclosed herein. In some instances, the one or more cells of interest can be the cell of one or more pest of interest. Additionally, the target sequence can be a target sequence that is specific to the pest of interest. The pest of interest can be a pathogenic bacterial species, such as a pathogenic bacterial species associated with plants or mammals. In some instances, the pathogenic bacterial species is associated with humans. The pathogenic bacterial species can be a bacterial species selected from the group consisting of Xanthomonas sp., Escherichia sp., Pseudomonas sp., Erwinia sp., Xylella sp., Clavibacter sp., Ralstonia sp., Pectobacterium sp., Streptomyces sp., Burkholderia sp., Phytoplasma sp., Acidovorax sp., Pantoea sp., Agrobacterium sp., Spiroplasma sp., Candidatus Liberibacter sp., Dickeya sp., Serratia sp., Sphingomonas sp., Rhizobacter sp., Rhizomonas sp., Xylophilus sp., Rickettsia sp., Bacillus sp., Clostridium sp., Arthrobacter sp., Curtobacterium sp., Leifsonia sp., Rhodococcus sp., Phytoplasma sp., Enterobacter sp., Citrobacter sp., Klebsiella sp., Hafnia sp., Corynebacterium sp., Mycoplasma sp., Serratia sp., Pasteurella sp., Proteus sp., Campylobacter sp., Salmonella sp., Pseudomonas sp., Brucella sp., Staphylococcus sp., Streptococcus sp., Trueperella sp., Clostridium sp., Listeria sp., Anthrax sp., Bartonella sp., Capnocytophaga sp., Streptobacillus sp., Rickettsia sp., Anaplasma sp., Shigella sp., Borrelia sp., Actinomyces sp., Bacteroides sp., Bordetella sp., Chlamydia sp., Chlamydophila sp., Ehrlichia sp., Enterococcus sp., Francisella sp., Haemophilus sp., Helicobacter sp., Klebsiella sp., Legionella sp., Leptospira sp., Mycobacterium sp., Neisseria sp., Nocardia sp., Treponema sp., Vibrio sp., Yersinia sp., Coxiella sp., Wolbachia sp., Liberibacter sp., Aeromonas sp., Edwardsiella sp., Flavobacterium sp., Tenacibaculum sp., Renibacterium sp., Piscirickettsia sp., Enterobacterium sp., Lactococcus sp., Aerococcus sp., and Hepatobacter sp.

Cas12a2 polypeptides can be wild type Cas12a2 polypeptides, modified Cas12a2 polypeptides, or a fragment of a wild type or modified Cas12a2 polypeptide. The Cas12a2 polypeptide can be modified to increase nucleic acid binding affinity and/or specificity, alter an enzymatic activity, and/or change another property of the protein. For example, nuclease (i.e., DNase, RNase) domains of the Cas12a2 polypeptide can be modified, deleted, or inactivated. Alternatively, the Cas12a2 polypeptide can be modified or truncated to alter or remove domains that are not essential for the function of the protein.

In some embodiments, the Cas12a2 polypeptide can be derived from a wild type Cas12a2 polypeptide or fragment thereof. In other embodiments, the Cas12a2 polypeptide can be derived from a modified Cas12a2 polypeptide. For example, the amino acid sequence of the Cas12a2 polypeptide can be modified to alter one or more properties (e.g., nuclease activity, affinity, stability, solubility, etc.) of the protein.

In general, a Cas12a2 polypeptide comprises at least one nuclease domain, but need not contain an HNH domain such as the one found in Cas9 proteins. For example, a Cas12a2 polypeptide can comprise a RuvC or RuvC-like nuclease domain. Without being limited by theory, the RuvC or RuvC-like domain may comprise three catalytic residues that are typically aspartate, glutamate, and aspartate, respectively, and may be responsible for the Cas12a2 nuclease activity.

In some embodiments, the Cas12a2 polypeptide can comprise at least one cell-penetrating domain. The cell-penetrating domain can be located at the N-terminus, the C-terminus, or in an internal location of the protein.

In still other embodiments, the Cas12a2 polypeptide can also comprise at least one marker domain. Non-limiting examples of marker domains include fluorescent proteins, purification tags, and epitope tags. In certain embodiments, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalama1, GFPuv, Sapphire, T-sapphire), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain can be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, biotin carboxyl carrier protein (BCCP), and calmodulin.

In certain embodiments, the Cas12a2 polypeptide may be part of a protein-RNA complex comprising a guide polynucleotide. In some embodiments, the guide polynucleotide may be a guide RNA. The guide polynucleotide interacts with the Cas12a2 polypeptide to direct the Cas12a2 polypeptide to a specific target site in a bacterial cell, where the target site comprises dsDNA that may be present in genomic DNA, plasmid DNA, or other DNA components in a bacterial cell of interest. If a suitable protospacer adjacent motif (PAM) sequence is present immediately 5′ of the target sequence, the Cas12a2-guide polynucleotide complex may hybridize with the dsDNA target sequence. Following this initial hybridization event, the Cas12a2 enzyme may cleave the target DNA. Without being limited by theory, the Cas12a2 enzyme may then undergo a structural change that may allow the Cas12a2 enzyme to cleave dsDNA and/or RNA in a non-sequence-specific manner (“secondary” or “collateral” activity). This secondary activity may result in bacterial cell death. As used herein, the term “DNA-targeting RNA” refers to a guide RNA that interacts with the Cas12a2 polypeptide and the target site of the nucleotide sequence of interest in the genome of a cell. A DNA-targeting RNA, or a DNA polynucleotide encoding a DNA-targeting RNA, can comprise: a first segment comprising a nucleotide sequence that is complementary to a sequence in the target DNA, and a second segment that interacts with a Cas12a2 polypeptide. The target sequence that is cleaved and/or modified by the Cas12a2 enzyme can be a target sequence specific to a pest of interest. In some instances, the target sequence is within one or more cells of interest. The cells of interest can be the cell of one or more pest of interest, which can be a pathogenic bacterial species, such as a pathogenic bacterial species associated with plants, mammals, and/or humans.

Cas12a2 proteins for use in the invention include, but are not limited to, Cas12a2 proteins that comprise at least one amino acid motif selected from the group consisting of SEQ ID NOs: 30-46. Any Cas12a2 nuclease protein having Cas12a2 activity can comprise at least one amino acid motif selected from the group consisting of SEQ ID NOs: 30-46. In certain preferred embodiments, a Cas12a2 protein comprises more than one amino acid motif selected from the group consisting of SEQ ID NOs: 30-46. In some embodiments, the Cas12a2 protein comprises a consensus motif selected from any one of SEQ ID NOs: 30-46. In one embodiment, the Cas12a2 protein comprises a consensus motif of SEQ ID NO: 30. In one embodiment, the Cas12a2 protein comprises a consensus motif of SEQ ID NO: 31. In one embodiment, the Cas12a2 protein comprises a consensus motif of SEQ ID NO: 32. In one embodiment, the Cas12a2 protein comprises a consensus motif of SEQ ID NO: 33. In one embodiment, the Cas12a2 protein comprises a consensus motif of SEQ ID NO: 34. In one embodiment, the Cas12a2 protein comprises a consensus motif of SEQ ID NO: 35. In one embodiment, the Cas12a2 protein comprises a consensus motif of SEQ ID NO: 36. In one embodiment, the Cas12a2 protein comprises a consensus motif of SEQ ID NO: 37. In one embodiment, the Cas12a2 protein comprises a consensus motif of SEQ ID NO: 38. In one embodiment, the Cas12a2 protein comprises a consensus motif of SEQ ID NO: 39. In one embodiment, the Cas12a2 protein comprises a consensus motif of SEQ ID NO: 40. In one embodiment, the Cas12a2 protein comprises a consensus motif of SEQ ID NO: 41. In one embodiment, the Cas12a2 protein comprises a consensus motif of SEQ ID NO: 42. In one embodiment, the Cas12a2 protein comprises a consensus motif of SEQ ID NO: 43. In one embodiment, the Cas12a2 protein comprises a consensus motif of SEQ ID NO: 44. In one embodiment, the Cas12a2 protein comprises a consensus motif of SEQ ID NO: 45. In one embodiment, the Cas12a2 protein comprises a consensus motif of SEQ ID NO: 46. The example consensus motifs are set forth as follows:

TABLE 1
Example Consensus Motifs

| SEQ | | |
|---|---|---|
| ID NO: | Description | Sequence |
| 30 | Sulf-type Cas12a2 | W-x(3)-(Y/F/L)-x(3)-(D/G/N)-(Q/L/F/M)-(I/L/V/M)-x-(L/I/V)-x-K- |
| | Conserved motif 1 | (D/E/S)-(Y/F)-Y-(K/R/L/S)-x-(L/I/M)-x-(K/R/S)-(K/E)-(A/I/L/V)-x- |
| | | F-(D/E/N/V)-(A/G/F/V)-(F/M/I)-W |
| | | Where x = any amino acid |
| 31 | Sulf-type Cas12a2 | F-K-(Y/V/P)-(K/I)-x-(I/V)-P-(F/A/V/I)-x-(V/A/L)-x(3)-(L/I/V)-(A/V) |
| | Conserved motif 2 | Where x = any amino acid |
| 32 | Sulf-type Cas12a2 | F-(N/S/D)-(L/I)-x-(K/N/H/A)-Y-P-(I/L)-K-(V/S)-A-F-(D/N)-(Y/F)- |
| | Conserved motif 3 | (A/S)-W-E-x-(L/C/V)-A |
| | | Where x = any amino acid |
| 33 | Sulf-type Cas12a2 | (I/L)-(I/V)-E-D-x(3)-(N/D)-(R/K)-(H/F/Y)-(I/L/V)-(I/L/F) |
| | Conserved motif 4 | Where x = any amino acid |
| 34 | Sulf-type Cas12a2 | (Y/C/S)-x-(I/V)-x-S-(F/L/I/V)-T-S-x(2)-(L/I)-x-K |
| | Conserved motif 5 | Where x = any amino acid |
| 35 | Sulf-type Cas12a2 | (E/A)-x-(I/L)-(E/K/I)-(K/H/R)-E-(I/V/L)-D-x-(K/N)-x-(Y/H)-x-(L/F) |
| | Conserved motif 6 | Where x = any amino acid |
| 36 | Sulf-type Cas12a2 | (L/S/F)-L-(L/F/V)-P-(I/F/L)-(I/V)-N-(Q/K)-D |
| | Conserved motif 7 | |
| 37 | Sulf-type Cas12a2 | (L/I)-(H/T)-P-E-F-x-(I/V/L/M)-(F/S/T)-Y |
| | Conserved motif 8 | Where x = any amino acid |
| 38 | Sulf-type Cas12a2 | (N/K)-R-(Y/F)-(S/G/W)-(R/K/S)-(F/L/V)-(Q/E)-(M/L/F/I)-x- |
| | Conserved motif 9 | (A/C/G)-x-(F/L/I)-x(2)-(E/D/H)-(F/Y/I/V)-(I/L/V/K)-(P/K) |
| | | Where x = any amino acid |
| 39 | Sulf-type Cas12a2 | G-I-D-(R/S)-(G/W)-(I/Q/L)-(K/N)-(E/Q)-L-A-(T/V)-L-C-(I/L/V) |
| | Conserved motif 10 | |
| 40 | Sulf-type Cas12a2 | (R/E)-x-I-L-D-L-(S/T)-(N/D/Y)-(L)-(R/K)-(V/I/A)-E-(T/S/K)-(T/D)- |
| | Conserved motif 11 | x-(E/D/N/K)-(G/K/N)-(K/N/E/T)-(K/S/Q)-(V/R/F/Y)-L-V-D-(L/Q)- |
| | | (S/A) |
| | | Where x = any amino acid |
| 41 | Sulf-type Cas12a2 | (L/M)-x(2)-(L/M/Y)-(A/S/P)-(Y/S)-(I/V/D)-(R/S)-x-(L/N/V)-(Q/T) |
| | Conserved motif 12 | Where x = any amino acid |
| 42 | Sulf-type Cas12a2 | (E/Q)-L-(D/E)-x(2)-(D/E/Q)-(N/D/Y/S)-(L/F)-K-x-G-(V/I/A)- |
| | Conserved motif 13 | (V/I)-A-N-(M/I)-(I/V)-G-(V/I)-(I/V)-(A/V/N)-(Y/F/H) |
| | | Where x = any amino acid |
| 43 | Sulf-type Cas12a2 | Y-x-(V/A/G)-(Y/K/R/V)-(I/V)-x-(L/F/I)-E-(D/N)-(L/I) |
| | Conserved motif 14 | Where x = any amino acid |
| 44 | Sulf-type Cas12a2 | A-(G/W)-(L/V)-(G/W/E)-(T/L)-(Y/M)-x-(F/Y)-(F/L/M)-E-x-(Q/L)- |
| | Conserved motif 15 | L-(L/V)-x-K |
| | | Where x = any amino acid |
| 45 | Sulf-type Cas12a2 | F-x(2)-G-(I/V)-(I/F/V)-x-(F/Y)-(V/I/T)-x-(P/A)-x(2)-T-(S/T)- |
| | Conserved motif 16 | x(2)-C-P-x-C |
| | | Where x = any amino acid |
| 46 | Sulf-type Cas12a2 | I-x(2)-(G/W)-D-(D/Q/E)-(N/S)-(G/A)-A-(Y/F)-(H/L/I/N)-I |
| | Conserved motif 17 | Where x = any amino acid |

The consensus motifs disclosed herein can contribute to or exhibit nuclease activity. As such, the presence of a consensus motif, or an active fragment or variant thereof, in a Cas12a2 protein can be sufficient for the Cas12a2 protein or fragment or variant thereof to exhibit nuclease activity. Accordingly, a person having skill in the art, in selecting active Cas12a2 proteins or fragments or variants thereof, would understand that any modifications or mutations within the disclosed consensus motifs is likely to reduce or eliminate Cas12a2 activity. Thus, it would be readily understood that any mutation or modification made to a polypeptide or protein located outside of a conserved motif or domain of the Cas12a2 protein or fragment or variant thereof should maintain Cas12a2 activity of the Cas12a2 protein or fragment or variant thereof.

In some instances, the conserved motifs disclosed herein are comprised within a nuclease protein or fragment or variant thereof. For example, an active Cas12a2 or fragment or variant thereof protein can comprise at least one of the conserved motifs disclosed herein. The active Cas12a2 protein or fragment or variant thereof can comprise at least 1, 2, 3, 4, 5, 6, 7, 8, 9, 10, 11, 12, 13, 14, 15, 16, or 17 conserved motifs disclosed herein as SEQ ID NOs: 30-46.

Disclosed herein are Cas12a2 proteins or fragments or variants thereof comprising at least one amino acid motif selected from the group consisting of SEQ ID NOs: 30-46, wherein said Cas12a2 protein has Cas12a2 activity. The Cas12a2 proteins or fragments or variants thereof comprising at least one amino acid motif selected from the group consisting of SEQ ID NOs: 30-46, can include any known Cas12a2 protein comprising mutation or modification of at least one amino acid residue located outside of a conserved motif, whereby the Cas12a2 protein or fragment or variant thereof retains nuclease activity.

In some embodiments, the Cas12a2 protein comprises a consensus motif having at least 90% identity (e.g., 90%, 91%, 92%, 93%, 94%, 95%, 96%, 97%, 98%, 99% or more identity) with a sequence selected from the group consisting of any one of SEQ ID NOs: 30-46 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 30 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 31 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 32 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 33 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 34 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 35 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 36 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 37 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 38 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 39 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 40 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 41 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 42 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 43 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 44 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 45 (e.g., wherein the Cas12a2 protein retains nuclease activity). In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 46 (e.g., wherein the Cas12a2 protein retains nuclease activity). In the embodiments described above, the Cas12a protein can comprise any Cas12a protein known in the art.

Particular Cas12a2 protein sequences are set forth in SEQ ID NOs:1-14 and 55; particular Cas12a2 protein-encoding polynucleotide sequences are set forth in SEQ ID NOs:16-29 In certain embodiments, a Cas12a2 protein has at least about 80% identity with a sequence selected from the group consisting of SEQ ID NOs:1-14 and 55. In certain embodiments, Cas12a2 proteins for use in the invention include, but are not limited to, Cas12a2 proteins comprising at least about 80%, 85%, 86%, 87%, 88%, 89%, 90%, 91%, 92%, 93%, 94%, 95%, 96%, 97%, 98%, 99% or more identity with a sequence selected from the group consisting of SEQ ID NOs: 1-14 and 55, wherein said Cas12a2 proteins comprise at least one amino acid residue selected from any one of the following positions corresponding with the SuCas12a2 protein (SEQ ID NO: 15): F370, Y375, P376, K378, A380, F381, W385, E386, A389, I898, L899, D900, L901, L904, E907, L916, V917, D918, L1029, K1036, G1038, A1041, N1042, and G1045. In certain embodiments, a Cas12a2 protein comprises at least about 80%, 85%, 86%, 87%, 88%, 89%, 90%, 91%, 92%, 93%, 94%, 95%, 96%, 97%, 98%, 99% or more identity with a sequence selected from the group consisting of SEQ ID NOs: 1-14 and 55, and comprises the following amino acid residues at the positions corresponding with the SuCas12a2 protein (SEQ ID NO: 15): F370, Y375, P376, K378, A380, F381, W385, E386, A389, I898, L899, D900, L901, L904, E907, L916, V917, D918, L1029, K1036, G1038, A1041, N1042, and G1045. In certain embodiments, the Cas12a2 protein comprises at least about 80%, 85%, 86%, 87%, 88%, 89%, 90%, 91%, 92%, 93%, 94%, 95%, 96%, 97%, 98%, 99% or more identity with a sequence selected from the group consisting of SEQ ID NOs: 1-14 and 55, and comprises a Sulf-type Cas12a2 conserved motif selected from any one of SEQ ID NOs: 30-46 (e.g., wherein the Cas12a2 protein retains nuclease activity). In certain embodiments, the Cas12a2 protein comprises at least about 80%, 85%, 86%, 87%, 88%, 89%, 90%, 91%, 92%, 93%, 94%, 95%, 96%, 97%, 98%, 99% or more identity with a sequence selected from the group consisting of SEQ ID NOs: 1-14 and 55, and comprises a Sulf-type Cas12a2 conserved motif selected from any one of SEQ ID NOs: 32, 40, and 42 (e.g., wherein the Cas12a2 protein retains nuclease activity).

The polynucleotides encoding Cas12a2 polypeptides disclosed herein can be used to isolate corresponding sequences from other prokaryotic or eukaryotic organisms, or from metagenomically-derived sequences whose native host organism is unclear or unknown. In this manner, methods such as PCR, hybridization, and the like can be used to identify such sequences based on their sequence homology or identity to the sequences set forth herein. Sequences isolated based on their sequence identity to the entire Cas12a2 sequences set forth herein or to variants and fragments thereof are encompassed by the present invention. Such sequences include sequences that are orthologs of the disclosed Cas12a2 sequences. “Orthologs” is intended to mean genes derived from a common ancestral gene and which are found in different species as a result of speciation. Genes found in different species are considered orthologs when their nucleotide sequences and/or their encoded protein sequences share at least about 75%, about 80%, about 85%, about 90%, about 91%, about 92%, about 93%, about 94%, about 95%, about 96%, about 97%, about 98%, about 99%, or greater sequence identity. Functions of orthologs are often highly conserved among species. Thus, isolated polynucleotides that encode polypeptides having Cas12a2 endonuclease activity and which share at least about 75% or more sequence identity to the sequences disclosed herein, are encompassed by the present invention.

Fragments and variants of the Cas12a2 polynucleotides and Cas12a2 amino acid sequences encoded thereby that retain Cas12a2 nuclease activity are encompassed herein. By “Cas12a2 nuclease activity” or “Cas12a2 activity” is intended the binding of and hybridization with a pre-determined nucleotide sequence (the “target sequence”) as mediated by a guide RNA. Cas12a2 nuclease activity can comprise double-strand break production of the target sequence (“primary activity”), and can further comprise non-sequence-specific nuclease activity directed against dsDNA and/or RNA (“secondary activity”) following the primary activity. Cas12a2 activity can encompass primary activity that can result in an initial site-specific single or double-strand cut to a polynucleotide followed by secondary activity that can result in a non-specific cleavage and/or degradation of polynucleotides in a cell. The primary activity can produce (i) a single-strand or double-strand break in dsDNA or dsRNA, or (ii) a single-strand break in ssRNA or ssDNA. This site-specific primary activity occurs at a target sequence adjacent to a recognition sequence, which may be referred to as a protospacer adjacent motif (PAM), a protospacer flanking motif (PFM), or a protospacer flanking sequence (PFS). While, in certain embodiments, the term PAM is used in the context of DNA targets and the terms PFM and PFS are used in the context of RNA targets, the terms PAM, PFM, and PFS may be used interchangeably in the context of DNA and RNA targets.

In certain embodiments, an RNA target sequence comprises the reverse complement of a corresponding DNA target sequence, such that the reverse complement of any DNA target sequence disclosed herein can function as an RNA target sequence. Moreover, DNA target sequences can be located 3′ from a PAM and, thus, an RNA target sequence can be located 5′ of a PFM, PFS, or PAM. As used herein, target sequences can refer to a DNA or RNA target sequence that results in site-specific cleavage of the polynucleotide and precedes non-specific cleavage and/or degradation of other DNA or RNA in the cell.

By “fragment” is intended a portion of the polynucleotide or a portion of the amino acid sequence. “Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. Generally, variants of a particular polynucleotide of the invention will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

“Variant” amino acid or protein is intended to mean an amino acid or protein derived from the native amino acid or protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present invention are biologically active, that is they continue to possess the desired biological activity of the native protein. Biologically active variants of a native polypeptide will have at least about 80%, 85%, 90%, 91%, 92%, 93%, 94%, 95%, 96%, 97%, 98%, 99% or more sequence identity to the amino acid sequence for the native sequence as determined by sequence alignment programs and parameters described herein. A biologically active variant of a protein of the invention may differ from that protein by as few as 1-15 amino acid residues, as few as 1-10, such as 6-10, as few as 5, as few as 4, 3, 2, or even 1 amino acid residue. Biologically active variants of a native polypeptide will have at least about 80%, 85%, 90%, 91%, 92%, 93%, 94%, 95%, 96%, 97%, 98%, 99% or more of the activity of the native sequence. Activity of Cas12a2 variant polypeptides can be measured by the ability of the polypeptide to bind and/or cleave a target site in the presence of the appropriate guide RNA. In some embodiments, a variant Cas12a2 polypeptide comprises at least about 80% identity with a sequence selected from the group consisting of SEQ ID NOs: 1-14 and 55. In such instances, the variant Cas12a2 polypeptide can comprise one or more of the following amino acid residues at the positions corresponding with the SuCas12a2 protein (SEQ ID NO: 15): F370, Y375, P376, K378, A380, F381, W385, E386, A389, I898, L899, D900, L901, L904, E907, L916, V917, D918, L1029, K1036, G1038, A1041, N1042, and G1045. In some instances, the variant Cas12a2 polypeptide comprises all of the following amino acid residues at the positions corresponding with the SuCas12a2 protein (SEQ ID NO: 15): F370, Y375, P376, K37880, A380, F381, W385, E386, A389, I898, L899, D900, L901, L904, E907, L916, V917, D918, L1029, K1036, G1038, A1041, N1042, and G1045. The polynucleotides disclosed herein can encode a Cas12a2 polypeptide variant, wherein said variant Cas12a2 polypeptide comprises at least about 80% identity with a sequence selected from the group consisting of SEQ ID NOs: 1-14 and 55. In some instances, the polynucleotide encodes a variant Cas12a2 polypeptide, wherein the variant Cas12a2 comprises one or more of the following amino acid residues at the positions corresponding with the SuCas12a2 protein (SEQ ID NO: 15): F370, Y375, P376, K378, A380, F381, W385, E386, A389, I898, L899, D900, L901, L904, E907, L916, V917, D918, L1029, K1036, G1038, A1041, N1042, and G1045. In some instances, the variant Cas12a2 polypeptide encoding by the polynucleotide comprises all of the following amino acid residues at the positions corresponding with the SuCas12a2 protein (SEQ ID NO: 15): F370, Y375, P376, K378, A380, F381, W385, E386, A389, I898, L899, D900, L901, L904, E907, L916, V917, D918, L1029, K1036, G1038, A1041, N1042, and G1045.

Variant sequences may also be identified by analysis of existing databases of sequenced genomes. In this manner, corresponding sequences can be identified and used in the methods of the invention.

With respect to an amino acid sequence that is optimally aligned with a reference sequence, an amino acid residue “corresponds to” the position in the reference sequence with which the residue is paired in the alignment. The “position” is denoted by a number that sequentially identifies each amino acid in the reference sequence based on its position relative to the N-terminus when the two proteins are subjected to standard sequence alignments (e.g., using the BLASTp program) and aligned for maximum sequence identity across the entire protein. Owing to deletions, insertion, truncations, fusions, etc., that must be taken into account when determining an optimal alignment, in general the amino acid residue number in a test sequence as determined by simply counting from the N-terminus will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions there can be stretches of amino acids in either the reference or aligned sequence that do not correspond to any amino acid in the corresponding sequence.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 87:2264-2268, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, California, USA). Alignments using these programs can be performed using the default parameters. The CLUSTAL program is well described by Higgins et al. (1988) Gene 73:237-244; Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The MUSCLE algorithm for multiple sequence alignment may be used for comparisons of multiple nucleic acid or protein sequences (Edgar (2004) Nucleic Acids Research 32:1792-1797). The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See the website at www.ncbi.nlm.nih.gov. Alignment may also be performed manually by inspection.

The nucleic acid molecules encoding Cas12a2 polypeptides, or fragments or variants thereof, can be codon optimized for expression in an organism of interest (e.g., a prokaryotic cell or a eukaryotic cell). A “codon-optimized gene” is a gene having its frequency of codon usage designed to mimic the frequency of preferred codon usage of the host cell. Nucleic acid molecules can be codon optimized, either wholly or in part. Because any one amino acid (except for methionine and tryptophan) is encoded by a number of codons, the sequence of the nucleic acid molecule may be changed without changing the encoded amino acid. Codon optimization is when one or more codons are altered at the nucleic acid level such that the amino acids are not changed but expression in a particular host organism is increased. Those having ordinary skill in the art will recognize that codon tables and other references providing preference information for a wide range of organisms are available in the art (see, e.g., Zhang et al. (1991) Gene 105:61-72; Murray et al. (1989) Nucl. Acids Res. 17:477-508).

In some embodiments, DNA encoding the Cas12a2 polypeptides of the invention, and DNA encoding guide polynucleotide(s) of the invention, may be included as part of a bacteriophage or modified bacteriophage, or may be included as part of a plasmid (for example a conjugative plasmid), phagemid, cosmid, or other DNA molecule capable of replication in a bacterial cell or cells of interest. The terms phage and bacteriophage may be used interchangeably. In some embodiments, a phage or a phagemid derived from M13, lambda, p22, T7, Mu, T4 phage, PBSX, P1Puna-like, P2, 13, Bcep 1, Bcep 43, Bcep 78, T5 phage, phi, C2, L5, HK97, N15, T3 phage, P37, MS2, Q.beta., or Phi X 174, T2 phage, T12 phage, R17 phage, M13 phage, G4 phage, Enterobacteria phage P2, P4 phage, N4 phage, Pseudomonas phage .PHI.6, .PHI.29 phage or 186 phage may be used to deliver a polynucleotide encoding a Cas12a2 polypeptide of the invention and/or one or more guide polynucleotide(s) of the invention, to the bacterial cell(s) of interest. Bacteriophage may be engineered, for example, to have a broad or narrow host range using methods known in the art (Yehl et al 2019 BioRxiv dx.doi.org/10.1101/699090).

### II. Nucleic Acids Encoding Cas12a2 Polypeptides

Nucleic acids encoding any of the Cas12a2 polypeptides or fusion proteins described herein are provided. The nucleic acid can be RNA or DNA. Examples of polynucleotides that encode Cas12a2 polypeptides are set forth in the group consisting of SEQ ID NOs:16-29 In one embodiment, the nucleic acid encoding the Cas12a2 polypeptide is mRNA. The mRNA can be 5′ capped and/or 3′ polyadenylated. In another embodiment, the nucleic acid encoding the Cas12a2 polypeptide is DNA. The DNA can be present in a phage, plasmid, or other vector.

Nucleic acids encoding the Cas12a2 polypeptide or fusion proteins can be codon optimized for efficient translation into protein in the cell of interest. Programs for codon optimization are available in the art (e.g., OPTIMIZER at genomes.urv.es/OPTIMIZER; OptimumGene™ from GenScript at www.genscript.com/codon_opt.html).

In certain embodiments, DNA encoding the Cas12a2 polypeptide can be operably linked to at least one promoter sequence. The DNA coding sequence can be operably linked to a promoter control sequence for expression in a host cell of interest, for example a bacterial cell. “Operably linked” is intended to mean a functional linkage between two or more elements. For example, an operable linkage between a promoter and a coding region of interest (e.g., region coding for a Cas12a2 polypeptide or guide RNA) is a functional link that allows for expression of the coding region of interest. Operably linked elements may be contiguous or non-contiguous. When used to refer to the joining of two protein coding regions, by operably linked is intended that the coding regions are in the same reading frame.

The promoter sequence can be derived from bacterial sequences, viral sequences, synthetically-designed sequences, or other sources. It is recognized that different applications can be enhanced by the use of different promoters in the nucleic acid molecules to modulate the timing, location and/or level of expression of the Cas12a2 polypeptide and/or guide RNA. Such nucleic acid molecules may also contain, if desired, a promoter regulatory region (e.g., one conferring inducible, constitutive, or environmentally- or developmentally-regulated expression), a transcription initiation start site, a ribosome binding site, an RNA processing signal, a transcription termination site, and/or a polyadenylation signal.

The nucleic acid sequences encoding the Cas12a2 polypeptide can be operably linked to a promoter sequence that is recognized by a phage RNA polymerase for in vitro mRNA synthesis. In such embodiments, the in vitro-transcribed RNA can be purified for use in the methods of genome modification and/or cell elimination described herein. For example, the promoter sequence can be a T7, T3, or SP6 promoter sequence or a variation of a T7, T3, or SP6 promoter sequence. In some embodiments, the sequence encoding the Cas12a2 polypeptide can be operably linked to a promoter sequence for in vitro expression of the Cas12a2 polypeptide. In such embodiments, the expressed protein and/or guide polynucleotide such as a guide RNA can be purified for use in the methods described herein.

The DNA encoding the Cas12a2 polypeptide or fusion protein can be present in a vector. Suitable vectors include engineered bacteriophages, plasmid vectors (for example conjugative plasmid vectors), phagemids, cosmids, artificial/mini-chromosomes, transposons, and viral vectors (e.g., lentiviral vectors, adeno-associated viral vectors, etc.). In one embodiment, the DNA encoding the Cas12a2 polypeptide is present in a plasmid vector. Non-limiting examples of suitable plasmid vectors include pUC, pBR322, pET, pBluescript, pCAMBIA, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001. In some embodiments, the DNA encoding the Cas12a2 polypeptide is present in an engineered bacteriophage, where the native bacteriophage sequence is derived from a bacteriophage that is capable of infecting the bacterial cell(s) of interest.

In some embodiments, the expression vector comprising the sequence encoding the Cas12a2 polypeptide can further comprise a sequence encoding a guide RNA. The sequence encoding the guide RNA can be operably linked to at least one transcriptional control sequence for expression of the guide RNA in the cell of interest.

### III. Methods for Targeting a Nucleotide Sequence in a Cell

Methods are provided herein for targeting a nucleotide sequence in a cell of interest, such as a bacterial cell or a eukaryotic cell. The cell of interest can be the cell of one or more pest of interest. Additionally, the target sequence can be a target sequence that is specific to the pest of interest. The pest of interest can be a pathogenic bacterial species, such as a pathogenic bacterial species associated with plants or mammals. In some instances, the pathogenic bacterial species is associated with humans. The pathogenic bacterial species can be a bacterial species selected from the group consisting of Xanthomonas sp., Escherichia sp., Pseudomonas sp., Erwinia sp., Xylella sp., Clavibacter sp., Ralstonia sp., Pectobacterium sp., Streptomyces sp., Burkholderia sp., Phytoplasma sp., Acidovorax sp., Pantoea sp., Agrobacterium sp., Spiroplasma sp., Candidatus Liberibacter sp., Dickeya sp., Serratia sp., Sphingomonas sp., Rhizobacter sp., Rhizomonas sp., Xylophilus sp., Rickettsia sp., Bacillus sp., Clostridium sp., Arthrobacter sp., Curtobacterium sp., Leifsonia sp., Rhodococcus sp., Phytoplasma sp., Enterobacter sp., Citrobacter sp., Klebsiella sp., Hafnia sp., Corynebacterium sp., Mycoplasma sp., Serratia sp., Pasteurella sp., Proteus sp., Campylobacter sp., Salmonella sp., Pseudomonas sp., Brucella sp., Staphylococcus sp., Streptococcus sp., Trueperella sp., Clostridium sp., Listeria sp., Anthrax sp., Bartonella sp., Capnocytophaga sp., Streptobacillus sp., Rickettsia sp., Anaplasma sp., Shigella sp., Borrelia sp., Actinomyces sp., Bacteroides sp., Bordetella sp., Chlamydia sp., Chlamydophila sp., Ehrlichia sp., Enterococcus sp., Francisella sp., Haemophilus sp., Helicobacter sp., Klebsiella sp., Legionella sp., Leptospira sp., Mycobacterium sp., Neisseria sp., Nocardia sp., Treponema sp., Vibrio sp., Yersinia sp., Coxiella sp., Wolbachia sp., Liberibacter sp., Aeromonas sp., Edwardsiella sp., Flavobacterium sp., Tenacibaculum sp., Renibacterium sp., Piscirickettsia sp., Enterobacterium sp., Lactococcus sp., Aerococcus sp., and Hepatobacter sp.

The methods comprise introducing into a cell one or more DNA-targeting polynucleotides such as, for example, a DNA-targeting RNA (“guide RNA,” “gRNA,” “CRISPR RNA,” or “crRNA”) or a DNA polynucleotide encoding a DNA-targeting RNA, wherein the DNA-targeting polynucleotide comprises: (a) a first segment comprising a nucleotide sequence that is complementary to a sequence in the target DNA; and (b) a second segment that interacts with a Cas12a2 polypeptide and also introducing to the cell a Cas12a2 polypeptide, or a polynucleotide such as a DNA molecule or an RNA molecule encoding a Cas12a2 polypeptide, wherein the a Cas12a2 polypeptide comprises: (a) a polynucleotide-binding portion that interacts with the gRNA or other DNA-targeting polynucleotide; and (b) an activity portion that may comprise a catalytic domain such as a RuvC domain that exhibits site-directed enzymatic activity.

The guide polynucleotide can be fully complementary to the target sequence. In other embodiments, the guide polynucleotide is partially complementary to the target sequence, and has a sequence differing by no more than 4 (i.e., 1, 2, 3, or 4) nucleotides from a nucleic acid sequence that is fully complementary to the target sequence. The partial complementarity to the target sequences (e.g., mismatches) within the guide polynucleotide may improve specificity of the guide polynucleotide and the Cas12a2 system comprising the guide polynucleotide to the target sequence.

In some embodiments, these methods result in the partial or complete killing and elimination of the cell or cells into which the Cas12a2 or encoding polynucleotide and guide polynucleotide have been introduced. For example, the methods described herein can result in a 5%, 10%, 15%, 20%, 25%, 30%, 50%, 75%, 80%, 85%0, 90%, 95%, 96%, 97%, 98%, 99%, 100% or 5-20%, 25-50%, 50-60%, 60-75%, 50-80%, 80-90%, 80-95%, 80-99%, 90-95%, 90-99%, or more decrease in the viable bacterial population in which the Cas12a2 or encoding polynucleotide and guide polynucleotide have been introduced.

The methods disclosed herein comprise introducing into a cell of interest at least one Cas12a2 polypeptide or a nucleic acid encoding at least one Cas12a2 polypeptide, as described herein. In some embodiments, the Cas12a2 polypeptide can be introduced into the cell as an isolated protein. In such embodiments, the Cas12a2 polypeptide can further comprise at least one cell-penetrating domain, which facilitates cellular uptake of the protein. In some embodiments, the Cas12a2 polypeptide can be introduced into the cell as a nucleoprotein in complex with a guide polynucleotide (for instance, as a ribonucleoprotein in complex with a guide RNA). In other embodiments, the Cas12a2 polypeptide can be introduced into the genome host as an mRNA molecule that encodes the Cas12a2 polypeptide. In still other embodiments, the Cas12a2 polypeptide can be introduced into the cell or cells as a DNA molecule comprising an open reading frame that encodes the Cas12a2 polypeptide. In general, DNA sequences encoding the Cas12a2 polypeptide or fusion protein described herein are operably linked to a promoter sequence that will function in the cell or cells of interest. The DNA sequence can be linear, or the DNA sequence can be part of a vector. In still other embodiments, the Cas12a2 polypeptide can be introduced into the cell or cells as an RNA-protein complex comprising the guide RNA. In certain embodiments, the Cas12a2 polypeptide, Cas12a2-gRNA ribonucleoprotein complex, and/or Cas12a2-encoding polynucleotide can be introduced into the cell or cells of interest via nanoparticle-aided transformation (Kumari et al 2017 FEMS Microbiol Lett 364:fnx081; French 2019 BioRxiv dx.doi.org/10.1101/559252).

In certain embodiments, DNA encoding the Cas12a2 polypeptide can further comprise a sequence encoding one or more guide RNAs. In general, each of the sequences encoding the Cas12a2 polypeptide and the guide RNA(s) is operably linked to one or more appropriate promoter sequences that enable expression of the Cas12a2 polypeptide and the guide RNA(s), respectively, in the cell or cells of interest. The DNA sequence encoding the Cas12a2 polypeptide and the guide RNA(s) can further comprise additional expression control, regulatory, and/or processing sequence(s). The DNA sequence encoding the Cas12a2 polypeptide and the guide RNA(s) can be linear or can be part of a vector.

Methods described herein further can also comprise introducing into a cell or cells at least one guide RNA or DNA encoding at least one polynucleotide such as a guide RNA. A guide RNA interacts with the Cas12a2 polypeptide to direct the Cas12a2 polypeptide to a specific target site, at which site the guide RNA base pairs with a specific DNA sequence in the targeted site. Guide RNAs can comprise three regions: a first region that is complementary to the target site in the targeted DNA sequence, a second region that forms a stem loop structure, and a third region that remains essentially single-stranded. The first region of each guide RNA is different such that each guide RNA guides a Cas12a2 polypeptide to a specific target site. The second and third regions of each guide RNA can be the same in all guide RNAs.

One region of the guide RNA is complementary to a sequence (i.e., protospacer sequence) at the target site in the targeted DNA such that the first region of the guide RNA can base pair with the target site. In various embodiments, the first region of the guide RNA can comprise from about 8 nucleotides to more than about 30 nucleotides. For example, the region of base pairing between the first region of the guide RNA and the target site in the nucleotide sequence can be about 8, about 9, about 10, about 11, about 12, about 13, about 14, about 15, about 16, about 17, about 18, about 19, about 20, about 22, about 23, about 24, about 25, about 27, about 30 or more than 30 nucleotides in length. In an exemplary embodiment, the first region of the guide RNA is about 20, 21, 22, 23, 24, or 25 nucleotides in length. The guide RNA also can comprise a second region that forms a secondary structure. In some embodiments, the secondary structure comprises a stem or hairpin. The length of the stem can vary. For example, the stem can range from about 5, to about 6, to about 10, to about 15, to about 20, to about 25 base pairs in length. The stem can comprise one or more bulges of 1 to about 10 nucleotides. In some preferred embodiments, the hairpin structure comprises the sequence UCUACN₃₋₅GUAGAU (SEQ ID NOs: 47-49, encoded by SEQ ID NOs: 50-52), with “UCUAC” and “GUAGA” base-pairing to form the stem. “N₃₋₅” indicates 3, 4, or 5 nucleotides. Thus, the overall length of the second region can range from about 14 to about 25 nucleotides in length. In certain embodiments, the loop is about 3, 4, or 5 nucleotides in length and the stem comprises about 5, 6, 7, 8, 9, or 10 base pairs.

The guide RNA can also comprise a third region that remains essentially single-stranded. Thus, the third region has no complementarity to any nucleotide sequence in the cell of interest and has no complementarity to the rest of the guide RNA. The length of the third region can vary. In general, the third region is more than about 4 nucleotides in length. For example, the length of the third region can range from about 5 to about 60 nucleotides in length. The combined length of the second and third regions (also called the universal or scaffold region) of the guide RNA can range from about 30 to about 120 nucleotides in length. In one aspect, the combined length of the second and third regions of the guide RNA range from about 40 to about 45 nucleotides in length.

In a preferred embodiment, the guide RNA comprises a single molecule comprising all three regions. In other embodiments, the guide RNA can comprise two separate molecules. The first RNA molecule can comprise the first region of the guide RNA and one half of the “stem” of the second region of the guide RNA. The second RNA molecule can comprise the other half of the “stem” of the second region of the guide RNA and the third region of the guide RNA. Thus, in this embodiment, the first and second RNA molecules each contain a sequence of nucleotides that are complementary to one another. For example, in one embodiment, the first and second RNA molecules each comprise a sequence (of about 6 to about 25 nucleotides) that base pairs to the other sequence to form a functional guide RNA. In specific embodiments, the guide RNA is a single molecule (i.e., crRNA) that interacts with the target site in the chromosome and the Cas12a2 polypeptide without the need for a second guide RNA (i.e., a tracrRNA).

In certain embodiments, the guide RNA(s) can be introduced into the cell as an RNA molecule. The RNA molecule can be transcribed in vitro. Alternatively, the RNA molecule can be chemically synthesized. In other embodiments, the guide RNA can be introduced into the genome host as a DNA molecule that encodes the guide RNA. In such cases, the DNA encoding the guide RNA can be operably linked to one or more promoter sequences for expression of the guide RNA in the cell or cells of interest.

In some embodiments, multiple guide RNAs may be designed to target multiple target sequences in the cell(s) of interest and may be introduced into the cell(s) of interest in the form of a CRISPR array in the format direct repeat-spacer-direct repeat-spacer, etc., repeating for the number of desired spacers. In these CRISPR arrays, the direct repeat sequences represent the portion of the gRNA that is recognized by Cas12a2. The direct repeat is processed by Cas12a2 enzymes to generate mature crRNAs that associate with the Cas12a2 protein to form the ribonucleoprotein complex that hybridizes with the target sequences in the cell(s) of interest. Direct repeat sequences for use with Cas12a2 enzymes may take the form, for example, of one or more of the sequences set forth in SEQ ID NOs: 47-52. In some embodiments, multiple guide RNAs may be designed to target multiple target sequences in the bacterial cell(s) of interest and may be introduced into the bacterial cell(s) of interest in the form of a CRISPR array in which the mature gRNAs are processed by ribozymes or by tRNA processing pathways (WO 2019/138052; Port and Bullock (2016) BioRxiv dx.doi.org/10.1101/046417).

The DNA molecule encoding the Cas12a2 enzyme and/or the guide RNA(s) can be linear or circular. In some embodiments, the DNA sequence encoding the Cas12a2 enzyme and/or the guide RNA(s) can be part of a vector. Suitable vectors include plasmid vectors (for example conjugative plasmid vectors), phagemids, cosmids, artificial/mini-chromosomes, transposons, and viral vectors. In an exemplary embodiment, the DNA encoding the Cas12a2 enzyme and/or the guide RNA(s) is present in a plasmid vector. Non-limiting examples of suitable plasmid vectors include pUC, pBR322, pET, pBluescript, pCALMBIA, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In another exemplary embodiment, the DNA encoding the Cas12a2 enzyme and/or the guide RNA(s) can be part of a phagemid.

In embodiments in which both the Cas12a2 polypeptide and the guide RNA(s) are introduced into the genome host as DNA molecules, each can be part of a separate molecule (e.g., one vector containing Cas12a2 polypeptide or fusion protein coding sequence and a second vector containing guide RNA coding sequence(s)) or both can be part of the same molecule (e.g., one vector containing coding (and regulatory) sequence for both the Cas12a2 polypeptide and the guide RNA(s)).

A Cas12a2 polypeptide in conjunction with a guide RNA is directed to a target site (i.e., a targeted DNA sequence or target sequence) in a cell, wherein the Cas12a2 polypeptide hybridizes with the targeted DNA sequence (the “initial hybridization event”) and produces a double-stranded break (i.e., cleavage) in the targeted DNA sequence. The cleavage site can be located anywhere within the target sequence. Without being limited by theory, this initial hybridization event triggers a conformational change in the Cas12a2 polypeptide that allows the Cas12a2 polypeptide to degrade RNA and/or dsDNA in a non-sequence-specific manner. The target site has no sequence limitation except that the sequence is immediately preceded (upstream) or followed (downstream) by a consensus sequence. This consensus sequence is also known as a protospacer adjacent motif (PAM), a protospacer flanking motif (PFM), or a protospacer flanking sequence (PFS). Examples of PAM sequences include, but are not limited to, TTTN, NTTN, TTTV, and NTTV (wherein N is defined as any nucleotide and V is defined as A, G, or C). Further, example PAM3′ sequences for the Cas12a2 nucleases can include TTNV (e.g., TTAA, TTAC, TTAG, TTCA, TTCC, TTGG, TTGA, TTGC, TTGG, TTTA, TTTC, TTTG), VTTV (e.g., ATTA, ATTC, ATTG, CTTA, CTTC, CTTG, GTTA, GTTC, GTTG), and TCTV (e.g., TCTA, TCTC, TCTG). It is well-known in the art that a suitable PAM sequence must be located at the correct location relative to the targeted DNA sequence to allow the Cas12a2 nuclease to produce the desired double-stranded break. For Cas12a2 nucleases characterized to date, the PAM sequence is located immediately 5′ of the targeted DNA sequence or immediately 3′ of the target RNA sequence. Thus, the target sequence can be immediately downstream (3′) or upstream (5′) of the PAM sequence (e.g., within 1-10 nucleotides of target sequence). “Adjacent” or “immediately adjacent” refers to the target DNA sequence being about 1 nucleotide to 50 nucleotides, about 5 nucleotides to 45 nucleotides, or about 7 nucleotides to 40 nucleotides either upstream (5′) or downstream (3′) of the PAM sequence. In some embodiments, the target DNA sequence is adjacent or immediately adjacent to the PAM sequence when it is 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides upstream (5′) or downstream (3′) of the PAM sequence. For example, PAM can be located 1, 2, 3, 4, 5, 6, 7, 8, 9, or 10 nucleotides 5′ side (upstream) or 3′ side (downstream) of the target sequence. The PAM site requirements for a given Cas12a2 nuclease cannot at present be predicted computationally, and instead must be determined experimentally using methods available in the art (Zetsche et al. (2015) Cell 163:759-771; Marshall et al. (2018) Mol Cell 69:146-157). It is well-known in the art that PAM sequence specificity for a given nuclease enzyme is affected by enzyme concentration (Karvelis et al. (2015) Genome Biol 16:253). Thus, modulating the concentrations of Cas12a2 protein delivered to the cell or in vitro system of interest represents a way to alter the PAM site requirements associated with that Cas12a2 enzyme. Modulating Cas12a2 protein concentration in the system of interest may be achieved, for instance, by altering the promoter used to express the Cas12a2-encoding gene, by altering the concentration of ribonucleoprotein delivered to the cell or in vitro system, or by adding or removing introns that may play a role in modulating gene expression levels. As detailed herein, the first region of the guide RNA is complementary to the protospacer of the target sequence. Typically, the first region of the guide RNA is about 19 to 25 nucleotides in length.

The target site can be in the coding region of a gene, in an intron of a gene, in a control region of a gene, in a non-coding region between genes, etc. The gene can be a protein coding gene or an RNA coding gene. The gene can be any gene of interest as described herein. Cas12a2 collateral activity against RNA and/or dsDNA may be activated through an in initial hybridization event with any DNA sequence(s) in the cell(s) of interest as long as a suitable PAM site is located 5′ of the target sequence(s).

In some embodiments, the Cas12a2 protein, or Cas12a2 protein-encoding polynucleotide, and guide RNA(s), or DNA encoding the guide RNA(s), are introduced into a plurality of cells with the guide RNA(s) designed to target sequences that are present only in a certain fraction of the cells. In some embodiments, this will result in the elimination or reduction of those cells that comprise the target sequence(s) that the guide RNA(s) are designed to hybridize with.

By “predetermined” or “target sequence” is intended a nucleotide (e.g., DNA or RNA) sequence in the cell of interest that can be unique to that cell. The predetermined or target sequence may be genomic DNA, chromosomal DNA, and/or plasmid or other extrachromosomal DNA sequences present in the cell or cells of interest. Methods are available in the art to find unique sequences within genomes and include using a Pan-Core genome approach to find accessory genes of organisms. Additionally using a Best Bi-directional Blast analysis or using OrthoMCL etc, would identify accessory genes. Additionally, unique regions between a pair of genomes can be extracted from a pair-wise global alignment performed using any of the popular programs like Nucmer (MUMmer), Mauve, BLAST, and the like.

### III.B. Methods for Targeting a Eukaryotic Cell

Methods are provided herein for modifying a nucleotide sequence of a eukaryotic cell, or eukaryotic organelle.

Methods are provided herein for targeting a nucleotide sequence in a eukaryotic cell of interest, such as a mammalian cell. The methods comprise introducing into a eukaryotic cell one or more DNA-targeting polynucleotides such as, for example, a DNA-targeting RNA (“guide RNA,” “gRNA,” “CRISPR RNA,” or “crRNA”) or a DNA polynucleotide encoding a DNA-targeting RNA, wherein the DNA-targeting polynucleotide comprises: (a) a first segment comprising a nucleotide sequence that is complementary to a sequence in the target DNA; and (b) a second segment that interacts with a Cas12a2 polypeptide and also introducing to the eukaryotic cell a Cas12a2 polypeptide, or a polynucleotide such as a DNA molecule or an RNA molecule encoding a Cas12a2 polypeptide, wherein the a Cas12a2 polypeptide comprises: (a) a polynucleotide-binding portion that interacts with the gRNA or other DNA-targeting polynucleotide; and (b) an activity portion that may comprise a catalytic domain such as a RuvC domain that exhibits site-directed enzymatic activity.

In some embodiments, these methods result in the partial or complete killing and elimination of the eukaryotic cell or cells into which the Cas12a2 or encoding polynucleotide and guide polynucleotide have been introduced. For example, the methods described herein can result in a 5%, 10%, 15%, 20%, 25%, 30%, 50%, 75%, 80%, 85%, 90%, 95%, 96%, 97%, 98%, 99%, 100% or 5-20%, 25-50%, 50-60%, 60-75%, 50-80%, 80-90%, 80-95%, 80-99%, 90-95%, 90-99%, or more decrease in the viable cell population in which the Cas12a2 or encoding polynucleotide and guide polynucleotide have been introduced. In specific embodiments, the methods prevent growth or expansion of the eukaryotic cell. Cell viability can be measured by any method known in the art, including tetrazolium reduction, resazurin reduction, protease markers, ATP detection, flow cytometry and high content imaging, or any other method known in the art.

The methods disclosed herein comprise introducing into a eukaryotic cell of interest at least one Cas12a2 polypeptide or a nucleic acid encoding at least one Cas12a2 polypeptide, as described herein. In some embodiments, the Cas12a2 polypeptide can be introduced into the eukaryotic cell as an isolated protein. In such embodiments, the Cas12a2 polypeptide can further comprise at least one cell-penetrating domain, which facilitates cellular uptake of the protein. In some embodiments, the Cas12a2 polypeptide can be introduced into the eukaryotic cell as a nucleoprotein in complex with a guide polynucleotide (for instance, as a ribonucleoprotein in complex with a guide RNA). In other embodiments, the Cas12a2 polypeptide can be introduced into the genome host as an mRNA molecule that encodes the Cas12a2 polypeptide. In still other embodiments, the Cas12a2 polypeptide can be introduced into the eukaryotic cell or cells as a DNA molecule comprising an open reading frame that encodes the Cas12a2 polypeptide. In general, DNA sequences encoding the Cas12a2 polypeptide or fusion protein described herein are operably linked to a promoter sequence that will function in the eukaryotic cell or cells of interest. The DNA sequence can be linear, or the DNA sequence can be part of a vector. In still other embodiments, the Cas12a2 polypeptide can be introduced into the eukaryotic cell or cells as an RNA-protein complex comprising the guide RNA. In certain embodiments, the Cas12a2 polypeptide, Cas12a2-gRNA ribonucleoprotein complex, and/or Cas12a2-encoding polynucleotide can be introduced into the eukaryotic cell or cells of interest via nanoparticle-aided transformation (Kumari et al 2017 FEMS Microbiol Lett 364:fnx081; French 2019 BioRxiv dx.doi.org/10.1101/559252).

In certain embodiments, DNA encoding the Cas12a2 polypeptide can further comprise a sequence encoding one or more guide RNAs. In general, each of the sequences encoding the Cas12a2 polypeptide and the guide RNA(s) is operably linked to one or more appropriate promoter sequences that enable expression of the Cas12a2 polypeptide and the guide RNA(s), respectively, in the eukaryotic cell or cells of interest. The DNA sequence encoding the Cas12a2 polypeptide and the guide RNA(s) can further comprise additional expression control, regulatory, and/or processing sequence(s). The DNA sequence encoding the Cas12a2 polypeptide and the guide RNA(s) can be linear or can be part of a vector.

Methods described herein further can also comprise introducing into a eukaryotic cell or cells at least one guide RNA or DNA encoding at least one polynucleotide such as a guide RNA. A guide RNA interacts with the Cas12a2 polypeptide to direct the Cas12a2 polypeptide to a specific target site, at which site the guide RNA base pairs with a specific DNA sequence in the targeted site. Guide RNAs can comprise three regions: a first region that is complementary to the target site in the targeted DNA sequence, a second region that forms a stem loop structure, and a third region that remains essentially single-stranded. The first region of each guide RNA is different such that each guide RNA guides a Cas12a2 polypeptide to a specific target site. The second and third regions of each guide RNA can be the same in all guide RNAs.

In certain embodiments, the guide RNA(s) can be introduced into the eukaryotic cell as an RNA molecule. The RNA molecule can be transcribed in vitro. Alternatively, the RNA molecule can be chemically synthesized. In other embodiments, the guide RNA can be introduced into the genome host as a DNA molecule that encodes the guide RNA. In such cases, the DNA encoding the guide RNA can be operably linked to one or more promoter sequences for expression of the guide RNA in the eukaryotic cell or cells of interest.

In some embodiments, multiple guide RNAs may be designed to target multiple target sequences in the eukaryotic cell(s) of interest and may be introduced into the eukaryotic cell(s) of interest in the form of a CRISPR array in the format direct repeat-spacer-direct repeat-spacer, etc., repeating for the number of desired spacers. In these CRISPR arrays, the direct repeat sequences represent the portion of the gRNA that is recognized by Cas12a2. The direct repeat is processed by Cas12a2 enzymes to generate mature crRNAs that associate with the Cas12a2 protein to form the ribonucleoprotein complex that hybridizes with the target sequences in the eukaryotic cell(s) of interest. Direct repeat sequences for use with Cas12a2 enzymes may take the form, for example, of one or more of the sequences set forth in SEQ ID NOs:35-40. In some embodiments, multiple guide RNAs may be designed to target multiple target sequences in the cell(s) of interest and may be introduced into the cell(s) of interest in the form of a CRISPR array in which the mature gRNAs are processed by ribozymes or by tRNA processing pathways (WO 2019/138052; Port and Bullock (2016) BioRxiv dx.doi.org/10.1101/046417).

The DNA molecule encoding the Cas12a2 enzyme and/or the guide RNA(s) can be linear or circular. In some embodiments, the DNA sequence encoding the Cas12a2 enzyme and/or the guide RNA(s) can be part of a vector. Suitable vectors include plasmid vectors (for example conjugative plasmid vectors), phagemids, cosmids, artificial/mini-chromosomes, transposons, and viral vectors. In an exemplary embodiment, the DNA encoding the Cas12a2 enzyme and/or the guide RNA(s) is present in a plasmid vector. Non-limiting examples of suitable plasmid vectors include pUC, pBR322, pET, pBluescript, pCAMBIA, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In another exemplary embodiment, the DNA encoding the Cas12a2 enzyme and/or the guide RNA(s) can be part of a phagemid.

In embodiments in which both the Cas12a2 polypeptide and the guide RNA(s) are introduced into the genome host as DNA molecules, each can be part of a separate molecule (e.g., one vector containing Cas12a2 polypeptide or fusion protein coding sequence and a second vector containing guide RNA coding sequence(s)) or both can be part of the same molecule (e.g., one vector containing coding (and regulatory) sequence for both the Cas12a2 polypeptide and the guide RNA(s)).

A Cas12a2 polypeptide in conjunction with a guide RNA is directed to a target site (i.e., a targeted DNA sequence or target sequence) in a eukaryotic cell, wherein the Cas12a2 polypeptide hybridizes with the targeted DNA sequence (the “initial hybridization event”) and produces a double-stranded break (i.e., cleavage) in the targeted DNA sequence. The cleavage site can be located anywhere within the target sequence. Without being limited by theory, this initial hybridization event triggers a conformational change in the Cas12a2 polypeptide that allows the Cas12a2 polypeptide to degrade RNA and/or dsDNA in a non-sequence-specific manner. The target site has no sequence limitation except that the sequence is immediately preceded (upstream) or followed (downstream) by a consensus sequence. This consensus sequence is also known as a protospacer adjacent motif (PAM), a protospacer flanking motif (PFM), or a protospacer flanking sequence (PFS). Examples of PAM sequences include, but are not limited to, TTTN, NTTN, TTTV, and NTTV (wherein N is defined as any nucleotide and V is defined as A, G, or C). Further, example PAM sequences for the Cas12a2 nucleases can include TTNV (e.g., TTAA, TTAC, TTAG, TTCA, TTCC, TTGG, TTGA, TTGC, TTGG, TTTA, TTTC, TTTG), VTTV (e.g., ATTA, ATTC, ATTG, CTTA, CTTC, CTTG, GTTA, GTTC, GTTG), and TCTV (e.g., TCTA, TCTC, TCTG).

It is well-known in the art that a suitable PAM sequence must be located at the correct location relative to the targeted DNA sequence to allow the Cas12a2 nuclease to produce the desired double-stranded break. For Cas12a2 nucleases characterized to date, the PAM sequence is located immediately 5′ of the targeted DNA sequence or immediately 3′ of the target RNA sequence. Thus, the target sequence can be immediately downstream (3′) or upstream (5′) of the PAM sequence (e.g., within 1-10 nucleotides of target sequence). The PAM site requirements for a given Cas12a2 nuclease cannot at present be predicted computationally, and instead must be determined experimentally using methods available in the art (Zetsche et al. (2015) Cell 163:759-771; Marshall et al. (2018) Mol Cell 69:146-157). It is well-known in the art that PAM sequence specificity for a given nuclease enzyme is affected by enzyme concentration (Karvelis et al. (2015) Genome Biol 16:253). Thus, modulating the concentrations of Cas12a2 protein delivered to the cell or in vitro system of interest represents a way to alter the PAM site requirements associated with that Cas12a2 enzyme. Modulating Cas12a2 protein concentration in the system of interest may be achieved, for instance, by altering the promoter used to express the Cas12a2-encoding gene, by altering the concentration of ribonucleoprotein delivered to the cell or in vitro system, or by adding or removing introns that may play a role in modulating gene expression levels. As detailed herein, the first region of the guide RNA is complementary to the protospacer of the target sequence. Typically, the first region of the guide RNA is about 19 to 25 nucleotides in length.

The target site can be in the coding region of a gene, in an intron of a gene, in a control region of a gene, in a non-coding region between genes, etc. The gene can be a protein coding gene or an RNA coding gene. The gene can be any gene of interest as described herein. Cas12a2 collateral activity against RNA and/or dsDNA may be activated through an in initial hybridization event with any DNA sequence(s) in the eukaryotic cell(s) of interest as long as a suitable PAM site is located 5′ of the target sequence(s).

In some embodiments, the Cas12a2 protein, or Cas12a2 protein-encoding polynucleotide, and guide RNA(s), or DNA encoding the guide RNA(s), are introduced into a plurality of eukaryotic cells with the guide RNA(s) designed to target sequences that are present only in a certain fraction of the cells. In some embodiments, this will result in the elimination or reduction of those cells that comprise the target sequence(s) that the guide RNA(s) are designed to hybridize with.

The present invention may be used for transformation of any eukaryotic species, including, but not limited to animals (including but not limited to mammals, insects, fish, birds, and reptiles), plants, fungi, amoeba, and yeast.

Methods for the introduction of nuclease proteins, DNA or RNA molecules encoding nuclease proteins, guide RNAs or DNA molecules encoding guide RNAs, and optional donor sequence DNA molecules into eukaryotic cells or organelles are known in the art, for instance in U.S. Patent Application 2016/0208243, herein incorporated by reference. Exemplary genetic modifications to eukaryotic cells or organelles that may be of particular value for industrial applications are also known in the art, for instance in U.S. Patent Application 2016/0208243, herein incorporated by reference.

### III.A.1. Methods for Targeting a Plant Pathogen

The compositions provided herein may be delivered to a plant, where the Cas12a2 polypeptide (in combination with an appropriate guide polynucleotide) may in turn selectively target or eliminate a plant pathogen. In certain embodiments, the plant may be modified to express the Cas12a2 polypeptide and a guide polynucleotide specific for one or more plant pathogens. In alternative embodiments, the composition comprising the Cas12a2 polypeptide and a guide polynucleotide may be applied to the surface of a plant (e.g., a surface that may come into contact with a plant pathogen).

The Cas12a2 polypeptide (or encoding nucleic acid), the guide RNA(s) (or encoding DNA), and the optional donor polynucleotide(s) can be introduced into a plant cell, organelle, or plant embryo by a variety of means, including transformation. Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda etal. (1996) Nature Biotechnology 14:745-750 (maize via Agrobacterium tumefaciens); all of which are herein incorporated by reference. Site-specific genome editing of plant cells by biolistic introduction of a ribonucleoprotein comprising a nuclease and suitable guide RNA has been demonstrated (Svitashev et al (2016) Nat Commun doi: 10.1038/ncomms13274); these methods are herein incorporated by reference. “Stable transformation” is intended to mean that the nucleotide construct introduced into a plant integrates into the genome of the plant and is capable of being inherited by the progeny thereof. The nucleotide construct may be integrated into the nuclear, plastid, or mitochondrial genome of the plant. Methods for plastid transformation are known in the art (see, e.g., Chloroplast Biotechnology: Methods and Protocols (2014) Pal Maliga, ed. and U.S. Patent Application 2011/0321187), and methods for plant mitochondrial transformation have been described in the art (see, e.g., U.S. Patent Application 2011/0296551), herein incorporated by reference.

The cells that have been transformed may be grown into plants (i.e., cultured) in accordance with conventional ways. See, for example, McCormick et al. (1986) Plant Cell Reports 5:81-84. In this manner, the present invention provides transformed seed (also referred to as “transgenic seed”) having a nucleic acid modification stably incorporated into their genome.

“Introduced” in the context of inserting a nucleic acid fragment (e.g., a recombinant DNA construct) into a cell, means “transfection” or “transformation” or “transduction” and includes reference to the incorporation of a nucleic acid fragment into a plant cell where the nucleic acid fragment may be incorporated into the genome of the cell (e.g., nuclear chromosome, plasmid, plastid chromosome or mitochondrial chromosome), converted into an autonomous replicon, or transiently expressed (e.g., transfected mRNA).

The present invention may be used for transformation of any plant species, including, but not limited to, monocots and dicots (i.e., monocotyledonous and dicotyledonous, respectively). Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers.

The Cas12a2 polypeptides (or encoding nucleic acid), the guide RNA(s) (or DNAs encoding the guide RNA), and the optional donor polynucleotide(s) can be introduced into the plant cell, organelle, or plant embryo simultaneously or sequentially. The ratio of the Cpf1 polypeptides (or encoding nucleic acid) to the guide RNA(s) (or encoding DNA) generally will be about stoichiometric such that the two components can form an RNA-protein complex with the target DNA. In one embodiment, DNA encoding a Cpf1 polypeptide and DNA encoding a guide RNA are delivered together within the plasmid vector.

The compositions and methods disclosed herein can be used to alter expression of genes of interest in a plant, such as genes involved in photosynthesis. Therefore, the expression of a gene encoding a protein involved in photosynthesis may be modulated as compared to a control plant. A “subject plant or plant cell” is one in which genetic alteration, such as a mutation, has been effected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell. Thus, the expression levels are higher or lower than those in the control plant depending on the methods of the invention.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

While the invention is described in terms of transformed plants, it is recognized that transformed organisms of the invention also include plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

Derivatives of coding sequences can be made using the methods disclosed herein to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Illinois), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Further provided herein is a method for increasing resistance or tolerance of a plant to one or more plant pathogens, the method comprising contacting a plant, plant part, or plant cell with (i) a composition comprising a Cas12a2 polypeptide provided herein or a polynucleotide encoding said Cas12a2 polypeptide and (ii) a guide polynucleotide, or a polynucleotide encoding a guide polynucleotide, wherein said guide polynucleotide binds said Cas12a2 polypeptide and hybridizes with a target sequence in one or more cells of interest, wherein said target sequence is located adjacent to a PAM sequence that is recognized by said Cas12a2 polypeptide to produce a modified plant, plant part, or plant cell. In some embodiments, the guide polynucleotide is designed to bind to the Cas12a2 polypeptide and hybridize to a target sequence in a plant pathogen, thereby increasing resistance or tolerance of the plant to the plant pathogen, as compared to resistance or tolerance of a control plant to the plant pathogen.

Further provided herein is a method for producing a modified plant with increased resistance or tolerance to one or more plant pathogens, the method comprising contacting a plant, plant part, or plant cell with (i) a composition comprising a Cas12a2 polypeptide provided herein or a polynucleotide encoding said Cas12a2 polypeptide and (ii) a guide polynucleotide, or a polynucleotide encoding a guide polynucleotide to produce a modified plant, plant part, or plant cell, and selecting for a modified plant, plant part, or plant cell that expresses the Cas12a2 polypeptide and the at least one guide polynucleotide. In some embodiments, the guide polynucleotide is capable of binding the Cas12a2 polypeptide and hybridizing to a target sequence in a plant pathogen, thereby producing a modified plant with increased resistance or tolerance to the plant pathogen, as compared to resistance or tolerance of a control plant to the plant pathogen.

In some embodiments, the selection step involves growing the plant, plant part, or plant cell in media comprising a selectable agent. The selectable agent may be, for example, an herbicide, an antibiotic, a carbohydrate, an amino acid, or a metabolite. In some embodiments, the control plant is a corresponding plant or population of plants that does not comprise the composition. In some embodiments, the modified plant comprises an improved agronomic trait (e.g., improved biomass yield and/or seed yield) as compared to the control plant.

The guide polynucleotide may be designed to hybridize with a target sequence specific to a plant pathogen (e.g., a target sequence not found in plant cells), thereby promoting selective elimination of the plant pathogen while keeping the plant cells unharmed. Examples of plant pathogens include a plant parasitic nematode, an insect, a fungus, a virus, a mollusk, a spider, a scorpion, a caterpillar, an animal, a mite, or a tick. Accordingly, in some embodiments, the guide polynucleotide is designed to hybridize to a target sequence specific to a plant parasitic nematode, an insect, a fungus, a virus, a mollusk, a spider, a scorpion, a caterpillar, an animal, a mite, or a tick. Alternatively, the target sequence may be one specific for a prokaryotic plant pathogen, as further described herein.

A plant described herein can be exposed to any of the compositions described herein in any suitable manner that permits the Cas12a2 composition to target a plant pathogen cell. In some embodiments, the method involves contacting the plant with a virus or viral nucleic acid molecule comprising the composition, microinjection, electroporation, Agrobacterium-mediated transformation, direct gene transfer, particle mediated delivery, topical application, silicon carbide fiber mediated delivery, delivery via cell-penetrating peptides, or a combination thereof. In some embodiments, the contacting step comprises introducing into the plant cell a composition provided herein, and culturing the plant cell to regenerate a plant or plant part comprising the composition.

In certain embodiments, the plant, plant part, or plant cell is corn (Zea mays), soybean (Glycine max), Brassica species, Brassica napus, Brassica rapa, Brassica juncea, rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

### III.A.2. Methods for Targeting Cancer Cells

Further provided are compositions and methods for modifying genomic DNA sequences and selectively killing cancer cells using Cas12a2 CRISPR systems. In some embodiments, the methods result in genome modification and/or cell death for cancer cells that harbor particular pre-determined and targeted DNA sequences leaving other cells that do not comprise the target DNA sequences unharmed.

In some embodiments, the present invention provides methods for eliminating particular types of cancer cells, including but not limited to cells associated lung cancers, head and neck squamous cancers, prostate cancer, and breast cancer in humans and other mammals in need of such treatment. These methods comprise administering a therapeutically effective amount of a Cas12a2 protein, or Cas12a2 protein-encoding polynucleotide, and guide RNA(s), or DNA encoding the guide RNA(s) to a subject in need thereof. In some embodiments, the Cas12a2 protein, or Cas12a2 protein-encoding polynucleotide, may be administered alone or in combination with a therapeutically effective amount of one or more additional anti-cancer compounds

The compositions may be administered to a mammalian subject ex vivo or in vivo. For in vivo administration, the composition may be administered systemically or locally to a mammalian subject in an amount effective to achieve selective depletion, inhibition, or killing of the target cells. The Cas12a2 polypeptide and a guide polynucleotide capable of targeting a target sequence in a cell of interest may be incorporated into any suitable delivery vector for mammals, such as a viral vector (e.g., AAV vector) or non-viral mode of delivery (e.g., lipid nanoparticle).

In some embodiments, the methods and compositions of the present invention can be used to treat common cancers, including but not limited to bladder cancer, breast cancer, colorectal cancer, endometrial cancer, head and neck cancer, leukemia, lung cancer, lymphoma, melanoma, ovarian cancer, and prostate cancer. Accordingly, in some embodiments, the methods involve delivering a Cas12a2 protein, or Cas12a2 protein-encoding polynucleotide, and a guide RNA(s), or DNA encoding the guide RNA(s) to a subject, wherein the guide RNA is one specific to a target sequence specific to cells associated with bladder cancer, breast cancer, colorectal cancer, endometrial cancer, head and neck cancer, leukemia, lung cancer, lymphoma, melanoma, ovarian cancer, and prostate cancer. In some embodiments the target sequence is specific to the cancer cell or other cell type that is targeted for removal.

### III.C. Methods for Targeting a Prokaryotic Cell

Methods are provided herein for targeting a nucleotide sequence in a bacterial cell. The methods comprise introducing into a bacterial cell one or more DNA-targeting polynucleotides such as, for example, a DNA-targeting RNA (“guide RNA,” “gRNA,” “CRISPR RNA,” or “crRNA”) or a DNA polynucleotide encoding a DNA-targeting RNA, wherein the DNA-targeting polynucleotide comprises: (a) a first segment comprising a nucleotide sequence that is complementary to a sequence in the target DNA; and (b) a second segment that interacts with a Cas12a2 polypeptide and also introducing to the bacterial cell a Cas12a2 polypeptide, or a polynucleotide such as a DNA molecule or an RNA molecule encoding a Cas12a2 polypeptide, wherein the a Cas12a2 polypeptide comprises: (a) a polynucleotide-binding portion that interacts with the gRNA or other DNA-targeting polynucleotide; and (b) an activity portion that may comprise a catalytic domain such as a RuvC domain that exhibits site-directed enzymatic activity. In some embodiments, these methods result in the partial or complete killing and elimination of the bacterial cell or cells into which the Cas12a2 or encoding polynucleotide and guide polynucleotide have been introduced. For example, the methods described herein can result in a 5%, 10%, 15%, 20%, 25%, 30%, 50%, 75%, 80%, 85%, 90%, 95%, 96%, 97%, 98%, 99%, 100% or 5-20%, 25-50%, 50-60%, 60-75%, 50-80%, 80-90%, 80-95%, 80-99%, 90-95%, 90-99%, or more decrease in the viable bacterial population in which the Cas12a2 or encoding polynucleotide and guide polynucleotide have been introduced. Bacterial cell viability can be measured by any method known in the art, including plate count (e.g., CFU, CFU/g, CFU/mL), turbidity measurement, cell lysis, or any other method known in the art. In specific embodiments, bacterial cell killing as used herein refers to a bacteriostatic elimination of future bacterial growth.

The methods disclosed herein comprise introducing into a bacterial cell at least one Cas12a2 polypeptide or a nucleic acid encoding at least one Cas12a2 polypeptide, as described herein. In some embodiments, the Cas12a2 polypeptide can be introduced into the bacterial cell as an isolated protein. In such embodiments, the Cas12a2 polypeptide can further comprise at least one cell-penetrating domain, which facilitates cellular uptake of the protein. In some embodiments, the Cas12a2 polypeptide can be introduced into the bacterial cell as a nucleoprotein in complex with a guide polynucleotide (for instance, as a ribonucleoprotein in complex with a guide RNA). In other embodiments, the Cas12a2 polypeptide can be introduced into the genome host as an mRNA molecule that encodes the Cas12a2 polypeptide. In still other embodiments, the Cas12a2 polypeptide can be introduced into the bacterial cell or cells as a DNA molecule comprising an open reading frame that encodes the Cas12a2 polypeptide. In general, DNA sequences encoding the Cas12a2 polypeptide or fusion protein described herein are operably linked to a promoter sequence that will function in the bacterial cell or cells of interest. The DNA sequence can be linear, or the DNA sequence can be part of a vector. In still other embodiments, the Cas12a2 polypeptide can be introduced into the bacterial cell or cells as an RNA-protein complex comprising the guide RNA. In certain embodiments, the Cas12a2 polypeptide, Cas12a2-gRNA ribonucleoprotein complex, and/or Cas12a2-encoding polynucleotide can be introduced into the bacterial cell or cells of interest via nanoparticle-aided transformation (Kumari et al 2017 FEMS Microbiol Lett 364:fnx081; French 2019 BioRxiv dx.doi.org/10.1101/559252).

In certain embodiments, DNA encoding the Cas12a2 polypeptide can further comprise a sequence encoding one or more guide RNAs. In general, each of the sequences encoding the Cas12a2 polypeptide and the guide RNA(s) is operably linked to one or more appropriate promoter sequences that enable expression of the Cas12a2 polypeptide and the guide RNA(s), respectively, in the bacterial cell or cells of interest. The DNA sequence encoding the Cas12a2 polypeptide and the guide RNA(s) can further comprise additional expression control, regulatory, and/or processing sequence(s). The DNA sequence encoding the Cas12a2 polypeptide and the guide RNA(s) can be linear or can be part of a vector.

Methods described herein further can also comprise introducing into a bacterial cell or cells at least one guide RNA or DNA encoding at least one polynucleotide such as a guide RNA. A guide RNA interacts with the Cas12a2 polypeptide to direct the Cas12a2 polypeptide to a specific target site, at which site the guide RNA base pairs with a specific DNA sequence in the targeted site. Guide RNAs can comprise three regions: a first region that is complementary to the target site in the targeted DNA sequence, a second region that forms a stem loop structure, and a third region that remains essentially single-stranded. The first region of each guide RNA is different such that each guide RNA guides a Cas12a2 polypeptide to a specific target site. The second and third regions of each guide RNA can be the same in all guide RNAs.

One region of the guide RNA is complementary to a sequence (i.e., protospacer sequence) at the target site in the targeted DNA such that the first region of the guide RNA can base pair with the target site. In various embodiments, the first region of the guide RNA can comprise from about 8 nucleotides to more than about 30 nucleotides. For example, the region of base pairing between the first region of the guide RNA and the target site in the nucleotide sequence can be about 8, about 9, about 10, about 11, about 12, about 13, about 14, about 15, about 16, about 17, about 18, about 19, about 20, about 22, about 23, about 24, about 25, about 27, about 30 or more than 30 nucleotides in length. In an exemplary embodiment, the first region of the guide RNA is about 20, 21, 22, 23, 24, or 25 nucleotides in length. The guide RNA also can comprise a second region that forms a secondary structure. In some embodiments, the secondary structure comprises a stem or hairpin. The length of the stem can vary. For example, the stem can range from about 5, to about 6, to about 10, to about 15, to about 20, to about 25 base pairs in length. The stem can comprise one or more bulges of 1 to about 10 nucleotides. In some preferred embodiments, the hairpin structure comprises the sequence UCUACN₃₋₅GUAGAU (SEQ ID NOs: 47-49, encoded by SEQ ID NOs: 50-52), with “UCUAC” and “GUAGA” base-pairing to form the stem. “N₃₋₅” indicates 3, 4, or 5 nucleotides. Thus, the overall length of the second region can range from about 14 to about 25 nucleotides in length. In certain embodiments, the loop is about 3, 4, or 5 nucleotides in length and the stem comprises about 5, 6, 7, 8, 9, or 10 base pairs.

The guide RNA can also comprise a third region that remains essentially single-stranded. Thus, the third region has no complementarity to any nucleotide sequence in the cell of interest and has no complementarity to the rest of the guide RNA. The length of the third region can vary. In general, the third region is more than about 4 nucleotides in length. For example, the length of the third region can range from about 5 to about 60 nucleotides in length. The combined length of the second and third regions (also called the universal or scaffold region) of the guide RNA can range from about 30 to about 120 nucleotides in length. In one aspect, the combined length of the second and third regions of the guide RNA range from about 40 to about 45 nucleotides in length.

In a preferred embodiment, the guide RNA comprises a single molecule comprising all three regions. In other embodiments, the guide RNA can comprise two separate molecules. The first RNA molecule can comprise the first region of the guide RNA and one half of the “stem” of the second region of the guide RNA. The second RNA molecule can comprise the other half of the “stem” of the second region of the guide RNA and the third region of the guide RNA. Thus, in this embodiment, the first and second RNA molecules each contain a sequence of nucleotides that are complementary to one another. For example, in one embodiment, the first and second RNA molecules each comprise a sequence (of about 6 to about 25 nucleotides) that base pairs to the other sequence to form a functional guide RNA. In specific embodiments, the guide RNA is a single molecule (i.e., crRNA) that interacts with the target site in the chromosome and the Cas12a2 polypeptide without the need for a second guide RNA (i.e., a tracrRNA).

In certain embodiments, the guide RNA(s) can be introduced into the bacterial cell as an RNA molecule. The RNA molecule can be transcribed in vitro. Alternatively, the RNA molecule can be chemically synthesized. In other embodiments, the guide RNA can be introduced into the genome host as a DNA molecule that encodes the guide RNA. In such cases, the DNA encoding the guide RNA can be operably linked to one or more promoter sequences for expression of the guide RNA in the bacterial cell or cells of interest.

In some embodiments, multiple guide RNAs may be designed to target multiple target sequences in the bacterial cell(s) of interest and may be introduced into the bacterial cell(s) of interest in the form of a CRISPR array in the format direct repeat-spacer-direct repeat-spacer, etc., repeating for the number of desired spacers. In these CRISPR arrays, the direct repeat sequences represent the portion of the gRNA that is recognized by Cas12a2. The direct repeat is processed by Cas12a2 enzymes to generate mature crRNAs that associate with the Cas12a2 protein to form the ribonucleoprotein complex that hybridizes with the target sequences in the bacterial cell(s) of interest. Direct repeat sequences for use with Cas12a2 enzymes may take the form, for example, of one or more of the sequences set forth in SEQ ID NOs: 47-52. In some embodiments, multiple guide RNAs may be designed to target multiple target sequences in the bacterial cell(s) of interest and may be introduced into the bacterial cell(s) of interest in the form of a CRISPR array in which the mature gRNAs are processed by ribozymes or by tRNA processing pathways (WO 2019/138052; Port and Bullock (2016) BioRxiv dx.doi.org/10.1101/046417).

The DNA molecule encoding the Cas12a2 enzyme and/or the guide RNA(s) can be linear or circular. In some embodiments, the DNA sequence encoding the Cas12a2 enzyme and/or the guide RNA(s) can be part of a vector. Suitable vectors include plasmid vectors (for example conjugative plasmid vectors), phagemids, cosmids, artificial/mini-chromosomes, transposons, and viral vectors. In an exemplary embodiment, the DNA encoding the Cas12a2 enzyme and/or the guide RNA(s) is present in a plasmid vector. Non-limiting examples of suitable plasmid vectors include pUC, pBR322, pET, pBluescript, pCAMBIA, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In another exemplary embodiment, the DNA encoding the Cas12a2 enzyme and/or the guide RNA(s) can be part of a phagemid.

In embodiments in which both the Cas12a2 polypeptide and the guide RNA(s) are introduced into the genome host as DNA molecules, each can be part of a separate molecule (e.g., one vector containing Cas12a2 polypeptide or fusion protein coding sequence and a second vector containing guide RNA coding sequence(s)) or both can be part of the same molecule (e.g., one vector containing coding (and regulatory) sequence for both the Cas12a2 polypeptide and the guide RNA(s)).

A Cas12a2 polypeptide in conjunction with a guide RNA is directed to a target site (i.e., a targeted DNA sequence or target sequence) in a bacterial cell, wherein the Cas12a2 polypeptide hybridizes with the targeted DNA sequence (the “initial hybridization event”) and produces a double-stranded break (i.e., cleavage) in the targeted DNA sequence. The cleavage site can be located anywhere within the target sequence. Without being limited by theory, this initial hybridization event triggers a conformational change in the Cas12a2 polypeptide that allows the Cas12a2 polypeptide to degrade RNA and/or dsDNA in a non-sequence-specific manner. The target site has no sequence limitation except that the sequence is immediately preceded (upstream) by a consensus sequence. This consensus sequence is also known as a protospacer adjacent motif (PAM), a protospacer flanking motif (PFM), or a protospacer flanking sequence (PFS). Examples of PAM sequences include, but are not limited to, TTTN, NTTN, TTTV, and NTTV (wherein N is defined as any nucleotide and V is defined as A, G, or C). Further, example PAM sequences for the Cas12a2 nucleases can include TTNV (e.g., TTAA, TTAC, TTAG, TTCA, TTCC, TTGG, TTGA, TTGC, TTGG, TTTA, TTTC, TTTG), VTTV (e.g., ATTA, ATTC, ATTG, CTTA, CTTC, CTTG, GTTA, GTTC, GTTG), and TCTV (e.g., TCTA, TCTC, TCTG).

It is well-known in the art that a suitable PAM sequence must be located at the correct location relative to the targeted DNA sequence to allow the Cas12a2 nuclease to produce the desired double-stranded break. For Cas12a2 nucleases characterized to date, the PAM sequence is located immediately 5′ of the targeted DNA sequence or immediately 3′ of the target RNA sequence. Thus, the target sequence can be immediately downstream (3′) or upstream (5′) of the PAM sequence (e.g., within 1-10 nucleotides of target sequence). The PAM site requirements for a given Cas12a2 nuclease cannot at present be predicted computationally, and instead must be determined experimentally using methods available in the art (Zetsche et al. (2015) Cell 163:759-771; Marshall et al. (2018) Mol Cell 69:146-157). It is well-known in the art that PAM sequence specificity for a given nuclease enzyme is affected by enzyme concentration (Karvelis et al. (2015) Genome Biol 16:253). Thus, modulating the concentrations of Cas12a2 protein delivered to the cell or in vitro system of interest represents a way to alter the PAM site requirements associated with that Cas12a2 enzyme. Modulating Cas12a2 protein concentration in the system of interest may be achieved, for instance, by altering the promoter used to express the Cas12a2-encoding gene, by altering the concentration of ribonucleoprotein delivered to the cell or in vitro system, or by adding or removing introns that may play a role in modulating gene expression levels. As detailed herein, the first region of the guide RNA is complementary to the protospacer of the target sequence. Typically, the first region of the guide RNA is about 19 to 25 nucleotides in length. In some embodiments, the first region of the guide RNA is fully complementary to the target sequence. In other embodiments, the first region of the guide RNA is partially complementary to the target sequence, and has a sequence differing by no more than 4 (i.e., 1, 2, 3, or 4) nucleotides from a nucleic acid sequence that is fully complementary to the target sequence. The partial complementarity to the target sequences (e.g., mismatches) introduced to the guide RNA may improve specificity of the guide RNA and the Cas12a2 system comprising the guide RNA to the target sequence.

The target site can be in the coding region of a gene, in an intron of a gene, in a control region of a gene, in a non-coding region between genes, etc. The gene can be a protein coding gene or an RNA coding gene. The gene can be any gene of interest as described herein. Cas12a2 collateral activity against RNA and/or dsDNA may be activated through an in initial hybridization event with any DNA sequence(s) in the bacterial cell(s) of interest as long as a suitable PAM site is located 5′ of the target sequence(s).

In some embodiments, the Cas12a2 protein, or Cas12a2 protein-encoding polynucleotide, and guide RNA(s), or DNA encoding the guide RNA(s), are introduced into a plurality of bacterial cells with the guide RNA(s) designed to target sequences that are present only in a certain fraction of the cells. In some embodiments, this will result in the elimination or reduction of those cells that comprise the target sequence(s) that the guide RNA(s) are designed to hybridize with.

By “predetermined” or “target sequence” is intended a nucleotide (e.g., DNA or RNA) sequence in the microbe of interest that is unique to that microbe. The predetermined or target sequence may be genomic DNA, chromosomal DNA, and/or plasmid or other extrachromosomal DNA sequences present in the cell or cells of interest. Methods are available in the art to find unique sequences within genomes and include using a Pan-Core genome approach to find accessory genes of organisms. Additionally using a Best Bi-directional Blast analysis or using OrthoMCL etc, would identify accessory genes. Additionally, unique regions between a pair of genomes can be extracted from a pair-wise global alignment performed using any of the popular programs like Nucmer (MUMmer), Mauve, BLAST, and the like. In some embodiments, a target sequence of interest is a sequence that is part of an antibiotic resistance gene. Antibiotic resistance gene sequences are known in the art and include, for example and without limitation, GyrB, ParE, ParY, AAC(1), AAC(2′), AAC(3), AAC(6′), ANT(2″), ANT(3″), ANT(4′), ANT(6), ANT(9), APH(2″), APH(3″), APH(3′), APH(4), APH(6), APH(7″), APH(9), ArmA, RmtA, RmtB, RmtC, Sgm, AER, BLA1, CTX-M, KPC, SHV, TEM, BlaB, CcrA, IMP, NDM, VIM, ACT, AmpC, CMY, LAT, PDC, OXA β-lactamase, mecA, Omp36, OmpF, PIB (por), bla (blaI, blaR1) and mec (mecI, mecR1) operons, Chloramphenicol acetyltransferase (CAT), Chloramphenicol phosphotransferase, EmbB, Mupirocin-resistant isoleucyl-tRNA synthetases MupA, MupB, MprF, Cfr 23S rRNA methyltransferase, Rifampin ADP-ribosyltransferase (Arr), Rifampin glycosyltransferase, Rifampin monooxygenase, Rifampin phosphotransferase, Rifampin resistance RNA polymerase-binding proteins DnaA, RbpA, Rifampin-resistant beta-subunit of RNA polymerase (RpoB), Cfr 23S rRNA methyltransferase, Erm 23S rRNA methyltransferases (e.g., ErmA, ErmB, Erm(31)), Streptogramin resistance ATP-binding cassette (ABC) efflux pumps (e.g., Lsa, MsrA, Vga, VgaB), Streptogramin Vgb lyase, Vat acetyltransferase, Fluoroquinolone acetyltransferase, Fluoroquinolone-resistant DNA topoisomerases, Fluoroquinolone-resistant GyrA, GyrB, ParC, Quinolone resistance protein (Qnr), FomA, FomB, FosC, FosA, FosB, FosX, VanA, VanB, VanD, VanR, VanS, EreA, EreB, GimA, Mgt, Ole, MPH(2′)-I, MPH(2′)-II, MefA, MefE, Mel, sat, Sul1, Sul2, Sul3, sulfonamide-resistant FolP, TetX, TetA, TetB, TetC, Tet30, Tet31, TetM, TetO, TetQ, Tet32, Tet36, MacAB-TolC, MsbA, MsrA, VgaB, EmrD, EmrAB-TolC, NorB, GepA, MepA, AdeABC, AcrD, MexAB-OprM, mtrCDE, adeR, acrR, baeSR, mexR, phoPQ, mtrR, and other such genes known to those of skill in the art (see, e.g., McArthur et al 2013 Antimicrobial Agents and Chemotherapy 57:3348-3357). In some embodiments, a target sequence is present in a plasmid, for example and without limitation a sequence that is present in a pOXA-48, pKpQIL, IncFII, p202c, HI2, HI1, I1-γ, X, L/M, N, FIA, FIB, FIC, W, Y, P, A/C, T, K, B/O, pAM830, pAM831 plasmid, and other such plasmids known to those of skill in the art.

### IV. Organisms Comprising a Target Sequence

The methods and compositions provided herein may be adapted to selectively modify and/or eliminate cells of interest by associating a Cas12a2 polypeptide of the invention (i.e., SEQ ID NOs: 1-14 and 55) with a guide polynucleotide that hybridizes with a target sequence in one or more cells of interest or to cleave a target sequence in the cells of interest. The target sequence may be a sequence in an eukaryotic or prokaryotic cell, as further outlined herein. The cells of interest can be the cell of one or more pest of interest. In such instances, the target sequence can be a target sequence that is specific to the pest of interest. Pests of interest are further described herein, and can include a pathogenic bacterial species, such as a pathogenic bacterial species associated with plants or mammals. In some instances, the pathogenic bacterial species is associated with humans.

### IV.A. Eukaryotes

A variety of eukaryotic cells, such as eukaryotic plant pathogens or eukaryotic cells associated with diseases in plants or animals may be targeted and/or selectively eliminated by the compositions and methods of the present invention. Accordingly, in one aspect, provided herein are eukaryotic cells comprising the Cas12a2 polypeptides of the invention or polynucleotides encoding said Cas12a2 polypeptides.

### Plant Pathogens

In some embodiments, the methods or compositions herein may be used to target eukaryotic cells belongs to one or more plant pathogen or plant pests. In certain embodiments, said one or more plant pathogens is a plant parasitic nematode, a bacterium, an insect, a fungus, a virus, a mollusk, a spider, a scorpion, a caterpillar, an animal, a mite, a tick, or a combination thereof. The terms plant “pathogen” and plant “pest” are used interchangeably to refer to organisms (e.g., insects, nematodes, or mollusks) that cause damage to plants or otherwise are detrimental to human agricultural methods or products. In such embodiments, the Cas12a2 polypeptide of the invention (i.e., SEQ ID NOs: 1-14 and 55) is complexed with a guide polynucleotide that hybridizes with a target sequence specific to the plant pathogen.

The methods of the present invention may be applied pre-harvest (i.e., during plant growth) or post-harvest, or may be applied to seeds or isolated plant cells or cell cultures, plant parts, and may be applied, for example, to leaves, flowers, seeds, roots, stems, or other plant tissues. In some embodiments, the compositions and methods of the present invention may be used to reduce the number of cells of a given plant pathogen, or to eliminate all or nearly all of the cells of a given plant pathogen. In some embodiments, the compositions and methods of the present invention may be used to selectively target and eliminate (in whole or in part) only those cells of a given plant pathogen that harbor certain target sequences in the genome of the plant pathogen.

### Mammalian Cells

In some embodiments, the methods or compositions herein may be used to target mammalian cells, such as those associated with diseases in mammalian subjects. In certain embodiments, the mammalian cell is a human mammalian cell. In certain embodiments, the mammalian cell is a non-human mammalian cell (e.g., a mouse, rat, pig, or non-human primate cell). In certain embodiments, the eukaryotic cells are ones associated with disease states, such as cancer cells. In such embodiments, the Cas12a2 polypeptide of the invention (i.e., SEQ ID NOs: 1-14 and 55) is complexed with a guide polynucleotide that hybridizes with a target sequence specific to the mammalian cell of interest (e.g., a target sequence specific to a cancer cell).

The methods of the present invention may be administered to a mammalian subject ex vivo or in vivo. For in vivo administration, the composition may be administered systemically or locally to a mammalian subject in an amount effective to achieve selective depletion, inhibition, or killing of the target cells. The Cas12a2 polypeptide and a guide polynucleotide capable of targeting a target sequence in a cell of interest may be incorporated into any suitable delivery vector for mammals, such as a viral vector (e.g., AAV vector) or non-viral mode of delivery (e.g., lipid nanoparticle). In some embodiments, the cancer cells are cells associated with bladder cancer, breast cancer, colorectal cancer, endometrial cancer, head and neck cancer, leukemia, lung cancer, lymphoma, melanoma, ovarian cancer, and prostate cancer.

### IV.B. Prokaryotes

A variety of prokaryotes may be targeted and/or selectively eliminated by the compositions and methods of the present invention. In particular instances, bacterial species that are bacterial pathogens or otherwise undesirable may be targeted, including plant-associated bacteria, animal-associated bacteria, fungus-associated bacteria, and arthropod-associated bacteria. Examples of a variety of bacterial species that may be targeted by the present invention are further delineated herein. Accordingly, in one aspect, provided herein are bacterial cells comprising the Cas12a2 polypeptides of the invention or polynucleotides encoding said Cas12a2 polypeptides.

### Plant-Associated Bacteria

Bacterial species that grow on plants or plant material may be targeted and selectively eliminated by the compositions and methods of the present invention. Non-limiting examples of plant- or plant-material associated bacterial species of interest include Xanthomonas sp., Escherichia sp., Pseudomonas sp., Erwinia sp., Xylella sp., Clavibacter sp., Ralstonia sp., Pectobacterium sp., Streptomyces sp., Burkholderia sp., Phytoplasma sp., Acidovorax sp., Pantoea sp., Agrobacterium sp., Spiroplasma sp., Candidatus Liberibacter sp., Dickeya sp., Serratia sp., Sphingomonas sp., Rhizobacter sp., Rhizomonas sp., Xylophilus sp., Rickettsia sp., Bacillus sp., Clostridium sp., Arthrobacter sp., Curtobacterium sp., Leifsonia sp., Rhodococcus sp., and Phytoplasma sp. Plant-associated bacteria may include, for example, plant pathogens, modulating bacteria, bacteria that grow on plants and may harm humans or other animals that consume the plant material, or other bacteria. The methods of the present invention may be applied pre-harvest (i.e., during plant growth) or post-harvest, or may be applied to seeds or isolated plant cells or cell cultures, plant parts, and may be applied, for example, to leaves, flowers, seeds, roots, stems, or other plant tissues. In some embodiments, the compositions and methods of the present invention may be used to reduce the number of cells of a given bacterial strain or species, or to eliminate all or nearly all of the cells of a given bacterial strain or species. In some embodiments, the compositions and methods of the present invention may be used to selectively target and eliminate (in whole or in part) only those cells of a given bacterial strain or species that harbor certain target sequences, whether those sequences are present in the bacterial chromosomal genome, in plasmids, in viruses or phages that have infected or are otherwise present in the bacteria, or in other DNA-containing components found in those bacterial cells.

### Animal-Associated Bacteria

Bacterial species that grow in or on animals or animal parts (e.g., meat, bones, teeth, organs, etc.) may be targeted and selectively eliminated by the compositions and methods of the present invention. Non-limiting examples of such animal-associated bacterial species of interest include Escherichia sp., Enterobacter sp., Citrobacter sp., Klebsiella sp., Hafnia sp., Corynebacterium sp., Mycoplasma sp., Serratia sp., Pasteurella sp., Proteus sp., Campylobacter sp., Salmonella sp., Pseudomonas sp., Brucella sp., Staphylococcus sp., Streptococcus sp., Trueperella sp., Clostridium sp., Listeria sp., Anthrax sp., Bartonella sp., Capnocytophaga sp., Streptobacillus sp., Rickettsia sp., Anaplasma sp., Shigella sp., Borrelia sp., Actinomyces sp., Bacteroides sp., Bordetella sp., Chlamydia sp., Chlamydophila sp., Ehrlichia sp., Enterococcus sp., Francisella sp., Haemophilus sp., Helicobacter sp., Legionella sp., Leptospira sp., Mycobacterium sp., Neisseria sp., Nocardia sp., Treponema sp., Vibrio sp., and Yersinia sp. Animal-associated bacteria may include, for example, bacteria that live in or on oral cavities, gut tissues (e.g., stomach, intestines, etc.), stool, genitalia, skin, hair, eyes, ears, nasal cavities, the bloodstream, and/or the tissues of the respiratory system, and the like. Animal-associated bacteria may also live on animal parts in dead animals, for example, in animal meat, skin, bones, organs, brain, and/or other tissues. The methods of the present invention may be applied to living animals, for example to reduce or eliminate harmful bacteria such as pathogenic bacteria that may cause health problems for the animal that harbors the bacterial cell(s) of interest. The methods of the present invention may be applied to animal parts such as, for example, meat or other products intended for consumption by humans or other animals, for example to reduce or eliminate the presence of harmful or potentially harmful bacteria such as those that may cause disease in humans or animals that consume the animal parts. In some embodiments, the compositions and methods of the present invention may be used to selectively target and eliminate (in whole or in part) only those cells of a given bacterial strain or species that harbor certain target sequences, whether those sequences are present in the bacterial chromosomal genome, in plasmids, in viruses or phages that have infected or are otherwise present in the bacteria, or in other DNA-containing components found in those bacterial cells.

### Human-Associated Bacteria

Bacterial species that grow in or on humans represent a subset of those bacteria that grow in or on animals or animal parts and may be targeted and selectively eliminated by the compositions and methods of the present invention. Non-limiting examples of such human-associated bacterial species of interest include Escherichia sp., Enterobacter sp., Citrobacter sp., Klebsiella sp., Hafnia sp., Corynebacterium sp., Mycoplasma sp., Serratia sp., Pasteurella sp., Proteus sp., Campylobacter sp., Salmonella sp., Pseudomonas sp., Brucella sp., Staphylococcus sp., Streptococcus sp., Trueperella sp., Clostridium sp., Listeria sp., Anthrax sp., Bartonella sp., Capnocytophaga sp., Streptobacillus sp., Rickettsia sp., Anaplasma sp., Shigella sp., Borrelia sp., Actinomyces sp., Bacteroides sp., Bordetella sp., Chlamydia sp., Chlamydophila sp., Ehrlichia sp., Enterococcus sp., Francisella sp., Haemophilus sp., Helicobacter sp., Klebsiella sp., Legionella sp., Leptospira sp., Mycobacterium sp., Neisseria sp., Nocardia sp., Treponema sp., Vibrio sp., and Yersinia sp. Human-associated bacteria may include, for example, bacteria that live in or on oral cavities, gut tissues (e.g., stomach, intestines, etc.), stool, genitalia, skin, hair, eyes, ears, nasal cavities, the bloodstream, and/or the tissues of the respiratory system, and the like. The methods of the present invention may be applied therapeutically, for example to reduce or eliminate harmful bacteria such as pathogenic bacteria that may cause health problems for the human that harbors the bacterial cell(s) of interest. The compositions of the present invention may be delivered to humans through various routes of administration, for example through inhalation, ingestion, injection, or other routes of administration. In some embodiments, the compositions and methods of the present invention may be used to selectively target and eliminate (in whole or in part) only those cells of a given bacterial strain or species that harbor certain target sequences, whether those sequences are present in the bacterial chromosomal genome, in plasmids, in viruses or phages that have infected or are otherwise present in the bacteria, or in other DNA-containing components found in those bacterial cells.

### Fungus-Associated Bacteria

Bacterial species that grow in close contact with fungal organisms or cells may be targeted and selectively eliminated by the compositions and methods of the present invention. For example, bacteria that interfere with fungal culture and/or fungal fermentation may be targeted for control, elimination, or reduction by the compositions and methods of the present invention. Non-limiting examples of such fungus-associated bacterial species of interest include Enterobacter sp., Pseudomonas sp., Klebsiella sp., Serratia sp., Staphylococcus sp., Escherichia sp., Clostridium sp., Enterococcus sp., and other such bacterial species. In some embodiments, the compositions and methods of the present invention may be used to selectively target and eliminate (in whole or in part) only those cells of a given bacterial strain or species that harbor certain target sequences, whether those sequences are present in the bacterial chromosomal genome, in plasmids, in viruses or phages that have infected or are otherwise present in the bacteria, or in other DNA-containing components found in those bacterial cells.

### Arthropod-Associated Bacteria

Bacterial species that grow in close contact with arthropods or other insects may be targeted and selectively eliminated by the compositions and methods of the present invention. For example, some arthropods are known to harbor symbiotic bacteria that may be selectively reduced or eliminated using the compositions and methods of the present invention. Some arthropod species that may be of particular interest for use with the compositions and methods of the present invention include those that transmit disease to humans or animals (non-limiting examples include ticks and mosquitoes), those that transmit disease to plants (non-limiting examples include aphids and psyllids), and arthropods that are farmed, for example for human consumption (non-limiting examples include shrimp, crabs, and lobsters). In some embodiments, bacteria that enable disease transmission to plants, humans, or other animals by arthropods, or bacteria that are required for disease transmission to plants, humans, or other animals by arthropods, may be targeted and selectively eliminated using the compositions and methods of the present invention. In some embodiments, bacteria that contaminate cultivated aquacultural arthropods (e.g., shrimp, crabs, lobsters, and other arthropods) may be targeted and selectively eliminated using the compositions and methods of the present invention. Non-limiting examples of such arthropod-associated bacteria include Borrelia sp., Rickettsia sp., Anaplasma sp., Francisella sp., Coxiella sp., Wolbachia sp., Ehrlichia sp., Liberibacter sp., Aeromonas sp., Vibrio sp., Edwardsiella sp., Streptococcus sp., Yersinia sp., Flavobacterium sp., Tenacibaculum sp., Renibacterium sp., Piscirickettsia sp., Mycobacterium sp., Pseudomonas sp., Clostridium sp., Enterobacterium sp., Nocardia sp., Lactococcus sp., Aerococcus sp., Hepatobacter sp., Chlamydia sp., and other such bacterial species. In some embodiments, the compositions and methods of the present invention may be used to selectively target and eliminate (in whole or in part) only those cells of a given bacterial strain or species that harbor certain target sequences, whether those sequences are present in the bacterial chromosomal genome, in plasmids, in viruses or phages that have infected or are otherwise present in the bacteria, or in other DNA-containing components found in those bacterial cells.

### Environmental Bacteria

Bacterial species that grow in the environment may be targeted and selectively eliminated by the compositions and methods of the present invention. Environments of particular interest may include, without limitation, wastewater, water intended for treatment to render it potable, surgical instruments and other materials in hospitals or other environments where sterility is required, and other such environments. In some embodiments, bacteria living in these and other environments may be targeted for reduction or elimination through the use of the compositions and methods of the present invention. In some embodiments, the compositions and methods of the present invention may be used to selectively target and eliminate (in whole or in part) only those cells of a given bacterial strain or species that harbor certain target sequences, whether those sequences are present in the bacterial chromosomal genome, in plasmids, in viruses or phages that have infected or are otherwise present in the bacteria, or in other DNA-containing components found in those bacterial cells.

### V. Enrichment of Cell Types

The compositions and methods of the present invention may be used to reduce or eliminate the presence of cells, including prokaryotic cells (e.g., bacterial cells) or eukaryotic cells that comprise undesirable DNA sequence(s). In some embodiments, the compositions and methods of the present invention may be used to enrich for cells, cell lines, cell types, or other groupings of cells that do not comprise undesirable DNA sequence(s). Enrichment of certain cell types may be desirable, for example, following genome editing experiments or other experiments designed to modify certain known regions of a genome or other DNA molecule. In such embodiments, the genome editing experiment may be performed to produce a desired genomic modification, resulting in a pool of cells in which a portion of the cells remain wild-type while a portion of the cells comprises the desired DNA sequence modification(s). The compositions and methods of the present invention may be used to target, through the appropriate design of guide RNA(s) or other guide polynucleotides designed to hybridize with wild-type, but not with modified sequences. Introduction of a Cas12a2 polypeptide, or encoding polynucleotide, along with one or more appropriately designed guide RNA(s) or encoding DNA molecules, into the pool of cells (for example through the use of engineered phages or phagemids, or through the use of conjugative plasmids), results in an initial hybridization event in cells that retain the undesirable wild-type sequence(s). This initial hybridization event triggers secondary, collateral activity of the Cas12a2 enzyme targeted against dsDNA and/or RNA, resulting in cell death among those cells that comprise the undesirable wild-type sequence(s). The result of the targeted elimination of wild-type cells is the enrichment of cells in the cell pool that comprise the desired DNA sequence(s). Such experiments may be used, for example, to increase the likelihood of identifying and recovering cells that comprise a desirable allele or other genetic sequence, particularly in cases when such a desirable allele is relatively rare among the cells in the cell pool prior to introduction of the Cas12a2 polypeptide and guide RNA(s) or guide polynucleotides.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### EXAMPLES

### Example 1—Sequence Analyses of Cas12a2 Nucleases

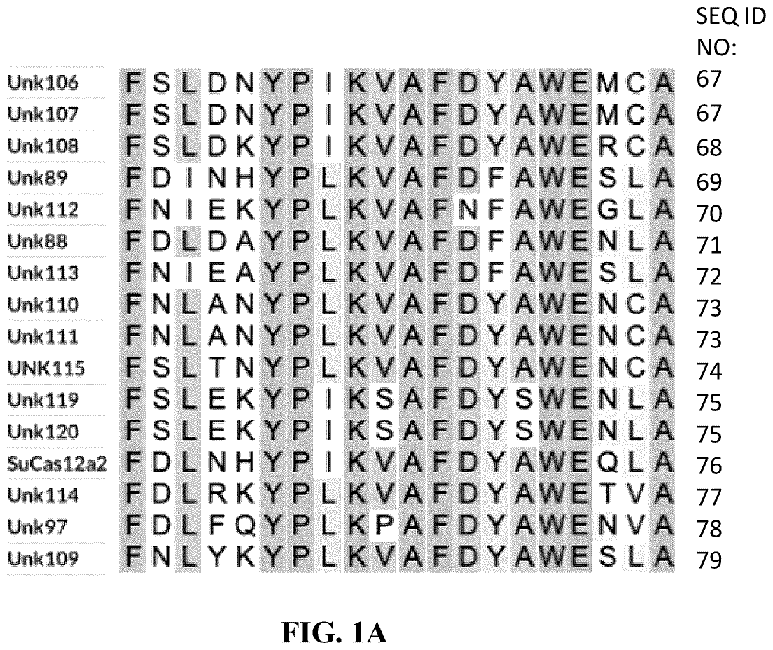

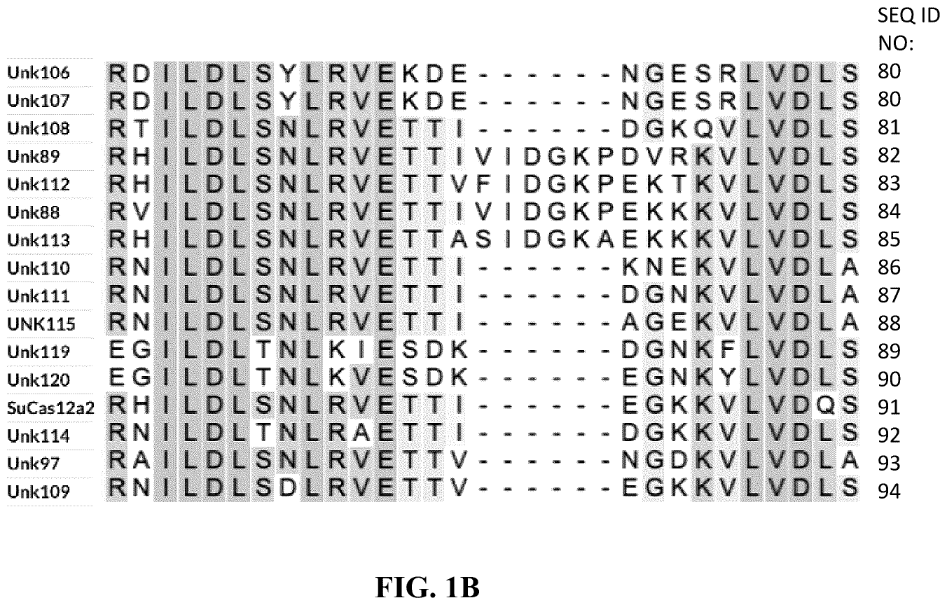

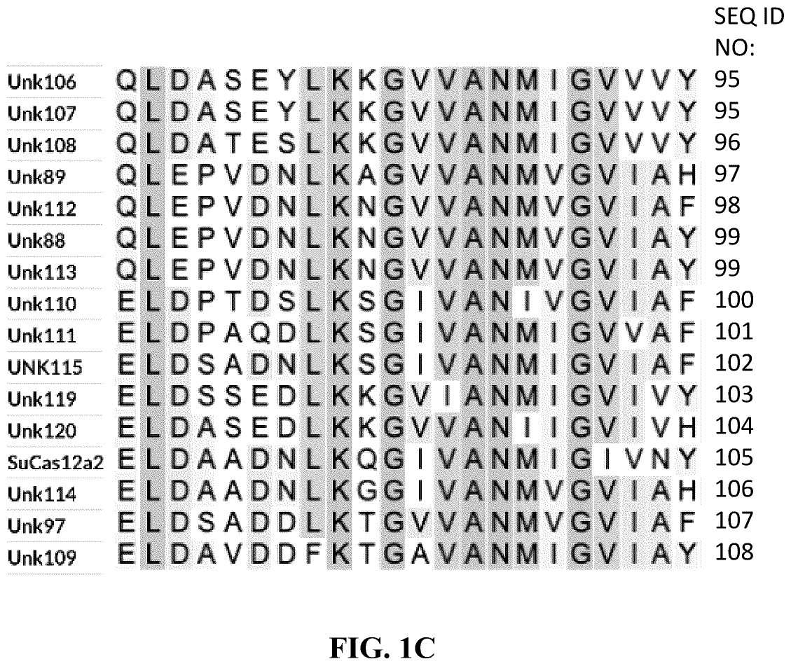

Cas12a2 nuclease amino acid sequence alignments were examined to identify amino acid residues within the protein sequences that are well-conserved among these nucleases. SulfCas12a2 (SuCas12a2; SEQ ID NO: 15) and SulfCas12a2 variant nucleases, including those proteins in the group consisting of SEQ ID NOs: 1-14 and 55, were aligned to identify partially and/or completely conserved amino acid residues among these nucleases. FIGS. 1A-1C show an alignment of three domains (FIG. 1A, residues 370 to 389; FIG. 1B, residues 896 to 921; and FIG. 1C, residues 1028 to 1049) within the SulfCas12a2-like nucleases. Within these three domains, individual amino acid residues were identified as being partially (lighter shading) or completely (darker shading) conserved between the SulfCas12a2-like nucleases.

### Example 2—Cytotoxicity of Cas12a2 Nucleases in E. coli Assays

The toxic activity of Cas12a2 was validated in E. coli by conducting a toxicity assay. Two plasmids were transformed into BL21-AI E. coli cells and maintained through selection. Plasmid A, conferring Chloramphenicol resistance, contained arabinose+IPTG inducible expression cassettes for a Cas12a2 peptide and a guide RNA corresponding to a target of interest. Plasmid B, conferring Kanamycin resistance, contained a fragment of the target gene of interest or a non-target gene fragment. In this experiment, plasmid A included an expression cassette for the guide RNA CAO1-1 containing the spacer sequence tggagcaacacctgaaggaaggct (SEQ ID NO: 53), and plasmid B included a CAO1 gene fragment from Oryza sativa corresponding to the CAO1-1 guide RNA as the target gene fragment.

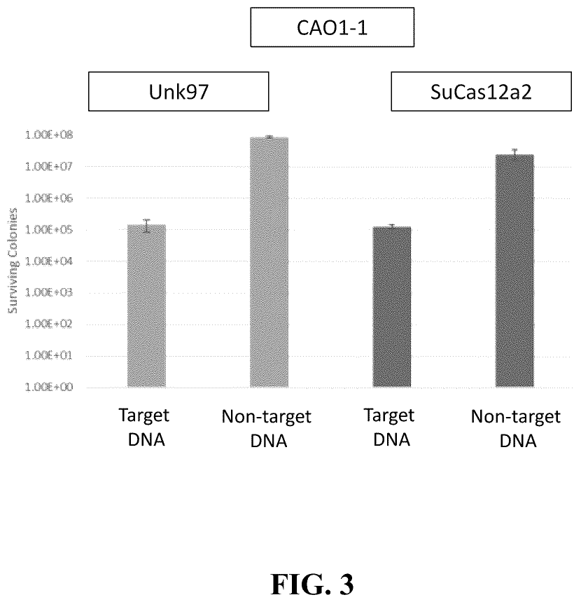

The E. coli strains containing the two plasmids were grown overnight, fresh cultures were inoculated, and expression was induced for two hours before plating a 10-fold dilution series of each Cas12a2 and guide with its target and non-target control. Treatments were plated without selection for plasmid B. Colonies were counted the following day (FIG. 3) and the percent reduction of surviving colonies were recorded (Table 1). Toxicity was assessed by measuring the percent reduction in colony survival relative to control colonies containing a non-target control gene fragment.

As described hereinabove, the Cas12a2-guide RNA complex hybridizes with the targeted nucleotide sequence (the “initial hybridization event”), at which site the Cas12a2 endonuclease introduces a double-stranded break (DSB). The initial hybridization event and DSB production lead to a change in the structure of the Cas12a2 protein, resulting in a protein that is capable of degrading double-stranded DNA (dsDNA) and/or RNA in a non-sequence-specific manner, leading to cell death. If the Cas12a2-guide RNA complex from plasmid A targets and cleaves the target gene fragment from plasmid B, followed by non-sequence-specific degradation of nucleotides by the Cas12a2 peptide, the E. coli cell dies.

Table 2 describes the toxicity of various Cas12a2 peptides provided herein. SuCas12a2, Unk114, Unk110, Unk109, Unk97, Unk108, Unk113, Unk119, Unk111, and Unk115 showed strong toxicity. In contrast, Unk88, Unk107, Unk71, Unk112, Unk120, and Unk17 exhibited minimal or negative reductions in colony survival and were classified as non-toxic. These results provide activity of various Cas12a2 peptides for potential further characterization.

TABLE 2
Results of Cas12a2 toxicity assay in E. coli targeting
SEQ ID NO: 53 of Oryza sativa with guide RNA CAO1-1

| Nuclease | % Reduction vs non-target | p-value | Toxicity |
|---|---|---|---|
| SuCas12a2 | **99.97 | 0.022 | Toxic |
| Unk114 | **99.95 | 0.031 | Toxic |
| Unk110 | **99.88 | 0.052 | Toxic |
| Unk109 | **99.78 | 0.003 | Toxic |
| Unk113 | 99.750 | 0.205 | Toxic |
| Unk97 | **99.71 | 0.010 | Toxic |
| Unk108 | **99.679 | 0.036 | Toxic |
| Unk119 | **99.000 | 0.071 | Toxic |
| Unk111 | 93.182 | 0.337 | Toxic |
| Unk115 | *90.500 | 0.062 | Toxic |
| Unk88 | 20.078 | 0.185 | Not toxic |
| Unk107 | 13.010 | 0.817 | Not toxic |
| Unk71 | −5.000 | 0.798 | Not toxic |
| Unk112 | −11.110 | single rep | Not toxic |
| Unk120 | −17.190 | 0.239 | Not toxic |
| Unk17 | −52.000 | 0.339 | Not toxic |

*= p < 0.1,
**= p < 0.05

### Example 3: Specificity of Cas12a2 Nucleases

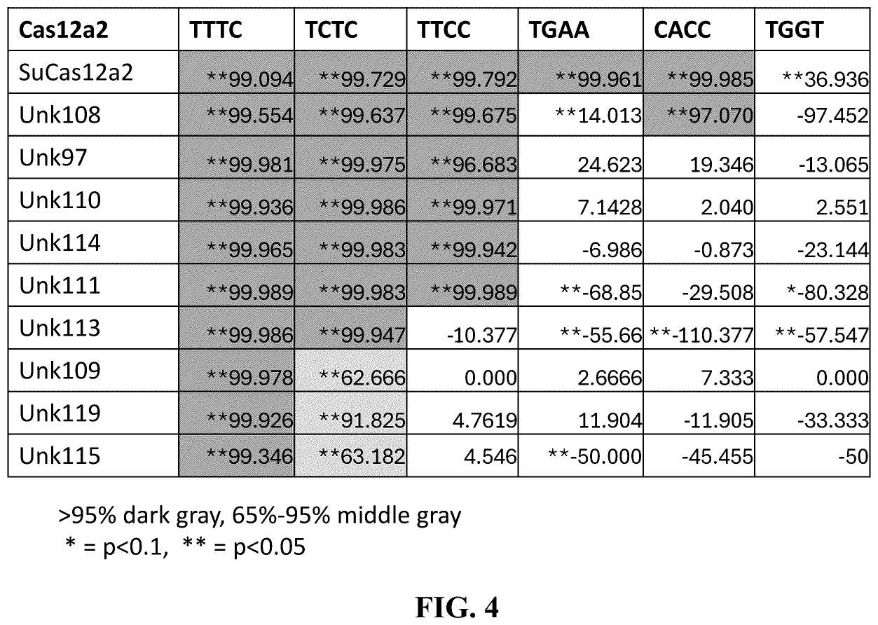

Specificity of the activity of Cas12a2 peptides provided herein at various PAM sequences was assessed using the E. coli assay described in Example 2. Plasmid B carrying the target fragment was mutated to contain each of the tested PAM variants. E. coli cells were co-transformed with Plasmid A (carrying the nuclease and guide RNA) and each of the plasmid B PAM variants and survival was measured relative to the non-target control. The results are shown in FIG. 4. High toxicity of the Cas12a2 peptide across several PAM sequences (such as SuCas12a2 or Unk108 in FIG. 4) indicates broad activity capable of targeting many locations associated with different PAMs, and thus may indicate greater potential off-target effects. In contrast, high toxicity of the Cas12a2 peptide limited to one or two PAM sequences (such as Unk113, Unk109, Unk119, and Unk115 in FIG. 4) indicates focused activity capable of targeting limited locations associated with one or two PAMs, and thus may indicate less potential off-target effects.

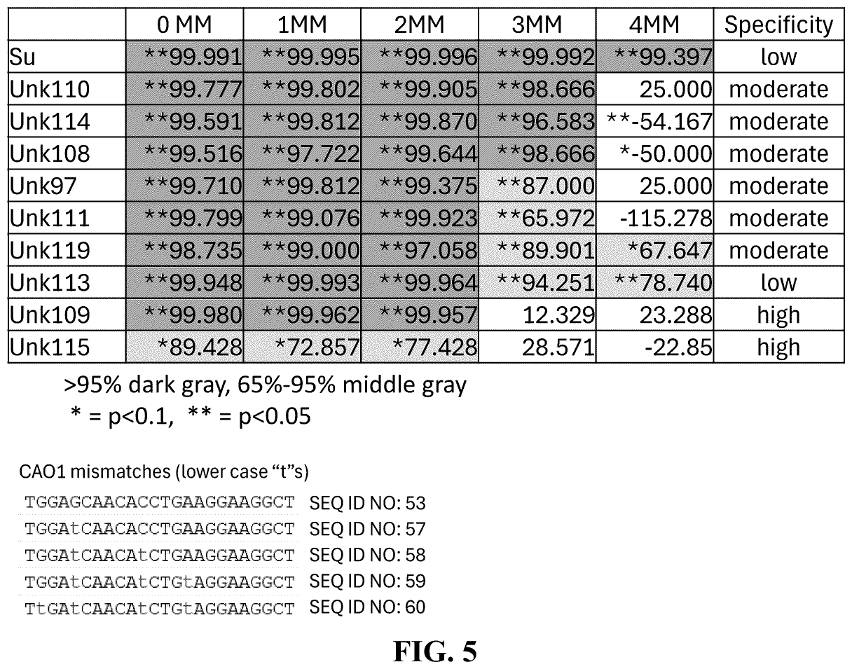

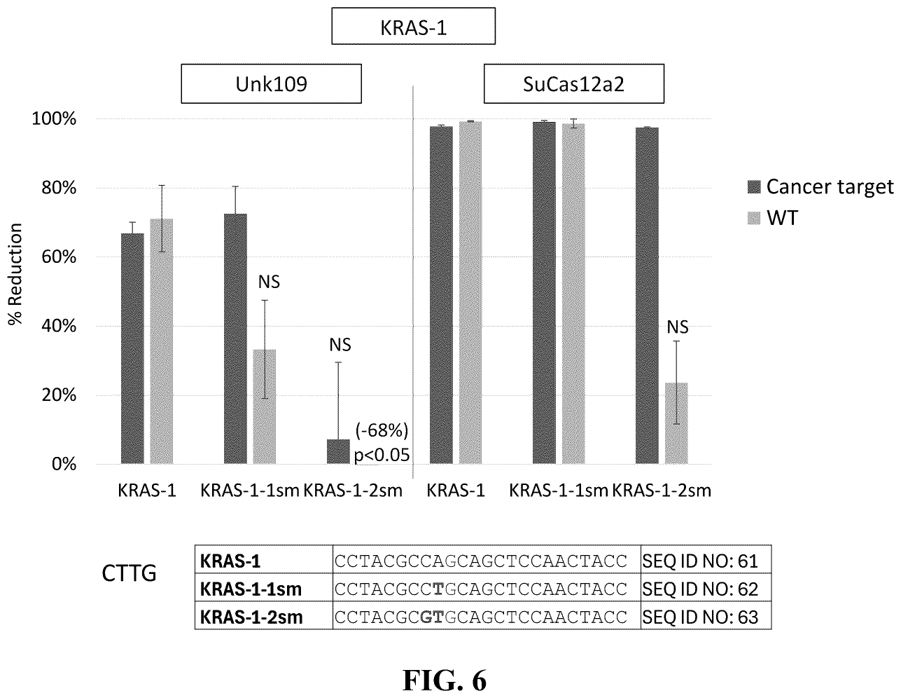

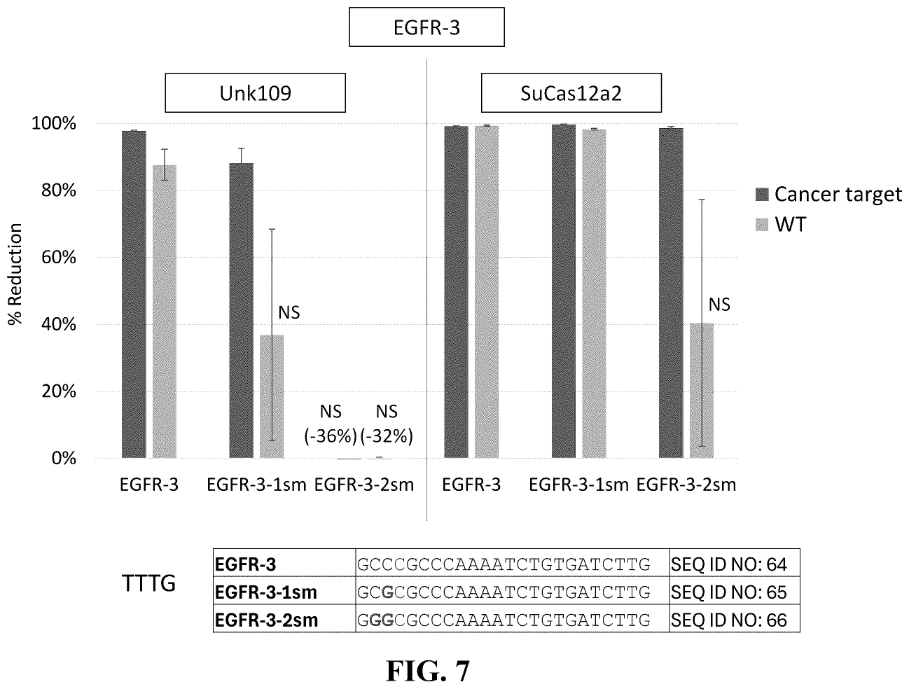

Next, specificity of the Cas12a2 system to the target cells was tested using the guide RNA CAO1-1 fully complementary to the target sequence in the Oryza sativa CAO1 gene as well as guide RNAs having 1 to 4 mismatches to the nucleic acid sequence fully complementary to the target sequence in the Oryza sativa CAO1 gene, using the E. coli assay. As shown in FIG. 5, SuCas12a2 showed toxicity when used with the CAO1-1 gRNA with 0, 1, 2, 3, or 4 mismatches, indicating low specificity to the target sequence. In contrast, Unk109 and Unk115 showed strong toxicity when used with the CAO1-1 gRNA with 0, 1, or 2 mismatches, but not with the CAO1-1 gRNA with 3 or more mismatches. Indicating high specificity to the target sequence.

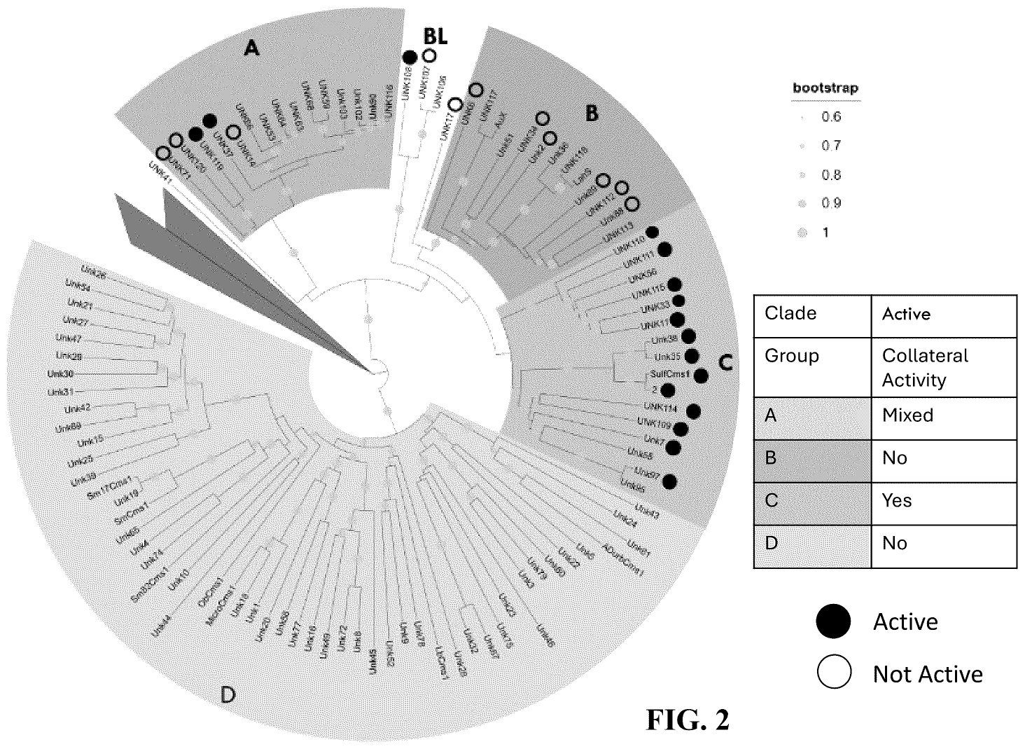

Specificity of the Cas12a2 system to the target cells comprising target sequences was further tested using the Unk109 and SuCas12a2 peptides and guide RNAs targeting KRAS-1 or EGFR-3 oncogenes with synthetic mismatches using the E. coli assay described in Example 2. As shown in FIG. 2 (targeting KRAS-1) and FIG. 3 (targeting EGFR-3), the Cas12a2 system demonstrated toxicity (targeting and cleaving effect) at the KRAS-1 and EGFR-3 cancer targets, and introduction of a 1 or 2 bp mismatch into the guide RNA tend to improve specificity for the cancer targets, reducing off-target effects on WT sequences. As shown in FIG. 2, the guide RNAs targeted the KRAS-1 sequence adjacent to the PAM CTTG. Unk109 showed less robust toxicity at KRAS-1 relative to SuCas12a2, with WT off-target toxicity significantly reduced with addition of one synthetic mismatch to the guide RNA. SuCas12a2 showed more robust toxicity at KRAS-1 relative to Unk109, with WT off-target toxicity significantly reduced with addition of two synthetic mismatches to the guide RNA. As shown in FIG. 3, the guide RNAs targeted the EGFR-3 sequence adjacent to the PAM TTTG. Unk109 showed significant reduction in off-target toxicity with addition of one synthetic mismatch to the guide RNA. SuCas12a2 showed significant reduction of off-target toxicity with addition of two synthetic mismatches to the guide RNA.

TABLE 3
Sequence table

| SEQ | | |
|---|---|---|
| ID | | |
| NO. | Description | Sequence |
| 1 | Amino acid sequence of the | MIMENIKNNYQLSKTLRFGLTQKQNGNSSNTDNVYHSHSA |
| | Unk89 peptide | LKELVDISENRIKKNVSTEGATEMQLSIESIRKCMIMIEQ |
| | | FIKDWKRVYYRSDQISLDKDFYKKLSKKIGFEAFWFERNK |
| | | KTQQRIKKPQSCIIALSELSKRDNFGKERQEYIVEYWENN |
| | | LLKSTERYEEVSEKLEQFELALKINRTDNRPNEVELRKMF |
| | | LSLVNMVREVVEPLCLGQISFPKLEKLADNSKNKQLRKFA |
| | | TDYQSKSDLLTQISELKKYFEENGGNVPYCRATLNPLTAV |
| | | KNPKSTDSSILDEIKKLKLDVILRDYQSVALFDNSIRDLT |
| | | ASQKMQLLNQNNEGLIKRGLLFKYKPIPAIVQYEIAKVLS |
| | | AELNKDEQELRNFLRDIGQVKSPAKDYAELQDKKDFDINH |
| | | YPLKVAFDFAWESLAKSVYHPDIDFPEEQCKTLLREVFQV |
| | | DENNENFKFYAQLLELRSLLATLEHGKPTEVITIENEVKK |
| | | ILENIDWSKFGDRGKNYKSAIENWIHNRNKKDFKGDYFKK |
| | | AKQQIGLTRGRQKNLIKKYDEITKSYKDIAMKMGKTFAEM |
| | | RDKITGAAELNKVSHYAMIVEDTNQDKYVLLQEFVENNKD |
| | | RIYAKSTPQNEDFKAYSVNSVTSSAIAKMIRKIRIDKLQA |
| | | NERNNNRQQAPELSETQKEARNIKEWKDFIAEKRWNYEFD |
| | | LKLDNKNFEQIKKEIDSKCFKLETKYMSEEVLVDLVKNQN |
| | | CLLLPIINQDLAKKIKSESNQFTKDWNAIFAQNTPWRLTP |
| | | EFRVSYRKPTPNYPKSERGDKRYSRFQMIGHFLCDFIPKT |
| | | ADYISNREMIANFKDDEKQKQTIINFHERLNPKSENEKMN |
| | | MLLAKFGNKNSNQSKETKKEEKFYVFGIDRGQKELATLCV |
| | | IDQDKKIIGDFNIYTRSFNTQNKQWEHQLLDKRHILDLSN |
| | | LRVETTIVIDGKPDVRKVLVDLSEVKVKDKNGNYTKTDKM |
| | | QVKMQQLAYIRKLQFQMQTNPDTVLEWYNKNQTKEAILNN |
| | | FVDKPNGEKGLVSFYGSAVEELKDTLPIERIEKMLQQFKS |
| | | LKNEEKEGKDVKAEIDKLIQLEPVDNLKAGVVANMVGVIA |
| | | HLLKEFNYQVYISLEDLSNPFGSHVIDGTTGTHSKINKGE |
| | | GKRADVEKYAGLGLYNFFEMQLLKKLFRIQQDSQNILHLV |
| | | PAFRAVKNYENIIAGKDKIKNQFGIVFFVDANSTSKMCPV |
| | | CNSTNETNREYPNAKKGTSKDDKEVWVERDKSNGDDIIRC |
| | | FVCGFDTTKKYEENPLKFIKSGDDNAAYIISAFGIKAYEL |
| | | AKSVIDNK |
| 2 | Amino acid sequence of the | MEAIKNNYQLSKTLRFGLTQKSKTRKDGFTGEIYQSHNEL |
| | Unk88 peptide | KDLVKSSEDRIKKSVSTDEKSEMSLSVDKIRCCLVMISDF |
| | | LSSWQQVYSRADQIALDKDYYKILCKKIGFDGFWVDERYD |
| | | RKNDKTVRTKKPQSRTINLSELDKKDDKGIERRQYLLTYW |
| | | RDNLINAADKFEVVTEKLKQFEDALNINRTYNKPNEVELR |
| | | KLFLSLTNIVQETLQPLCLGQICFPKLEKIDDSRTENKHL |
| | | IDFATDYQSKSDLLSEISELKKYFEENGGNVPYCRATLNQ |
| | | KTAVKNPNSTDNSIDSEIKKLGLDKILKENKDALYFANKI |
| | | YSLSAKEKLSKLDDKTTGLIERSLLFKYKPVPAIVQYEIA |
| | | KTLSETINKSEEDLLEFLRSIGQTKSPTKDYADLQDKNDF |
| | | DLDAYPLKVAFDFAWENLARSIYHSDADIPVAVCEGFLKK |
| | | NFGIDKSNADFKLYAQLQELKAVLATLEYGNPTNRQTFIN |
| | | EATKLLSPISWDKIGRNGNQNKYSIEKWLKTLTKDDKDYK |
| | | DAKQQIALFRGRLKNNIKTFDDITKYFKSVAMEMGRTFAQ |
| | | MRDKITGAAELNKVTHYAIIIEDQNFDKYVLLQEFVDKKE |
| | | NRIYAKTDRHHSDFTTYSVNSVTSGSIAKMLRKKRMDELN |
| | | RNNRNSFEQKPELSEEQKEQRNIREWKEFIEDKRWDLEFQ |
| | | LNLSNKTFEQIKKEVDAKCYELDINYISQETLSDLVNKKG |
| | | CLLLPIVSQDIAKENKTEGNQFTKDWNAIFTQETPWRLTP |
| | | EFRVSYRKPTPNYPVSDKGDKRYSRFQMIAHFLCDYLPKS |
| | | DSYISNWEQIANYKDDKLQEKAVKEFNADLRGRTEEEKQS |
| | | ESVNALLAHFGNQNKKQKPVERPKEKFYVFGIDRGQKELA |
| | | TLCVIDQDKKIVGDFDIYTRSFNSEAKQWEHKLLEKRVIL |
| | | DLSNLRVETTIVIDGKPEKKKVLVDLSEVKVKDKDGKYSK |
| | | PNKMQVKMQQLAYIRKLQFQMQTNPDAVLEWYANNKTKEQ |
| | | IMSNFVDNENGDKGLVSFYGTAVEELNETLPIDKIEEILK |
| | | KFQELKDKEKQGETVKLEIDKLVQLEPVDNLKNGVVANMV |
| | | GVIAYLLQNLDYQVYISLEDLSKPFSGQIIGGIAGVPTKT |
| | | NKEEGRRADVEKYAGLGLYNFFEMQLLKKLFRIQQDSQNI |
| | | LHLVPPFRAMKNYDHVAVGKGKVKNQFGIVFFVDADATSK |
| | | TCPCCGSSNNKPNLKMYPNAKKGLSKEGKEVWVERDKSEG |
| | | NDIIRCFVCGFDTTKDYSENPLRYIKSGDDNAAYLISAEG |
| | | IKAYELATTLVNNK |
| 3 | Amino acid sequence of the | METIEYQFSKTLRFGLTLKDHERKNKTHQTFNDLIGVSAQ |
| | Unk97 peptide | RIKEDASRDHDKTEQQLVTSVAACVKLMHQYLDAWEKIYR |
| | | RTDQLALTKDFYKQMAKKACFDAYWLDNKERKQPQSQIIA |
| | | ISSLRKKHDEKERKDYILDYWADNISITKQRIHEFEKVID |
| | | QFKKALENKSMAHNKPHLVDFRKMFLSLTRLCNETLIPIC |
| | | NDSICFPALDKLQDNARHEAIKTFASEDEREERKGLSLSI |
| | | KDIKEYFEENGGYVPLGKVTLNRYTAEQKPNNFKEDIKKK |
| | | INDLRLTDLIQKLINLSDEEIKEYFEFNGKQKKQLIDDTR |
| | | LSVVERVQLFKYKPIPAAVRFMLAEYLHKNNLLDKERVMT |
| | | LFEIIGKPRSIGEEYTKLKDTSDFDLFQYPLKPAFDYAWE |
| | | NVARNLRNDKANAYPKEQCIRFLENIFEVSTSTTAFILYA |
| | | DLLFIKDNLSTLEHEKNSPKDKDQFIENIKRTFKNINYGI |
| | | EQKEYIKHQQTILDWINKKEDAQKELKNANDNSYKNYENA |
| | | KQQFGLLRGRQKNSIRQYKDLTETFKTLAVSFGKNFAELR |
| | | EKLREENEINKITHFGIIVEDSKLERYVLLSKLDEDKTLS |
| | | IDHLLIDEPKGELKSYQVKSLMPKSLEKIIKNKGGYKDFH |
| | | TSSKYINFIEMKRDWANYKNKPELVDYVKDCLQHSTMSKD |
| | | QHWAAFGCDFTTCNSYEAIERELEIKAYRLKASHISITTV |
| | | QKLVNEENCILLPFVNQDITSAKRELKNQFSKDWDMLFEN |
| | | NNDYRLHPEFRIVYRQPTPDYPLNKRYSRFQLIAHLMCEY |
| | | IPQSVEYISRKQQIQIFNDKNEQKKQVDAFNERVKPSGEY |
| | | YVLGIDRGLKQLATLCVLNNEGQIQGGFEVFTRTFDSVKQ |
| | | EWKHNLLEKRAILDLSNLRVETTVNGDKVLVDLASILVKD |
| | | EQHNYTKDNQQKIKLKQLAYIRKLQFQMQHEPQKVLNFIK |
| | | DYTTPQAVGEKIGELITPYKEGTHYDDLPIEKIYDMLQQF |
| | | HQFTDEGNETAKKELTELDSADDLKTGVVANMVGVIAFML |
| | | EKYHYNAFVSLENLCRAFGFAKDGLNGELLVSTAVDNTVD |
| | | FKDQENLVLAGLGTYHYFEMQLLKKLFRIQTGNDIIHLVP |
| | | AFRSVDNYETIRKLSTKTKDALYTCKPFGVVHFIDPMYTS |
| | | KKCPACDSINVSRFSKQGDIISCNKCGFQTRWDTQTTLKN |
| | | NALLQSYKKQNLNLHYILNGDDNGAYRIALKTFANLT |
| 4 | Amino acid sequence of the | MTKYQLTKTLRFGLTKVRKKTKLVAGKEVDAKYLSHEELD |
| | Unk 106 peptide | DLVMRSEYNLIKRNVLEWSKKKESDKSNYIFDELDKRFEE |
| | | IIEIEDRDQRNAEYVEFLNEFFKEVNHQTINQLDESTFIN |
| | | KIGDCSKAIKEYLLSWGKVCRRIDKITVRKDYFKILARKT |
| | | FFKYESKVGKKRTPLPSEVKLSGQKGNNYFDEPINEGISQ |
| | | FWQNRVAKALNLHSQLESMLFDYKKAIETEKHNQENPKGD |
| | | NGSFDKLHLVDFRKMFLSVCSLVMDSLRPIVNELIIVSDN |
| | | VSKDEDKYILDFVNDKKTQWDLFQQIENLQTICKDNGENI |
| | | FFGKATFNKYTSEQAPNHRNNDIAKVLRELKIEKFVSDYI |
| | | DLDQEAINRKIYQSTQSRLENLNNPQISPIIRAQYFKYKP |
| | | IPTLVRFGLAKELAKQQGKKYSDRLKGIQELFRIFGSSKS |
| | | PALDYKNNRTDFSLDNYPIKVAFDYAWEMCARSEYAQKPV |
| | | DFPKSICEKFLEKFFECKSNEKYQQSFVTYARLLKINEDL |
| | | ATLEHFENEPPKDIESIYQDAQRYLDEVGNLCSNEDRAAI |
| | | AKWYEEYNKLWTKGDHKKLKEWIVSKSSIITNFTQAKMHL |
| | | GQKRGSQKTFVLKSYFHSSYGKIRDNNRFVNSNVTEVFKT |
| | | IASTFGKSFATIREYFNEESEVNKIEYGAVIIKDKNGDKY |
| | | LLLQKKNEGGIDMPIFNKSDENGDCDLYQVKSLTSKTVRK |
| | | IIASPNKYNDFFVNNDGKKIIYPDKTDFKYKINPYDKEEV |
| | | KKRKRELYNNDLVRPIIYSLTQSKFANKQNFEKYFDWTKA |
| | | LKQCSNIEQLYKTIDQKGYSLNPSKISKEQIADLVNNMNC |
| | | YLLPIVNQNITAKTKNDTNQFTKDWNKIFNEVDKDYRIHP |
| | | EFTMFYRYPTPDYPKFGEKRYSRFQMNVNFLMEVIPADGE |
| | | YCSRKEQIEIYNAPKDNENCQKNVVERFNNKIKALKPSYF |
| | | IGIDRGINELATLCVIDKEGKIVGDFEIYKREFDSNLKRQ |
| | | KYTSIETRDILDLSYLRVEKDENGESRLVDLSESEVWIDS |
| | | LDHDKGKRANKQIVHLKHLYYLRCIAHLLQSSDYKSIVLE |
| | | KLKDCNNLKDEEIKKVFEKDKFVDSYKGGEAYTDLPYDEI |
| | | RKLISDYQEIEQSNQTESEKSKALNTLCQLDASEYLKKGV |
| | | VANMIGVVVYVLKKYNYDAYISLENLCYAYGYSKDTLSGY |
| | | SITSTKEDPYLDFKDQENAKLAGLGTYSFFEVQLLKKLFK |
| | | LQIEENTELIPAFRSVDNYEKIFLLKNIDNKIYQFGIVYF |
| | | VDPKYTSLCCPICGEHGKKNVDRKKHTKKYDEDELVCKQC |
| | | GFHTNLSHIETRVMEDKTIKNSYDECNLKAIVSGDANAAY |
| | | NIAIRLGKNIYSTIADKVKDLHHEGKKYIIVKG |
| 5 | Amino acid sequence of the | MAGKEVDAKYLSHEELDDLVMRSEYNLIKRNVLEWSKKKE |
| | Unk 107 peptide | SDKSNYIFDELDKRFEEIIEIEDRDQRNAEYVEFLNEFFK |
| | | EVNHQTINQLDESTFINKIGDCSKAIKEYLLSWGKVCRRI |
| | | DKITVRKDYFKILARKTFFKYESKVGKKRTPLPSEVKLSG |
| | | QKGNNYFDEPINEGISQFWQNRVAKALNLHSQLESMLFDY |
| | | KKAIETEKHNQENPKGDNGSFDKLHLVDFRKMFLSVCSLV |
| | | MDSLRPIVNELIIVSDNVSKDEDKYILDFVNDKKTQWDLF |
| | | QQIENLQTICKDNGENIFFGKATFNKYTSEQAPNHRNNDI |
| | | AKVLRELKIEKFVSDYIDLDQEAINRKIYQSTQSRLENLN |
| | | NPQISPIIRAQYFKYKPIPTLVRFGLAKELAKQQGKKYSD |
| | | RLKGIQELFRIFGSSKSPALDYKNNRTDFSLDNYPIKVAF |
| | | DYAWEMCARSEYAQKPVDFPKSICEKFLEKFFECKSNEKY |
| | | QQSFVTYARLLKINEDLATLEHFENEPPKDIESIYQDAQR |
| | | YLDEVGNLCSNEDRAAIAKWYEEYNKLWTKGDHKKLKEWI |
| | | VSKSSIITNFTQAKMHLGQKRGSQKTFVLKSYFHSSYGKI |
| | | RDNNRFVNSNVTEVFKTIASTFGKSFATIREYFNEESEVN |
| | | KIEYGAVIIKDKNGDKYLLLQKKNEGGIDMPIFNKSDENG |
| | | DCDLYQVKSLTSKTVRKIIASPNKYNDFFVNNDGKKIIYP |
| | | DKTDFKYKINPYDKEEVKKRKRELYNNDLVRPIIYSLTQS |
| | | KFANKQNFEKYFDWTKALKQCSNIEQLYKTIDQKGYSLNP |
| | | SKISKEQIADLVNNMNCYLLPIVNQNITAKTKNDTNQFTK |
| | | DWNKIFNEVDKDYRIHPEFTMFYRYPTPDYPKFGEKRYSR |
| | | FQMNVNFLMEVIPADGEYCSRKEQIEIYNAPKDNENCQKN |
| | | VVERFNNKIKALKPSYFIGIDRGINELATLCVIDKEGKIV |
| | | GDFEIYKREFDSNLKRQKYTSIETRDILDLSYLRVEKDEN |
| | | GESRLVDLSESEVWIDSLDHDKGKRANKQIVHLKHLYYLR |
| | | CIAHLLQSSDYKSIVLEKLKDCNNLKDEEIKKVFEKDKFV |
| | | DSYKGGEAYTDLPYDEIRKLISDYQEIEQSNQTESEKSKA |
| | | LNTLCQLDASEYLKKGVVANMIGVVVYVLKKYNYDAYISL |
| | | ENLCYAYGYSKDTLSGYSITSTKEDPYLDFKDQENAKLAG |
| | | LGTYSFFEVQLLKKLFKLQIEENTELIPAFRSVDNYEKIF |
| | | LLKNIDNKIYQFGIVYFVDPKYTSLCCPICGEHGKKNVDR |
| | | KKHTKKYDEDELVCKQCGFHTNLSHIETRVMEDKTIKNSY |
| | | DECNLKAIVSGDANAAYNIAIRLGKNIYSTIADKVKDLHH |
| | | EGKKYIIVKG |
| 6 | Amino acid sequence of the | MEQYQLTKTIRFGLTKVRKEKKHLSHEELDELVMVSEERI |
| | Unk108 peptide | KKEHPQAENQLDEQSFVKKIGDCSKAIKEYLLSWGKVCRR |
| | | IDKITVRKEFFKILARKTFFKYESKVGKKRTPLPSEVKLS |
| | | GQKGNNYYDEPINEGISQFWQNRVSKALKLHSQLESMLFD |
| | | YKKAIETEKHNQENPKENNDQFDKLHLVDFRKMFLSVCSL |
| | | VMDSLRPIVNELIIVSDNVSKDEDKYILDFVNDKSKQWDL |
| | | FKQIEDLQNLCKDNGGNIPFGKATFNKYTSEQAPNHRDND |
| | | IHKVIRELKIEEFVSDFIGLEQEDIYRKIYQSTQHSLVNL |
| | | NKPSISPIIRAQFFKYKPIPVLVRFGLANYLNKQQGKKYS |
| | | NRLKDIQELFRIFGTSKSPELDYSDKNNRTEFSLDKYPIK |
| | | VAFDYAWERCARSKYAQKPVDFPKEICVTFLETYFEYNSK |
| | | EKNREAFEIYAHLLKVNECLATLEHFENEPPKDIKSLWQD |
| | | VQNHLDKVGKLCSNEDRKAITQWYEEYKNLWAKGNYKKLK |
| | | KWIESKSYTVTNFTQAKMHLGQKRGSQKTMVLKSYFHPTY |
| | | GKIKDGNRFINSNVTEVFKNIASTFGKSFATIRDYFNEES |
| | | EVNKIEYGAVIIKDKKGDKYLLLQKKNEGGIDMPVFNESD |
| | | GNGDYDVYQVQSLTYKTVNKIYNSTKYPEFFAINGEKAIY |
| | | APNRPQRFKDDQEKNTFNEKKLQSLKKCLTESDFMTNTTE |
| | | NYLQKFNWTEEINNCTDFEPLAKIVDQKGYYLKSYKISKE |
| | | QIAELVNNQNCYLLPIVNQNITAKTKNDTNQFTKDWNKIF |
| | | NDEYKDYRLHPEFTMFYRYPTPDYPCPGEKRYSRFQMNVN |
| | | FLMEVIPSEGEYVSRKEQIESFNTPKQDKEDNDNENSQAK |
| | | KVAQFNDNINTKKPSYIIGIDRGINELATLCVINSEGKIV |
| | | AVDENGLIKDEFDIYVKHFDKDNKCWIHNIKPKTATDNKP |
| | | RTILDLSNLRVETTIDGKQVLVDLSSDENGIQVNSKQIVH |
| | | LKRLYYLRCLSYLLQSSDYKSIILEKLKDVNNMTDDAIYE |
| | | VFKNDKFIDSYKGGVQYTDLPYDEIRHLISTYQEIDQSNK |
| | | TDSEKQSELNTLCQLDATESLKKGVVANMIGVVVYILKKL |
| | | NYDAYISLENLCRALYFSKDSLSGYTIENTSVNPDLDFKD |
| | | QENAKLAGLGTYSYFEIQLLKKLFKLQIDEKQFLVPGFRS |
| | | VENYEKIVKLGKVKHSIYQFGVVHFVEPANTSLKCPICGA |
| | | NGKRIKYNPNYDEDELVCKKCGFRSNISKIQNSKIMEDSV |
| | | IKTYYDNHNYKAIISGDTNAGENIALRLLMNLNTQIENAI |
| | | NHLHKTGKNYHSVNK |
| 7 | Amino acid sequence of the | MKNLTQFVNLYQLSKTLKFGLTLRNKIRKNGFEGEIYESH |
| | Unk 109 peptide | TELQELIKISEQKIIKETTDKNKEIMTFTELPLDEIRKCL |
| | | DDMHKYLDDWEQFYNRYDQIAVLKDYYRKLERKARFDGFW |
| | | REKNIKNKIQNNESETIKKPQSQVIKLSSLNNEYENKKRR |
| | | DYITDYWNENIQKAKRKFYEVNSVLKQFEVANEQNRDDKK |
| | | LNEVVLRKLFLSFTNLINDTLEPLCNGSICFPDIEKLTNS |
| | | KTDEQLQRFVFDDGFKKILSEQIENLKIYFAINGGYVQYG |
| | | RVTLNKYTALQQPNKVDEDIKNIIKELGLLEFVKKYENTE |
| | | QIINYIKNIKDKKQELNANNLSLIEKVQLFKYKTIPAGVQ |
| | | PSLIAYLARTEKKDKKTLRELFYAIGQPQSPSKDYKELQN |
| | | KTDFNLYKYPLKVAFDYAWESLAKSKYNPHIDFPDVKCKE |
| | | FLKDIFGTDISVNDNFKLYSALLFVRENLATLDHGNPNDK |
| | | NIHVNKVENTFKEIKDRLAKKEYKKEYKALEIICKWHKNS |
| | | ATIEQSEYEAAKQTIYEAAKQTIGQLRGRQKNQISKFKEL |
| | | TDSFKKLAPKFGKAFANLRDKFNEEYEINKISHCGVIVED |
| | | RNNDQYLLLSQLNDNRENASDIFELEADPNGELKIYQVKS |
| | | LTSKTLLKFLKNKKGSNTGFHINENWTFPKGKWDVINKDK |
| | | IFLNYVIQRITNSSMAKEQKWSNFKWDFRRCDSYEAIAKE |
| | | VDAKGYILESVNISKLTLNKLITEKKCLLLPIVNQDLTRQ |
| | | DKKTKNQFTKDWIKIFESNNCYRLHPEFKISYRYPTPNYP |
| | | KPEEKRYSRFQMISHLLCEYIPQNDNYKSRKEQVKIFNDK |
| | | VAQKESVEQFNQQFEITDDYYIFGIDRGIKQLATLCILNK |
| | | NGQIQGDFEIYTREFDKVNKQWKHTILEKRNILDLSDLRV |
| | | ETTVEGKKVLVDLSKVKLHSGNENKQTIKLKRLAYIRMLQ |
| | | YQMQHEQDKVLRFINQYKTIDEIEKNIRDLISPFKEGKQY |
| | | ADLPTEKIKDMLIQFGELSKNDSDKSKKELCTLCELDAVD |
| | | DFKTGAVANMIGVIAYLLEKYKYNVYISLEDLTRAFRLQR |
| | | DRLTNNILQSTNKDNTVDFKDQENLVLAGLGTYHYFEIQL |
| | | LRKLFRIQRNSEGDILHLVPTFRSVDNYEKIVRRDKKTDN |
| | | DKYVNYPFGIVRFVDPKYTSKKCPICDKTNTTRKDNVLIC |
| | | NSCNAVSGEYETDNENRHYITNGDDNGAYHIALKALSLRK |
| | | SKNLEKKK |
| 8 | Amino acid sequence of the | MEKYQITKTVRFGLTATNSNLYSDELKDLIETSEIKIKES |
| | Unk110 peptide | LKNKSHNSLQIEQLRSCLNGVKEYLKTWNNVYSQIDFLGI |
| | | SKDYYKVISRKARFDFDNKGLGSEVKLASLQSKYNSKKRI |
| | | QYILDFWEDNFQKTEILYRKSDELLKVFEEAEKQKRDDKK |
| | | LNEVELRKTFLSLFNLVNESLKPLVEGNLFTINDDKIDSR |
| | | NQNHEVIADFISNTKVRTELYESITELQNFFRDNGGYVPF |
| | | GRATFNQWTALQKADKNGEREIDKIIKQLNLETVSMANID |
| | | YKYNTFTKNFEQGGQVWKIKQNAKSVIELCQFFKYKKVSI |
| | | TTRLNLAKRLNKTNNFLSEFGISKSPALDYKKDKENFNLA |
| | | NYPLKVAFDYAWENCAKAKHESITFPELQCKDYLHNVFGV |
| | | DANKDKNGKIKNEELNKYADLLQFKILLGRLKAEFHKAAE |
| | | ETNKNNIRKLKNIFENLDYSGVQDFNKNKIKEIVEVWFAN |
| | | KEKNIGKKKEEMIPLTEKKKDDFSKAMQIIGQERGGLKSR |
| | | IKKYKTLTEMFKVCASRFGKLFADLRDYFNEAHEVDKIKY |
| | | RSWILEDGKQNRFVLLVDKAKDLELENEENGELKLYEVKS |
| | | LTSKSLIKFIKNKGAYPDFHSLNSFNSDEIKKNWTNHKAN |
| | | INFLKNLKSALENSLMAINQNWKEFNFDFSRCDTYEQIEK |
| | | EIDRKGYILKQQNISLNTIKKSINEEKSEKINNSKKLPSL |
| | | LFPIVNQDINREAKQEKNQFTKDWFEIFAEENNLHKKRLH |
| | | PEFHLFYRFPTKNYPNTKFKNGKEKSKRYSRFQMLAHFGL |
| | | EVFPQGDYISKKEQIEIFNDDKKQKEAVEKYNNSIVSEVE |
| | | YIIGIDRGIKQLATLCVLNKNGVIQSGFQIYTPSFNHDTK |
| | | QWEHSFLGKRNILDLSNLRVETTIKNEKVLVDLASIQTKK |
| | | GENQQKIKLKQLAYIRELQYSMQTRQVELLEYAKTLNSAE |
| | | DITEEKIKIFISPFKEGSHYEHLPKQEIYNLLNEWQNADE |
| | | TRKRKIQELDPTDSLKSGIVANIVGVIAFFCEKYNYKVRI |
| | | SLEDLTRAFSIQKDALTGTPIHRNDEDFKEQENRRLAGVG |
| | | TMQFLEMQLLKKLFKLQSEKNKHLIPAFRSVANYEKIVRR |
| | | DKENGGDEFVNYPFGIVTFVDPRNTSQKCPYCNNIARKED |
| | | DAFYRNAGENKNSLLCKKCGLSTIKGKENKSNQDDSKNQF |
| | | NIHFITDGDQNGAYHIALKTLENLHRLNTPKVTKHTKTKW |
| | | KK |
| 9 | Amino acid sequence of the | METNKTTKAINEYQTQKTIRFGLTVTNNNLYSENIVKLLK |
| | Unk111 peptide | CSEEKIKEQLKKTQTDDLQNQRLRCCLIEIKEYLKTWNNV |
| | | FSQIDFLAITKDYYKVLSRKAKFDYDKGNGSEIKLSSLQS |
| | | KQSKYNDKKRYQYILDFWHENFIKVENLYRKSDDLLKVFE |
| | | EAANQNQDDKKLNKVDLRKTFLSLFNLVNETLKPLIEGNL |
| | | FIVNDDKIDEHNSKHNFVSDFIVKTEERKQLHDCITDLQD |
| | | LFKANGGYVPFGRATINKWTALQKSNHKDDEIKRIIRELK |
| | | IENISMQNIDYKYKYDSFAENFKQIYNKEGEKVWVLQFDA |
| | | NSVIKVCQYFKYKKVPINARLNIAERLIKEKSWQREKKND |
| | | FLSEFGISKSPALDYKNDKENFNLANYPLKVAFDYAWENC |
| | | AKAIYETTTFPKEHCEKYLKEVFDLDIANNACFTKYALLL |
| | | RFKILICRIKSEETTQIQNIEAVRGILDEINKNISGRQDF |
| | | SKAKIITEINNWLSFKEKQTDKKEKYSNQDNFSLAMQIIG |
| | | QERGGLKSRIEKYKTLTDMFKVCASKFGKQFADLREYFQE |
| | | AYEVDKIKYRAWIIEDEKQNRFVLFANKEREIDLTSEEGN |
| | | LYFYEVKSLTSKSLVKFIKNRGAYADFHKLKNNFNYEKIK |
| | | RDWQYYKNDKYFIQNLKDALRNSKMAIDQNWAEFKFDFTK |
| | | FNTYEDIEKEIDRKGYKLVCKTVSLNTLKDFVENKGCLLL |
| | | PIINQDINKDDKQAKNQFTKDWNSIYDNKKRLHPEFNLFY |
| | | RFPTQDYPNTKFSNGTEKTKRYSRFQMLAHFGCESVPKGD |
| | | YLSKKEQIAIFNDDAKQKDAVEKFNNSIASDFEYIIGIDR |
| | | GIKQLATLCVLNKNGQIQGDFEIYTRTFENKQWKHTLSEK |
| | | RNILDLSNLRVETTIDGNKVLVDLASITTKNGENQQKIKL |
| | | KQLAYIRELQYSMQTRRDDLLDFAKGLQSADDILKDIRNF |
| | | IVPFKEGGQYADLPNERIYNLLKEWRDADDEAKRKIAELD |
| | | PAQDLKSGIVANMIGVVAFLCEKYGYKVRISLEDLTRAFG |
| | | IQKDALSGIAIAPNDEDFKEQENRRLAGVGTYQFFEMQLL |
| | | KKLFKTQVDKNLHLVPAFRSVDNYEKIVRRDKKTNGDEYV |
| | | NYPFGIVRFIDPKYTSKRCPKCGKTDVNRNQKTNIVKCNN |
| | | CEYETKAGNSSEANNIHFITDGDQNGAYHIAQKALKIQKE |
| | | Q |
| 10 | Amino acid sequence of the | MKQIKNQYQLSKTLRFGLTQKNKTKKENYAGEIYKSHSEL |
| | Unk112 peptide | SDLVEISEQRIKDSVSTNKNSESSLPVDAIGKCLNQISEF |
| | | LKGWQQVYQRTDQIALDKDYYKILCKKIGFDGFWFDKKNG |
| | | RKTKKPQARIISLLELEKKDDKETERKQYILDYWQENFIN |
| | | AVEKYNVVSEKLKQFEVALKINRTDNKPNEVEFRKLFLSL |
| | | VNIICDTLKPLCFQQICFPRLEKIDNSKIDNKNLIDFAID |
| | | YQSKNELLSLISKLKSYFEENGGNVPYCRATLNPKTAVKN |
| | | PESTDNSIESEIKKLGLDKIIKNNKDAFSFSYNLYNNTAE |
| | | DKKSKLKDDENGGLIERSLLFKYKSIPATVRFEIAKTLSK |
| | | PDGKTEEEILEFLRDIGQLESPAKDYADLKEKDNFNIEKY |
| | | PLKVAFNFAWEGLARAKYHPEAVFPTEICKQYLKNHFKIT |
| | | EDNKDFVMYAKLLELNAVLSTLEKAKPTDEKKFSVAAKKL |
| | | LEEIEWEKVGKNGSKNKEAITKWLQTKSKTDKNFKSAKQE |
| | | IGLFRGRIKNNIRIKNNIKSEYSEITNVFKNIAEEMGKTF |
| | | AEMRDKISGAAESNKISHYAMIIEDNNKDKYVLLQEFVEN |
| | | KNERIYAKSDSQKSDFKAYSVNSITSGAIVKMLKKIRTDK |
| | | LKESNNFANTQPELTSKEKEKRNIKEWKKFINEKGWNLEF |
| | | GLKLENKTLEEIKKEVDAKCYKFDIKYFDKETLSDLVKNK |
| | | NCLLLPIVNQDLAKKEKNESNQFTKDWNAVFPQDTPWRLT |
| | | PEFRISYRKPTPNYPKSDKGDKRYSRFQMIGHFLCDYIPK |
| | | TDSFISNRQQIENYKDDERQELAVKKFNAALRGRTKNEEY |
| | | KEQLNELAAKYSKNGQQKINVKTNEKFYVFGIDRGQKELA |
| | | TLCIIDQDKKIIGPHKIYTRSFNSEKKQWEHKFLEERHIL |
| | | DLSNLRVETTVFIDGKPEKTKVLVDLSEVKVKDKVTGEYT |
| | | KPDKMQIKMQQLAYIRKLQFKMQNEPEAVLAWYEKNSTED |
| | | LILKNFVDNEDGTNNGLVSFYGAAIEELKETLPIERIVDM |
| | | LKEFKTIKKEEGKLTKEDEEGREKNKRKMDKLVQLEPVDN |
| | | LKNGVVANMVGVIAFLLQKFDYQVYISLEDLSKPFSSKII |
| | | SGIDGVPIRVEKEEGRRADVEKYAGLGLYNFFEMQLLKKL |
| | | FRIQQDSENILHLVPAFRAMKNYDHIAVGKGKVKNQFGIV |
| | | FFVDAEATSKTCPRCGSTNQKPNKKDYPNAQQARLSNDKE |
| | | GWIDRDKSNGNDIIRCFVCGFDTTKEYTENPLKYINSGDD |
| | | NAAYLISAEGVKAYELATTLADNI |
| 11 | Amino acid sequence of the | MKNITNKYQITKTLRFGLSQKGKTKKEGFDGEIYQSHQEF |
| | Unk113 peptide | NKLVSVSEARIKKSVTTEQKTELALSIDNVARCLNNISDF |
| | | LINWQRVYYRTDQIALDKDYYKIMCKKIGFEGFWFETNRR |
| | | TQQKIKKPQSRIISLSALDKKDGLGKERKQYILDYWKENL |
| | | LSAAEKYEVVSEKLKQFQDALNINRTDNKPNEIELRKLFL |
| | | SLTHIVYDILQPLCYGQICFPKIEKLDNTKEDNKKLIEFA |
| | | SDYQSKSDLLSEIAELKQYFEENGGNVPFCRATLNPKTLV |
| | | KNPKSTDNSINEEIKDLGLKEILKTYKDVLNYNNYLESLS |
| | | AKQKLQLLNDRNTSIITRSLLFKYKPISANVQFDIAKTLS |
| | | PEVGKGEEDLRAFLRGIGQPKSPAKDYADLQNKSDFNIEA |
| | | YPLKVAFDFAWESLARAIYHADSDLPMDACKNFLQDNFKV |
| | | KNDDTNLKLYAQLQELKAVLSTLENGNPNNAAAFRLKATN |
| | | LLNEIPWKTVGNYGQQNKDEISKWLNNGKNKDDYKKAKQQ |
| | | IGLFRGRLKNNIQGFDNITQTNKNIAMKMGRTFATMRDKI |
| | | TGAAELNKVSHYAMIIEDRNTDRYVLLQPFTENEQDRIYS |
| | | QTDYNNGDYTTYEVNSITSGAIAKMLRKARIDELSKNDNN |
| | | RNLTSQPELTEEKKEKRNIKEWKNFIENKRWDLEFQLKLN |
| | | EKNFEQIKKEVDTKCYNLRTKKINKTTLEDLVNKSDCLLL |
| | | PIVNQDLAKEEKTNGNQFTKDWNSIFAQNTPWRLTPEFRV |
| | | SYRKPTPDYPISDKGDKRYSRFQMIGHFLCDYIPKSDKYI |
| | | SNREQILNYKNDELQKKAVKDFHEDLKGKTEEENQNESMN |
| | | ALMAKFGNVNKKQKATTVEKPKEKFYVFGIDRGQKELATL |
| | | CVIDQDKKIVGDFDIYTRSFNSERKEWEHTFFEKRHILDL |
| | | SNLRVETTASIDGKAEKKKVLVDLSEIKVKDKNGNYSKPD |
| | | KMQIKMQQLAYIRKLQFQMQTNPEGVLAWFKENSTKDLII |
| | | NNLVDKKNGEKGLISFYGSAIEKMEDTLPVDRIEEMLQKF |
| | | AALKKQEKEGEDVKLSIDQLVQLEPVDNLKNGVVANMVGV |
| | | IAYLLQKFNYQVYISLEDLSNPFGSQITGGIAGVPLKQGK |
| | | DEGRRMDVEKYAGLGLYNFFEMQLLKKLFRIQQDSCNILH |
| | | LVPAFRAQKNYDHVAVGKEKVKGQFGIVFFVDANATSKTC |
| | | PVCGTTNNKPNNQKYPNAKKGLSADGKEVWLERDKSNGND |
| | | IIRCFVCNFDTTKEYTENPLKYIKSGDDNAAYLISAAGIK |
| | | AYELATTLINNQ |
| 12 | Amino acid sequence of the | METLNQFTGLYSLSKTMRFGLTLKEKKPKNDSIAVESLYQ |
| | Unk114 peptide | SHQDLKELVELSDKRIIEEKKPEPPVENLGNPPIEKLRDC |
| | | LNSMQKYLNDWRKVYTRYDQLAVLKDFYRKLERKARFDGF |
| | | WKDKKGQNQPQSQEIKLSSLKHKSGEKEIKDCIVTYWGEN |
| | | IRKANEKWHQVDSVLKQFEEAKRKNRDDKKLNQVELRKLF |
| | | LSLANLVNDTLVPLCQRSITFPNADKLSDNARDKSVLDFI |
| | | GDNEIREHLLDKITKLKEYFQDNGGYVPFGRVTLNQYTAM |
| | | QKPNKTDKEIEDAIKNLGLSIIKSQNFDAFEHIEEATDKV |
| | | ERLNTVSLPLVERAQYFKDKTIPVGVRDSLAKYLAKDDTA |
| | | KEKELIDLFEKIGMPKRPAKDYSDPTLKEKFDLRKYPLKV |
| | | AFDYAWETVASKELHDDILKNKCKKYLKDIFDVDTDKSIF |
| | | FNIYSDLNYMKIILSRIEYPTQNQLSKDNFLEWNRKVITI |
| | | LDGDDFSHFNKNADGSTDKKMNTAKTYVKTWLDKLEANIE |
| | | QFDGQDFKKFYEDFKKKNKNSCKDFDDAKRDIGLKRGGLK |
| | | QIIEETETFTDKKTGKQKPKYKDSKYKELTEAFKSIAVDF |
| | | GKHFATLRDKFNEENEINKIEYYGVIVEDENADRYLLLSK |
| | | LSESREEIKNIFPDKAEGLKTYKVKSLTSKTLTKLVKNKG |
| | | AYKDFHISDMRVDFKKIKEEWSAYKNDQAFLKYLKKCLTD |
| | | SSMAQAQNWSEFGLDFDKCNTYEEVEKELDGKAYLLQETR |
| | | LSKATITNLVKNKGCYLLPIINQDLAREDRTAKNQFTKDW |
| | | KQIFENKKHYRLHPEFNMAYRQPTPNYPNSEIGDKRYSRF |
| | | QMIANFMCEIVPQSTSYATRKEQIQTFNDNNKQQKAVKDF |
| | | DSKFKLSDSYFIFGIDRGIKQLATLCVLDQGGVIRGGFEI |
| | | YTRHFDGNKKQWVHTSLERRNILDLTNLRAETTIDGKKVL |
| | | VDLSKVEIKNQTDNKQNIKLKQLAYIRKLQYQMQTNPEKV |
| | | KNMSDEDIENDLKDIITPYKEGTHYADLPIENIKAMLDRF |
| | | KVLYGKTDQQSKQELKELCELDAADNLKGGIVANMVGVIA |
| | | HLMEQYNYRVKISLENLTTSFVNQSDGLNEYFISRGMDFK |
| | | EQENAALAGLGTYQFFEMQLLKKIFRIQQDDGNVLHLVPA |
| | | FRSKEDYEKIIRRDKNDGDEYVNYPFGLVTFVDPRYTSRK |
| | | CPICGKTDVKRNDNIITCKKCGAVSGKYSFDDKNRQFITN |
| | | GDENGAYHIALKTRKEVHNEN |
| 13 | Amino acid sequence of the | MDKENSFKGFTNLYEVRKTVRFGLTQPNKKGELKTHLEFD |
| | Unk119 peptide | DLINKSFENIKKDVKSRDKPNFKEKELIEKINQFINGLEK |
| | | QLGNWKQIYERYDVISVNKDYYKILARKAKFDAFKKDKKP |
| | | QASQIKLSSLQKDNRKDNIIRYWGNIITRSDYLINIFKPK |
| | | LEQYLNAVNNPNNSSHTKPDLIDFRKVFLQFLKVNEEYLQ |
| | | PLFDKSIQFETGKKENSEEIKKINTFSGDENNKEINYLID |
| | | LGKEIREYFEANGSQVPYGKVSLNYYTALQKPNNFGEDIR |
| | | KGVENLGIIKFLNKSEEDIKNYLKQNSKEKINLLNNAKNH |
| | | YFIELIHLFKPKTIPFSVKYNLAKYLEKNFNLKYEDILNK |
| | | FDLLGKSVDIGKDYLECKEKEKFSLEKYPIKSAFDYSWEN |
| | | LARNLKRDVDFPKSVCEKFLKDNFDIIINNSSFNLYANLL |
| | | FIAENLATIEYGNPNNENEIIESIKNTFDDIKFESNKQEY |
| | | DGYKKEILNILNQEKSKRNYKNILTAKQRLGLLRGQQKNK |
| | | ISKYYNLTQSFKKIASFIGKTLATIREGLKEENELNKITD |
| | | YGIIIEDKNQDKYILTLKLDGKDIREKIKSKLWDGEYKVF |
| | | EINSFTSRALNKFIKNPLGEDSKKFHGDYKYKHKEVSIYK |
| | | DVKWIGYKEEFLIHLKDSLVNSQIAKEQNWKAFGWNFDNF |
| | | NTYEKIEKEIDKKGYKLIKNSISKENLEYLINEEKCLLFP |
| | | LINQDISSKKEQNKNEFTKDFNKAFLGIGYRIHPEFSIFY |
| | | RQPDEENKKINKSGIINRFGRLQLLANIGIEYIPQNNDYK |
| | | TRKEQNKISLDQTNQNELVQNFNKEKVNKYFDSLDDYYIF |
| | | GIDRGIKQLATLCITNKNGIIQSYEIYTKYFNNNSKKWEY |
| | | KKNRIEGILDLTNLKIESDKDGNKFLVDLSLFEAKDENGN |
| | | STGTNKQNIKLKQLAYIRKLQYQMSSNEKGVLNFLKKYQT |
| | | KEERQNNIKELITPYKEGHHFEDLPVNIFEEMFENYEKLK |
| | | NDKTLSEIEKQNLMKLTIELDSSEDLKKGVIANMIGVIVY |
| | | LMKKYDYKVKIAVENLNQSFMGQNDGLNNSYISIKTNFKD |
| | | QENGALAGMGTYHFFENQLLRKLYKVSVEEGILHLVPFFN |
| | | SLDNVNKLNFEKEKILWVQTENYRKFGIVSFVRPHNTSKR |
| | | CPICKSINVKRKDNITTCSDCGFITGKDNNIVIKKYKKEG |
| | | LNLDLIKNGDDNGAYNICCKIGL |
| 14 | Amino acid sequence of the | MRFGLTQPNKKGELKTHIEFSDLVNKSFENIKKEVNSKDK |
| | Unk 120 peptide | SKFDTRKELIDKINQFISGLENQLGDWKNMYERYDLISVN |
| | | KDYYKILARKAKFDAFKKDKKGVKQPQANQIKLSSLRYNK |
| | | ELIINYWGNIISRSDYLINVFKPKLEQYLNAVNNPNNSSH |
| | | TKPDLIDFRKVFLQLLKISEEYLQPLENKSIQFETGKKEN |
| | | SGDIKRVNDFSGNENNKEINDLLDLGKEIREYFEANGSQV |
| | | PYGKVSLNYYTAVQKPNNFDKEIKEGIKDLGIIEFLKKSE |
| | | EDIKNYLKQDSKEKIYLLNNSKNPYSIELIQLFKPKTIPF |
| | | SVKYNLSKYLEKNYNLKYEDILNKFDLLGKSVDIGKDYLE |
| | | CKDKEKFSLEKYPIKSAFDYSWENLARSLKRDVDFPKNVC |
| | | EKYLNDNFNINVGNSSFNLYANLLFIAENLATIEYGKPNN |
| | | EKEIIDSIKETFLELSDEIEKNNKKNEVENIIKYLNLNTD |
| | | ERKNIKDLQKKYFKNLDTKEQNILNIFDSFTKSKQSLGLL |
| | | RGQQKNKIDKYRNLTQKLVDKKDSHIGIASFIGRTLASIR |
| | | EGLKEENELNKITDYGIIIEDKNQDKYILTLKLNGKDTRE |
| | | KIKNNLGNGEYKVFEINSFTSKALNKFIKNPLGEDSKKFH |
| | | GYFQYKHREVSIYDENEKWVGYKEEFLKHLKHSLINSQIA |
| | | VEQNWKDFGWNFDNCDTYEKIEKEVDKKGYKLIETSISKE |
| | | NLENLIHKEDCLLFPLINQDISSKKEENKNDFTKNFEKVF |
| | | LGDGYRIHPEFSIFYRQPNEENLKPNKSGIINRFGRLQLL |
| | | ANIGVEYIPQNNDYTTRKEQNKISIDQTKQNESVQKFNKE |
| | | KVNPYFDSLEDYYIFGIDRGIKQLATLCITNKKGVIQNFD |
| | | IYTKHFNDNSKNWEYKNNRTEGILDLTNLKVESDKEGNKY |
| | | LVDLSLFEAKDENGNLTGTNKQNVKLKQLAYIRKLQYQMS |
| | | SNEEGVLSFLNKYKTKEERQNNIKELITPYKEGHHFEDLP |
| | | MNIFEEMFENYEKLKNNKTLSEGEKQNLMKLTTELDASED |
| | | LKKGVVANIIGVIVHLMKEYDYKVKIAIEDLSNAWYFSKD |
| | | GLSGDSILNSKIDEEMDLKKQDNLALAGVGTYHFFEMQLF |
| | | KKLFKISVEKGILHLVPSFGNVRNYTDLLKEKYKYQYQQF |
| | | GVIYFISPKFTSSKCPICGKGGKKHIKRENNVITCKECGF |
| | | VSGKDNSINIKNNKKEGLNLDLIKNGDDNGSYNIGGKIK |
| 15 | Amino acid sequence of the | MLHAFTNQYQLSKTLRFGATLKEDEKKCKSHEELKGFVDI |
| | SuCas12a2 peptide | SYENMKSSATIAESLNENELVKKCERCYSEIVKFHNAWEK |
| | | IYYRTDQIAVYKDFYRQLSRKARFDAGKQNSQLITLASLC |
| | | GMYQGAKLSRYITNYWKDNITRQKSFLKDFSQQLHQYTRA |
| | | LEKSDKAHTKPNLINFNKTFMVLANLVNEIVIPLSNGAIS |
| | | FPNISKLEDGEESHLIEFALNDYSQLSELIGELKDAIATN |
| | | GGYTPFAKVTLNHYTAEQKPHVFKNDIDAKIRELKLIGLV |
| | | ETLKGKSSEQIEEYFSNLDKFSTYNDRNQSVIVRTQCFKY |
| | | KPIPFLVKHQLAKYISEPNGWDEDAVAKVLDAVGAIRSPA |
| | | HDYANNQEGFDLNHYPIKVAFDYAWEQLANSLYTTVTFPQ |
| | | EMCEKYLNSIYGCEVSKEPVFKFYADLLYIRKNLAVLEHK |
| | | NNLPSNQEEFICKINNTFENIVLPYKISQFETYKKDILAW |
| | | INDGHDHKKYTDAKQQLGFIRGGLKGRIKAEEVSQKDKYG |
| | | KIKSYYENPYTKLTNEFKQISSTYGKTFAELRDKFKEKNE |
| | | ITKITHFGIIIEDKNRDRYLLASELKHEQINHVSTILNKL |
| | | DKSSEFITYQVKSLTSKTLIKLIKNHTTKKGAISPYADFH |
| | | TSKTGFNKNEIEKNWDNYKREQVLVEYVKDCLTDSTMAKN |
| | | QNWAEFGWNFEKCNSYEDIEHEIDQKSYLLQSDTISKQSI |
| | | ASLVEGGCLLLPIINQDITSKERKDKNQFSKDWNHIFEGS |
| | | KEFRLHPEFAVSYRTPIEGYPVQKRYGRLQFVCAFNAHIV |
| | | PQNGEFINLKKQIENFNDEDVQKRNVTEFNKKVNHALSDK |
| | | EYVVIGIDRGLKQLATLCVLDKRGKILGDFEIYKKEFVRA |
| | | EKRSESHWEHTQAETRHILDLSNLRVETTIEGKKVLVDQS |
| | | LTLVKKNRDTPDEEATEENKQKIKLKQLSYIRKLQHKMQT |
| | | NEQDVLDLINNEPSDEEFKKRIEGLISSFGEGQKYADLPI |
| | | NTMREMISDLQGVIARGNNQTEKNKIIELDAADNLKQGIV |
| | | ANMIGIVNYIFAKYSYKAYISLEDLSRAYGGAKSGYDGRY |
| | | LPSTSQDEDVDFKEQQNQMLAGLGTYQFFEMQLLKKLQKI |
| | | QSDNTVLRFVPAFRSADNYRNILRLEETKYKSKPFGVVHF |
| | | IDPKFTSKKCPVCSKTNVYRDKDDILVCKECGFRSDSQLK |
| | | ERENNIHYIHNGDDNGAYHIALKSVENLIQMK |
| 16 | Nucleic acid sequence | ATGGAAGCAATAAAAAACAACTATCAATTAAGCAAAACCC |
| | encoding the Unk88 | TGCGTTTTGGATTAACACAAAAAAGTAAAACAAGAAAAGA |
| | polynucleotide | TGGTTTTACAGGAGAAATCTACCAAAGTCATAACGAATTA |
| | | AAAGACTTGGTAAAAAGTTCTGAAGACCGAATTAAAAAAT |
| | | CCGTATCAACAGACGAAAAATCCGAGATGAGTTTGTCTGT |
| | | TGATAAAATTAGATGTTGTCTTGTGATGATTTCGGATTTT |
| | | CTTAGTAGCTGGCAACAAGTTTATTCTCGTGCCGACCAGA |
| | | TTGCCTTAGATAAAGACTATTACAAAATCCTTTGCAAAAA |
| | | AATTGGCTTTGACGGTTTCTGGGTTGATGAGCGATATGAT |
| | | AGAAAAAACGACAAAACAGTTAGAACAAAAAAGCCACAAT |
| | | CCCGAACCATAAATCTTTCCGAATTAGACAAGAAAGATGA |
| | | CAAAGGCATCGAACGCAGACAATATCTCCTCACTTATTGG |
| | | CGGGATAATCTGATAAATGCAGCAGATAAATTTGAAGTAG |
| | | TTACTGAAAAGTTGAAACAGTTTGAAGATGCTTTGAATAT |
| | | TAACCGAACCTATAATAAACCGAATGAAGTAGAATTGCGA |
| | | AAACTATTCTTGTCATTGACGAATATTGTTCAAGAAACAC |
| | | TGCAACCGCTTTGTCTGGGACAAATTTGTTTTCCTAAATT |
| | | GGAAAAAATAGATGATTCAAGAACTGAAAATAAACACTTG |
| | | ATTGATTTTGCAACCGATTATCAATCCAAAAGTGATTTGC |
| | | TTTCTGAAATTTCAGAATTAAAAAAATACTTTGAAGAAAA |
| | | TGGTGGTAACGTGCCTTATTGCCGAGCAACTCTTAATCAA |
| | | AAAACAGCTGTAAAGAATCCCAATTCTACCGACAATAGCA |
| | | TAGACTCTGAAATTAAAAAGCTCGGTCTTGACAAAATATT |
| | | GAAAGAGAATAAAGATGCTTTGTATTTTGCCAATAAAATT |
| | | TACAGTTTGTCTGCCAAAGAAAAACTCTCAAAATTAGATG |
| | | ATAAAACTACCGGATTGATAGAACGTAGCTTACTGTTTAA |
| | | ATATAAGCCTGTTCCTGCTATTGTACAATACGAAATAGCT |
| | | AAAACTTTAAGCGAAACCATCAATAAAAGCGAAGAAGATT |
| | | TATTGGAATTTCTTCGCAGTATTGGACAAACGAAAAGTCC |
| | | TACAAAGGATTATGCCGATTTACAGGATAAAAACGATTTC |
| | | GATTTAGATGCTTATCCGCTAAAAGTAGCATTTGATTTCG |
| | | CTTGGGAGAACTTGGCAAGAAGTATTTATCATAGTGATGC |
| | | GGATATTCCGGTTGCTGTTTGTGAAGGGTTTCTTAAAAAA |
| | | AACTTTGGCATTGATAAAAGCAATGCAGATTTCAAGTTAT |
| | | ATGCACAATTACAGGAATTAAAGGCTGTGTTGGCAACATT |
| | | GGAATACGGAAATCCAACAAATAGGCAAACGTTTATAAAT |
| | | GAAGCAACAAAATTGTTATCCCCAATATCTTGGGATAAAA |
| | | TCGGTCGAAACGGAAATCAAAATAAATATTCTATTGAAAA |
| | | ATGGCTGAAAACTCTAACAAAAGATGATAAAGATTATAAA |
| | | GATGCCAAACAGCAAATAGCCTTGTTTAGAGGACGATTGA |
| | | AGAATAATATTAAGACTTTTGATGACATTACGAAATATTT |
| | | TAAATCCGTTGCTATGGAAATGGGCAGAACCTTTGCCCAA |
| | | ATGCGTGATAAAATAACCGGTGCGGCAGAACTTAACAAAG |
| | | TTACCCATTATGCCATAATTATCGAAGACCAAAATTTTGA |
| | | TAAATATGTTTTATTACAGGAGTTTGTCGATAAAAAGGAG |
| | | AATAGAATATATGCAAAAACAGACAGACATCACAGCGATT |
| | | TTACGACTTATTCGGTTAATTCGGTTACTTCAGGCTCTAT |
| | | TGCCAAAATGCTCAGGAAAAAGAGAATGGATGAATTGAAT |
| | | AGAAATAACAGAAATAGTTTTGAACAGAAACCTGAATTAT |
| | | CTGAAGAACAAAAAGAACAGCGTAACATTAGAGAATGGAA |
| | | AGAGTTTATAGAAGATAAACGCTGGGACTTGGAATTCCAA |
| | | TTAAATCTCAGCAATAAAACGTTTGAGCAAATTAAAAAAG |
| | | AAGTTGATGCCAAATGCTATGAATTGGATATAAACTATAT |
| | | CAGCCAAGAAACTTTATCCGATTTGGTAAATAAAAAGGGT |
| | | TGTTTGCTATTGCCGATTGTTAGTCAGGACATAGCAAAAG |
| | | AAAATAAAACAGAAGGCAATCAATTTACCAAAGATTGGAA |
| | | CGCTATTTTCACTCAAGAAACTCCTTGGAGACTAACTCCG |
| | | GAATTTAGAGTTTCTTATAGAAAACCAACGCCAAATTATC |
| | | CGGTTTCCGACAAAGGAGACAAACGCTACTCCCGCTTCCA |
| | | AATGATAGCTCATTTCTTATGCGATTATCTTCCAAAATCC |
| | | GATAGCTATATTTCTAATTGGGAACAAATTGCAAATTATA |
| | | AAGATGATAAATTGCAGGAAAAAGCGGTAAAAGAATTTAA |
| | | TGCAGATTTGAGAGGACGAACGGAAGAAGAAAAACAAAGT |
| | | GAATCGGTGAATGCGTTGCTTGCCCATTTTGGAAACCAGA |
| | | ACAAAAAACAAAAACCTGTTGAGCGACCTAAAGAAAAATT |
| | | TTATGTCTTTGGCATTGACCGCGGACAAAAAGAATTGGCT |
| | | ACGCTGTGTGTAATAGACCAAGACAAGAAAATTGTCGGTG |
| | | ATTTTGATATTTATACCCGTTCATTTAACTCCGAAGCAAA |
| | | ACAATGGGAACATAAATTACTTGAGAAACGTGTTATTCTC |
| | | GATTTATCTAATTTGCGTGTGGAAACAACTATTGTAATAG |
| | | ACGGAAAGCCGGAAAAGAAAAAGGTTTTGGTGGATTTGAG |
| | | CGAAGTAAAGGTAAAAGACAAAGATGGTAAGTATTCAAAG |
| | | CCGAATAAAATGCAGGTAAAAATGCAGCAGTTGGCGTATA |
| | | TTCGTAAACTGCAATTCCAAATGCAGACCAATCCGGATGC |
| | | TGTATTAGAATGGTATGCAAACAACAAGACCAAAGAACAA |
| | | ATCATGTCTAACTTTGTAGATAATGAAAACGGTGATAAAG |
| | | GGTTGGTTTCTTTCTATGGAACTGCCGTTGAGGAACTGAA |
| | | TGAAACCTTGCCGATTGATAAAATCGAAGAAATACTCAAA |
| | | AAATTTCAAGAGCTGAAAGATAAAGAAAAACAAGGTGAAA |
| | | CCGTTAAATTGGAAATTGATAAACTTGTTCAGCTAGAGCC |
| | | GGTAGATAATTTAAAAAACGGAGTGGTTGCCAATATGGTT |
| | | GGCGTTATTGCTTATTTACTTCAAAATCTTGATTATCAGG |
| | | TCTATATTTCACTCGAAGATTTATCAAAACCATTCAGTGG |
| | | TCAAATTATAGGAGGAATCGCCGGTGTGCCAACAAAAACA |
| | | AATAAAGAGGAAGGTCGGCGTGCCGATGTGGAAAAATATG |
| | | CCGGATTAGGTCTTTACAATTTTTTTGAAATGCAACTGCT |
| | | CAAAAAACTATTCCGCATTCAGCAAGACAGCCAAAATATT |
| | | TTGCATTTAGTGCCGCCTTTCAGAGCAATGAAAAATTATG |
| | | ACCATGTTGCCGTTGGCAAAGGCAAAGTAAAAAATCAGTT |
| | | TGGTATAGTCTTTTTTGTGGATGCTGATGCCACTTCTAAA |
| | | ACCTGTCCATGCTGCGGTTCATCAAATAATAAGCCAAATC |
| | | TGAAGATGTATCCAAACGCTAAAAAGGGACTATCAAAAGA |
| | | AGGGAAGGAAGTTTGGGTAGAGCGTGATAAATCGGAAGGT |
| | | AATGACATTATCAGATGCTTCGTTTGCGGGTTTGATACCA |
| | | CAAAAGATTATTCCGAAAATCCGCTTAGATACATTAAAAG |
| | | CGGAGATGATAATGCCGCCTATCTGATTTCTGCAGAAGGA |
| | | ATTAAGGCTTATGAATTAGCAACAACATTAGTGAACAACA |
| | | AATAA |
| 17 | Nucleic acid sequence | ATGGAAAATATCAAAAACAATTATCAATTATCCAAAACAC |
| | encoding the Unk89 | TTCGTTTTGGCTTAACACAAAAACAAAATGGAAATAGCTC |
| | polynucleotide | AAATACAGATAATGTTTATCATAGCCATAGTGCTTTAAAA |
| | | GAATTAGTGGACATTTCCGAAAACAGAATTAAAAAAAATG |
| | | TTTCTACAGAGGGAGCAACCGAAATGCAATTATCCATTGA |
| | | AAGCATTAGAAAATGTATGATTATGATTGAGCAGTTTATT |
| | | AAAGACTGGAAAAGAGTATATTATAGATCAGACCAAATTT |
| | | CTTTAGATAAAGATTTTTACAAAAAGCTAAGTAAGAAAAT |
| | | TGGGTTTGAAGCATTTTGGTTTGAAAGAAATAAAAAAACT |
| | | CAACAAAGGATAAAAAAACCTCAATCTTGCATAATTGCTT |
| | | TGTCTGAACTCTCAAAAAGAGATAATTTTGGAAAAGAACG |
| | | TCAAGAATATATTGTTGAATATTGGGAAAATAACTTGCTA |
| | | AAATCTACAGAAAGATACGAAGAAGTAAGTGAAAAATTAG |
| | | AACAGTTTGAATTAGCTCTTAAAATCAATCGAACAGACAA |
| | | TCGCCCGAATGAGGTAGAATTGCGAAAAATGTTTCTATCG |
| | | CTTGTAAATATGGTTCGAGAAGTAGTTGAGCCACTCTGTT |
| | | TGGGACAAATTTCTTTTCCTAAATTAGAGAAATTAGCAGA |
| | | CAATTCTAAAAATAAACAACTACGAAAATTTGCAACAGAT |
| | | TATCAATCAAAAAGTGATTTATTGACACAAATTTCTGAAT |
| | | TGAAAAAATATTTTGAAGAGAACGGTGGCAATGTGCCGTA |
| | | TTGCAGAGCTACGCTCAATCCGCTTACTGCTGTAAAAAAT |
| | | CCTAAATCTACTGATAGTAGTATTCTTGATGAAATCAAGA |
| | | AATTAAAATTAGATGTTATCTTAAGAGACTACCAAAGTGT |
| | | TGCTCTTTTTGATAATTCCATTCGAGATTTGACAGCATCA |
| | | CAGAAAATGCAATTGCTTAACCAAAATAACGAAGGCCTTA |
| | | TAAAGCGTGGTTTACTATTCAAGTACAAACCCATTCCTGC |
| | | TATTGTGCAATATGAAATTGCAAAAGTATTGAGTGCTGAA |
| | | CTCAACAAAGACGAACAAGAATTACGAAATTTTTTAAGAG |
| | | ATATTGGTCAGGTTAAAAGCCCTGCCAAAGACTACGCAGA |
| | | ATTGCAAGATAAAAAAGATTTTGACATCAATCATTATCCT |
| | | TTAAAAGTTGCTTTTGACTTTGCGTGGGAATCGCTTGCTA |
| | | AATCTGTTTATCATCCCGATATAGATTTCCCAGAGGAACA |
| | | ATGCAAAACATTACTAAGGGAAGTTTTCCAAGTAGATGAA |
| | | AACAACGAAAATTTTAAATTTTATGCCCAACTTTTAGAGC |
| | | TACGGTCGTTACTTGCCACTTTAGAACACGGAAAACCAAC |
| | | AGAGGTAATAACAATTGAAAACGAAGTAAAGAAAATTCTC |
| | | GAAAATATTGACTGGAGTAAATTTGGAGATAGAGGCAAAA |
| | | ACTACAAATCGGCTATTGAAAATTGGATACACAATAGAAA |
| | | CAAAAAAGATTTTAAAGGCGACTATTTCAAAAAAGCCAAG |
| | | CAACAGATAGGTTTAACACGTGGTAGACAAAAAAATTTAA |
| | | TAAAAAAGTATGACGAAATAACAAAGTCTTACAAAGACAT |
| | | TGCAATGAAAATGGGCAAAACTTTTGCCGAAATGCGGGAT |
| | | AAAATTACCGGTGCAGCCGAACTCAATAAAGTATCGCATT |
| | | ACGCAATGATTGTGGAAGATACCAATCAAGACAAGTATGT |
| | | TTTATTGCAAGAATTTGTAGAAAATAATAAGGATAGAATT |
| | | TATGCAAAAAGTACTCCTCAAAATGAAGATTTTAAAGCAT |
| | | ATTCGGTCAATTCTGTTACTTCTTCGGCTATCGCAAAAAT |
| | | GATTAGAAAAATAAGAATTGACAAGCTACAAGCAAATGAA |
| | | CGAAACAATAATAGACAACAAGCACCAGAACTATCAGAAA |
| | | CTCAAAAAGAGGCAAGAAATATCAAAGAATGGAAAGATTT |
| | | TATTGCGGAAAAACGATGGAATTATGAGTTCGATTTAAAA |
| | | TTGGACAATAAAAATTTTGAGCAAATAAAAAAAGAAATAG |
| | | ACTCGAAATGCTTTAAATTAGAAACCAAATATATGAGCGA |
| | | GGAAGTACTTGTTGATTTGGTCAAAAATCAAAATTGCCTG |
| | | CTTTTACCAATTATCAATCAAGATTTAGCTAAAAAAATAA |
| | | AATCCGAGAGTAACCAGTTTACCAAAGATTGGAATGCCAT |
| | | TTTTGCACAAAACACACCTTGGCGACTTACGCCAGAATTT |
| | | AGAGTGTCTTACAGAAAACCTACTCCAAATTATCCCAAAT |
| | | CCGAAAGAGGTGATAAAAGATATTCTCGTTTTCAAATGAT |
| | | AGGTCATTTCTTATGCGATTTTATCCCTAAAACTGCTGAT |
| | | TATATTTCAAATAGAGAGATGATTGCTAATTTTAAAGATG |
| | | ATGAAAAACAAAAACAAACGATTATAAATTTTCATGAAAG |
| | | ATTAAACCCTAAATCTGAAAATGAAAAAATGAACATGTTG |
| | | TTAGCTAAATTTGGAAATAAAAATTCAAATCAATCTAAAG |
| | | AAACAAAAAAAGAAGAAAAATTTTATGTGTTTGGAATTGA |
| | | CCGCGGGCAAAAAGAACTTGCAACACTTTGCGTAATAGAC |
| | | CAAGATAAAAAAATTATTGGAGATTTTAATATTTACACCC |
| | | GCTCCTTCAACACCCAAAACAAGCAATGGGAGCATCAACT |
| | | TTTAGACAAAAGACATATTTTAGACTTATCTAATCTGCGA |
| | | GTCGAAACAACTATTGTAATTGATGGTAAGCCTGATGTGC |
| | | GAAAAGTATTAGTTGATTTGAGCGAAGTGAAAGTGAAAGA |
| | | CAAAAATGGGAATTACACGAAAACGGATAAAATGCAAGTG |
| | | AAAATGCAACAGTTAGCATACATACGCAAACTTCAATTTC |
| | | AGATGCAAACGAATCCTGATACTGTGTTGGAATGGTACAA |
| | | TAAAAATCAAACAAAGGAGGCGATTTTAAATAACTTTGTT |
| | | GATAAACCAAATGGCGAAAAAGGCTTAGTCTCTTTTTATG |
| | | GGTCTGCTGTTGAGGAATTAAAAGATACTTTGCCGATTGA |
| | | AAGAATTGAAAAGATGCTGCAACAATTTAAGTCTCTAAAA |
| | | AATGAAGAAAAAGAAGGGAAAGATGTAAAGGCTGAAATTG |
| | | ACAAATTGATACAACTTGAACCTGTAGATAACTTGAAAGC |
| | | AGGTGTAGTGGCTAATATGGTTGGTGTGATTGCTCATTTA |
| | | TTAAAAGAATTTAATTATCAAGTATATATATCATTAGAAG |
| | | ATTTATCTAATCCTTTTGGTAGCCATGTTATAGATGGAAC |
| | | CACCGGAACTCATTCAAAAACGAATAAAGGTGAAGGTAAA |
| | | AGAGCCGATGTAGAAAAATATGCAGGGCTGGGTTTGTATA |
| | | ACTTTTTTGAAATGCAATTACTCAAAAAACTGTTCCGAAT |
| | | ACAGCAAGATAGCCAAAACATTTTACATTTAGTACCCGCA |
| | | TTTAGAGCCGTAAAAAATTATGAAAACATCATTGCGGGAA |
| | | AAGATAAAATTAAAAACCAATTTGGAATAGTATTTTTTGT |
| | | AGATGCCAATTCTACTTCAAAAATGTGTCCTGTTTGTAAT |
| | | TCTACCAATGAAACTAATAGAGAGTACCCAAATGCAAAAA |
| | | AAGGAACTTCTAAAGATGATAAAGAAGTTTGGGTAGAACG |
| | | AGATAAATCAAACGGAGACGACATAATTCGCTGTTTTGTG |
| | | TGTGGGTTTGACACAACTAAAAAATATGAAGAAAATCCAC |
| | | TAAAATTCATTAAAAGTGGTGATGATAATGCAGCGTATAT |
| | | AATTTCTGCTTTTGGCATAAAGGCTTATGAATTAGCTAAA |
| | | TCAGTAATTGATAACAAGTAA |
| 18 | Nucleic acid sequence | ATGAAAAATTTAACACAATTTGTAAATTTGTACCAACTCT |
| | encoding the Unk97 | CAAAAACATTGAAATTCGGATTAACATTACGAAATAAAAT |
| | polynucleotide | AAGAAAAAATGGTTTTGAAGGAGAAATTTATGAAAGTCAT |
| | | ACTGAATTACAGGAACTTATAAAAATTTCAGAGCAAAAAA |
| | | TCATTAAGGAAACAACTGATAAAAATAAAGAAATAATGAC |
| | | ATTTACAGAATTGCCCTTAGATGAAATTCGTAAATGTCTT |
| | | GACGACATGCATAAATATCTCGATGATTGGGAACAATTTT |
| | | ATAACAGATATGACCAAATAGCAGTACTTAAAGATTATTA |
| | | TCGAAAATTGGAACGTAAAGCAAGATTTGACGGTTTTTGG |
| | | AGAGAAAAAAATATTAAGAACAAAATACAAAATAATGAAT |
| | | CTGAAACTATCAAAAAACCGCAGTCTCAAGTTATCAAATT |
| | | GTCAAGTTTGAACAATGAATATGAAAATAAGAAACGTCGA |
| | | GATTACATAACCGACTACTGGAATGAAAATATTCAAAAAG |
| | | CAAAACGAAAATTTTATGAAGTTAATTCTGTATTAAAACA |
| | | ATTTGAAGTAGCTAATGAACAAAATAGAGACGATAAAAAA |
| | | TTGAACGAAGTAGTGTTACGTAAATTATTTTTATCTTTTA |
| | | CAAATCTTATTAACGATACACTTGAACCTCTTTGTAATGG |
| | | CTCTATTTGTTTTCCTGATATTGAAAAATTAACAAACAGC |
| | | AAAACTGACGAACAATTACAGAGATTCGTTTTTGATGACG |
| | | GATTCAAAAAAATATTATCAGAACAAATAGAAAATTTGAA |
| | | AATTTACTTTGCAATAAACGGCGGATATGTCCAATATGGT |
| | | AGAGTAACATTAAATAAATATACCGCGTTACAACAACCCA |
| | | ATAAGGTAGATGAAGATATAAAGAATATAATTAAAGAACT |
| | | GGGTTTGTTGGAATTTGTAAAAAAGTATGAAAATACTGAA |
| | | CAAATCATTAACTATATAAAAAATATTAAAGATAAAAAAC |
| | | AAGAACTAAACGCTAATAATTTGTCATTGATAGAAAAAGT |
| | | ACAATTATTTAAATACAAAACTATTCCAGCTGGAGTACAA |
| | | CCTTCGCTTATAGCATATCTGGCACGAACAGAAAAAAAAG |
| | | ATAAAAAAACACTCAGAGAACTATTTTACGCAATCGGTCA |
| | | GCCACAAAGTCCGTCAAAAGATTATAAAGAATTACAAAAT |
| | | AAAACGGATTTTAATTTGTACAAATATCCTTTGAAGGTTG |
| | | CATTCGATTATGCGTGGGAGTCATTGGCAAAAAGTAAATA |
| | | TAACCCACATATAGATTTTCCAGATGTCAAATGTAAAGAA |
| | | TTTTTAAAAGATATTTTTGGTACGGATATATCTGTTAATG |
| | | ATAATTTCAAGTTATATTCCGCACTTTTGTTTGTTCGTGA |
| | | AAATCTTGCAACATTAGATCATGGTAATCCAAACGATAAA |
| | | AATATTCATGTTAACAAAGTAGAAAATACATTTAAAGAGA |
| | | TCAAAGATAGATTGGCAAAAAAAGAATATAAAAAAGAATA |
| | | TAAAGCCTTAGAAATTATTTGTAAATGGCATAAAAATTCA |
| | | GCAACCATTGAACAATCAGAATATGAGGCAGCGAAACAAA |
| | | CCATATATGAGGCAGCGAAACAAACCATTGGACAATTAAG |
| | | AGGGCGGCAAAAAAACCAAATATCTAAATTTAAAGAATTG |
| | | ACGGATTCATTCAAAAAGTTGGCTCCAAAATTTGGTAAAG |
| | | CATTTGCTAATCTTAGGGATAAATTTAACGAAGAATATGA |
| | | AATAAATAAAATTTCTCATTGCGGAGTTATTGTAGAAGAT |
| | | CGCAATAACGATCAATATTTGTTATTGTCTCAATTAAATG |
| | | ATAATAGAGAAAATGCATCTGATATTTTTGAGTTAGAAGC |
| | | TGATCCCAACGGTGAGTTGAAAATTTATCAGGTAAAGTCA |
| | | TTGACCTCTAAAACGTTGTTGAAATTTCTCAAAAACAAAA |
| | | AAGGTTCCAATACTGGATTTCATATCAATGAAAATTGGAC |
| | | GTTTCCAAAAGGGAAATGGGATGTTATTAATAAGGATAAA |
| | | ATTTTTCTTAATTATGTAATACAACGTATTACAAATTCGA |
| | | GTATGGCGAAAGAGCAAAAATGGAGTAACTTTAAATGGGA |
| | | TTTTAGGCGATGTGATTCATACGAAGCGATAGCCAAAGAA |
| | | GTAGATGCCAAAGGATATATTTTAGAATCTGTCAATATTT |
| | | CTAAATTGACACTGAACAAATTGATAACAGAAAAGAAATG |
| | | TCTGTTACTACCTATTGTTAATCAAGACTTAACAAGACAA |
| | | GATAAAAAAACAAAAAATCAATTTACGAAAGATTGGATAA |
| | | AGATTTTTGAGAGTAATAATTGTTACCGATTACATCCTGA |
| | | ATTTAAAATATCTTATCGATATCCAACTCCTAATTATCCT |
| | | AAACCGGAAGAAAAGCGTTATTCCCGTTTTCAGATGATTT |
| | | CGCATTTACTTTGCGAATATATTCCACAAAACGATAATTA |
| | | TAAATCACGTAAAGAACAAGTTAAAATCTTTAATGATAAA |
| | | GTTGCTCAAAAAGAATCTGTAGAACAATTTAACCAACAAT |
| | | TTGAAATAACAGATGATTATTATATTTTTGGAATTGATCG |
| | | CGGCATAAAACAATTAGCAACACTTTGTATATTAAATAAA |
| | | AATGGACAAATACAAGGAGACTTTGAAATATATACTCGTG |
| | | AATTTGATAAAGTCAATAAACAATGGAAACACACTATTCT |
| | | TGAAAAACGAAATATTTTAGATTTGTCTGATTTACGGGTT |
| | | GAGACAACAGTTGAGGGTAAAAAAGTATTGGTCGATCTGA |
| | | GTAAAGTGAAGTTACACAGTGGAAATGAAAATAAGCAAAC |
| | | TATAAAACTTAAACGGTTGGCATATATTCGTATGTTACAA |
| | | TATCAAATGCAGCATGAACAAGATAAAGTATTAAGATTTA |
| | | TAAATCAATACAAAACAATTGATGAGATAGAAAAAAATAT |
| | | TAGAGATTTAATTTCACCTTTTAAGGAAGGAAAACAATAT |
| | | GCCGACTTACCTACAGAAAAAATAAAAGATATGCTTATAC |
| | | AATTTGGTGAATTATCAAAGAATGATAGCGATAAATCTAA |
| | | AAAAGAATTGTGTACACTTTGTGAATTAGATGCCGTAGAT |
| | | GATTTTAAAACCGGTGCTGTTGCTAATATGATAGGTGTAA |
| | | TTGCTTATCTACTAGAAAAATATAAATATAACGTTTATAT |
| | | TTCGTTAGAAGATTTGACTCGTGCATTTAGACTACAAAGG |
| | | GATAGATTAACAAATAATATTTTACAAAGTACCAATAAAG |
| | | ACAATACTGTAGATTTCAAAGATCAAGAAAATTTAGTATT |
| | | AGCAGGATTGGGAACTTATCACTATTTTGAAATACAATTA |
| | | CTAAGAAAATTGTTTCGTATCCAGCGAAATAGTGAAGGAG |
| | | ACATTTTACATTTAGTTCCGACATTTCGTAGCGTAGATAA |
| | | TTACGAAAAAATTGTTCGCAGAGATAAAAAAACAGATAAT |
| | | GATAAATATGTGAACTATCCCTTTGGAATTGTGCGGTTTG |
| | | TTGATCCGAAATATACTTCTAAAAAATGTCCTATTTGTGA |
| | | TAAAACGAACACCACAAGGAAAGATAATGTTCTGATTTGT |
| | | AATTCTTGCAATGCAGTATCTGGAGAATATGAAACAGATA |
| | | ATGAAAATAGACATTATATTACCAATGGCGATGATAATGG |
| | | TGCATACCATATAGCTTTAAAAGCATTAAGTTTAAGGAAG |
| | | TCAAAAAATCTTGAAAAGAAAAAGTAA |
| 19 | Nucleic acid sequence | ATGACTAAGTATCAATTAACAAAGACCCTACGATTTGGAC |
| | encoding the Unk 106 | TCACCAAGGTGAGAAAAAAAACTAAACTTGTGGCAGGGAA |
| | polynucleotide | AGAGGTGGATGCAAAATACCTCAGCCATGAGGAGTTGGAT |
| | | GACTTGGTGATGAGGTCGGAGTATAATCTCATCAAAAGGA |
| | | ATGTGCTTGAATGGTCTAAAAAAAAGGAAAGCGACAAATC |
| | | TAATTATATCTTTGATGAACTTGACAAGAGATTTGAAGAA |
| | | ATAATCGAAATCGAAGACAGAGATCAACGCAATGCCGAAT |
| | | ACGTTGAATTTTTGAATGAGTTCTTCAAAGAGGTAAACCA |
| | | TCAGACTATAAACCAATTGGATGAATCGACTTTTATAAAT |
| | | AAAATTGGTGATTGTAGTAAAGCTATAAAGGAGTATTTGT |
| | | TAAGTTGGGGAAAAGTATGCAGGCGCATTGATAAAATCAC |
| | | AGTTAGAAAGGATTACTTCAAGATACTTGCTCGTAAGACT |
| | | TTTTTCAAATACGAATCAAAAGTTGGGAAAAAGAGGACGC |
| | | CTCTGCCTTCCGAAGTCAAACTATCGGGACAAAAAGGCAA |
| | | TAATTATTTTGATGAACCGATAAACGAAGGCATTTCTCAA |
| | | TTTTGGCAAAATAGAGTTGCAAAAGCATTAAACCTACATT |
| | | CGCAACTTGAGTCAATGCTTTTTGATTACAAAAAAGCAAT |
| | | AGAAACAGAAAAACACAATCAGGAAAACCCGAAAGGAGAT |
| | | AATGGTTCATTTGACAAGCTGCACCTTGTAGATTTTAGAA |
| | | AAATGTTTCTATCAGTATGCAGTCTTGTTATGGATAGTTT |
| | | GCGCCCAATTGTTAATGAACTTATCATAGTGTCGGATAAT |
| | | GTTTCGAAAGACGAGGATAAGTATATTTTGGACTTTGTCA |
| | | ATGACAAAAAAACGCAATGGGATTTGTTCCAACAAATAGA |
| | | GAATTTACAGACTATATGCAAAGATAACGGTGAAAATATC |
| | | TTCTTTGGGAAAGCAACGTTCAATAAATACACTTCTGAAC |
| | | AAGCCCCAAATCATCGAAATAATGATATTGCCAAGGTTCT |
| | | CCGAGAACTGAAAATTGAAAAATTTGTGTCTGATTATATT |
| | | GACTTGGATCAAGAAGCAATAAATCGAAAAATTTATCAAT |
| | | CAACCCAATCCCGTTTGGAGAACTTGAATAATCCACAGAT |
| | | TTCTCCTATTATCCGCGCACAGTATTTCAAATACAAACCA |
| | | ATTCCAACATTAGTAAGATTCGGACTTGCGAAAGAATTGG |
| | | CCAAACAGCAAGGAAAAAAATATTCCGATCGATTGAAAGG |
| | | CATCCAAGAATTGTTTAGAATTTTTGGATCTTCAAAAAGT |
| | | CCGGCATTGGATTACAAAAACAATAGAACTGATTTCTCAT |
| | | TGGACAATTATCCAATCAAAGTTGCATTTGATTATGCATG |
| | | GGAAATGTGTGCCCGTTCTGAATACGCTCAAAAACCAGTC |
| | | GATTTTCCAAAATCAATTTGTGAAAAATTCTTGGAAAAAT |
| | | TTTTTGAATGCAAAAGTAACGAAAAATATCAACAATCATT |
| | | TGTGACTTATGCCCGTTTATTGAAAATCAATGAAGACTTG |
| | | GCAACTTTGGAACATTTTGAAAATGAGCCACCCAAAGATA |
| | | TCGAATCAATTTACCAAGATGCGCAAAGATATCTCGATGA |
| | | AGTGGGGAATTTATGCTCAAATGAGGATAGAGCAGCAATC |
| | | GCAAAATGGTACGAAGAATACAACAAGTTATGGACAAAAG |
| | | GCGACCATAAAAAGTTAAAAGAATGGATTGTGTCAAAATC |
| | | ATCTATCATTACTAATTTTACGCAGGCGAAGATGCATTTG |
| | | GGACAAAAAAGAGGAAGCCAAAAAACTTTTGTTCTTAAAT |
| | | CATATTTCCATTCTTCTTATGGAAAAATTAGAGATAACAA |
| | | TCGATTTGTAAACTCTAATGTTACCGAAGTGTTTAAAACA |
| | | ATAGCCAGCACTTTCGGCAAATCGTTTGCCACAATCAGAG |
| | | AGTATTTTAACGAAGAAAGTGAAGTAAACAAAATAGAATA |
| | | CGGCGCTGTGATTATCAAAGATAAAAACGGTGACAAATAT |
| | | CTTTTGCTTCAAAAGAAAAATGAAGGTGGTATTGATATGC |
| | | CTATATTCAATAAATCTGATGAAAATGGTGATTGTGACCT |
| | | TTATCAGGTTAAATCTCTGACATCCAAAACAGTTAGGAAA |
| | | ATTATTGCATCACCCAACAAATATAACGATTTTTTTGTTA |
| | | ACAATGATGGCAAAAAAATCATTTATCCAGACAAAACAGA |
| | | TTTCAAATACAAGATTAATCCATATGACAAAGAAGAAGTC |
| | | AAAAAACGCAAAAGAGAATTGTACAATAATGATTTAGTGC |
| | | GACCAATTATATATAGTTTGACTCAATCTAAATTTGCCAA |
| | | CAAACAAAATTTTGAGAAATATTTTGATTGGACTAAGGCT |
| | | TTAAAACAATGCTCAAACATTGAGCAATTGTACAAAACAA |
| | | TCGACCAGAAAGGCTATTCCTTGAACCCTTCCAAAATCAG |
| | | CAAAGAGCAAATCGCGGATTTGGTTAACAATATGAATTGT |
| | | TATCTCTTGCCGATTGTAAATCAAAATATTACAGCAAAGA |
| | | CAAAGAACGACACAAATCAATTCACCAAAGATTGGAATAA |
| | | GATTTTCAATGAGGTAGATAAGGATTATCGTATTCATCCT |
| | | GAATTTACGATGTTCTATCGTTATCCTACGCCGGATTATC |
| | | CTAAATTTGGAGAAAAGAGGTATTCCCGTTTCCAAATGAA |
| | | TGTAAACTTTCTAATGGAAGTTATTCCGGCTGATGGCGAA |
| | | TACTGTTCACGAAAGGAGCAAATAGAAATATACAATGCTC |
| | | CCAAAGACAACGAAAATTGTCAAAAAAATGTAGTTGAGAG |
| | | ATTCAACAATAAAATTAAAGCCCTAAAGCCATCGTATTTC |
| | | ATAGGCATTGACCGTGGCATTAACGAATTGGCGACATTGT |
| | | GCGTAATAGATAAAGAAGGTAAAATCGTTGGCGATTTTGA |
| | | GATATACAAAAGAGAATTTGACTCAAATCTGAAACGTCAG |
| | | AAATATACATCAATAGAGACTCGCGACATTTTAGACCTGT |
| | | CGTACTTGCGTGTTGAAAAGGACGAAAATGGTGAATCTCG |
| | | TTTAGTAGATTTGTCAGAATCGGAGGTATGGATAGACAGC |
| | | CTTGATCACGACAAAGGAAAAAGAGCCAACAAACAAATAG |
| | | TTCACCTCAAACATCTGTACTATTTGCGTTGCATCGCACA |
| | | TTTGTTGCAGTCGTCAGATTACAAATCTATTGTATTAGAA |
| | | AAACTCAAAGATTGCAACAATCTGAAAGATGAGGAAATAA |
| | | AAAAGGTATTCGAGAAAGATAAATTTGTTGATTCCTACAA |
| | | AGGTGGCGAAGCATATACAGATTTGCCATACGATGAAATC |
| | | AGAAAGTTAATTTCTGATTATCAGGAAATTGAGCAATCAA |
| | | ACCAAACAGAATCGGAGAAGTCCAAAGCATTAAATACACT |
| | | TTGCCAGTTGGACGCATCTGAATATCTCAAAAAGGGTGTT |
| | | GTTGCCAATATGATAGGTGTTGTCGTATATGTTTTGAAAA |
| | | AGTATAATTACGATGCGTATATCTCTCTCGAAAATCTTTG |
| | | CTATGCATACGGATATAGCAAAGACACATTGTCAGGATAT |
| | | TCAATTACAAGTACTAAGGAAGATCCATATTTAGATTTCA |
| | | AAGATCAAGAAAACGCGAAATTAGCAGGATTAGGAACTTA |
| | | TAGTTTCTTTGAGGTTCAACTTCTGAAAAAACTTTTCAAA |
| | | CTTCAAATTGAAGAAAATACAGAGTTGATTCCAGCTTTCC |
| | | GCAGCGTGGACAACTACGAGAAAATATTTTTGTTAAAAAA |
| | | TATAGATAACAAAATTTATCAGTTCGGTATTGTGTATTTT |
| | | GTTGATCCAAAATATACAAGCCTATGTTGCCCTATTTGTG |
| | | GTGAACATGGCAAAAAAAATGTTGATAGGAAGAAACATAC |
| | | AAAAAAATATGACGAAGATGAATTAGTATGCAAGCAATGC |
| | | GGTTTTCATACAAATCTTTCCCATATCGAAACAAGGGTTA |
| | | TGGAAGACAAAACAATAAAAAATAGTTACGATGAGTGTAA |
| | | TTTGAAAGCCATTGTTTCTGGAGATGCAAATGCCGCTTAT |
| | | AACATTGCAATTAGGTTGGGCAAAAACATATACTCAACAA |
| | | TTGCAGACAAAGTAAAGGATTTGCATCACGAAGGGAAAAA |
| | | ATACATTATCGTAAAAGGATAA |
| 20 | Nucleic acid sequence | GTGGCAGGGAAAGAGGTGGATGCAAAATACCTCAGCCATG |
| | encoding the Unk107 | AGGAGTTGGATGACTTGGTGATGAGGTCGGAGTATAATCT |
| | polynucleotide | CATCAAAAGGAATGTGCTTGAATGGTCTAAAAAAAAGGAA |
| | | AGCGACAAATCTAATTATATCTTTGATGAACTTGACAAGA |
| | | GATTTGAAGAAATAATCGAAATCGAAGACAGAGATCAACG |
| | | CAATGCCGAATACGTTGAATTTTTGAATGAGTTCTTCAAA |
| | | GAGGTAAACCATCAGACTATAAACCAATTGGATGAATCGA |
| | | CTTTTATAAATAAAATTGGTGATTGTAGTAAAGCTATAAA |
| | | GGAGTATTTGTTAAGTTGGGGAAAAGTATGCAGGCGCATT |
| | | GATAAAATCACAGTTAGAAAGGATTACTTCAAGATACTTG |
| | | CTCGTAAGACTTTTTTCAAATACGAATCAAAAGTTGGGAA |
| | | AAAGAGGACGCCTCTGCCTTCCGAAGTCAAACTATCGGGA |
| | | CAAAAAGGCAATAATTATTTTGATGAACCGATAAACGAAG |
| | | GCATTTCTCAATTTTGGCAAAATAGAGTTGCAAAAGCATT |
| | | AAACCTACATTCGCAACTTGAGTCAATGCTTTTTGATTAC |
| | | AAAAAAGCAATAGAAACAGAAAAACACAATCAGGAAAACC |
| | | CGAAAGGAGATAATGGTTCATTTGACAAGCTGCACCTTGT |
| | | AGATTTTAGAAAAATGTTTCTATCAGTATGCAGTCTTGTT |
| | | ATGGATAGTTTGCGCCCAATTGTTAATGAACTTATCATAG |
| | | TGTCGGATAATGTTTCGAAAGACGAGGATAAGTATATTTT |
| | | GGACTTTGTCAATGACAAAAAAACGCAATGGGATTTGTTC |
| | | CAACAAATAGAGAATTTACAGACTATATGCAAAGATAACG |
| | | GTGAAAATATCTTCTTTGGGAAAGCAACGTTCAATAAATA |
| | | CACTTCTGAACAAGCCCCAAATCATCGAAATAATGATATT |
| | | GCCAAGGTTCTCCGAGAACTGAAAATTGAAAAATTTGTGT |
| | | CTGATTATATTGACTTGGATCAAGAAGCAATAAATCGAAA |
| | | AATTTATCAATCAACCCAATCCCGTTTGGAGAACTTGAAT |
| | | AATCCACAGATTTCTCCTATTATCCGCGCACAGTATTTCA |
| | | AATACAAACCAATTCCAACATTAGTAAGATTCGGACTTGC |
| | | GAAAGAATTGGCCAAACAGCAAGGAAAAAAATATTCCGAT |
| | | CGATTGAAAGGCATCCAAGAATTGTTTAGAATTTTTGGAT |
| | | CTTCAAAAAGTCCGGCATTGGATTACAAAAACAATAGAAC |
| | | TGATTTCTCATTGGACAATTATCCAATCAAAGTTGCATTT |
| | | GATTATGCATGGGAAATGTGTGCCCGTTCTGAATACGCTC |
| | | AAAAACCAGTCGATTTTCCAAAATCAATTTGTGAAAAATT |
| | | CTTGGAAAAATTTTTTGAATGCAAAAGTAACGAAAAATAT |
| | | CAACAATCATTTGTGACTTATGCCCGTTTATTGAAAATCA |
| | | ATGAAGACTTGGCAACTTTGGAACATTTTGAAAATGAGCC |
| | | ACCCAAAGATATCGAATCAATTTACCAAGATGCGCAAAGA |
| | | TATCTCGATGAAGTGGGGAATTTATGCTCAAATGAGGATA |
| | | GAGCAGCAATCGCAAAATGGTACGAAGAATACAACAAGTT |
| | | ATGGACAAAAGGCGACCATAAAAAGTTAAAAGAATGGATT |
| | | GTGTCAAAATCATCTATCATTACTAATTTTACGCAGGCGA |
| | | AGATGCATTTGGGACAAAAAAGAGGAAGCCAAAAAACTTT |
| | | TGTTCTTAAATCATATTTCCATTCTTCTTATGGAAAAATT |
| | | AGAGATAACAATCGATTTGTAAACTCTAATGTTACCGAAG |
| | | TGTTTAAAACAATAGCCAGCACTTTCGGCAAATCGTTTGC |
| | | CACAATCAGAGAGTATTTTAACGAAGAAAGTGAAGTAAAC |
| | | AAAATAGAATACGGCGCTGTGATTATCAAAGATAAAAACG |
| | | GTGACAAATATCTTTTGCTTCAAAAGAAAAATGAAGGTGG |
| | | TATTGATATGCCTATATTCAATAAATCTGATGAAAATGGT |
| | | GATTGTGACCTTTATCAGGTTAAATCTCTGACATCCAAAA |
| | | CAGTTAGGAAAATTATTGCATCACCCAACAAATATAACGA |
| | | TTTTTTTGTTAACAATGATGGCAAAAAAATCATTTATCCA |
| | | GACAAAACAGATTTCAAATACAAGATTAATCCATATGACA |
| | | AAGAAGAAGTCAAAAAACGCAAAAGAGAATTGTACAATAA |
| | | TGATTTAGTGCGACCAATTATATATAGTTTGACTCAATCT |
| | | AAATTTGCCAACAAACAAAATTTTGAGAAATATTTTGATT |
| | | GGACTAAGGCTTTAAAACAATGCTCAAACATTGAGCAATT |
| | | GTACAAAACAATCGACCAGAAAGGCTATTCCTTGAACCCT |
| | | TCCAAAATCAGCAAAGAGCAAATCGCGGATTTGGTTAACA |
| | | ATATGAATTGTTATCTCTTGCCGATTGTAAATCAAAATAT |
| | | TACAGCAAAGACAAAGAACGACACAAATCAATTCACCAAA |
| | | GATTGGAATAAGATTTTCAATGAGGTAGATAAGGATTATC |
| | | GTATTCATCCTGAATTTACGATGTTCTATCGTTATCCTAC |
| | | GCCGGATTATCCTAAATTTGGAGAAAAGAGGTATTCCCGT |
| | | TTCCAAATGAATGTAAACTTTCTAATGGAAGTTATTCCGG |
| | | CTGATGGCGAATACTGTTCACGAAAGGAGCAAATAGAAAT |
| | | ATACAATGCTCCCAAAGACAACGAAAATTGTCAAAAAAAT |
| | | GTAGTTGAGAGATTCAACAATAAAATTAAAGCCCTAAAGC |
| | | CATCGTATTTCATAGGCATTGACCGTGGCATTAACGAATT |
| | | GGCGACATTGTGCGTAATAGATAAAGAAGGTAAAATCGTT |
| | | GGCGATTTTGAGATATACAAAAGAGAATTTGACTCAAATC |
| | | TGAAACGTCAGAAATATACATCAATAGAGACTCGCGACAT |
| | | TTTAGACCTGTCGTACTTGCGTGTTGAAAAGGACGAAAAT |
| | | GGTGAATCTCGTTTAGTAGATTTGTCAGAATCGGAGGTAT |
| | | GGATAGACAGCCTTGATCACGACAAAGGAAAAAGAGCCAA |
| | | CAAACAAATAGTTCACCTCAAACATCTGTACTATTTGCGT |
| | | TGCATCGCACATTTGTTGCAGTCGTCAGATTACAAATCTA |
| | | TTGTATTAGAAAAACTCAAAGATTGCAACAATCTGAAAGA |
| | | TGAGGAAATAAAAAAGGTATTCGAGAAAGATAAATTTGTT |
| | | GATTCCTACAAAGGTGGCGAAGCATATACAGATTTGCCAT |
| | | ACGATGAAATCAGAAAGTTAATTTCTGATTATCAGGAAAT |
| | | TGAGCAATCAAACCAAACAGAATCGGAGAAGTCCAAAGCA |
| | | TTAAATACACTTTGCCAGTTGGACGCATCTGAATATCTCA |
| | | AAAAGGGTGTTGTTGCCAATATGATAGGTGTTGTCGTATA |
| | | TGTTTTGAAAAAGTATAATTACGATGCGTATATCTCTCTC |
| | | GAAAATCTTTGCTATGCATACGGATATAGCAAAGACACAT |
| | | TGTCAGGATATTCAATTACAAGTACTAAGGAAGATCCATA |
| | | TTTAGATTTCAAAGATCAAGAAAACGCGAAATTAGCAGGA |
| | | TTAGGAACTTATAGTTTCTTTGAGGTTCAACTTCTGAAAA |
| | | AACTTTTCAAACTTCAAATTGAAGAAAATACAGAGTTGAT |
| | | TCCAGCTTTCCGCAGCGTGGACAACTACGAGAAAATATTT |
| | | TTGTTAAAAAATATAGATAACAAAATTTATCAGTTCGGTA |
| | | TTGTGTATTTTGTTGATCCAAAATATACAAGCCTATGTTG |
| | | CCCTATTTGTGGTGAACATGGCAAAAAAAATGTTGATAGG |
| | | AAGAAACATACAAAAAAATATGACGAAGATGAATTAGTAT |
| | | GCAAGCAATGCGGTTTTCATACAAATCTTTCCCATATCGA |
| | | AACAAGGGTTATGGAAGACAAAACAATAAAAAATAGTTAC |
| | | GATGAGTGTAATTTGAAAGCCATTGTTTCTGGAGATGCAA |
| | | ATGCCGCTTATAACATTGCAATTAGGTTGGGCAAAAACAT |
| | | ATACTCAACAATTGCAGACAAAGTAAAGGATTTGCATCAC |
| | | GAAGGGAAAAAATACATTATCGTAAAAGGATAA |
| 21 | Nucleic acid sequence | ATGGAACAATATCAATTAACAAAGACAATACGATTCGGAC |
| | encoding the Unk108 | TCACCAAAGTAAGAAAAGAGAAGAAACACCTCAGCCACGA |
| | polynucleotide | GGAGTTGGATGAATTGGTGATGGTGTCAGAGGAAAGAATC |
| | | AAAAAAGAACATCCTCAGGCTGAAAACCAATTAGACGAAC |
| | | AGTCCTTCGTAAAAAAAATTGGTGATTGTAGCAAAGCCAT |
| | | AAAAGAATATTTGTTAAGTTGGGGAAAAGTATGCCGACGC |
| | | ATTGATAAAATTACAGTAAGAAAAGAGTTTTTCAAGATTC |
| | | TTGCTCGCAAGACCTTTTTCAAATATGAATCAAAAGTTGG |
| | | TAAAAAAAGGACACCTCTACCTTCCGAAGTTAAATTATCG |
| | | GGACAAAAAGGGAATAATTATTATGATGAACCGATTAACG |
| | | AAGGCATTTCTCAATTTTGGCAAAACAGAGTTTCAAAGGC |
| | | ATTGAAACTACATTCACAACTTGAATCAATGCTTTTTGAT |
| | | TACAAAAAAGCAATAGAAACAGAAAAACACAACCAAGAAA |
| | | ACCCCAAAGAGAACAATGATCAATTCGACAAACTGCATCT |
| | | TGTAGATTTTAGAAAAATGTTTCTATCTGTATGCAGCCTT |
| | | GTTATGGATAGTTTGCGCCCAATTGTAAATGAACTTATCA |
| | | TTGTGTCGGATAATGTATCGAAAGACGAGGATAAGTATAT |
| | | TTTGGACTTTGTTAATGACAAATCGAAACAATGGGATTTG |
| | | TTTAAACAAATAGAGGATTTACAGAATCTTTGCAAAGATA |
| | | ACGGCGGGAATATCCCCTTTGGAAAAGCAACGTTCAATAA |
| | | ATACACCTCGGAACAAGCCCCAAATCATCGAGATAATGAT |
| | | ATTCACAAAGTTATCCGAGAACTAAAAATTGAAGAATTTG |
| | | TTTCTGATTTTATAGGCTTAGAACAAGAGGATATCTATCG |
| | | AAAAATTTATCAATCGACTCAACACAGTTTGGTGAACTTG |
| | | AACAAACCAAGTATTTCTCCTATCATTCGCGCACAGTTTT |
| | | TTAAGTACAAACCAATTCCAGTATTAGTCAGATTCGGACT |
| | | CGCGAATTATTTAAACAAACAGCAAGGGAAAAAATACTCA |
| | | AATCGATTGAAAGACATCCAAGAATTATTTAGGATTTTTG |
| | | GAACATCAAAAAGTCCCGAGTTAGATTATTCTGACAAAAA |
| | | CAATAGAACTGAATTTTCTTTGGACAAGTATCCAATCAAA |
| | | GTTGCATTTGACTACGCTTGGGAGAGGTGTGCTCGTTCAA |
| | | AATATGCTCAAAAGCCTGTCGATTTTCCCAAAGAAATATG |
| | | TGTAACCTTTTTGGAAACATATTTCGAATACAATAGTAAA |
| | | GAAAAAAATCGTGAAGCATTTGAAATATATGCACATTTAT |
| | | TGAAAGTCAACGAATGTTTAGCGACTTTGGAGCACTTTGA |
| | | GAATGAGCCGCCCAAAGATATTAAATCACTTTGGCAAGAT |
| | | GTGCAAAACCATCTTGACAAAGTGGGGAAATTATGCTCAA |
| | | ATGAGGATAGAAAAGCAATAACCCAATGGTATGAAGAATA |
| | | CAAAAATCTTTGGGCGAAAGGCAACTATAAAAAGTTGAAA |
| | | AAATGGATTGAGTCAAAATCATATACCGTTACTAATTTCA |
| | | CTCAGGCGAAAATGCATTTGGGACAAAAAAGAGGAAGCCA |
| | | AAAAACAATGGTTCTGAAATCATATTTTCACCCTACTTAT |
| | | GGAAAAATAAAAGATGGCAACCGATTTATAAACTCTAATG |
| | | TAACCGAAGTGTTCAAAAACATAGCGAGTACTTTTGGTAA |
| | | ATCTTTTGCCACCATCAGAGATTATTTTAACGAGGAAAGT |
| | | GAAGTAAATAAAATAGAATATGGTGCTGTAATTATCAAAG |
| | | ATAAAAAAGGCGACAAGTATCTTTTGCTTCAAAAGAAAAA |
| | | TGAAGGCGGTATAGATATGCCTGTATTCAACGAATCTGAT |
| | | GGAAATGGTGATTATGATGTTTATCAAGTTCAATCTTTGA |
| | | CTTATAAAACAGTAAACAAAATTTACAATTCAACAAAATA |
| | | TCCTGAATTCTTTGCAATAAATGGCGAAAAAGCAATTTAC |
| | | GCGCCTAATAGGCCACAACGATTCAAAGATGATCAAGAGA |
| | | AGAATACTTTTAATGAAAAGAAATTGCAGTCGTTGAAAAA |
| | | GTGTTTGACAGAATCTGATTTTATGACAAACACCACCGAA |
| | | AATTATTTGCAAAAGTTCAATTGGACAGAAGAAATCAACA |
| | | ACTGCACAGATTTCGAACCACTTGCCAAAATAGTTGATCA |
| | | AAAGGGATATTACCTAAAATCCTACAAAATCAGCAAAGAG |
| | | CAAATAGCAGAATTGGTTAACAATCAGAATTGTTATCTCT |
| | | TGCCGATTGTAAACCAAAATATTACAGCAAAGACAAAGAA |
| | | CGACACAAATCAATTCACAAAAGATTGGAACAAGATTTTC |
| | | AATGATGAATATAAAGACTATCGTCTTCATCCTGAATTTA |
| | | CTATGTTCTATCGTTATCCTACGCCCGATTATCCTTGTCC |
| | | TGGCGAAAAAAGATATTCTCGGTTTCAAATGAATGTTAAC |
| | | TTCCTAATGGAAGTAATTCCTTCCGAAGGAGAATACGTTT |
| | | CACGAAAGGAACAGATAGAGTCTTTCAATACACCCAAACA |
| | | AGACAAAGAAGATAACGATAATGAAAATAGTCAAGCAAAA |
| | | AAAGTAGCACAATTTAACGATAACATTAATACAAAAAAGC |
| | | CTTCATACATTATTGGCATCGACCGAGGTATTAATGAACT |
| | | TGCCACTTTGTGTGTTATTAACTCTGAGGGCAAAATCGTC |
| | | GCAGTTGACGAAAATGGATTGATAAAAGACGAGTTTGATA |
| | | TTTATGTAAAGCATTTCGATAAAGACAACAAATGTTGGAT |
| | | TCATAACATCAAACCAAAAACAGCAACCGATAATAAACCA |
| | | AGAACAATACTTGACTTGTCGAATTTGAGGGTTGAGACAA |
| | | CTATTGACGGCAAACAAGTATTGGTTGACTTGTCATCCGA |
| | | CGAAAATGGTATACAAGTCAACAGCAAACAAATAGTTCAC |
| | | CTCAAACGTCTTTATTATTTACGTTGCCTATCCTATTTAT |
| | | TGCAATCATCTGATTACAAGTCAATAATTTTGGAAAAACT |
| | | GAAAGACGTTAATAATATGACCGATGACGCAATATATGAA |
| | | GTTTTCAAAAACGACAAATTTATTGATTCATACAAAGGAG |
| | | GTGTACAATATACAGATTTGCCATACGATGAAATTAGACA |
| | | TTTGATTTCTACTTATCAAGAAATTGACCAATCTAATAAA |
| | | ACAGATTCGGAGAAGCAAAGCGAATTAAACACACTTTGTC |
| | | AGTTAGATGCAACAGAATCTCTCAAAAAAGGTGTTGTCGC |
| | | CAATATGATAGGTGTTGTTGTATATATTCTAAAAAAACTA |
| | | AATTACGATGCATATATTTCTCTCGAAAATCTATGTCGGG |
| | | CTCTCTATTTTAGCAAAGATTCACTGTCAGGTTACACTAT |
| | | TGAAAATACCAGCGTAAATCCAGATTTAGATTTCAAGGAT |
| | | CAAGAGAATGCAAAATTGGCAGGCTTAGGAACTTATAGTT |
| | | ACTTTGAAATTCAACTCTTAAAGAAATTGTTTAAACTTCA |
| | | AATAGATGAAAAGCAATTTTTGGTTCCGGGATTTCGTAGC |
| | | GTTGAAAACTACGAAAAAATAGTAAAGTTAGGAAAGGTTA |
| | | AACATTCTATTTATCAATTTGGAGTAGTGCATTTTGTAGA |
| | | ACCCGCCAATACAAGTCTTAAATGCCCAATTTGTGGTGCT |
| | | AATGGTAAAAGGATAAAGTATAATCCTAATTATGACGAAG |
| | | ATGAATTAGTTTGTAAAAAATGTGGTTTCCGTTCCAATAT |
| | | TTCTAAAATTCAAAACAGCAAAATTATGGAAGACTCTGTA |
| | | ATTAAAACCTATTATGATAACCATAATTATAAGGCAATAA |
| | | TTTCAGGAGATACCAATGCCGGATTTAACATTGCTCTTAG |
| | | ATTGCTCATGAACCTCAACACACAAATTGAGAATGCAATT |
| | | AACCATTTGCATAAAACAGGAAAAAACTATCACAGTGTAA |
| | | ATAAGTAA |
| 22 | Nucleic acid sequence | ATGAAAAATTTAACACAATTTGTAAATTTGTACCAACTCT |
| | encoding the Unk109 | CAAAAACATTGAAATTCGGATTAACATTACGAAATAAAAT |
| | polynucleotide | AAGAAAAAATGGTTTTGAAGGAGAAATTTATGAAAGTCAT |
| | | ACTGAATTACAGGAACTTATAAAAATTTCAGAGCAAAAAA |
| | | TCATTAAGGAAACAACTGATAAAAATAAAGAAATAATGAC |
| | | ATTTACAGAATTGCCCTTAGATGAAATTCGTAAATGTCTT |
| | | GACGACATGCATAAATATCTCGATGATTGGGAACAATTTT |
| | | ATAACAGATATGACCAAATAGCAGTACTTAAAGATTATTA |
| | | TCGAAAATTGGAACGTAAAGCAAGATTTGACGGTTTTTGG |
| | | AGAGAAAAAAATATTAAGAACAAAATACAAAATAATGAAT |
| | | CTGAAACTATCAAAAAACCGCAGTCTCAAGTTATCAAATT |
| | | GTCAAGTTTGAACAATGAATATGAAAATAAGAAACGTCGA |
| | | GATTACATAACCGACTACTGGAATGAAAATATTCAAAAAG |
| | | CAAAACGAAAATTTTATGAAGTTAATTCTGTATTAAAACA |
| | | ATTTGAAGTAGCTAATGAACAAAATAGAGACGATAAAAAA |
| | | TTGAACGAAGTAGTGTTACGTAAATTATTTTTATCTTTTA |
| | | CAAATCTTATTAACGATACACTTGAACCTCTTTGTAATGG |
| | | CTCTATTTGTTTTCCTGATATTGAAAAATTAACAAACAGC |
| | | AAAACTGACGAACAATTACAGAGATTCGTTTTTGATGACG |
| | | GATTCAAAAAAATATTATCAGAACAAATAGAAAATTTGAA |
| | | AATTTACTTTGCAATAAACGGCGGATATGTCCAATATGGT |
| | | AGAGTAACATTAAATAAATATACCGCGTTACAACAACCCA |
| | | ATAAGGTAGATGAAGATATAAAGAATATAATTAAAGAACT |
| | | GGGTTTGTTGGAATTTGTAAAAAAGTATGAAAATACTGAA |
| | | CAAATCATTAACTATATAAAAAATATTAAAGATAAAAAAC |
| | | AAGAACTAAACGCTAATAATTTGTCATTGATAGAAAAAGT |
| | | ACAATTATTTAAATACAAAACTATTCCAGCTGGAGTACAA |
| | | CCTTCGCTTATAGCATATCTGGCACGAACAGAAAAAAAAG |
| | | ATAAAAAAACACTCAGAGAACTATTTTACGCAATCGGTCA |
| | | GCCACAAAGTCCGTCAAAAGATTATAAAGAATTACAAAAT |
| | | AAAACGGATTTTAATTTGTACAAATATCCTTTGAAGGTTG |
| | | CATTCGATTATGCGTGGGAGTCATTGGCAAAAAGTAAATA |
| | | TAACCCACATATAGATTTTCCAGATGTCAAATGTAAAGAA |
| | | TTTTTAAAAGATATTTTTGGTACGGATATATCTGTTAATG |
| | | ATAATTTCAAGTTATATTCCGCACTTTTGTTTGTTCGTGA |
| | | AAATCTTGCAACATTAGATCATGGTAATCCAAACGATAAA |
| | | AATATTCATGTTAACAAAGTAGAAAATACATTTAAAGAGA |
| | | TCAAAGATAGATTGGCAAAAAAAGAATATAAAAAAGAATA |
| | | TAAAGCCTTAGAAATTATTTGTAAATGGCATAAAAATTCA |
| | | GCAACCATTGAACAATCAGAATATGAGGCAGCGAAACAAA |
| | | CCATATATGAGGCAGCGAAACAAACCATTGGACAATTAAG |
| | | AGGGCGGCAAAAAAACCAAATATCTAAATTTAAAGAATTG |
| | | ACGGATTCATTCAAAAAGTTGGCTCCAAAATTTGGTAAAG |
| | | CATTTGCTAATCTTAGGGATAAATTTAACGAAGAATATGA |
| | | AATAAATAAAATTTCTCATTGCGGAGTTATTGTAGAAGAT |
| | | CGCAATAACGATCAATATTTGTTATTGTCTCAATTAAATG |
| | | ATAATAGAGAAAATGCATCTGATATTTTTGAGTTAGAAGC |
| | | TGATCCCAACGGTGAGTTGAAAATTTATCAGGTAAAGTCA |
| | | TTGACCTCTAAAACGTTGTTGAAATTTCTCAAAAACAAAA |
| | | AAGGTTCCAATACTGGATTTCATATCAATGAAAATTGGAC |
| | | GTTTCCAAAAGGGAAATGGGATGTTATTAATAAGGATAAA |
| | | ATTTTTCTTAATTATGTAATACAACGTATTACAAATTCGA |
| | | GTATGGCGAAAGAGCAAAAATGGAGTAACTTTAAATGGGA |
| | | TTTTAGGCGATGTGATTCATACGAAGCGATAGCCAAAGAA |
| | | GTAGATGCCAAAGGATATATTTTAGAATCTGTCAATATTT |
| | | CTAAATTGACACTGAACAAATTGATAACAGAAAAGAAATG |
| | | TCTGTTACTACCTATTGTTAATCAAGACTTAACAAGACAA |
| | | GATAAAAAAACAAAAAATCAATTTACGAAAGATTGGATAA |
| | | AGATTTTTGAGAGTAATAATTGTTACCGATTACATCCTGA |
| | | ATTTAAAATATCTTATCGATATCCAACTCCTAATTATCCT |
| | | AAACCGGAAGAAAAGCGTTATTCCCGTTTTCAGATGATTT |
| | | CGCATTTACTTTGCGAATATATTCCACAAAACGATAATTA |
| | | TAAATCACGTAAAGAACAAGTTAAAATCTTTAATGATAAA |
| | | GTTGCTCAAAAAGAATCTGTAGAACAATTTAACCAACAAT |
| | | TTGAAATAACAGATGATTATTATATTTTTGGAATTGATCG |
| | | CGGCATAAAACAATTAGCAACACTTTGTATATTAAATAAA |
| | | AATGGACAAATACAAGGAGACTTTGAAATATATACTCGTG |
| | | AATTTGATAAAGTCAATAAACAATGGAAACACACTATTCT |
| | | TGAAAAACGAAATATTTTAGATTTGTCTGATTTACGGGTT |
| | | GAGACAACAGTTGAGGGTAAAAAAGTATTGGTCGATCTGA |
| | | GTAAAGTGAAGTTACACAGTGGAAATGAAAATAAGCAAAC |
| | | TATAAAACTTAAACGGTTGGCATATATTCGTATGTTACAA |
| | | TATCAAATGCAGCATGAACAAGATAAAGTATTAAGATTTA |
| | | TAAATCAATACAAAACAATTGATGAGATAGAAAAAAATAT |
| | | TAGAGATTTAATTTCACCTTTTAAGGAAGGAAAACAATAT |
| | | GCCGACTTACCTACAGAAAAAATAAAAGATATGCTTATAC |
| | | AATTTGGTGAATTATCAAAGAATGATAGCGATAAATCTAA |
| | | AAAAGAATTGTGTACACTTTGTGAATTAGATGCCGTAGAT |
| | | GATTTTAAAACCGGTGCTGTTGCTAATATGATAGGTGTAA |
| | | TTGCTTATCTACTAGAAAAATATAAATATAACGTTTATAT |
| | | TTCGTTAGAAGATTTGACTCGTGCATTTAGACTACAAAGG |
| | | GATAGATTAACAAATAATATTTTACAAAGTACCAATAAAG |
| | | ACAATACTGTAGATTTCAAAGATCAAGAAAATTTAGTATT |
| | | AGCAGGATTGGGAACTTATCACTATTTTGAAATACAATTA |
| | | CTAAGAAAATTGTTTCGTATCCAGCGAAATAGTGAAGGAG |
| | | ACATTTTACATTTAGTTCCGACATTTCGTAGCGTAGATAA |
| | | TTACGAAAAAATTGTTCGCAGAGATAAAAAAACAGATAAT |
| | | GATAAATATGTGAACTATCCCTTTGGAATTGTGCGGTTTG |
| | | TTGATCCGAAATATACTTCTAAAAAATGTCCTATTTGTGA |
| | | TAAAACGAACACCACAAGGAAAGATAATGTTCTGATTTGT |
| | | AATTCTTGCAATGCAGTATCTGGAGAATATGAAACAGATA |
| | | ATGAAAATAGACATTATATTACCAATGGCGATGATAATGG |
| | | TGCATACCATATAGCTTTAAAAGCATTAAGTTTAAGGAAG |
| | | TCAAAAAATCTTGAAAAGAAAAAGTAA |
| 23 | Nucleic acid sequence | ATGGAAAAATACCAGATTACTAAGACAGTGAGATTTGGGT |
| | encoding the Unk110 | TGACAGCTACAAATTCAAATTTGTATTCCGATGAATTAAA |
| | polynucleotide | AGATTTGATTGAAACTTCGGAAATTAAAATCAAAGAATCA |
| | | TTAAAAAATAAAAGTCACAATTCTCTACAAATCGAACAGT |
| | | TAAGGAGTTGTTTGAACGGAGTAAAGGAATATCTGAAAAC |
| | | TTGGAATAATGTTTATAGCCAAATTGATTTTTTGGGAATA |
| | | TCTAAAGACTATTATAAAGTAATTTCAAGAAAAGCAAGAT |
| | | TTGATTTTGATAACAAAGGACTTGGTTCCGAAGTCAAACT |
| | | TGCTTCTCTGCAATCAAAGTATAATAGCAAAAAAAGGATT |
| | | CAATATATTTTGGATTTCTGGGAAGACAATTTCCAGAAAA |
| | | CAGAAATTCTATATCGCAAGTCAGATGAATTATTGAAAGT |
| | | TTTTGAAGAAGCAGAAAAGCAAAAACGTGATGATAAAAAA |
| | | CTGAATGAGGTAGAATTACGTAAAACTTTTTTAAGTTTAT |
| | | TTAATTTGGTGAATGAAAGTTTAAAACCTTTAGTAGAGGG |
| | | AAATTTATTTACTATAAACGATGATAAGATAGATAGCAGA |
| | | AACCAGAATCATGAAGTAATCGCCGATTTTATTTCAAACA |
| | | CTAAAGTCAGAACTGAATTATATGAGAGTATAACCGAATT |
| | | ACAAAATTTTTTCAGAGATAACGGTGGTTATGTTCCCTTT |
| | | GGTAGAGCGACCTTCAATCAATGGACAGCTTTGCAAAAAG |
| | | CAGATAAAAATGGAGAAAGGGAAATAGATAAAATTATAAA |
| | | ACAACTGAATCTAGAAACCGTTTCAATGGCGAACATTGAT |
| | | TATAAATATAATACTTTCACAAAAAATTTTGAACAAGGAG |
| | | GACAAGTTTGGAAAATCAAACAAAATGCAAAATCTGTTAT |
| | | TGAACTCTGCCAGTTTTTCAAATATAAAAAGGTATCGATT |
| | | ACAACTCGTTTAAATCTTGCCAAGCGACTGAATAAGACTA |
| | | ATAACTTTTTAAGTGAATTTGGAATTTCAAAATCACCTGC |
| | | TCTTGACTACAAAAAAGATAAAGAAAACTTCAATCTAGCA |
| | | AATTATCCACTGAAAGTAGCATTTGATTACGCTTGGGAAA |
| | | ATTGTGCAAAAGCAAAACACGAAAGCATTACATTTCCAGA |
| | | GCTGCAATGTAAGGATTATTTGCATAATGTGTTTGGTGTG |
| | | GATGCGAATAAGGATAAAAATGGAAAAATAAAAAATGAAG |
| | | AGTTAAACAAATATGCAGATTTATTGCAATTCAAAATATT |
| | | GCTTGGAAGACTAAAAGCAGAATTTCACAAAGCAGCTGAA |
| | | GAAACCAACAAAAATAATATTCGAAAGCTAAAAAATATTT |
| | | TTGAAAATTTAGATTACAGTGGTGTGCAAGATTTTAATAA |
| | | AAATAAAATCAAAGAGATTGTTGAAGTCTGGTTTGCCAAT |
| | | AAAGAAAAGAATATTGGAAAAAAGAAAGAGGAAATGATTC |
| | | CTTTAACAGAAAAAAAAAAAGATGATTTTTCCAAAGCCAT |
| | | GCAAATTATCGGACAAGAGCGTGGGGGGCTGAAAAGCAGA |
| | | ATCAAAAAATACAAAACATTAACGGAAATGTTTAAGGTTT |
| | | GTGCTTCAAGATTTGGGAAATTATTTGCCGATTTACGAGA |
| | | CTATTTTAATGAGGCACATGAAGTTGATAAAATAAAATAT |
| | | CGTTCTTGGATTCTAGAAGATGGAAAACAAAATCGATTTG |
| | | TTTTACTTGTCGATAAAGCAAAAGACTTGGAGTTAGAAAA |
| | | TGAAGAAAATGGTGAATTGAAACTTTATGAAGTAAAAAGT |
| | | TTAACTTCCAAATCACTAATAAAATTTATTAAAAACAAAG |
| | | GAGCCTATCCTGATTTTCACAGTTTAAATAGCTTCAATTC |
| | | TGATGAAATAAAGAAAAATTGGACAAACCATAAAGCCAAT |
| | | ATAAACTTTCTAAAAAATTTGAAATCTGCATTAGAAAATT |
| | | CCCTAATGGCTATTAACCAAAATTGGAAAGAATTTAATTT |
| | | TGATTTTTCAAGGTGTGATACTTATGAACAAATTGAAAAA |
| | | GAAATTGATAGAAAAGGATATATTCTTAAACAACAAAATA |
| | | TTTCATTGAATACTATCAAAAAATCAATCAATGAAGAAAA |
| | | ATCGGAGAAAATAAACAACAGCAAAAAATTACCAAGTTTA |
| | | TTATTTCCAATTGTAAACCAAGATATAAACAGAGAAGCCA |
| | | AGCAAGAAAAAAATCAATTCACAAAAGATTGGTTTGAAAT |
| | | ATTTGCTGAAGAAAATAATTTACACAAAAAGCGTTTGCAT |
| | | CCAGAGTTTCATTTATTCTATCGCTTTCCAACAAAAAACT |
| | | ATCCAAATACAAAATTTAAAAACGGCAAAGAAAAATCAAA |
| | | ACGATATTCTCGCTTTCAAATGCTTGCACATTTTGGTTTA |
| | | GAGGTATTTCCTCAGGGTGATTATATAAGTAAAAAAGAAC |
| | | AAATCGAAATTTTTAATGATGATAAGAAGCAAAAAGAAGC |
| | | AGTTGAAAAATACAATAATAGTATTGTTTCTGAAGTTGAA |
| | | TATATCATTGGTATTGATAGAGGAATAAAGCAATTAGCCA |
| | | CACTTTGCGTATTAAATAAAAATGGGGTGATTCAAAGTGG |
| | | TTTTCAAATTTACACACCCAGTTTTAATCATGACACAAAA |
| | | CAATGGGAACATTCTTTTTTAGGAAAAAGAAATATATTAG |
| | | ATTTATCTAATTTAAGAGTGGAGACTACTATTAAAAACGA |
| | | AAAAGTTCTAGTAGATTTAGCAAGTATTCAAACAAAGAAA |
| | | GGAGAAAATCAGCAAAAAATCAAACTTAAACAACTCGCTT |
| | | ACATTCGTGAGTTGCAATATTCTATGCAAACCAGACAAGT |
| | | GGAATTGTTAGAATATGCAAAGACTTTAAATTCCGCAGAA |
| | | GATATTACCGAAGAAAAAATTAAAATCTTTATTTCACCAT |
| | | TTAAAGAGGGAAGTCATTATGAACATTTACCCAAACAAGA |
| | | AATATATAATTTATTGAATGAATGGCAAAATGCAGATGAA |
| | | ACGAGAAAACGCAAGATACAAGAACTAGACCCCACTGATA |
| | | GTTTGAAATCTGGAATTGTGGCAAATATAGTTGGAGTGAT |
| | | TGCTTTCTTTTGTGAAAAATACAACTACAAAGTTCGAATT |
| | | TCATTAGAAGATTTAACTCGTGCTTTTAGCATTCAAAAAG |
| | | ATGCTTTAACTGGTACTCCCATTCACAGAAATGATGAAGA |
| | | TTTCAAAGAACAAGAAAATCGAAGACTTGCAGGTGTAGGA |
| | | ACAATGCAGTTTTTAGAAATGCAACTCTTGAAGAAGTTGT |
| | | TTAAACTTCAATCTGAAAAAAATAAACATTTAATTCCTGC |
| | | GTTTAGAAGTGTTGCTAACTATGAAAAAATTGTTCGTAGA |
| | | GATAAAGAGAACGGTGGTGATGAATTTGTTAATTATCCTT |
| | | TTGGAATAGTAACTTTTGTTGATCCAAGAAATACTAGTCA |
| | | AAAATGTCCTTATTGCAATAATATAGCACGAAAAGAAGAT |
| | | GATGCATTTTATAGAAATGCAGGGGAAAATAAAAATTCTC |
| | | TGTTATGCAAGAAATGTGGTTTATCAACTATTAAAGGAAA |
| | | AGAAAATAAGAGCAACCAAGATGATAGTAAAAATCAGTTT |
| | | AATATTCATTTCATCACTGACGGAGACCAAAATGGAGCTT |
| | | ATCATATTGCTTTGAAGACTTTAGAGAATCTTCATCGTTT |
| | | AAATACACCTAAAGTAACAAAGCATACTAAAACAAAATGG |
| | | AAAAAATGA |
| 24 | Nucleic acid sequence | ATGGAGACTAACAAAACAACAAAAGCAATTAATGAGTACC |
| | encoding the Unk111 | AAACTCAAAAAACGATTAGATTCGGACTAACAGTTACAAA |
| | polynucleotide | TAACAATTTGTATTCCGAAAACATTGTAAAATTATTAAAA |
| | | TGCTCTGAAGAAAAAATAAAAGAGCAATTAAAGAAAACAC |
| | | AAACCGATGATTTACAAAACCAGAGGTTAAGATGTTGTTT |
| | | GATTGAAATCAAAGAATATCTAAAAACGTGGAATAATGTT |
| | | TTTTCACAAATTGATTTTTTGGCAATAACAAAAGATTATT |
| | | ACAAAGTATTATCAAGAAAAGCAAAGTTTGATTATGATAA |
| | | AGGCAATGGTTCGGAAATCAAACTTTCTTCCTTACAATCA |
| | | AAACAATCAAAGTATAATGACAAAAAACGTTATCAGTATA |
| | | TTCTGGATTTTTGGCATGAAAATTTTATTAAAGTTGAAAA |
| | | TTTGTATCGCAAATCAGACGATTTATTAAAAGTATTTGAA |
| | | GAAGCCGCAAATCAAAATCAAGATGACAAAAAACTTAATA |
| | | AAGTGGATTTGCGAAAAACTTTTTTGAGTTTATTCAATCT |
| | | CGTAAATGAAACTCTAAAACCGCTGATTGAGGGTAATTTA |
| | | TTTATTGTCAATGACGATAAAATTGACGAACATAATTCAA |
| | | AGCACAATTTTGTATCAGATTTTATTGTAAAAACAGAAGA |
| | | AAGAAAACAATTGCATGATTGTATTACTGATTTACAGGAT |
| | | TTGTTCAAGGCTAATGGCGGATATGTGCCTTTTGGCAGGG |
| | | CGACCATTAACAAATGGACTGCTCTGCAAAAATCTAATCA |
| | | TAAAGATGATGAAATTAAAAGAATTATCAGAGAGTTAAAG |
| | | ATTGAAAATATCTCAATGCAAAATATTGATTATAAATATA |
| | | AATACGATAGTTTTGCTGAAAATTTTAAGCAAATATATAA |
| | | TAAAGAAGGGGAGAAAGTTTGGGTTTTACAATTTGATGCT |
| | | AATTCTGTTATCAAAGTATGCCAATACTTCAAATATAAAA |
| | | AAGTGCCAATTAATGCTCGTCTAAACATTGCAGAAAGGCT |
| | | GATAAAAGAAAAAAGTTGGCAAAGGGAAAAAAAGAATGAT |
| | | TTTTTAAGCGAATTTGGCATTTCAAAATCACCTGCTTTAG |
| | | ACTACAAAAACGATAAAGAAAACTTCAATCTCGCAAACTA |
| | | TCCGTTAAAAGTTGCTTTTGATTATGCTTGGGAAAATTGT |
| | | GCAAAAGCAATTTACGAAACAACAACATTCCCAAAAGAAC |
| | | ATTGTGAAAAATATTTGAAAGAGGTTTTTGATTTAGATAT |
| | | AGCAAACAATGCTTGTTTTACAAAATATGCTTTATTGTTA |
| | | AGATTCAAAATCTTAATTTGCAGAATAAAATCTGAAGAAA |
| | | CTACTCAAATACAAAATATTGAGGCAGTAAGAGGTATTTT |
| | | AGACGAAATCAATAAAAATATTAGTGGTAGGCAAGATTTT |
| | | TCAAAAGCCAAAATTATTACAGAAATCAATAATTGGCTTT |
| | | CCTTTAAAGAAAAACAAACCGACAAGAAAGAAAAATACTC |
| | | TAATCAAGATAACTTTTCTTTGGCAATGCAAATTATTGGG |
| | | CAAGAACGCGGTGGTTTAAAAAGTAGAATTGAAAAATACA |
| | | AAACTTTAACGGATATGTTTAAAGTATGCGCTTCAAAATT |
| | | TGGAAAACAATTTGCCGATTTGCGCGAATATTTCCAAGAA |
| | | GCGTATGAAGTTGATAAAATCAAATATCGCGCTTGGATTA |
| | | TTGAAGACGAAAAGCAAAACCGTTTTGTTTTATTTGCGAA |
| | | CAAAGAAAGGGAAATTGATTTAACGTCTGAAGAAGGTAAT |
| | | TTGTATTTTTATGAAGTAAAAAGTTTAACTTCTAAATCTC |
| | | TCGTGAAGTTTATCAAAAACAGAGGTGCTTATGCGGATTT |
| | | TCATAAACTAAAAAATAATTTCAATTATGAAAAAATAAAG |
| | | AGAGATTGGCAATACTATAAAAATGACAAGTATTTTATCC |
| | | AAAATCTGAAAGATGCTTTACGCAACTCCAAAATGGCTAT |
| | | TGACCAAAATTGGGCAGAGTTTAAATTTGATTTCACAAAG |
| | | TTTAATACTTACGAAGATATAGAAAAAGAAATTGATAGAA |
| | | AGGGGTATAAACTTGTCTGCAAGACAGTTTCACTTAATAC |
| | | GCTTAAAGATTTTGTTGAAAACAAAGGATGTCTCTTGTTG |
| | | CCAATTATAAATCAAGATATTAATAAAGACGATAAGCAAG |
| | | CCAAAAATCAATTTACAAAAGATTGGAATAGTATTTATGA |
| | | TAATAAAAAGCGTTTGCACCCTGAATTTAACTTATTCTAT |
| | | CGCTTCCCAACCCAAGATTATCCGAATACAAAATTCAGCA |
| | | ACGGAACGGAAAAGACAAAACGATATTCGCGTTTCCAAAT |
| | | GCTTGCTCATTTTGGTTGTGAATCTGTTCCGAAAGGAGAT |
| | | TATCTAAGCAAAAAAGAGCAGATTGCCATTTTCAACGATG |
| | | ACGCGAAACAAAAAGATGCAGTTGAAAAATTCAACAATAG |
| | | CATTGCTTCAGATTTTGAGTATATTATCGGGATTGACCGA |
| | | GGCATAAAACAACTTGCAACGCTTTGTGTTTTGAATAAAA |
| | | ACGGACAGATACAAGGAGATTTTGAAATATATACCCGAAC |
| | | ATTTGAAAATAAACAGTGGAAACATACTTTATCGGAAAAG |
| | | CGTAACATTTTAGATTTATCGAATTTAAGAGTTGAAACAA |
| | | CGATTGACGGCAACAAAGTTTTAGTTGATTTGGCGAGTAT |
| | | TACGACAAAAAACGGTGAAAATCAGCAAAAAATAAAACTC |
| | | AAACAACTCGCTTATATCAGGGAATTGCAATATTCAATGC |
| | | AAACAAGGAGAGATGATTTGCTTGATTTTGCAAAAGGATT |
| | | GCAATCTGCCGATGATATTTTGAAAGATATAAGAAATTTC |
| | | ATTGTGCCATTCAAGGAAGGAGGGCAATATGCAGATTTAC |
| | | CCAACGAAAGAATCTATAATTTACTAAAAGAATGGCGAGA |
| | | TGCCGATGATGAAGCAAAACGCAAAATAGCAGAACTTGAC |
| | | CCTGCGCAAGATTTGAAATCCGGAATTGTTGCCAATATGA |
| | | TTGGCGTGGTTGCATTTCTCTGCGAAAAATATGGATATAA |
| | | AGTTCGTATTTCTTTGGAGGATTTAACGAGAGCATTTGGC |
| | | ATTCAAAAAGACGCTTTAAGCGGAATAGCAATTGCTCCAA |
| | | ATGATGAAGATTTTAAAGAACAAGAAAATCGTAGGCTTGC |
| | | CGGTGTAGGAACTTATCAGTTTTTTGAAATGCAGTTGTTG |
| | | AAAAAGTTATTCAAAACGCAGGTTGATAAAAATTTACATT |
| | | TAGTTCCTGCTTTTCGAAGTGTGGATAATTATGAGAAAAT |
| | | TGTTCGTAGAGATAAAAAGACGAATGGCGATGAATATGTA |
| | | AATTACCCTTTCGGTATTGTGCGATTTATTGACCCAAAAT |
| | | ATACTTCAAAAAGATGCCCGAAATGTGGAAAAACAGATGT |
| | | TAATCGAAATCAAAAGACCAATATTGTAAAATGCAATAAT |
| | | TGTGAGTATGAAACAAAAGCAGGAAATTCTTCCGAAGCTA |
| | | ATAACATTCATTTTATTACAGATGGCGACCAAAATGGAGC |
| | | ATATCATATTGCCCAAAAAGCATTAAAAATTCAAAAAGAA |
| | | CAATAA |
| 25 | Nucleic acid sequence | ATGAAACAGATTAAAAATCAATATCAATTATCAAAAACGT |
| | encoding the Unk112 | TGCGTTTTGGATTGACACAAAAAAACAAAACAAAAAAAGA |
| | polynucleotide | AAATTATGCTGGGGAAATCTATAAGAGCCACAGTGAATTA |
| | | TCTGATTTAGTAGAAATCTCAGAGCAAAGGATTAAAGATT |
| | | CTGTATCAACAAATAAAAACTCAGAGTCGAGTTTACCTGT |
| | | TGATGCTATTGGTAAATGTCTTAATCAAATTTCTGAGTTC |
| | | CTTAAAGGCTGGCAACAAGTATATCAGCGTACCGACCAAA |
| | | TAGCATTAGATAAGGATTATTATAAAATCCTTTGCAAAAA |
| | | AATTGGTTTTGATGGTTTTTGGTTTGATAAGAAAAACGGA |
| | | AGAAAGACAAAAAAGCCACAAGCTCGTATTATTAGCCTTT |
| | | TAGAATTGGAAAAAAAGGACGACAAAGAAACTGAACGTAA |
| | | ACAATATATTCTTGATTATTGGCAAGAAAATTTTATAAAT |
| | | GCTGTGGAGAAATACAATGTTGTCAGCGAAAAATTAAAAC |
| | | AATTTGAAGTTGCGCTAAAGATAAATAGAACGGACAATAA |
| | | ACCCAATGAAGTGGAATTTAGGAAACTATTTCTATCGTTA |
| | | GTAAATATTATTTGCGACACACTTAAACCGCTTTGTTTTC |
| | | AACAGATTTGTTTTCCAAGGTTGGAAAAAATAGACAATTC |
| | | GAAAATAGACAATAAAAATTTAATTGATTTTGCGATAGAT |
| | | TACCAGTCCAAAAACGAATTGCTTTCGCTAATATCCAAAC |
| | | TCAAGAGTTACTTTGAAGAAAACGGAGGTAATGTGCCGTA |
| | | TTGCCGAGCTACACTCAATCCCAAAACAGCAGTTAAAAAT |
| | | CCGGAATCTACAGATAATAGCATAGAATCAGAGATAAAAA |
| | | AACTTGGACTTGATAAAATTATTAAAAATAATAAAGATGC |
| | | ATTTTCCTTTTCCTATAATTTATACAACAATACTGCCGAA |
| | | GATAAAAAATCAAAATTAAAAGATGATGAAAATGGTGGAT |
| | | TGATAGAACGCAGTTTACTGTTTAAATACAAGTCGATTCC |
| | | TGCAACTGTTCGATTCGAAATAGCCAAAACATTGAGCAAA |
| | | CCAGACGGTAAAACCGAAGAAGAGATATTGGAATTTTTAC |
| | | GCGATATAGGACAACTAGAAAGTCCTGCAAAAGATTATGC |
| | | TGATTTAAAGGAAAAAGACAATTTTAACATAGAGAAGTAT |
| | | CCACTGAAAGTTGCCTTTAATTTTGCATGGGAAGGACTTG |
| | | CCCGAGCAAAATATCATCCAGAAGCTGTTTTTCCGACCGA |
| | | AATATGTAAACAATATCTCAAAAATCATTTTAAGATTACA |
| | | GAGGATAATAAAGATTTTGTGATGTATGCAAAACTGCTGG |
| | | AGTTGAATGCTGTTTTATCTACATTAGAGAAAGCAAAGCC |
| | | TACCGATGAAAAGAAATTTAGTGTTGCCGCAAAAAAATTA |
| | | TTGGAAGAAATAGAATGGGAAAAAGTTGGGAAAAATGGAT |
| | | CCAAAAATAAAGAAGCTATAACAAAATGGTTGCAGACAAA |
| | | ATCTAAGACAGACAAAAATTTTAAATCAGCCAAACAAGAA |
| | | ATCGGTTTGTTTAGAGGTAGAATAAAAAACAATATTAGAA |
| | | TAAAAAACAATATTAAGAGTGAATACTCTGAAATTACGAA |
| | | TGTATTTAAGAACATAGCAGAGGAAATGGGTAAAACATTT |
| | | GCCGAAATGCGCGATAAAATAAGCGGTGCGGCAGAATCAA |
| | | ACAAGATTTCGCATTATGCTATGATTATAGAGGATAACAA |
| | | TAAGGACAAATATGTTCTACTACAAGAATTTGTTGAAAAT |
| | | AAAAATGAACGAATATATGCAAAATCAGATAGCCAAAAGA |
| | | GTGATTTTAAGGCATATTCGGTTAATTCTATCACTTCAGG |
| | | TGCAATTGTCAAAATGCTCAAAAAAATTAGAACTGACAAA |
| | | TTGAAGGAAAGTAATAATTTTGCCAATACACAACCGGAAT |
| | | TGACTAGCAAGGAAAAAGAAAAACGCAATATTAAAGAATG |
| | | GAAAAAGTTCATTAATGAAAAAGGATGGAACTTGGAATTT |
| | | GGACTAAAATTAGAAAATAAAACTTTAGAAGAAATTAAGA |
| | | AAGAAGTTGATGCCAAATGCTATAAATTTGATATTAAGTA |
| | | TTTTGACAAAGAGACTCTTTCCGATTTAGTGAAAAACAAA |
| | | AATTGTTTATTACTACCAATTGTCAATCAAGATTTGGCGA |
| | | AAAAAGAAAAAAACGAAAGTAACCAGTTTACGAAAGATTG |
| | | GAATGCTGTTTTTCCTCAAGATACGCCTTGGCGTTTAACT |
| | | CCGGAGTTCAGAATTTCTTATCGCAAGCCCACACCTAATT |
| | | ACCCTAAATCGGATAAGGGCGATAAGCGTTATTCGCGTTT |
| | | TCAGATGATTGGACATTTCCTTTGCGATTATATTCCGAAA |
| | | ACTGATAGCTTTATTTCCAACCGCCAACAAATTGAAAATT |
| | | ATAAAGATGATGAACGGCAAGAATTAGCAGTAAAAAAATT |
| | | TAATGCAGCTTTGCGAGGGAGAACAAAAAATGAGGAATAT |
| | | AAAGAGCAATTAAATGAATTGGCGGCAAAGTATTCTAAGA |
| | | ATGGACAGCAGAAAATAAATGTAAAAACTAACGAAAAATT |
| | | TTACGTTTTCGGTATCGACCGAGGGCAAAAAGAATTGGCA |
| | | ACGCTTTGTATTATTGACCAAGACAAAAAAATTATCGGTC |
| | | CCCATAAAATTTATACCCGTTCGTTCAACTCTGAAAAAAA |
| | | ACAATGGGAACATAAATTTTTAGAAGAACGGCATATTCTT |
| | | GACTTGTCTAATTTGCGTGTTGAAACTACCGTTTTTATTG |
| | | ATGGAAAACCAGAAAAAACAAAAGTGCTGGTTGATTTGAG |
| | | CGAGGTGAAAGTGAAAGACAAAGTTACCGGAGAATATACC |
| | | AAACCCGACAAAATGCAGATTAAAATGCAGCAACTTGCCT |
| | | ACATTCGTAAACTTCAATTCAAAATGCAGAACGAACCGGA |
| | | AGCTGTATTGGCATGGTATGAAAAAAATTCTACAGAAGAT |
| | | TTGATTTTGAAAAATTTTGTAGATAACGAAGATGGTACGA |
| | | ATAACGGATTGGTTTCTTTTTATGGTGCAGCCATAGAAGA |
| | | ACTGAAAGAGACTTTGCCCATAGAGCGAATTGTTGATATG |
| | | CTTAAGGAATTCAAAACTATAAAAAAAGAAGAGGGTAAAC |
| | | TCACTAAAGAGGATGAAGAGGGAAGGGAAAAAAATAAGCG |
| | | CAAAATGGATAAATTGGTACAATTAGAGCCTGTTGATAAT |
| | | TTGAAAAACGGTGTCGTTGCGAATATGGTTGGTGTGATTG |
| | | CCTTTTTGCTTCAAAAATTTGATTATCAAGTTTATATCTC |
| | | CCTCGAAGATTTGTCAAAACCATTTAGCAGTAAAATTATC |
| | | AGTGGTATTGACGGTGTTCCAATTAGAGTTGAAAAAGAAG |
| | | AAGGACGCCGTGCTGATGTTGAAAAATATGCCGGACTCGG |
| | | ACTTTATAATTTCTTTGAGATGCAATTGCTGAAAAAACTT |
| | | TTCCGCATTCAACAGGACAGTGAGAATATTCTACATTTGG |
| | | TACCGGCTTTCAGAGCTATGAAAAATTACGACCATATAGC |
| | | CGTTGGAAAGGGTAAAGTAAAAAATCAATTTGGTATTGTG |
| | | TTTTTTGTAGATGCGGAGGCTACTTCAAAAACTTGTCCGC |
| | | GCTGCGGTTCGACTAATCAAAAACCAAACAAAAAAGATTA |
| | | TCCTAATGCTCAACAAGCAAGGTTAAGCAATGACAAAGAA |
| | | GGGTGGATTGACCGTGACAAGTCAAATGGCAACGATATTA |
| | | TTCGTTGTTTTGTATGCGGTTTCGATACAACAAAGGAATA |
| | | TACCGAAAATCCATTGAAATACATAAATAGTGGTGATGAC |
| | | AATGCGGCGTATTTGATTTCTGCCGAAGGCGTCAAAGCTT |
| | | ATGAATTGGCAACAACGTTAGCTGATAATATATAA |
| 26 | Nucleic acid sequence | ATGAAAAACATTACAAACAAGTATCAAATTACTAAGACAT |
| | encoding the Unk113 | TACGTTTTGGCCTATCACAAAAAGGGAAAACAAAAAAAGA |
| | polynucleotide | AGGATTTGATGGAGAAATTTATCAAAGCCATCAAGAATTT |
| | | AATAAATTGGTTAGCGTTTCTGAAGCAAGGATTAAAAAAA |
| | | GTGTAACGACAGAACAAAAAACAGAATTGGCTTTATCAAT |
| | | TGATAATGTTGCACGTTGTTTAAATAACATAAGTGATTTT |
| | | CTTATAAATTGGCAACGGGTGTATTACCGAACTGACCAAA |
| | | TTGCATTAGACAAAGATTATTACAAAATTATGTGTAAGAA |
| | | GATTGGATTTGAAGGATTCTGGTTTGAAACAAATAGACGC |
| | | ACTCAACAAAAAATAAAGAAGCCACAATCACGTATAATCA |
| | | GTCTTTCTGCGCTTGATAAAAAGGATGGTTTAGGCAAGGA |
| | | ACGCAAACAATACATTTTAGATTATTGGAAAGAGAATCTT |
| | | TTGTCTGCCGCTGAAAAATATGAAGTTGTTAGCGAAAAAT |
| | | TGAAGCAATTTCAAGATGCATTAAATATTAATAGAACGGA |
| | | CAATAAACCAAACGAAATTGAGCTTCGTAAACTATTCTTA |
| | | TCGCTCACCCATATTGTTTATGATATACTTCAGCCACTTT |
| | | GTTATGGTCAAATTTGTTTTCCCAAAATCGAGAAACTTGA |
| | | CAACACAAAAGAAGACAACAAAAAGTTGATTGAATTTGCT |
| | | TCCGATTATCAATCAAAAAGCGATTTACTATCCGAAATCG |
| | | CAGAATTGAAACAATATTTTGAGGAAAATGGTGGTAATGT |
| | | ACCCTTTTGCCGAGCCACATTAAATCCAAAAACACTTGTA |
| | | AAAAATCCAAAATCAACTGACAATAGTATTAATGAAGAAA |
| | | TAAAGGATTTAGGATTAAAAGAGATCTTGAAAACATACAA |
| | | AGATGTCTTAAACTACAACAACTATCTCGAAAGTCTATCC |
| | | GCAAAACAAAAGCTCCAATTGCTTAACGACAGAAACACAA |
| | | GTATAATAACACGCAGTTTGCTGTTCAAATATAAACCAAT |
| | | TTCAGCCAATGTACAATTTGATATAGCTAAAACTCTAAGC |
| | | CCGGAAGTTGGCAAGGGTGAAGAAGATTTGCGTGCTTTCT |
| | | TACGTGGAATTGGTCAACCTAAAAGCCCTGCAAAAGATTA |
| | | TGCTGATTTACAAAACAAATCCGATTTCAATATTGAAGCC |
| | | TACCCGCTTAAAGTAGCTTTCGATTTTGCATGGGAAAGTT |
| | | TAGCAAGAGCAATATATCATGCCGATTCAGACTTGCCTAT |
| | | GGATGCATGCAAAAATTTTCTTCAAGACAATTTTAAAGTA |
| | | AAAAACGATGATACAAACCTCAAATTATATGCTCAACTGC |
| | | AAGAATTGAAAGCTGTGCTATCAACATTGGAAAATGGAAA |
| | | TCCGAATAATGCGGCTGCTTTTCGACTAAAAGCTACAAAT |
| | | TTGTTGAACGAGATACCTTGGAAGACGGTTGGAAATTATG |
| | | GACAACAAAATAAAGACGAAATTTCCAAATGGTTAAATAA |
| | | TGGTAAAAACAAGGATGACTATAAAAAAGCAAAACAACAA |
| | | ATAGGATTATTCAGAGGCCGATTAAAAAACAACATTCAAG |
| | | GTTTTGATAACATCACCCAAACGAACAAAAACATTGCCAT |
| | | GAAAATGGGCAGAACCTTTGCCACAATGCGCGATAAAATA |
| | | ACCGGCGCAGCCGAACTCAACAAAGTGAGCCACTATGCTA |
| | | TGATTATTGAAGACAGAAACACCGACAGATATGTTTTATT |
| | | GCAGCCGTTTACAGAAAACGAGCAAGACAGAATCTATTCA |
| | | CAAACAGATTACAACAACGGCGATTACACCACATACGAAG |
| | | TAAATTCTATAACGTCAGGTGCAATTGCCAAAATGCTACG |
| | | TAAAGCAAGAATAGACGAGTTGAGCAAAAATGACAATAAC |
| | | AGAAACCTCACTTCGCAACCCGAACTGACCGAAGAAAAAA |
| | | AAGAAAAACGCAATATTAAAGAGTGGAAAAATTTTATTGA |
| | | AAATAAACGTTGGGATTTAGAATTTCAGTTAAAATTAAAT |
| | | GAGAAAAATTTTGAGCAAATCAAGAAAGAAGTTGATACTA |
| | | AATGTTATAATTTGAGAACTAAAAAAATTAATAAAACAAC |
| | | GCTTGAAGATTTAGTAAACAAAAGTGATTGTTTGCTGTTG |
| | | CCTATTGTAAATCAGGATTTAGCGAAAGAAGAAAAAACTA |
| | | ACGGCAATCAATTTACTAAAGATTGGAATTCAATTTTTGC |
| | | ACAAAACACTCCGTGGCGTTTGACACCGGAATTCAGGGTT |
| | | TCGTATCGTAAACCAACTCCCGATTATCCAATATCGGATA |
| | | AGGGTGACAAACGTTATTCTCGTTTCCAAATGATAGGTCA |
| | | TTTTCTTTGTGATTATATTCCGAAAAGTGATAAATACATT |
| | | TCAAATAGAGAACAAATTTTAAACTACAAAAACGACGAGT |
| | | TACAAAAGAAGGCAGTCAAAGATTTTCATGAAGATTTAAA |
| | | AGGGAAAACCGAAGAAGAAAACCAAAATGAATCGATGAAT |
| | | GCGCTAATGGCTAAATTTGGCAATGTCAATAAAAAACAGA |
| | | AAGCAACAACCGTAGAAAAGCCCAAAGAAAAATTTTATGT |
| | | ATTTGGTATTGACCGTGGACAAAAGGAATTGGCTACGCTT |
| | | TGCGTGATTGACCAAGACAAAAAGATTGTGGGCGATTTTG |
| | | ATATTTACACCCGCAGTTTTAATTCAGAACGTAAAGAATG |
| | | GGAACATACATTCTTTGAAAAACGCCATATTTTGGATTTG |
| | | TCCAATTTGCGAGTGGAAACCACTGCTTCAATTGATGGAA |
| | | AAGCGGAAAAGAAAAAAGTTTTGGTCGATTTGAGCGAAAT |
| | | TAAAGTCAAGGATAAAAACGGCAACTATTCCAAACCCGAC |
| | | AAAATGCAAATAAAAATGCAGCAATTAGCATACATTCGCA |
| | | AGTTGCAGTTTCAGATGCAGACAAACCCTGAAGGTGTTTT |
| | | AGCGTGGTTCAAAGAGAATTCAACGAAGGACTTAATTATT |
| | | AATAATCTCGTTGATAAAAAAAATGGTGAAAAAGGTTTGA |
| | | TTTCGTTCTACGGTTCGGCTATTGAAAAAATGGAAGACAC |
| | | TTTGCCCGTTGACAGAATTGAAGAAATGCTTCAAAAATTT |
| | | GCAGCTTTGAAAAAACAGGAAAAAGAAGGTGAAGATGTAA |
| | | AACTATCCATTGATCAACTTGTGCAATTAGAGCCTGTTGA |
| | | TAATTTGAAAAACGGCGTCGTAGCAAATATGGTTGGCGTA |
| | | ATCGCCTATCTGTTGCAAAAATTTAATTATCAAGTATATA |
| | | TATCATTAGAAGACTTATCGAATCCATTTGGAAGTCAAAT |
| | | AACAGGTGGAATTGCAGGCGTACCGTTGAAGCAAGGTAAA |
| | | GACGAAGGAAGACGGATGGATGTAGAAAAATATGCCGGTC |
| | | TTGGTCTGTATAATTTCTTTGAAATGCAACTACTCAAAAA |
| | | GCTATTCCGTATTCAACAAGATAGTTGTAATATTTTACAT |
| | | TTGGTTCCTGCTTTTAGAGCCCAAAAAAACTATGACCACG |
| | | TTGCCGTAGGAAAAGAAAAGGTAAAAGGGCAATTTGGAAT |
| | | CGTTTTCTTTGTTGATGCCAATGCCACTTCAAAGACTTGC |
| | | CCTGTTTGCGGAACGACAAATAATAAGCCAAACAACCAAA |
| | | AGTATCCTAATGCGAAAAAAGGACTTTCAGCAGATGGAAA |
| | | AGAAGTTTGGTTGGAACGCGACAAATCGAATGGAAATGAT |
| | | ATTATCCGTTGTTTTGTTTGTAACTTTGATACTACAAAAG |
| | | AATATACAGAAAACCCTCTTAAATACATTAAAAGCGGCGA |
| | | CGACAATGCAGCTTATCTAATTTCGGCGGCTGGAATAAAA |
| | | GCATACGAATTAGCAACAACACTCATAAACAACCAATAA |
| 27 | Nucleic acid sequence | ATGGAAACATTAAATCAATTCACGGGACTTTATTCCCTGT |
| | encoding the Unk114 | CAAAAACTATGCGGTTTGGTTTGACGCTCAAAGAAAAGAA |
| | polynucleotide | ACCCAAAAACGATTCCATAGCAGTGGAATCCCTCTATCAA |
| | | AGCCACCAAGATTTGAAAGAGTTGGTTGAGTTATCGGACA |
| | | AGAGAATTATCGAAGAAAAAAAGCCAGAACCACCTGTTGA |
| | | AAATCTTGGCAATCCGCCGATTGAGAAACTACGCGATTGT |
| | | CTGAATTCGATGCAAAAGTATCTCAATGATTGGCGAAAAG |
| | | TCTATACAAGATATGACCAACTCGCAGTCTTGAAGGATTT |
| | | TTATAGAAAATTAGAAAGAAAAGCAAGATTTGACGGATTT |
| | | TGGAAAGACAAAAAAGGACAAAATCAGCCCCAATCGCAAG |
| | | AGATAAAACTCTCCTCACTCAAGCACAAAAGCGGAGAAAA |
| | | GGAAATCAAAGATTGCATTGTCACATATTGGGGAGAAAAC |
| | | ATACGAAAGGCAAACGAGAAATGGCATCAGGTTGATTCGG |
| | | TTTTAAAGCAATTTGAAGAGGCAAAGCGCAAAAACAGAGA |
| | | TGACAAAAAACTCAATCAAGTTGAACTTCGTAAGTTGTTT |
| | | CTGTCATTGGCGAACCTTGTCAATGATACACTTGTACCGT |
| | | TATGTCAGAGATCTATCACTTTCCCAAATGCGGATAAACT |
| | | CTCCGACAATGCAAGAGACAAAAGCGTACTCGACTTTATT |
| | | GGTGACAACGAGATCAGAGAACATCTGCTTGATAAGATTA |
| | | CCAAGCTCAAAGAGTATTTTCAAGACAATGGTGGCTATGT |
| | | GCCATTTGGTCGCGTAACTCTCAACCAATATACGGCCATG |
| | | CAGAAACCAAACAAGACCGACAAAGAGATAGAGGACGCAA |
| | | TCAAAAACTTAGGACTTTCAATCATAAAATCACAAAACTT |
| | | TGATGCCTTTGAGCACATAGAAGAGGCGACAGACAAAGTG |
| | | GAAAGGCTTAATACGGTATCTTTGCCCCTTGTGGAGCGGG |
| | | CGCAATACTTCAAGGACAAAACGATTCCTGTCGGAGTTCG |
| | | TGATTCATTGGCAAAATATTTGGCGAAAGACGACACTGCT |
| | | AAAGAAAAAGAACTTATCGATTTGTTTGAAAAAATAGGCA |
| | | TGCCCAAAAGACCCGCAAAAGACTACAGTGATCCAACTCT |
| | | CAAAGAGAAGTTTGACCTGCGCAAATATCCGCTCAAGGTT |
| | | GCATTTGATTACGCTTGGGAGACAGTAGCAAGCAAAGAGT |
| | | TACACGATGATATTTTGAAAAACAAATGCAAAAAATATTT |
| | | GAAAGATATTTTTGACGTCGATACTGATAAATCCATATTT |
| | | TTCAACATCTATTCCGATCTTAATTATATGAAAATCATTT |
| | | TATCAAGAATCGAATACCCAACTCAAAATCAACTATCAAA |
| | | AGATAATTTTCTTGAATGGAATAGAAAAGTAATAACTATC |
| | | TTAGATGGCGACGACTTTAGCCACTTCAATAAAAATGCAG |
| | | ATGGCTCAACCGACAAGAAAATGAATACAGCAAAAACCTA |
| | | TGTCAAAACGTGGCTTGACAAACTTGAAGCCAACATAGAA |
| | | CAATTCGACGGACAAGACTTCAAAAAATTTTATGAGGATT |
| | | TTAAAAAGAAAAATAAGAATTCATGTAAAGATTTTGATGA |
| | | CGCAAAAAGGGATATAGGATTAAAGCGCGGCGGATTAAAA |
| | | CAAATCATTGAAGAGACAGAAACTTTTACGGACAAAAAAA |
| | | CAGGAAAACAAAAACCAAAATACAAAGACAGCAAATACAA |
| | | GGAATTAACCGAGGCATTCAAGAGTATTGCCGTGGATTTT |
| | | GGCAAACATTTTGCCACCCTTCGCGACAAATTCAATGAAG |
| | | AAAACGAAATCAACAAGATTGAATACTACGGTGTTATCGT |
| | | CGAAGATGAAAATGCCGATCGCTATCTTTTGCTTTCAAAA |
| | | CTAAGCGAAAGTCGCGAGGAGATAAAAAATATCTTTCCTG |
| | | ATAAAGCAGAGGGGTTGAAAACCTACAAGGTAAAATCTCT |
| | | AACCTCAAAGACACTAACAAAGCTCGTCAAAAACAAGGGG |
| | | GCATACAAAGATTTTCATATATCCGACATGCGCGTAGATT |
| | | TTAAAAAAATCAAAGAAGAGTGGAGTGCCTACAAAAACGA |
| | | TCAGGCTTTTTTGAAATACCTCAAAAAATGCCTCACCGAT |
| | | TCCAGCATGGCACAAGCTCAGAATTGGTCTGAATTTGGCT |
| | | TGGATTTTGACAAATGCAACACTTACGAAGAGGTAGAAAA |
| | | AGAGCTCGACGGCAAAGCATATCTGCTGCAAGAAACGCGC |
| | | CTCTCCAAAGCAACAATCACTAACTTGGTCAAAAACAAAG |
| | | GCTGCTACCTCTTGCCTATCATCAATCAAGATTTGGCGCG |
| | | AGAAGACCGCACGGCAAAAAATCAATTTACCAAAGATTGG |
| | | AAGCAGATATTTGAAAACAAAAAACATTATCGCCTGCATC |
| | | CGGAGTTCAATATGGCATACAGACAGCCGACCCCGAACTA |
| | | CCCCAACTCAGAGATCGGCGACAAAAGATATTCGCGCTTT |
| | | CAGATGATTGCAAATTTTATGTGCGAGATAGTTCCGCAAA |
| | | GCACAAGCTACGCTACGCGCAAAGAGCAAATCCAAACCTT |
| | | CAACGACAACAATAAACAACAAAAAGCCGTTAAAGACTTT |
| | | GACAGCAAATTTAAACTCTCCGACAGCTATTTTATCTTTG |
| | | GTATCGACAGAGGCATCAAACAGTTGGCTACGCTTTGCGT |
| | | ATTGGATCAAGGCGGAGTTATACGGGGTGGATTTGAAATC |
| | | TACACGCGACATTTCGACGGTAATAAAAAGCAGTGGGTCC |
| | | ATACCTCTCTGGAGAGGCGAAATATTTTGGACTTAACAAA |
| | | TCTGCGTGCGGAAACCACAATAGATGGTAAAAAAGTGCTA |
| | | GTAGATTTGAGCAAAGTAGAGATCAAAAATCAAACAGACA |
| | | ACAAGCAAAATATCAAACTCAAGCAGCTTGCTTATATCAG |
| | | AAAATTGCAATATCAAATGCAAACCAACCCCGAAAAAGTA |
| | | AAGAATATGTCTGATGAAGATATCGAAAATGACCTAAAAG |
| | | ATATTATTACTCCATATAAAGAAGGAACTCATTATGCTGA |
| | | TTTGCCGATAGAGAATATCAAAGCAATGCTGGATCGCTTC |
| | | AAAGTTCTCTACGGCAAAACCGACCAGCAGTCCAAACAAG |
| | | AACTGAAAGAGCTTTGCGAGCTGGATGCCGCAGATAATCT |
| | | CAAAGGCGGAATAGTGGCAAACATGGTGGGTGTCATTGCG |
| | | CATCTGATGGAGCAATACAACTACAGGGTCAAGATTTCAC |
| | | TCGAAAACCTAACAACATCATTTGTCAACCAATCAGATGG |
| | | GCTTAACGAGTATTTCATTTCGCGAGGTATGGATTTCAAA |
| | | GAACAAGAGAATGCGGCATTGGCAGGTTTGGGAACATACC |
| | | AATTTTTTGAGATGCAACTGCTCAAAAAAATATTCCGCAT |
| | | ACAACAAGATGATGGTAATGTTTTACATTTAGTTCCGGCA |
| | | TTTAGAAGCAAAGAGGATTATGAAAAAATCATTCGGAGAG |
| | | ACAAAAATGATGGCGATGAGTATGTAAATTATCCGTTTGG |
| | | GCTGGTAACTTTTGTTGATCCAAGATATACTAGTCGCAAA |
| | | TGCCCTATATGCGGAAAAACAGATGTAAAAAGAAATGATA |
| | | ATATAATCACTTGCAAAAAATGCGGTGCCGTATCAGGAAA |
| | | ATACTCATTCGATGATAAAAATAGGCAATTCATCACCAAC |
| | | GGCGACGAAAACGGCGCATATCATATAGCATTAAAAACAA |
| | | GAAAGGAGGTGCACAATGAAAACTAA |
| 28 | Nucleic acid sequence | ATGGATAAAGAAAATAGTTTTAAAGGGTTTACGAATTTGT |
| | encoding the Unk119 | ATGAAGTAAGGAAAACAGTGAGGTTTGGACTAACACAACC |
| | polynucleotide | AAATAAAAAATGAGAATTAAAAACTCATTTAGAATTTGAT |
| | | GATTTAATAAATAAATCTTTTGAAAACATAAAAAAAGATG |
| | | TAAAATCAAGAGATAAACCAAATTTTAAAGAAAAAGAACT |
| | | AATTGAAAAAATAAATCAGTTTATTAATTGATTAGAAAAA |
| | | CAATTATGAAATTGGAAACAAATTTATGAAAGATATGATG |
| | | TAATATCTGTAAATAAAGATTACTATAAAATACTTGCAAG |
| | | AAAAGCAAAATTTGATGCTTTTAAAAAAGATAAAAAACCA |
| | | CAAGCAAGTCAAATTAAATTATCATCATTACAGAAAGATA |
| | | ATAGAAAAGATAATATAATAAGATATTGGTGAAACATTAT |
| | | TACAAGAAGTGATTATTTAATAAATATTTTTAAACCAAAA |
| | | TTAGAACAATATTTAAATGCTGTTAATAATCCAAATAATA |
| | | GTTCTCATACTAAACCTGATTTAATAGATTTTAGAAAAGT |
| | | ATTTTTACAATTTTTAAAAGTAAATGAAGAATATTTACAA |
| | | CCTCTATTTGATAAATCTATACAATTTGAAACTTGAAAAA |
| | | AAGAAAATTCTGAAGAGATCAAAAAAATTAATACTTTTTC |
| | | TTGAGATGAAAATAATAAAGAAATTAATTATTTGATTGAT |
| | | TTATGAAAAGAAATTAGAGAATATTTTGAAGCAAATTGAA |
| | | GTCAAGTGCCTTACTGAAAAGTGAGTTTAAATTATTATAC |
| | | AGCATTACAAAAACCAAATAATTTTGGTGAAGATATTCGA |
| | | AAATGAGTTGAAAATTTATGAATAATAAAATTTTTGAATA |
| | | AAAGTGAAGAAGATATAAAAAATTATTTAAAACAAAATTC |
| | | AAAAGAAAAAATAAATTTATTAAATAATGCAAAAAATCAT |
| | | TACTTTATTGAATTAATACATCTTTTTAAGCCAAAAACAA |
| | | TTCCTTTTTCAGTAAAGTATAATTTAGCAAAATATTTAGA |
| | | AAAAAATTTTAATTTAAAATATGAAGATATTTTAAATAAA |
| | | TTTGATTTACTTTGAAAGTCAGTTGATATTTGAAAAGATT |
| | | ATCTTGAATGTAAAGAAAAAGAAAAGTTTTCACTTGAAAA |
| | | ATATCCTATTAAATCAGCTTTTGATTATTCTTGGGAAAAC |
| | | TTAGCAAGAAATTTAAAAAGAGATGTTGATTTTCCAAAAA |
| | | GTGTTTGTGAAAAGTTTTTAAAAGATAATTTTGATATAAT |
| | | TATTAATAATAGTAGCTTTAATTTATATGCAAATTTGCTT |
| | | TTTATTGCTGAAAATTTGGCAACAATAGAATATTGAAATC |
| | | CAAATAACGAAAATGAAATTATAGAGAGTATAAAAAATAC |
| | | ATTTGATGATATAAAATTTGAATCTAATAAACAAGAGTAT |
| | | GATTGATATAAAAAAGAAATTTTAAATATTCTAAATCAAG |
| | | AGAAAAGTAAAAGAAATTATAAAAATATATTAACAGCAAA |
| | | ACAAAGATTATGATTATTAAGATGACAACAAAAAAATAAA |
| | | ATTTCAAAATATTATAATTTAACACAATCTTTCAAAAAAA |
| | | TAGCAAGTTTTATTTGAAAAACTTTGGCTACAATAAGAGA |
| | | ATGATTAAAAGAAGAAAACGAATTAAATAAAATTACTGAT |
| | | TATTGAATAATAATTGAGGATAAAAATCAAGATAAATATA |
| | | TTTTAACTTTAAAACTTGATTGAAAAGATATAAGAGAAAA |
| | | AATAAAAAGTAAGTTATGGGATTGAGAATATAAAGTTTTT |
| | | GAAATTAATTCTTTTACATCAAGGGCACTCAATAAATTTA |
| | | TAAAAAATCCCTTATGAGAAGACTCAAAAAAATTTCATTG |
| | | AGATTATAAATATAAACATAAGGAAGTTTCAATTTACAAA |
| | | GATGTAAAATGGATTTGATATAAAGAAGAATTTTTAATTC |
| | | ATTTAAAAGATTCTTTAGTAAATTCTCAAATTGCAAAAGA |
| | | ACAAAATTGGAAAGCTTTTTGATGGAATTTTGATAATTTT |
| | | AATACTTATGAAAAAATTGAAAAAGAAATTGATAAAAAAT |
| | | GATATAAATTAATAAAAAACTCTATTTCAAAAGAAAACTT |
| | | AGAATACTTAATAAATGAAGAAAAATGTTTATTATTTCCA |
| | | TTAATAAATCAAGATATTTCAAGTAAAAAAGAACAAAATA |
| | | AAAATGAATTTACAAAAGATTTTAACAAAGCATTTTTATG |
| | | AATTTGATATAGAATACATCCAGAATTTAGTATTTTTTAC |
| | | AGACAACCTGATGAAGAAAACAAAAAAATAAATAAATCTT |
| | | GAATTATAAACCGTTTCTGAAGATTGCAATTACTTGCAAA |
| | | TATTTGAATTGAATATATTCCACAAAATAATGATTATAAA |
| | | ACAAGAAAAGAACAAAATAAAATTTCATTAGACCAAACAA |
| | | ATCAAAACGAATTAGTTCAAAATTTTAATAAAGAAAAAGT |
| | | AAATAAATATTTTGATAGTTTAGATGATTATTATATTTTT |
| | | TGAATTGATAGGTGAATAAAACAATTAGCTACATTATGTA |
| | | TTACAAACAAAAATTGAATTATTCAAAGTTATGAGATTTA |
| | | TACAAAATATTTTAATAATAATTCTAAAAAATGGGAATAT |
| | | AAAAAGAATAGAATTGAATGAATTTTAGATTTAACAAATT |
| | | TAAAAATTGAATCAGATAAAGATTGAAATAAATTTTTAGT |
| | | TGATTTGTCTCTATTTGAAGCAAAAGATGAAAATTGAAAT |
| | | TCAACTTGAACAAATAAACAAAATATAAAATTAAAGCAAT |
| | | TAGCTTATATAAGAAAGTTACAATATCAAATGTCTTCAAA |
| | | TGAAAAATGAGTTTTGAATTTTTTAAAAAAGTATCAAACA |
| | | AAAGAAGAAAGACAAAATAATATAAAAGAATTAATAACTC |
| | | CTTACAAAGAATGACATCATTTTGAAGATTTGCCAGTAAA |
| | | TATTTTTGAAGAAATGTTTGAAAACTATGAAAAGTTGAAA |
| | | AATGATAAAACTTTATCAGAAATAGAAAAACAAAATTTAA |
| | | TGAAACTTACAATTGAGCTTGATTCTAGTGAAGATTTAAA |
| | | AAAATGAGTTATTGCAAATATGATTTGAGTGATAGTTTAT |
| | | TTAATGAAAAAATATGATTATAAAGTAAAAATTGCAGTTG |
| | | AAAATTTAAATCAATCTTTTATGTGACAAAATGATTGATT |
| | | AAATAACAGTTATATTTCAATAAAAACAAATTTTAAAGAT |
| | | CAAGAAAATTGAGCTTTAGCTTGAATGTGAACTTATCATT |
| | | TTTTTGAAAATCAGTTATTAAGAAAGTTATATAAAGTTTC |
| | | GGTTGAAGAATGAATATTACATTTAGTTCCATTTTTTAAT |
| | | TCTTTAGATAATGTAAATAAATTAAATTTTGAAAAAGAAA |
| | | AAATTTTATGGGTTCAAACTGAAAACTATAGAAAGTTTTG |
| | | AATAGTTAGTTTTGTAAGACCACATAACACAAGTAAAAGA |
| | | TGTCCTATTTGTAAATCAATAAATGTAAAGAGAAAAGATA |
| | | ATATTACAACTTGTAGTGACTGTTGATTTATAACTTGAAA |
| | | AGATAATAATATAGTTATAAAAAAATATAAAAAAGAATGA |
| | | TTAAATTTAGATTTAATTAAAAATTGAGATGATAATTGAG |
| | | CTTATAATATATGTTGTAAAATTTGACTCTAA |
| 29 | Nucleic acid sequence | GTGAGGTTTGGGCTTACCCAACCAAATAAAAAATGAGAAT |
| | encoding the Unk 120 | TAAAAACTCATATAGAATTTAGTGATTTAGTAAATAAATC |
| | polynucleotide | TTTTGAGAATATAAAAAAAGAGGTAAACTCAAAAGATAAA |
| | | TCAAAATTTGATACTAGAAAAGAATTGATTGATAAAATAA |
| | | ATCAGTTTATTTCTTGATTAGAAAATCAGTTATGAGACTG |
| | | GAAGAATATGTATGAAAGATATGATTTAATATCTGTAAAT |
| | | AAAGATTATTATAAAATACTTGCAAGAAAAGCAAAATTTG |
| | | ATGCTTTTAAGAAAGATAAAAAATGAGTTAAACAACCACA |
| | | AGCTAATCAAATTAAGTTGTCATCATTAAGATATAATAAA |
| | | GAATTAATAATAAATTATTGGTGAAATATCATTTCAAGAA |
| | | GTGATTATTTAATAAATGTTTTTAAACCAAAATTAGAACA |
| | | ATATCTAAATGCTGTTAATAATCCAAATAATAGTTCTCAT |
| | | ACAAAACCTGATTTAATAGATTTTAGAAAAGTATTTTTAC |
| | | AACTGTTAAAAATAAGTGAAGAATATTTACAACCTTTATT |
| | | TAATAAATCTATACAATTTGAAACATGAAAAAAAGAAAAT |
| | | TCTTGAGATATTAAAAGAGTGAATGATTTTTCTTGAAATG |
| | | AAAATAATAAAGAAATTAATGATTTGCTTGATTTATGAAA |
| | | AGAAATTAGAGAATACTTTGAAGCAAATTGAAGTCAAGTT |
| | | CCTTATTGAAAAGTTAGTTTAAACTATTATACAGCAGTTC |
| | | AAAAACCAAATAATTTTGATAAAGAAATCAAAGAATGAAT |
| | | TAAAGATTTATGAATAATAGAGTTTTTAAAGAAAAGCGAA |
| | | GAGGATATAAAAAATTATTTAAAACAAGATTCAAAAGAAA |
| | | AAATATATTTATTAAATAATTCAAAAAATCCTTACTCTAT |
| | | TGAGTTAATACAACTTTTTAAACCAAAAACAATTCCTTTT |
| | | TCGGTAAAATATAATTTATCTAAATATTTAGAGAAGAATT |
| | | ATAATTTAAAATATGAAGATATTTTAAATAAATTTGATTT |
| | | ACTTTGAAAATCTGTTGATATTTGAAAAGATTATCTTGAA |
| | | TGCAAAGATAAAGAAAAATTTTCACTTGAAAAATATCCTA |
| | | TTAAATCAGCTTTTGATTATTCTTGGGAAAATTTAGCAAG |
| | | AAGTCTAAAAAGAGATGTTGATTTTCCAAAAAATGTTTGT |
| | | GAAAAATACTTAAATGATAATTTTAATATAAATGTTTGAA |
| | | ATTCAAGTTTTAATTTATATGCAAATTTACTTTTTATTGC |
| | | TGAAAATTTAGCAACAATAGAGTATTGAAAACCAAATAAT |
| | | GAAAAAGAAATTATTGATAGTATTAAAGAAACTTTCTTAG |
| | | AATTATCAGATGAAATAGAAAAAAATAATAAAAAAAATGA |
| | | AGTTGAAAATATTATAAAATACTTAAATTTAAACACCGAT |
| | | GAAAGAAAAAATATTAAAGACTTACAAAAAAAGTATTTTA |
| | | AAAATTTAGATACTAAAGAACAAAATATTCTAAATATATT |
| | | TGATAGTTTTACAAAATCAAAGCAATCTTTATGACTTCTA |
| | | AGATGACAACAAAAAAATAAAATTGATAAATATAGAAATT |
| | | TAACACAAAAGTTAGTTGATAAAAAGGATTCTCATATTTG |
| | | AATAGCAAGTTTTATTTGAAGAACTTTGGCTTCAATAAGG |
| | | GAATGATTAAAAGAAGAAAATGAACTAAATAAAATTACTG |
| | | ACTATTGAATAATAATTGAAGATAAAAATCAAGACAAATA |
| | | TATTTTAACTCTAAAACTTAACGGAAAAGATACAAGAGAA |
| | | AAAATAAAAAATAATTTATGAAATTGAGAATATAAAGTTT |
| | | TTGAAATAAATTCTTTTACATCAAAAGCACTCAATAAATT |
| | | TATAAAAAATCCTTTATGAGAAGATTCAAAGAAATTTCAT |
| | | TGATATTTTCAATATAAACATAGAGAAGTTTCAATATATG |
| | | ATGAAAATGAAAAATGGGTTTGATATAAAGAAGAGTTTTT |
| | | GAAACATTTGAAACATTCTTTAATAAATTCTCAAATTGCA |
| | | GTAGAACAAAATTGGAAAGATTTTTGATGGAATTTTGATA |
| | | ATTGTGATACTTATGAAAAGATTGAAAAAGAAGTTGATAA |
| | | AAAATGATATAAATTAATAGAAACCTCTATTTCAAAAGAA |
| | | AATTTAGAGAATTTAATACATAAAGAAGATTGTTTATTAT |
| | | TTCCATTGATAAATCAAGATATTTCTAGCAAAAAAGAGGA |
| | | AAATAAAAATGACTTTACAAAAAATTTTGAAAAAGTATTT |
| | | TTATGAGATTGATATAGAATACATCCAGAGTTTAGTATAT |
| | | TTTATAGACAACCAAATGAAGAAAATTTAAAACCAAACAA |
| | | ATCTTGAATTATAAATCGTTTTTGAAGATTACAATTACTT |
| | | GCAAATATTTGAGTTGAGTATATTCCACAAAACAATGATT |
| | | ACACAACAAGAAAAGAACAAAATAAAATTTCAATAGATCA |
| | | AACAAAACAAAATGAATCAGTTCAAAAATTTAACAAAGAA |
| | | AAAGTAAATCCATATTTTGATAGTTTAGAAGATTATTATA |
| | | TTTTTTGAATTGATAGATGAATTAAACAACTTGCAACTTT |
| | | GTGTATTACAAATAAAAAATGAGTTATTCAAAACTTTGAT |
| | | ATTTATACAAAACATTTTAATGATAATTCTAAAAATTGGG |
| | | AATATAAAAATAATAGAACAGAATGAATTTTGGATTTAAC |
| | | AAATTTAAAAGTTGAGTCAGACAAAGAATGAAATAAATAT |
| | | TTAGTTGATTTATCTTTATTTGAAGCAAAAGATGAAAATT |
| | | GAAATCTAACTTGAACGAATAAGCAAAATGTAAAATTAAA |
| | | GCAGTTAGCTTATATTAGAAAACTTCAATATCAAATGTCT |
| | | TCCAATGAAGAATGAGTTTTAAGTTTTTTAAATAAATATA |
| | | AAACAAAAGAAGAAAGACAAAATAATATAAAAGAGTTAAT |
| | | AACACCATATAAAGAGTGACATCATTTTGAAGACTTACCG |
| | | ATGAATATTTTTGAAGAAATGTTTGAAAATTATGAAAAAT |
| | | TGAAAAATAATAAAACTTTATCAGAATGAGAAAAACAAAA |
| | | TTTAATGAAACTAACAACTGAACTTGATGCAAGTGAAGAT |
| | | TTGAAAAAATGAGTTGTTGCAAATATAATTTGAGTAATAG |
| | | TTCATTTAATGAAAGAATATGATTATAAAGTAAAAATTGC |
| | | AATTGAAGATTTATCAAATGCTTGGTATTTTTCAAAAGAT |
| | | TGATTATCTTGAGATTCAATACTAAATTCCAAAATTGATG |
| | | AAGAAATGGATTTAAAAAAACAAGATAATTTGGCTTTAGC |
| | | TTGAGTTTGAACTTACCATTTTTTTGAAATGCAGTTATTT |
| | | AAAAAATTATTTAAAATTTCTGTTGAAAAATGAATTTTAC |
| | | ATTTAGTTCCAAGTTTTTGAAATGTAAGAAATTATACAGA |
| | | TTTATTGAAAGAAAAATACAAATACCAATATCAACAATTT |
| | | TGAGTTATTTATTTTATAAGCCCAAAGTTTACAAGTTCAA |
| | | AGTGTCCTATCTGCTGAAAATGATGAAAAAAACATATTAA |
| | | GAGAGAAAACAATGTAATAACTTGTAAAGAATGCTGATTT |
| | | GTTTCTTGAAAAGATAATTCAATAAATATTAAGAACAATA |
| | | AAAAAGAATGACTAAATTTAGATTTAATTAAAAATTGAGA |
| | | TGATAATGGATCTTATAATATTTGATGAAAAATTAAGTAA |
| 30 | Sulf-type Cas12a2 | W-x(3)-(Y/F/L)-x(3)-(D/G/N)-(Q/L/F/M)-(I/L/V/M |
| | Conserved motif 1 | )-x-(L/I/V)-x-K-(D/E/S)-(Y/F)-Y-(K/R/L/S)-x-( |
| | | L/I/M)-x-(K/R/S)-(K/E)-(A/I/L/V)-x-F-(D/E/N/V |
| | | )-(A/G/F/V)-(F/M/I)-W |
| | | Where x = any amino acid |
| 31 | Sulf-type Cas12a2 | F-K-(Y/V/P)-(K/I)-x-(I/V)-P-(F/A/V/I)-x-(V/A/L |
| | Conserved motif 2 | )-x(3)-(L/I/V)-(A/V) |
| | | Where x = any amino acid |
| 32 | Sulf-type Cas12a2 | F-(N/S/D)-(L/I)-x-(K/N/H/A)-Y-P-(I/L)-K-(V/S)- |
| | Conserved motif 3 | A-F-(D/N)-(Y/F)-(A/S)-W-E-x-(L/C/V)-A |
| | | Where x = any amino acid |
| 33 | Sulf-type Cas12a2 | (I/L)-(I/V)-E-D-x(3)-(N/D)-(R/K)-(H/F/Y)-(I/L |
| | Conserved motif 4 | /V)-(I/L/F)Where x = any amino acid |
| 34 | Sulf-type Cas12a2 | (Y/C/S)-x-(I/V)-x-S-(F/L/I/V)-T-S-x(2)-(L/I)- |
| | Conserved motif 5 | x-K |
| | | Where x = any amino acid |
| 35 | Sulf-type Cas12a2 | (E/A)-x-(I/L)-(E/K/I)-(K/H/R)-E-(I/V/L)-D-x- |
| | Conserved motif 6 | (K/N)-x-(Y/H)-x-(L/F) |
| | | Where x = any amino acid |
| 36 | Sulf-type Cas12a2 | (L/S/F)-L-(L/F/V)-P-(I/F/L)-(I/V)-N-(Q/K)-D |
| | Conserved motif 7 | |
| 37 | Sulf-type Cas12a2 | (L/I)-(H/T)-P-E-F-x-(I/V/L/M)-(F/S/T)-Y |
| | Conserved motif 8 | Where x = any amino acid |
| 38 | Sulf-type Cas12a2 | (N/K)-R-(Y/F)-(S/G/W)-(R/K/S)-(F/L/V)-(Q/E)- |
| | Conserved motif 9 | (M/L/F/I)-x-(A/C/G)-x-(F/L/I)-x(2)-(E/D/H)- |
| | | (F/Y/I/V)-(I/L/V/K)-(P/K) |
| | | Where x = any amino acid |
| 39 | Sulf-type Cas12a2 | G-I-D-(R/S)-(G/W)-(I/Q/L)-(K/N)-(E/Q)-L-A-(T/V)-L |
| | Conserved motif 10 | -C-(I/L/V) |
| 40 | Sulf-type Cas12a2 | (R/E)-x-I-L-D-L-(S/T)-(N/D/Y)-(L)-(R/K)-(V/I/A)-E- |
| | Conserved motif 11 | (T/S/K)-(T/D)-x-(E/D/N/K)-(G/K/N)-(K/N/E/T)-( |
| | | K/S/Q)-(V/R/F/Y)-L-V-D-(L/Q)-(S/A) |
| | | Where x = any amino acid |
| 41 | Sulf-type Cas12a2 | (L/M)-x(2)-(L/M/Y)-(A/S/P)-(Y/S)-(I/V/D)-(R/S)-x-(L |
| | Conserved motif 12 | /N/V)-(Q/T) |
| | | Where x = any amino acid |
| 42 | Sulf-type Cas12a2 | (E/Q)-L-(D/E)-x(2)-(D/E/Q)-(N/D/Y/S)-(L/F)-K-x-G-(V |
| | Conserved motif 13 | /I/A)-(V/I)-A-N-(M/I)-(I/V)-G-(V/I)-(I/V)-(A/ |
| | | V/N)-(Y/F/H) |
| | | Where x = any amino acid |
| 43 | Sulf-type Cas12a2 | Y-x-(V/A/G)-(Y/K/R/V)-(I/V)-x-(L/F/I)-E-(D/N)-(L/I) |
| | Conserved motif 14 | Where x = any amino acid |
| 44 | Sulf-type Cas12a2 | A-(G/W)-(L/V)-(G/W/E)-(T/L)-(Y/M)-x-(F/Y)-(F/L/M)- |
| | Conserved motif 15 | E-x-(Q/L)-L-(L/V)-x-K |
| | | Where x = any amino acid |
| 45 | Sulf-type Cas12a2 | F-x(2)-G-(I/V)-(I/F/V)-x-(F/Y)-(V/I/T)-x-(P/A)-x(2) |
| | Conserved motif 16 | -T-(S/T)-x(2)-C-P-x-C |
| | | Where x = any amino acid |
| 46 | Sulf-type Cas12a2 | I-x(2)-(G/W)-D-(D/Q/E)-(N/S)-(G/A)-A-(Y/F)- |
| | Conserved motif 17 | (H/L/I/N)-I |
| | | Where x = any amino acid |
| 47 | gRNA Stem loop 1 (N3) | UCUACNNNGUAGAU |
| 48 | gRNA Stem loop 2 (N4) | UCUACNNNNGUAGAU |
| 49 | gRNA Stem loop 3 (N5) | UCUACNNNNNGUAGAU |
| 50 | DNA Sequence encoding | TCTACNNNGTAGAT |
| | gRNA stem loop 1 | |
| 51 | DNA Sequence encoding | TCTACNNNNGTAGAT |
| | gRNA stem loop 2 | |
| 52 | DNA Sequence encoding | TCTACNNNNNGTAGAT |
| | gRNA stem loop 2 | |
| 53 | Nucleic acid sequence | TGGAGCAACACCTGAAGGAAGGCT |
| | encoding the spacer region of | |
| | the gRNA “CAO1-1” that | |
| | targets a CAO1 gene | |
| 54 | Nucleic acid sequence | ATGGCCCCCAAGAAGAAGCGGAAAGTGATGCTGCACGCCTTCACC |
| | encoding SuCas12a2 peptide | AACCAGTACCAGCTGAGCAAGACCCTGAGATTTGGCGCCACACTG |
| | | AAGGAGGACGAGAAGAAGTGTAAGTCTCACGAGGAGCTGAAGGGC |
| | | TTCGTGGATATCAGCTATGAGAACATGAAGAGCTCCGCCACAATC |
| | | GCCGAGTCTCTGAACGAGAATGAGCTGGTGAAGAAGTGCGAGCGG |
| | | TGTTACAGCGAGATCGTGAAGTTTCACAATGCCTGGGAGAAGATC |
| | | TACTATAGGACCGATCAGATCGCCGTGTACAAGGACTTCTATCGC |
| | | CAGCTGTCCAGGAAGGCCCGCTTTGACGCCGGCAAGCAGAACTCC |
| | | CAGCTGATCACACTGGCCTCTCTGTGCGGCATGTATCAGGGCGCC |
| | | AAGCTGTCTCGGTACATCACCAACTATTGGAAGGATAATATCACA |
| | | AGACAGAAGAGCTTCCTGAAGGACTTTTCCCAGCAGCTGCACCAG |
| | | TACACCAGAGCCCTGGAGAAGTCCGATAAGGCCCACACCAAGCCA |
| | | AACCTGATCAACTTCAACAAGACCTTCATGGTGCTGGCCAACCTG |
| | | GTGAATGAGATCGTGATCCCCCTGTCTAACGGCGCCATCAGCTTC |
| | | CCTAATATCTCCAAGCTGGAGGATGGCGAGGAGAGCCACCTGATC |
| | | GAGTTTGCCCTGAATGACTACTCCCAGCTGTCTGAGCTGATCGGC |
| | | GAGCTGAAGGATGCCATCGCCACCAACGGCGGCTATACACCATTC |
| | | GCCAAGGTGACCCTGAATCACTACACAGCCGAGCAGAAGCCCCAC |
| | | GTGTTTAAGAACGACATCGATGCCAAGATCCGGGAGCTGAAGCTG |
| | | ATCGGCCTGGTGGAGACCCTGAAGGGCAAGTCTAGCGAGCAGATC |
| | | GAGGAGTACTTTTCTAATCTGGATAAGTTCAGCACCTATAACGAC |
| | | AGGAATCAGTCCGTGATCGTGCGCACACAGTGTTTCAAGTATAAG |
| | | CCCATCCCTTTTCTGGTGAAGCACCAGCTGGCCAAGTACATCTCC |
| | | GAGCCAAACGGATGGGACGAGGATGCAGTGGCAAAGGTGCTGGAC |
| | | GCAGTGGGAGCCATCCGGTCTCCTGCCCACGATTATGCCAACAAT |
| | | CAGGAGGGCTTCGACCTGAACCACTACCCAATCAAGGTGGCCTTT |
| | | GATTATGCCTGGGAGCAGCTGGCCAATAGCCTGTACACCACAGTG |
| | | ACCTTCCCCCAGGAGATGTGCGAGAAGTACCTGAATAGCATCTAT |
| | | GGCTGTGAGGTGTCCAAGGAGCCCGTGTTCAAGTTCTACGCCGAC |
| | | CTGCTGTATATCAGAAAGAACCTGGCCGTGCTGGAGCACAAGAAC |
| | | AATCTGCCAAGCAATCAGGAGGAGTTCATCTGCAAGATCAACAAT |
| | | ACCTTTGAGAACATCGTGCTGCCCTACAAGATCTCCCAGTTCGAG |
| | | ACATATAAGAAGGACATCCTGGCCTGGATCAATGATGGCCACGAC |
| | | CACAAGAAGTACACCGATGCCAAGCAGCAGCTGGGCTTTATCAGG |
| | | GGAGGCCTGAAGGGACGCATCAAGGCAGAGGAGGTGAGCCAGAAG |
| | | GACAAGTATGGCAAGATCAAGTCCTACTATGAGAACCCCTACACC |
| | | AAGCTGACAAATGAGTTCAAGCAGATCTCCTCTACCTATGGCAAG |
| | | ACATTCGCCGAGCTGAGGGATAAGTTTAAGGAGAAGAACGAGATC |
| | | ACCAAGATCACACACTTTGGCATCATCATCGAGGATAAGAATCGG |
| | | GACAGATACCTGCTGGCCTCCGAGCTGAAGCACGAGCAGATCAAC |
| | | CACGTGTCTACCATCCTGAATAAGCTGGACAAGAGCTCCGAGTTC |
| | | ATCACATATCAGGTGAAGAGCCTGACCTCCAAGACACTGATCAAG |
| | | CTGATCAAGAACCACACCACAAAGAAGGGCGCCATCTCCCCTTAC |
| | | GCCGACTTCCACACCTCTAAGACAGGCTTTAACAAGAATGAGATC |
| | | GAGAAGAACTGGGATAATTACAAGCGCGAGCAGGTGCTGGTGGAG |
| | | TATGTGAAGGATTGCCTGACCGACTCTACAATGGCCAAGAACCAG |
| | | AATTGGGCCGAGTTCGGCTGGAACTTTGAGAAGTGTAATAGCTAT |
| | | GAGGATATCGAGCACGAGATCGACCAGAAGTCTTACCTGCTGCAG |
| | | AGCGACACCATCTCTAAGCAGAGCATCGCCTCCCTGGTGGAGGGA |
| | | GGATGCCTGCTGCTGCCAATCATCAACCAGGATATCACATCTAAG |
| | | GAGAGGAAGGATAAGAACCAGTTCAGCAAGGACTGGAATCACATC |
| | | TTTGAGGGCTCCAAGGAGTTCCGGCTGCACCCAGAGTTTGCCGTG |
| | | TCCTACAGAACCCCAATCGAGGGCTACCCCGTGCAGAAGCGGTAT |
| | | GGCAGACTGCAGTTCGTGTGCGCCTTTAACGCCCACATCGTGCCC |
| | | CAGAACGGCGAGTTCATCAATCTGAAGAAGCAGATCGAGAACTTT |
| | | AATGACGAGGATGTGCAGAAGCGCAATGTGACCGAGTTCAACAAG |
| | | AAAGTGAATCACGCCCTGAGCGATAAGGAGTATGTGGTCATCGGC |
| | | ATCGACAGGGGCCTGAAGCAGCTGGCCACACTGTGCGTGCTGGAT |
| | | AAGCGCGGCAAGATCCTGGGCGACTTCGAGATCTACAAGAAGGAG |
| | | TTTGTGAGGGCCGAGAAGCGGAGCGAGTCTCACTGGGAGCACACC |
| | | CAGGCCGAGACAAGGCACATCCTGGACCTGTCCAACCTGCGCGTG |
| | | GAGACCACAATCGAGGGCAAGAAGGTGCTGGTGGATCAGTCTCTG |
| | | ACCCTGGTGAAGAAGAACAGGGATACCCCCGACGAGGAGGCCACA |
| | | GAGGAGAATAAGCAGAAGATCAAGCTGAAGCAGCTGTCTTATATC |
| | | CGCAAGCTGCAGCACAAGATGCAGACAAACGAGCAGGATGTGCTG |
| | | GACCTGATCAACAATGAGCCCAGCGACGAGGAGTTCAAGAAGCGG |
| | | ATCGAGGGCCTGATCTCTAGCTTTGGCGAGGGCCAGAAGTACGCC |
| | | GATCTGCCTATCAATACCATGAGGGAGATGATCAGCGACCTGCAG |
| | | GGCGTGATCGCAAGGGGCAACAATCAGACAGAGAAGAACAAGATC |
| | | ATCGAGCTGGATGCCGCCGACAATCTGAAGCAGGGCATCGTGGCC |
| | | AACATGATCGGCATCGTGAATTACATCTTCGCCAAGTACAGCTAT |
| | | AAGGCCTATATCTCCCTGGAGGACCTGTCTAGGGCATACGGAGGA |
| | | GCAAAGTCCGGATACGATGGCAGATATCTGCCTAGCACCTCCCAG |
| | | GACGAGGATGTGGACTTTAAGGAGCAGCAGAACCAGATGCTGGCC |
| | | GGCCTGGGCACCTACCAGTTCTTTGAGATGCAGCTGCTGAAGAAG |
| | | CTGCAGAAGATCCAGAGCGACAATACAGTGCTGCGGTTCGTGCCT |
| | | GCCTTTAGATCCGCCGATAACTATCGGAATATCCTGAGACTGGAG |
| | | GAGACCAAGTACAAGTCCAAGCCATTCGGCGTGGTGCACTTTATC |
| | | GACCCTAAGTTCACCTCTAAGAAGTGCCCCGTGTGCAGCAAGACA |
| | | AACGTGTACAGAGACAAGGACGATATCCTGGTGTGCAAGGAGTGT |
| | | GGCTTCCGGTCTGATAGCCAGCTGAAGGAGAGAGAGAACAATATC |
| | | CACTATATCCACAACGGCGACGATAATGGCGCCTACCACATCGCC |
| | | CTGAAGAGCGTGGAGAATCTGATCCAGATGAAGCACCACCACCAC |
| | | CACCACtaa |
| 55 | Amino acid sequence of the | MNQEVQGKQYQFSKTLRFGLTSTNQNLYSEETMRLLKVSQEKIEK |
| | Unk115 peptide | QVKKENNNTDKTNQLRNCLVQIKEYLKTWDNTYPQIDFLAITKDY |
| | | YKVISRKARFDFDKGNGSEIKLSSLQSMYNNKKRYQYITDFWKEN |
| | | LHKTENLYRKSDDLLRIFEEAEKQNREDKKLNKVELRKTFLSLFN |
| | | LVNESLKPLIEGNLFIVNDEKIDEQNPKHNYVSDFILKAEARKPL |
| | | YNCIGNLQNYFKDNGGYVPFGRVTLNKWTALQKSNNRDTKINRII |
| | | KELKINSFLIKNINYKYNEFTSNFKEKKDKKGKIVKNKDGDIVWE |
| | | LEPNDKSVIELCQFFKYKKIPINACLNLAKRLIKENKLEKEKENT |
| | | FLSELGVSKSPALDYKKDQSNFSLTNYPLKVAFDYAWENCAKAKY |
| | | EDIPFPKKQCEKYLRDVFDLDIETNADFAKYALLLRFKILIGRIK |
| | | VEETTRIENIATIKEFFNDVKSNLTKEKDKTVAEINNWLTFKENQ |
| | | TDKKAKYSNQDEFSEAMKTIGEERGGLKSKISRYKALTDMFKVCS |
| | | SKFGKQFADLRDYFNEAYEVDKIKYRAWIIEDDKKNRFVLLADKG |
| | | KEVGLTSGNGDLYFYEVKSLTSKSLVKFIKNKGAYPDFHNKKSED |
| | | GFCQIYLNSENKENKDRFIDDVKIHWSTYKNDQEFLKKLKECLKN |
| | | SKMAIEQNWNEFNFDFSECDNYEKLEKEIDRKGYKFERKAISLTD |
| | | ITDLVENKECLLLPIVNHDINKEKQTENQSQFTKDWFAIFKNKKH |
| | | LHPEFNIFYRFQTKDYLKTKFKNGTEKTKRYSRFQMLAHFGCEVI |
| | | PQGDYLSKKEQIAIFNDDEKQKKEVENFKENISSDFDYVIGIDRG |
| | | IKQLATLCVLDKKGVIQGDFQIFTRKFNDITKKWEHKELEKRNIL |
| | | DLSNLRVETTIAGEKVLVDLASIKTKKGENQQKIKLKELAYIREL |
| | | QYAMQTRKDELLDFANKINSADDITEDSIKNFISPYKEGTRYADL |
| | | PKSEFFNRLTEWKNADDKGKLKVAELDSADNLKSGIVANMIGVIA |
| | | FLCEKYKYKVRISLEDLTRAYGIQKDALSGTAIYQNDEDFKEQEN |
| | | RRLAGVGTMQFFEMQLLRKLFKIQIDEKLCLIPSFRSVANYEKIV |
| | | RRDRKSSGDKFVNYPFGIVCFVDPSYTSQKCPYCDNKHKKNDKET |
| | | GKKAFYRDKGENKNSLLCKQCGVSTIKGQEKPSNKNDSKKQFNIH |
| | | FITNGDENGAYHIAKKTLNNLIPNNKNNKNQPSDFPIGTCT |
| 56 | Nucleic acid sequence | MNQEVQGKQYQFSKTLRFGLTSTNQNLYSEETMRLLKVSQEKIEK |
| | encoding the Unk115 peptide | QVKKENNNTDKTNQLRNCLVQIKEYLKTWDNTYPQIDFLAITKDY |
| | | YKVISRKARFDFDKGNGSEIKLSSLQSMYNNKKRYQYITDFWKEN |
| | | LHKTENLYRKSDDLLRIFEEAEKQNREDKKLNKVELRKTFLSLFN |
| | | LVNESLKPLIEGNLFIVNDEKIDEQNPKHNYVSDFILKAEARKPL |
| | | YNCIGNLQNYFKDNGGYVPFGRVTLNKWTALQKSNNRDTKINRII |
| | | KELKINSFLIKNINYKYNEFTSNFKEKKDKKGKIVKNKDGDIVWE |
| | | LEPNDKSVIELCQFFKYKKIPINACLNLAKRLIKENKLEKEKENT |
| | | FLSELGVSKSPALDYKKDQSNFSLTNYPLKVAFDYAWENCAKAKY |
| | | EDIPFPKKQCEKYLRDVFDLDIETNADFAKYALLLRFKILIGRIK |
| | | VEETTRIENIATIKEFFNDVKSNLTKEKDKTVAEINNWLTFKENQ |
| | | TDKKAKYSNQDEFSEAMKTIGEERGGLKSKISRYKALTDMFKVCS |
| | | SKFGKQFADLRDYFNEAYEVDKIKYRAWIIEDDKKNRFVLLADKG |
| | | KEVGLTSGNGDLYFYEVKSLTSKSLVKFIKNKGAYPDFHNKKSED |
| | | GFCQIYLNSENKENKDRFIDDVKIHWSTYKNDQEFLKKLKECLKN |
| | | SKMAIEQNWNEFNFDFSECDNYEKLEKEIDRKGYKFERKAISLTD |
| | | ITDLVENKECLLLPIVNHDINKEKQTENQSQFTKDWFAIFKNKKH |
| | | LHPEFNIFYRFQTKDYLKTKFKNGTEKTKRYSRFQMLAHFGCEVI |
| | | PQGDYLSKKEQIAIFNDDEKQKKEVENFKENISSDFDYVIGIDRG |
| | | IKQLATLCVLDKKGVIQGDFQIFTRKFNDITKKWEHKELEKRNIL |
| | | DLSNLRVETTIAGEKVLVDLASIKTKKGENQQKIKLKELAYIREL |
| | | QYAMQTRKDELLDFANKINSADDITEDSIKNFISPYKEGTRYADL |
| | | PKSEFFNRLTEWKNADDKGKLKVAELDSADNLKSGIVANMIGVIA |
| | | FLCEKYKYKVRISLEDLTRAYGIQKDALSGTAIYQNDEDFKEQEN |
| | | RRLAGVGTMQFFEMQLLRKLFKIQIDEKLCLIPSFRSVANYEKIV |
| | | RRDRKSSGDKFVNYPFGIVCFVDPSYTSQKCPYCDNKHKKNDKET |
| | | GKKAFYRDKGENKNSLLCKQCGVSTIKGQEKPSNKNDSKKQFNIH |
| | | FITNGDENGAYHIAKKTLNNLIPNNKNNKNQPSDFPIGTCT |
| 57 | Nucleic acid sequence | TGGAtCAACACCTGAAGGAAGGCT |
| | encoding the spacer region of | |
| | the gRNA “CAO1-1-1sm” | |
| | that targets a CAO1 gene | |
| | with 1 mismatch | |
| 58 | Nucleic acid sequence | TGGAtCAACAtCTGAAGGAAGGCT |
| | encoding the spacer region of | |
| | the gRNA “CAO1-1-2sm” | |
| | that targets a CAO1 gene | |
| | with 2 mismatches | |
| 59 | Nucleic acid sequence | TGGAtCAACAtCTGtAGGAAGGCT |
| | encoding the spacer region of | |
| | the gRNA “CAO1-1-3sm” | |
| | that targets a CAO1 gene | |
| | with 3 mismatches | |
| 60 | Nucleic acid sequence | TtGAtCAACAtCTGtAGGAAGGCT |
| | encoding the spacer region of | |
| | the gRNA “CAO1-1-4sm” | |
| | that targets a CAO1 gene | |
| | with 4 mismatches | |
| 61 | Nucleic acid sequence | CCTACGCCAGCAGCTCCAACTACC |
| | encoding the spacer region of | |
| | the gRNA “KRAS-1” that | |
| | targets a mutated KRAS | |
| | gene | |
| 62 | Nucleic acid sequence | CCTACGCCTGCAGCTCCAACTACC |
| | encoding the spacer region of | |
| | the gRNA “KRAS-1-1sm” | |
| | that targets a mutated KRAS | |
| | gene | |
| 63 | Nucleic acid sequence | CCTACGCGTGCAGCTCCAACTACC |
| | encoding the spacer region of | |
| | the gRNA “KRAS-1-2sm” | |
| | that targets a mutated KRAS | |
| | gene | |
| 64 | Nucleic acid sequence | GCCCGCCCAAAATCTGTGATCTTG |
| | encoding the spacer region of | |
| | the gRNA “EGFR-3” that | |
| | targets a mutated EGFR gene | |
| 65 | Nucleic acid sequence | GCGCGCCCAAAATCTGTGATCTTG |
| | encoding the spacer region of | |
| | the gRNA “EGFR-3-1sm” | |
| | that targets a mutated EGFR | |
| | gene | |
| 66 | Nucleic acid sequence | GGGCGCCCAAAATCTGTGATCTTG |
| | encoding the spacer region of | |
| | the gRNA “EGFR-3-2sm” | |
| | that targets a mutated EGFR | |
| | gene | |
| 67 | Amino acid sequence for | FSLDNYPIKVAFDYAWEMCA |
| | Unk106 and Unk107 | |
| | corresponding to amino acid | |
| | residues 370 to 389 of | |
| | SuCas 12a2 | |
| 68 | Amino acid sequence for | FSLDKYPIKVAFDYAWERCA |
| | Unk 108 corresponding to | |
| | amino acid residues 370 to | |
| | 389 of SuCas 12a2 | |
| 69 | Amino acid sequence for | FDINHYPLKVAFDFAWESLA |
| | Unk89 corresponding to | |
| | amino acid residues 370 to | |
| | 389 of SuCas 12a2 | |
| 70 | Amino acid sequence for | FNIEKYPLKVAFNFAWEGLA |
| | Unk112 corresponding to | |
| | amino acid residues 370 to | |
| | 389 of SuCas 12a2 | |
| 71 | Amino acid sequence for | FDLDAYPLKVAFDFAWENLA |
| | Unk88 corresponding to | |
| | amino acid residues 370 to | |
| | 389 of SuCas12a2 | |
| 72 | Amino acid sequence for | FNIEAYPLKVAFDFAWESLA |
| | Unk113 corresponding to | |
| | amino acid residues 370 to | |
| | 389 of SuCas12a2 | |
| 73 | Amino acid sequence for | FNLANYPLKVAFDYAWENCA |
| | Unk110 and Unk111 | |
| | corresponding to amino acid | |
| | residues 370 to 389 of | |
| | SuCas 12a2 | |
| 74 | Amino acid sequence for | FSLTNYPLKVAFDYAWENCA |
| | Unk115 corresponding to | |
| | amino acid residues 370 to | |
| | 389 of SuCas12a2 | |
| 75 | Amino acid sequence for | FSLEKYPIKSAFDYSWENLA |
| | Unk119 and Unk120 | |
| | corresponding to amino acid | |
| | residues 370 to 389 of | |
| | SuCas 12a2 | |
| 76 | Amino acid sequence of | FDLNHYPIKVAFDYAWEQLA |
| | residues 370 to 389 of | |
| | SuCas 12a2 | |
| 77 | Amino acid sequence for | FDLRKYPLKVAFDYAWETVA |
| | Unk114 corresponding to | |
| | amino acid residues 370 to | |
| | 389 of SuCas12a2 | |
| 78 | Amino acid sequence for | FDLFQYPLKPAFDYAWENVA |
| | Unk97 corresponding to | |
| | amino acid residues 370 to | |
| | 389 of SuCas 12a2 | |
| 79 | Amino acid sequence for | FNLYKYPLKVAFDYAWESLA |
| | Unk 109 corresponding to | |
| | amino acid residues 370 to | |
| | 389 of SuCas12a2 | |
| 80 | Amino acid sequence for | RDILDLSYLRVEKDENGESRLVDLS |
| | Unk106 and Unk107 | |
| | corresponding to amino acid | |
| | residues 896 to 919 of | |
| | SuCas12a2 | |
| 81 | Amino acid sequence for | RTILDLSNLRVETTIDGKQVLVDLS |
| | Unk108 corresponding to | |
| | amino acid residues 896 to | |
| | 919 of SuCas12a2 | |
| 82 | Amino acid sequence for | RHILDLSNLRVETTIVIDGKPDVRKVLVDLS |
| | Unk89 corresponding to | |
| | amino acid residues 896 to | |
| | 919 of SuCas 12a2 | |
| 83 | Amino acid sequence for | RHILDLSNLRVETTVFIDGKPEKTKVLVDLS |
| | Unk112 corresponding to | |
| | amino acid residues 896 to | |
| | 919 of SuCas 12a2 | |
| 84 | Amino acid sequence for | RVILDLSNLRVETTIVIDGKPEKKKVLVDLS |
| | Unk88 corresponding to | |
| | amino acid residues 896 to | |
| | 919 of SuCas 12a2 | |
| 85 | Amino acid sequence for | RHILDLSNLRVETTASIDGKAEKKKVLVDLS |
| | Unk113 corresponding to | |
| | amino acid residues 896 to | |
| | 919 of SuCas 12a2 | |
| 86 | Amino acid sequence for | RNILDLSNLRVETTIKNEKVLVDLA |
| | Unk110 corresponding to | |
| | amino acid residues 896 to | |
| | 919 of SuCas 12a2 | |
| 87 | Amino acid sequence for | RNILDLSNLRVETTIDGNKVLVDLA |
| | Unk111 corresponding to | |
| | amino acid residues 896 to | |
| | 919 of SuCas 12a2 | |
| 88 | Amino acid sequence for | RNILDLSNLRVETTIAGEKVLVDLA |
| | Unk115 corresponding to | |
| | amino acid residues 896 to | |
| | 919 of SuCas 12a2 | |
| 89 | Amino acid sequence for | EGILDLTNLKIESDKDGNKFLVDLS |
| | Unk119 corresponding to | |
| | amino acid residues 896 to | |
| | 919 of SuCas12a2 | |
| 90 | Amino acid sequence for | EGILDLTNLKVESDKEGNKYLVDLS |
| | Unk 120 corresponding to | |
| | amino acid residues 896 to | |
| | 919 of SuCas 12a2 | |
| 91 | Amino acid sequence of | RHILDLSNLRVETTIEGKKVLVDQS |
| | residues 896 to 919 of | |
| | SuCas12a2 | |
| 92 | amino acid sequence for | RNILDLTNLRAETTIDGKKVLVDLS |
| | Unk114 corresponding to | |
| | amino acid residues 896 to | |
| | 919 of SuCas12a2 | |
| 93 | Amino acid sequence for | RAILDLSNLRVETTVNGDKVLVDLA |
| | Unk97 corresponding to | |
| | amino acid residues 896 to | |
| | 919 of SuCas 12a2 | |
| 94 | Amino acid sequence for | RNILDLSDLRVETTVEGKKVLVDLS |
| | Unk 109 corresponding to | |
| | amino acid residues 896 to | |
| | 919 of SuCas 12a2 | |
| 95 | Amino acid sequence for | QLDASEYLKKGVVANMIGVVVY |
| | Unk106 and Unk107 | |
| | corresponding to amino acid | |
| | residues 1028 to 1049 of | |
| | SuCas12a2 | |
| 96 | Amino acid sequence for | QLDATESLKKGVVANMIGVVVY |
| | Unk108 corresponding to | |
| | amino acid residues 1028 to | |
| | 1049 of SuCas12a2 | |
| 97 | Amino acid sequence for | QLEPVDNLKAGVVANMVGVIAH |
| | Unk89 corresponding to | |
| | amino acid residues 1028 to | |
| | 1049 of SuCas12a2 | |
| 98 | Amino acid sequence for | QLEPVDNLKNGVVANMVGVIAF |
| | Unk112 corresponding to | |
| | amino acid residues 1028 to | |
| | 1049 of SuCas 12a2 | |
| 99 | Amino acid sequence for | QLEPVDNLKNGVVANMVGVIAY |
| | Unk 88 and Unk113 | |
| | corresponding to amino acid | |
| | residues 1028 to 1049 of | |
| | SuCas12a2 | |
| 100 | Amino acid sequence for | ELDPTDSLKSGIVANIVGVIAF |
| | Unk110 corresponding to | |
| | amino acid residues 1028 to | |
| | 1049 of SuCas12a2 | |
| 101 | Amino acid sequence for | ELDPAQDLKSGIVANMIGVVAF |
| | Unk111 corresponding to | |
| | amino acid residues 1028 to | |
| | 1049 of SuCas12a2 | |
| 102 | Amino acid sequence for | ELDSADNLKSGIVANMIGVIAF |
| | Unk115 corresponding to | |
| | amino acid residues 1028 to | |
| | 1049 of SuCas 12a2 | |
| 103 | Amino acid sequence for | ELDSSEDLKKGVIANMIGVIVY |
| | Unk119 corresponding to | |
| | amino acid residues 1028 to | |
| | 1049 of SuCas12a2 | |
| 104 | amino acid sequence for | ELDASEDLKKGVVANIIGVIVH |
| | Unk 120 corresponding to | |
| | amino acid residues 1028 to | |
| | 1049 of SuCas 12a2 | |
| 105 | Amino acid sequence of | ELDAADNLKQGIVANMIGIVNY |
| | residues 1028 to 1049 of | |
| | SuCas12a2 | |
| 106 | Amino acid sequence for | ELDAADNLKGGIVANMVGVIAH |
| | Unk114 corresponding to | |
| | amino acid residues 1028 to | |
| | 1049 of SuCas12a2 | |
| 107 | Amino acid sequence for | ELDSADDLKTGVVANMVGVIAF |
| | Unk97 corresponding to | |
| | amino acid residues 1028 to | |
| | 1049 of SuCas12a2 | |
| 108 | Amino acid sequence for | ELDAVDDFKTGAVANMIGVIAY |
| | Unk109 corresponding to | |
| | amino acid residues 1028 to | |
| | 1049 of SuCas 12a2 | |

1. A composition comprising a Cas12a2 polypeptide or a polynucleotide encoding the Cas12a2 polypeptide, wherein the Cas12a2 polypeptide shares at least 85% identity with a sequence selected from the group consisting of SEQ ID NOs: 3, 1, 2, 4-14, and 55, or wherein the Cas12a2 polypeptide comprises a sequence selected from the group consisting of SEQ ID NOs: 3, 1, 2, 4-14, and 55.

2. The composition of any one of claim 1, wherein said Cas12a2 polypeptide comprises one or more amino acid motifs (i) having at least 90% sequence identity with a sequence selected from the group consisting of SEQ ID NOs: 32, 40, 42, 30, 31, 33-39, 41, and 43-46, or (ii) selected from the group consisting of SEQ ID NOs: 32, 40, 42, 30, 31, 33-39, 41, and 43-46.

3. A composition comprising:
(i) the Cas12a2 polypeptide or a polynucleotide encoding the Cas12a2 polypeptide of claim 1, and
(ii) a guide polynucleotide, or a polynucleotide encoding a guide polynucleotide, wherein said guide polynucleotide is designed to bind said Cas12a2 polypeptide and hybridize with a target sequence in one or more cells of interest, wherein said target sequence is located adjacent to a protospacer adjacent motif (PAM) sequence or a protospacer flanking motif (PFM) sequence that is recognized by said Cas12a2 polypeptide.

4. The composition of claim 3, wherein said one or more cells of interest are cells of one or more pests of interest, and the target sequence is a target sequence specific to the one or more pests of interest.

5. The composition of claim 4, wherein said one or more pest of interest is a pathogenic bacterial species.

6. The composition of claim 5, wherein said pathogenic bacterial species is associated with plants, mammals, or humans.

7. The composition of claim 3, wherein said one or more cells of interest are one or more eukaryotic cells.

8. The composition of claim 7, wherein said one or more eukaryotic cells are one or more cells of at least one plant pathogen, and wherein the target sequence is a target sequence specific to said one or more plant pathogens.

9. The composition of claim 8, wherein said at least one plant pathogen is a plant parasitic nematode, an insect, a fungus, a virus, a mollusk, a spider, a scorpion, a caterpillar, an animal, a mite, a tick, or a combination thereof.

10. The composition of claim 7, wherein said one or more eukaryotic cells are one or more mammalian cells or human cells.

11. The composition of claim 10, wherein said one or more mammalian cells or human cells are one or more cancer cells, and wherein said target sequence is a cancer cell-specific target sequence.

12. The composition of claim 3, wherein said guide polynucleotide is a guide RNA.

13. The composition of claim 3, wherein said PAM sequence comprises TTNV, VTTV, or TCTV, wherein N is A, G, C, or T, and wherein V is A, G, or C.

14. The composition of claim 3, wherein the guide polynucleotide comprises a spacer comprising a nucleic acid sequence differing by no more than 4 nucleotides from a nucleic acid sequence fully complementary to the target sequence.

15. A vector comprising the polynucleotide encoding a Cas12a2 polypeptide of claim 1.

16. The vector of claim 15, wherein said vector is selected from the group consisting of phages, phagemids, and conjugative plasmids.

17. The vector of claim 16, wherein said phage or phagemid is derived from a phage selected from the group consisting of M13, lambda, p22, T7, Mu, T4 phage, PBSX, P1Puna-like, P2, 13, Bcep 1, Bcep 43, Bcep 78, T5 phage, phi, C2, L5, HK97, N15, T3 phage, P37, MS2, Q.beta., or Phi X 174, T2 phage, T12 phage, R17 phage, M13 phage, G4 phage, Enterobacteria phage P2, P4 phage, N4 phage, Pseudomonas phage .PHI.6, .PHI.29 phage and 186 phage.

18. The vector of claim 16, wherein said vector is a viral vector.

19. The vector of claim 18, wherein the viral vector is an adeno-associated virus (AAV) vector.

20. The vector of claim 16, wherein said polynucleotide encoding the Cas12a2 polypeptide and a polynucleotide encoding a guide polynucleotide are part of the same polynucleotide.

21. A method for binding, cleaving, and/or modifying a target sequence in one or more cells of interest comprising delivering to said one or more cells
(a) a composition comprising:
(i) a Cas12a2 polypeptide or a polynucleotide encoding the Cas12a2 polypeptide, wherein the Cas12a2 polypeptide shares at least 85% identity with a sequence selected from the group consisting of SEQ ID NOs: 3, 1, 2, 4-14, and 55, or wherein the Cas12a2 polypeptide comprises a sequence selected from the group consisting of SEQ ID NOs: 3, 1, 2, 4-14, and 55; and
(ii) a guide polynucleotide, or a polynucleotide encoding a guide polynucleotide, wherein said guide polynucleotide is designed to bind said Cas12a2 polypeptide and hybridize with a target sequence in one or more cells of interest, wherein said target sequence is located adjacent to a protospacer adjacent motif (PAM) sequence or a protospacer flanking motif (PFM) sequence that is recognized by said Cas12a2 polypeptide, or
(b) a vector comprising the composition, thereby binding, cleaving, and/or modifying said target sequence with the Cas12a2 polypeptide of the composition.

22. The method of claim 21, wherein the one or more cells of interest is one or more bacterial cells or eukaryotic cells.

23. The method of claim 22, wherein said one or more eukaryotic cells belongs to one or more plant pathogens.

24. The method of claim 23, wherein said one or more plant pathogens is a plant parasitic nematode, an insect, a fungus, a virus, a mollusk, a spider, a scorpion, a caterpillar, an animal, a mite, a tick, or a combination thereof.

25. The method of claim 24, wherein said one or more eukaryotic cells is one or more mammalian cells, human cells, or cancer cells.

26. The method of claim 21, wherein delivering comprises delivering said one or more cells with a phage or a phagemid engineered to comprise:
(i) a polynucleotide encoding said Cas12a2 polypeptide, and
(ii) a polynucleotide encoding a guide polynucleotide.

27. The method of claim 21, wherein delivering comprises delivering said one or more cells with a viral vector engineered to comprise:
(i) a polynucleotide encoding said Cas12a2 polypeptide, and
(ii) a polynucleotide encoding a guide polynucleotide.

28. The method of claim 27, wherein the viral vector is an adeno-associated virus (AAV) vector.

29. A modified cell produced by the method of claim 21.

30. A cell, a plant, a plant part, or a plant pathogen comprising a composition comprising a Cas12a2 polypeptide or a polynucleotide encoding the Cas12a2 polypeptide, wherein the Cas12a2 polypeptide shares at least 85% identity with a sequence selected from the group consisting of SEQ ID NOs: 3, 1, 2, 4-14, and 55, or wherein the Cas12a2 polypeptide comprises a sequence selected from the group consisting of SEQ ID NOs: 3, 1, 2, 4-14, and 55.
